SEQUENCE LISTING (151 sequences)

SEQ ID NO: 1 - protein, 5 aa, Artificial Sequence
  Synthetic
TYGMA

SEQ ID NO: 2 - protein, 17 aa, Artificial Sequence
  Synthetic
WINTLSGEPTYADDFKG

SEQ ID NO: 3 - protein, 5 aa, Artificial Sequence
  Synthetic
EPMDY

SEQ ID NO: 4 - protein, 16 aa, Artificial Sequence
  Synthetic
KSSQSLLDSDGKTYLS

SEQ ID NO: 5 - protein, 7 aa, Artificial Sequence
  Synthetic
LVSKLDS

SEQ ID NO: 6 - protein, 9 aa, Artificial Sequence
  Synthetic
WQGAHFPLT

SEQ ID NO: 7 - protein, 114 aa, Artificial Sequence
  Synthetic
QIQLVQSGPELKKPGETVKISCKTSGSTFTTYGMAWMKQAPGKGLTWMGWINTLSGEPTYADDFKGRFAFSLKTSANTAYLQINNLKNEDAATYFCAREPMDYWGQGTSVIVSS

SEQ ID NO: 8 - protein, 112 aa, Artificial Sequence
  Synthetic
DVVMTQTPLTLSITIGQPASISCKSSQSLLDSDGKTYLSWLLQRPDQSPKRLISLVSKLDSGVPDRITGSGSGTDFTLKISRVEAEDLGIYYCWQGAHFPLTFGAGTKLELK

SEQ ID NO: 9 - DNA, 342 bp, Artificial Sequence
  Synthetic
cagatccagttggtacagtctggacctgaactgaagaagcctggagagacagtcaagatctcctgcaagacttctggatctacgttcacaacctatggaatggcctggatgaagcaggctccaggaaagggtttaacgtggatgggctggataaacaccctctctggagagccaacatatgctgatgacttcaagggacggtttgccttctctttgaaaacctctgccaacactgcctatttgcagatcaacaacctcaaaaatgaggacgcggctacatatttctgtgcacgagaaccaatggactactggggtcaaggaacctcagtcatcgtctcctca

SEQ ID NO: 10 - DNA, 336 bp, Artificial Sequence
  Synthetic
gatgttgtgatgacccagactccactcactttgtcgattaccattggacaaccagcctccatctcttgcaaatcaagtcagagcctcttagatagtgatggaaagacatatttgagttggttgttacagaggccagaccagtctccaaagcgcctaatctctctggtgtccaaactggactctggagtccctgacaggatcactggcagtggatcagggacagatttcacactgaaaatcagcagagtggaggctgaagatttgggcatctattattgctggcaaggtgcacattttccgctcacgttcggtgctgggaccaagctggagctgaaa

SEQ ID NO: 11 - protein, 5 aa, Artificial Sequence
  Synthetic
DYYMN

SEQ ID NO: 12 - protein, 17 aa, Artificial Sequence
  Synthetic
DINPKDGDSGYSHKFKG

SEQ ID NO: 13 - protein, 11 aa, Artificial Sequence
  Synthetic
GFTTVVARGDY

SEQ ID NO: 14 - protein, 16 aa, Artificial Sequence
  Synthetic
KSSQSLLDSDGKTYLN

SEQ ID NO: 15 - protein, 7 aa, Artificial Sequence
  Synthetic
LVSKLDS

SEQ ID NO: 16 - protein, 9 aa, Artificial Sequence
  Synthetic
WQGTHFPYT

SEQ ID NO: 17 - protein, 120 aa, Artificial Sequence
  Synthetic
EAQLQQSGPELVKPGASVKISCKASGYSFTDYYMNWLKQSHGKSLEWIGDINPKDGDSGYSHKFKGKATLTVDKSSSTAYMELRSLTSEDSAVYYCASGFTTVVARGDYWGQGTTLTVSS

SEQ ID NO: 18 - protein, 112 aa, Artificial Sequence
  Synthetic
DVVMTQTPLTLSVTIGQPASISCKSSQSLLDSDGKTYLNWLLQRPGQSPKRLIYLVSKLDSGFPDRFTGSGSGTDFTLKISRVEAEDLGVYYCWQGTHFPYTFGGGTKLEIK

SEQ ID NO: 19 - DNA, 360 bp, Artificial Sequence
  Synthetic
gaggcccagctgcaacaatctggacctgaactggtgaagcctggggcttcagtgaagatatcctgtaaggcttctggatactcgttcactgactactacatgaactggctgaagcagagccatggaaagagccttgagtggattggagatattaatcctaaagatggtgatagtggttacagccataagttcaagggcaaggccacattgactgtagacaagtcctccagcacagcctacatggagctccgcagcctgacatctgaggactctgcagtctattactgtgcaagcggatttaccacggtagtagctaggggggactactggggccaaggcaccactctcacagtctcctca

SEQ ID NO: 20 - DNA, 336 bp, Artificial Sequence
  Synthetic
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatttggtgtctaaactggactctggattccctgacaggttcactggcagtggatcagggacagatttcacactgaaaatcagcagagtggaggctgaggatttgggagtttattattgctggcaaggtacacattttccgtacacgttcggaggggggaccaagctggaaataaaa

SEQ ID NO: 21 - protein, 5 aa, Artificial Sequence
  Synthetic
DDYMH

SEQ ID NO: 22 - protein, 17 aa, Artificial Sequence
  Synthetic
WIDPENGDTEYASKFQG

SEQ ID NO: 23 - protein, 9 aa, Artificial Sequence
  Synthetic
WATVPDFDY

SEQ ID NO: 24 - protein, 17 aa, Artificial Sequence
  Synthetic
KSSQSLLNRSNQKNYLA

SEQ ID NO: 25 - protein, 7 aa, Artificial Sequence
  Synthetic
FTSTRES

SEQ ID NO: 26 - protein, 9 aa, Artificial Sequence
  Synthetic
QQHYSTPFT

SEQ ID NO: 27 - protein, 118 aa, Artificial Sequence
  Synthetic
EVQLQQSGAELVRPGASVKLSCTASGFNIKDDYMHWVKRRPEQGLEWIGWIDPENGDTEYASKFQGKATITADTSSNTAYLQLSSLTSEDTAVYYCTTWATVPDFDYWGQGTTLTVSS

SEQ ID NO: 28 - protein, 113 aa, Artificial Sequence
  Synthetic
DIVMTQSPSSLAMSVGQKVTMSCKSSQSLLNRSNQKNYLAWYQQKPGQSPKLLVHFTSTRESGVPDRFIGSGSGTDFTLTISNLQAEDLADYFCQQHYSTPFTFGSGTKLEIK

SEQ ID NO: 29 - DNA, 354 bp, Artificial Sequence
  Synthetic
gaggtgcagctgcaacagtccggcgctgaactggtgaggcctggagcctccgtgaagctgtcctgcaccgccagcggcttcaacatcaaggacgactacatgcactgggtgaagaggaggcctgagcagggcctggagtggatcggctggatcgaccccgagaacggcgacaccgagtacgcctccaagttccagggcaaggccaccatcaccgccgacacctcctccaacaccgcctacctgcagctgagctccctgacctccgaggacaccgccgtgtactattgcaccacctgggccaccgtgcccgacttcgactactggggacagggcaccaccctgaccgtgtccagc

SEQ ID NO: 30 - DNA, 339 bp, Artificial Sequence
  Synthetic
gatatcgtgatgacccagtctccttcctctctggctatgtcagtgggacagaaagtgaccatgtcttgcaagtcctctcagtctctgctgaacaggtccaaccagaagaactacctggcttggtaccagcagaaaccaggacagtctcctaagctgctggtgcattttacctctaccagggaatccggagtgccagatagatttatcggctctggctccggcacagattttacactgaccatctccaatctgcaggcagaagatctggctgactacttttgccagcagcactactccaccccttttacctttggctccggcaccaagctggagatcaag

SEQ ID NO: 31 - protein, 5 aa, Artificial Sequence
  Synthetic
DFYMN

SEQ ID NO: 32 - protein, 17 aa, Artificial Sequence
  Synthetic
DINPNNGGTSYNQKFKG

SEQ ID NO: 33 - protein, 12 aa, Artificial Sequence
  Synthetic
DPIYYDYDEVAY

SEQ ID NO: 34 - protein, 16 aa, Artificial Sequence
  Synthetic
RSSQSLVHSNGNTYLH

SEQ ID NO: 35 - protein, 7 aa, Artificial Sequence
  Synthetic
KVSNRFS

SEQ ID NO: 36 - protein, 9 aa, Artificial Sequence
  Synthetic
SQSTHVPLT

SEQ ID NO: 37 - protein, 121 aa, Artificial Sequence
  Synthetic
EVQLQQSGPELVKPGASVKISCKASGYTFTDFYMNWVKQSHGKSLEWIGDINPNNGGTSYNQKFKGKATLTVDKSSSTAYMELRSLTSEDSAVYYCARDPIYYDYDEVAYWGQGTLVTVSA

SEQ ID NO: 38 - protein, 112 aa, Artificial Sequence
  Synthetic
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELK

SEQ ID NO: 39 - DNA, 363 bp, Artificial Sequence
  Synthetic
gaggtgcagctgcagcagtccggccctgagctggtgaagcctggagcctccgtgaagatctcctgtaaggcctccggctacaccttcaccgacttctacatgaactgggtgaagcagtcccacggcaagtccctggagtggatcggcgacatcaatcccaacaacggcggcacctcctacaaccagaagttcaagggcaaggccaccctgacagtggacaagtcctccagcaccgcctacatggagctgaggtccctgacctccgaggactccgccgtgtactactgcgccagggaccccatctactacgactacgacgaggtggcctactggggccagggaaccctggtgacagtgtccgcc

SEQ ID NO: 40 - DNA, 336 bp, Artificial Sequence
  Synthetic
gatgtggtgatgacacagacacctctgtctctgccagtgtctctcggagatcaggcttctatctcttgcagatcctctcagtctctggtgcattccaacggaaacacctacctgcattggtacctgcagaaaccaggacagtctcctaagctgctgatctacaaggtgtccaacaggttctccggagtgccagatagattttccggatctggatctggcaccgattttaccctgaagatctctagagtggaagcagaggatctgggagtgtacttttgtagccagtctacccacgtgcctctgacatttggagcaggaacaaagctggagctgaag

SEQ ID NO: 41 - protein, 114 aa, Artificial Sequence
  Synthetic
QVQLVQSGSELKKPGASVKVSCKASGYTFTTYGMAWMRQAPGQGLEWMGWINTLSGEPTYADDFKGRFVFSLDTSVSTAYLQISSLKAEDTAVYYCAREPMDYWGQGTMVTVSS

SEQ ID NO: 42 - DNA, 342 bp, Artificial Sequence
  Synthetic
caggtgcagctggtgcagtccggctccgagctgaagaagcctggcgcctccgtgaaggtgtcctgcaaggcctccggctacaccttcaccacctacggcatggcctggatgaggcaggctcctggccagggactggagtggatgggctggatcaacaccctgtccggcgaacccacctacgccgacgacttcaagggcaggttcgtgttctccctggacaccagcgtgtccaccgcctacctgcagatctcctccctgaaggccgaggacaccgccgtgtactactgcgccagggagcccatggactactggggccagggcaccatggtgaccgtgtcctcc

SEQ ID NO: 43 - protein, 114 aa, Artificial Sequence
  Synthetic
QIQLVQSGSELKKPGASVKVSCKASGYTFTTYGMAWMRQAPGQGLEWMGWINTLSGEPTYADDFKGRFAFSLDTSVSTAYLQISSLKAEDTAVYYCAREPMDYWGQGTMVTVSS

SEQ ID NO: 44 - DNA, 342 bp, Artificial Sequence
  Synthetic
cagatccagctggtgcagagcggcagcgagctgaagaagcccggcgctagcgtgaaggtgtcctgcaaggccagcggctacaccttcaccacctacggcatggcctggatgaggcaggctcctggacagggcctggagtggatgggctggatcaacaccctgtccggcgagcctacctacgccgacgacttcaagggcaggttcgccttctccctggacacctccgtgagcaccgcctacctgcagatctccagcctgaaggccgaggacaccgccgtgtactactgcgccagggagcctatggactactggggccagggcaccatggtgaccgtgtccagc

SEQ ID NO: 45 - protein, 114 aa, Artificial Sequence
  Synthetic
QIQLVQSGSELKKPGASVKVSCKASGSTFTTYGMAWMKQAPGQGLTWMGWINTLSGEPTYADDFKGRFAFSLDTSVSTAYLQISSLKAEDTAVYYCAREPMDYWGQGTMVTVSS

SEQ ID NO: 46 - DNA, 342 bp, Artificial Sequence
  Synthetic
cagatccagctggtgcagtccggcagcgagctcaagaagcccggagccagcgtgaaggtgtcctgcaaggccagcggctccaccttcaccacatacggcatggcctggatgaagcaggctcctggccagggcctgacctggatgggatggatcaacaccctgtccggcgagcctacctacgccgatgacttcaagggcaggttcgccttctccctggacacctccgtgtccaccgcttacctgcagatctcctccctgaaggccgaggacaccgccgtgtactactgcgccagggagcccatggactactggggccagggcaccatggtgaccgtgtcctcc

SEQ ID NO: 47 - protein, 112 aa, Artificial Sequence
  Synthetic
DVVMTQSPLSLPVTLGQPASISCKSSQSLLDSDGKTYLSWLQQRPGQSPRRLIYLVSKLDSGVPDRFSGSGSGTDFTLKISRVEAEDVGVYYCWQGAHFPLTFGQGTKLEIK

SEQ ID NO: 48 - DNA, 336 bp, Artificial Sequence
  Synthetic
gatgtggtgatgacacagtctcctctgtctctgccagtgacactgggacagccagcttctatctcttgcaagtcctctcagtctctgctggattccgacggaaagacctatctgtcttggctgcagcagagaccaggacagtctcctagaagactgatctacctggtgtccaagctggattctggagtgccagatagattttccggctccggctctggcacagatttcaccctgaagatctctagagtggaggcagaagacgtgggagtgtactattgttggcagggagctcacttccctctgacatttggacagggaacaaagctggagatcaag

SEQ ID NO: 49 - protein, 112 aa, Artificial Sequence
  Synthetic
DVVMTQSPLSLPVTLGQPASISCKSSQSLLDSDGKTYLSWLQQRPGQSPRRLISLVSKLDSGVPDRFSGSGSGTDFTLKISRVEAEDVGVYYCWQGAHFPLTFGQGTKLEIK

SEQ ID NO: 50 - DNA, 336 bp, Artificial Sequence
  Synthetic
gatgtggtgatgacacagtctcctctgtctctgccagtgacactgggacagccagcttctatctcttgcaagtcctctcagtctctgctggattccgacggaaagacctatctgtcttggctgcagcagagaccaggacagtctcctagaagactgatctccctggtgtctaagctggattccggagtgccagatagattttccggatctggatctggcaccgattttaccctgaagatctctagagtggaggcagaagacgtgggagtgtactattgttggcagggagctcacttccctctgacatttggacagggaacaaagctggagatcaag

SEQ ID NO: 51 - protein, 120 aa, Artificial Sequence
  Synthetic
QVQLVQSGAEVKKPGASVKVSCKASGYSFTDYYMNWVRQAPGQGLEWMGDINPKDGDSGYSHKFKGRVTMTRDTSTSTVYMELSSLRSEDTAVYYCASGFTTVVARGDYWGQGTTVTVSS

SEQ ID NO: 52 - DNA, 360 bp, Artificial Sequence
  Synthetic
caagttcagctggtgcagtccggagccgaggtgaagaagcccggcgcttccgtgaaggtgtcttgtaaggcctccggctactccttcaccgattactacatgaactgggtgaggcaagctcccggtcaaggtctggagtggatgggcgacatcaaccccaaggacggcgactccggctattcccacaagttcaagggtcgtgtgaccatgaccagggacacgtccaccagcaccgtgtacatggagctgtcctctttaaggtccgaggacaccgccgtgtactactgcgccagcggattcaccaccgtggtggctaggggcgactattggggccaaggtaccaccgtgacagtgtccagc

SEQ ID NO: 53 - protein, 120 aa, Artificial Sequence
  Synthetic
QVQLVQSGAEVKKPGASVKVSCKASGYSFTDYYMNWVRQAPGQGLEWMGDINPKDGDSGYSHKFKGRVTMTVDKSTSTVYMELSSLRSEDTAVYYCASGFTTVVARGDYWGQGTTVTVSS

SEQ ID NO: 54 - DNA, 360 bp, Artificial Sequence
  Synthetic
caagttcagctggtgcagtccggagccgaggtgaagaagcccggcgcttccgtgaaggtgtcttgtaaggcctccggctactccttcaccgattactacatgaactgggtgaggcaagctcccggtcaaggtctggagtggatgggcgacatcaaccccaaggacggcgactccggctattcccacaagttcaagggtcgtgtgaccatgaccgtggacaagtccaccagcaccgtgtacatggagctgtcctctttaaggtccgaggacaccgccgtgtactactgcgccagcggattcaccaccgtggtggctaggggcgactattggggccaaggtaccaccgtgacagtgtccagc

SEQ ID NO: 55 - protein, 120 aa, Artificial Sequence
  Synthetic
QAQLVQSGAEVKKPGASVKVSCKASGYSFTDYYMNWVRQAPGQGLEWMGDINPKDGDSGYSHKFKGRVTLTVDKSTSTVYMELRSLRSEDTAVYYCASGFTTVVARGDYWGQGTTVTVSS

SEQ ID NO: 56 - DNA, 360 bp, Artificial Sequence
  Synthetic
caagctcagctggtgcagtccggcgctgaggtgaaaaagcccggcgccagcgtgaaggtgtcttgtaaggcctccggctactccttcaccgactactacatgaactgggtgaggcaagctcccggtcaaggtctggagtggatgggcgacatcaaccccaaggacggcgacagcggctactcccacaagttcaagggtcgtgtgactttaaccgtggacaagtccacctccaccgtctacatggagctgaggtctttaaggtccgaggataccgccgtgtactactgcgctagcggcttcaccaccgtggtggctcgtggcgattactggggacaaggtaccaccgtgaccgtgtcctcc

SEQ ID NO: 57 - protein, 120 aa, Artificial Sequence
  Synthetic
QAQLVQSGAEVKKPGASVKVSCKASGYSFTDYYMNWLRQAPGQGLEWIGDINPKDGDSGYSHKFKGRATLTVDKSTSTVYMELRSLRSEDTAVYYCASGFTTVVARGDYWGQGTTVTVSS

SEQ ID NO: 58 - DNA, 360 bp, Artificial Sequence
  Synthetic
caagctcaactggtgcagtccggcgccgaggtgaaaaagcccggtgcctccgtgaaggtgagctgcaaggcctccggctactcctttaccgactactacatgaactggctgaggcaagctcccggtcaaggtctggagtggatcggcgatatcaaccccaaggacggcgactccggctacagccataagttcaagggtcgtgccactttaaccgtggacaagtccaccagcaccgtgtacatggagctgaggtctttaaggtccgaggacaccgccgtgtactactgcgcctccggcttcaccacagtggtggctcgtggcgactattggggccaaggtaccaccgtgaccgtgagctcc

SEQ ID NO: 59 - protein, 112 aa, Artificial Sequence
  Synthetic
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPYTFGGGTKVEIK

SEQ ID NO: 60 - DNA, 336 bp, Artificial Sequence
  Synthetic
gatgtggtgatgacacagtctcctctgtctctgccagtgacactgggacagccagcttctatctcttgcaagtcctctcagtctctgctggattccgacggaaagacctacctgaattggctgcagcagagaccaggacagtctcctagaagactgatctacctggtgtccaagctggattctggagtgccagatagattttccggctccggctctggcacagatttcaccctgaagatctctagagtggaggcagaagacgtgggagtgtactattgttggcagggaacccacttcccttacacatttggaggaggcacaaaggtggagatcaag

SEQ ID NO: 61 - protein, 112 aa, Artificial Sequence
  Synthetic
DVVMTQSPLSLPVTLGQPASISCKSSQSLLDSDGKTYLNWLQQRPGQSPRRLIYLVSKLDSGFPDRFSGSGSGTDFTLKISRVEAEDVGVYYCWQGTHFPYTFGGGTKVEIK

SEQ ID NO: 62 - DNA, 336 bp, Artificial Sequence
  Synthetic
gatgtggtgatgacacagtctcctctgtctctgccagtgacactgggacagccagcttctatctcttgcaagtcctctcagtctctgctggattccgacggaaagacctacctgaattggctgcagcagagaccaggacagtctcctagaagactgatctacctggtgtccaagctggattctggattcccagatagattttccggctccggctctggcacagatttcaccctgaagatctctagagtggaggcagaagacgtgggagtgtactattgttggcagggaacccacttcccttacacatttggaggaggcacaaaggtggagatcaag

SEQ ID NO: 63 - protein, 21 aa, Homo sapiens
NSFYIPRHIRKEEGSFQSCSF

SEQ ID NO: 64 - protein, 21 aa, Pan troglodytes
FSYSVPNTFPQSTESLVHCDS

SEQ ID NO: 65 - protein, 17 aa, Artificial Sequence
  Synthetic
MGWSCIILFLVATGVHS

SEQ ID NO: 66 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 67 - protein, 184 aa, Mus musculus
MNRTAYTVGALLLLLGTLLPTAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 68 - protein, 184 aa, Artificial Sequence
  Synthetic
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCFSYSVPNTFPQSTESLVHCDSCKPKKFTTMMVTLNCPELQPPTKKKRVTRVKQCRCISIDLD

SEQ ID NO: 69 - protein, 160 aa, Homo sapiens
KKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 70 - protein, 160 aa, Mus musculus
KKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 71 - protein, 24 aa, Homo sapiens
MSRTAYTVGALLLLLGTLLPAAEG

SEQ ID NO: 72 - protein, 24 aa, Mus musculus
MNRTAYTVGALLLLLGTLLPTAEG

SEQ ID NO: 73 - protein, 30 aa, Artificial Sequence
  Synthetic
QVQLVQSGAEVKKPGASVKVSCKASGYSFT

SEQ ID NO: 74 - protein, 14 aa, Artificial Sequence
  Synthetic
WVRQAPGQGLEWMG

SEQ ID NO: 75 - protein, 32 aa, Artificial Sequence
  Synthetic
RVTMTRDTSTSTVYMELSSLRSEDTAVYYCAS

SEQ ID NO: 76 - protein, 11 aa, Artificial Sequence
  Synthetic
WGQGTTVTVSS

SEQ ID NO: 77 - protein, 30 aa, Artificial Sequence
  Synthetic
QAQLVQSGAEVKKPGASVKVSCKASGYSFT

SEQ ID NO: 78 - protein, 32 aa, Artificial Sequence
  Synthetic
RVTMTVDKSTSTVYMELSSLRSEDTAVYYCAS

SEQ ID NO: 79 - protein, 32 aa, Artificial Sequence
  Synthetic
RVTLTVDKSTSTVYMELRSLRSEDTAVYYCAS

SEQ ID NO: 80 - protein, 14 aa, Artificial Sequence
  Synthetic
WLRQAPGQGLEWIG

SEQ ID NO: 81 - protein, 32 aa, Artificial Sequence
  Synthetic
RATLTVDKSTSTVYMELRSLRSEDTAVYYCAS

SEQ ID NO: 82 - protein, 30 aa, Artificial Sequence
  Synthetic
QVQLVQSGSELKKPGASVKVSCKASGYTFT

SEQ ID NO: 83 - protein, 14 aa, Artificial Sequence
  Synthetic
WMRQAPGQGLEWMG

SEQ ID NO: 84 - protein, 32 aa, Artificial Sequence
  Synthetic
RFVFSLDTSVSTAYLQISSLKAEDTAVYYCAR

SEQ ID NO: 85 - protein, 11 aa, Artificial Sequence
  Synthetic
WGQGTMVTVSS

SEQ ID NO: 86 - protein, 30 aa, Artificial Sequence
  Synthetic
QIQLVQSGSELKKPGASVKVSCKASGYTFT

SEQ ID NO: 87 - protein, 32 aa, Artificial Sequence
  Synthetic
RFAFSLDTSVSTAYLQISSLKAEDTAVYYCAR

SEQ ID NO: 88 - protein, 30 aa, Artificial Sequence
  Synthetic
QIQLVQSGSELKKPGASVKVSCKASGSTFT

SEQ ID NO: 89 - protein, 14 aa, Artificial Sequence
  Synthetic
WMKQAPGQGLTWMG

SEQ ID NO: 90 - protein, 30 aa, Artificial Sequence
  Synthetic
QVQLVQSGAEVKKPGASVKVSCKASGYTFT

SEQ ID NO: 91 - protein, 32 aa, Artificial Sequence
  Synthetic
RVTMTRDTSISTAYMELSRLRSDDTAVYYCAR

SEQ ID NO: 92 - protein, 11 aa, Artificial Sequence
  Synthetic
WGQGTLVTVSS

SEQ ID NO: 93 - protein, 32 aa, Artificial Sequence
  Synthetic
RVTMTVDKSISTAYMELSRLRSDDTAVYYCAR

SEQ ID NO: 94 - protein, 32 aa, Artificial Sequence
  Synthetic
RVTLTVDKSISTAYMELSRLRSDDTAVYYCAR

SEQ ID NO: 95 - protein, 14 aa, Artificial Sequence
  Synthetic
WVRQAPGQGLEWIG

SEQ ID NO: 96 - protein, 14 aa, Artificial Sequence
  Synthetic
WVKQAPGKGLEWIG

SEQ ID NO: 97 - protein, 30 aa, Artificial Sequence
  Synthetic
  misc_feature (2)..(2): Xaa is Val, Ile or Ala.
  misc_feature (9)..(9): Xaa is Ala or Ser.
  misc_feature (11)..(11): Xaa is Val or Leu.
  misc_feature (27)..(28): Xaa at position 27 is Tyr or Ser, and Xaa at position 28 is Thr or Ser.
QXQLVQSGXEXKKPGASVKVSCKASGXXFT

SEQ ID NO: 98 - protein, 14 aa, Artificial Sequence
  Synthetic
  misc_feature (2)..(3): Xaa at position 2 is Val, Leu or Met, and  Xaa at position 3 is Arg or Lys.
  misc_feature (8)..(8): Xaa is Gln or Lys.
  misc_feature (11)..(11): Xaa is Glu or Thr.
  misc_feature (13)..(13): Xaa is Met or Ile.
WXXQAPGXGLXWXG

SEQ ID NO: 99 - protein, 32 aa, Artificial Sequence
  Synthetic
  misc_feature (2)..(2): Xaa is Val or Ala.
  misc_feature (4)..(4): Xaa is Met or Leu.
  misc_feature (6)..(6): Xaa is  Arg or Val.
  misc_feature (8)..(8): Xaa is Thr or Lys.
  misc_feature (18)..(18): Xaa is Ser or Arg.
RXTXTXDXSTSTVYMELXSLRSEDTAVYYCAS

SEQ ID NO: 100 - protein, 11 aa, Artificial Sequence
  Synthetic
  misc_feature (6)..(6): Xaa is Thr, Met or Leu.
WGQGTXVTVSS

SEQ ID NO: 101 - protein, 23 aa, Artificial Sequence
  Synthetic
DVVMTQSPLSLPVTLGQPASISC

SEQ ID NO: 102 - protein, 15 aa, Artificial Sequence
  Synthetic
WLQQRPGQSPRRLIY

SEQ ID NO: 103 - protein, 32 aa, Artificial Sequence
  Synthetic
GVPDRFSGSGSGTDFTLKISRVEAEDVGVYYC

SEQ ID NO: 104 - protein, 10 aa, Artificial Sequence
  Synthetic
FGGGTKVEIK

SEQ ID NO: 105 - protein, 32 aa, Artificial Sequence
  Synthetic
GFPDRFSGSGSGTDFTLKISRVEAEDVGVYYC

SEQ ID NO: 106 - protein, 10 aa, Artificial Sequence
  Synthetic
FGQGTKLEIK

SEQ ID NO: 107 - protein, 15 aa, Artificial Sequence
  Synthetic
WLQQRPGQSPRRLIS

SEQ ID NO: 108 - protein, 15 aa, Artificial Sequence
  Synthetic
WFQQRPGQSPRRLIY

SEQ ID NO: 109 - protein, 15 aa, Artificial Sequence
  Synthetic
WFQQRPGQSPRLLIY

SEQ ID NO: 110 - protein, 15 aa, Artificial Sequence
  Synthetic
WYQQRPGQSPRLLIY

SEQ ID NO: 111 - protein, 15 aa, Artificial Sequence
  Synthetic
  misc_feature (2)..(2): Xaa is Leu, Phe or Tyr.
  misc_feature (12)..(12): Xaa is Arg or Leu.
  misc_feature (15)..(15): Xaa is Tyr or Ser.
WXQQRPGQSPRXLIX

SEQ ID NO: 112 - protein, 32 aa, Artificial Sequence
  Synthetic
  misc_feature (2)..(2): Xaa is Val or Phe.
GXPDRFSGSGSGTDFTLKISRVEAEDVGVYYC

SEQ ID NO: 113 - protein, 10 aa, Artificial Sequence
  Synthetic
  misc_feature (3)..(3): Xaa is Gly or Gln.
  misc_feature (7)..(7): Xaa is Val or Leu.
FGXGTKXEIK

SEQ ID NO: 114 - protein, 5 aa, Artificial Sequence
  Synthetic
SSGIG

SEQ ID NO: 115 - protein, 17 aa, Artificial Sequence
  Synthetic
EIYPRSGNTYNNEKFKG

SEQ ID NO: 116 - protein, 12 aa, Artificial Sequence
  Synthetic
EAYSHHYYAMDY

SEQ ID NO: 117 - protein, 16 aa, Artificial Sequence
  Synthetic
RSSQSLLHSNGNTYLE

SEQ ID NO: 118 - protein, 9 aa, Artificial Sequence
  Synthetic
FQGSHVPFT

SEQ ID NO: 119 - protein, 5 aa, Artificial Sequence
  Synthetic
SYGIG

SEQ ID NO: 120 - protein, 12 aa, Artificial Sequence
  Synthetic
EGYSNNYYAMDY

SEQ ID NO: 121 - protein, 16 aa, Artificial Sequence
  Synthetic
ISSQSLVHSNGNTYLE

SEQ ID NO: 122 - protein, 16 aa, Artificial Sequence
  Synthetic
RSSQSLVHSNGNTYLE

SEQ ID NO: 123 - protein, 5 aa, Artificial Sequence
  Synthetic
  misc_feature (2)..(2): Xaa is Ser or Tyr.
SXGIG

SEQ ID NO: 124 - protein, 12 aa, Artificial Sequence
  Synthetic
  misc_feature (2)..(2): Xaa is Ala or Gly.
  misc_feature (5)..(6): Xaa at position 5 is His or Asn, and Xaa at position 6 is His or Asn.
EXYSXXYYAMDY

SEQ ID NO: 125 - protein, 16 aa, Artificial Sequence
  Synthetic
  misc_feature (1)..(1): Xaa is Arg or Ile.
  misc_feature (7)..(7): Xaa is Leu or Val.
XSSQSLXHSNGNTYLE

SEQ ID NO: 126 - protein, 121 aa, Artificial Sequence
  Synthetic
QVQLQQSGAELARPGASVKLSCKASGYSFTSSGIGWVKQRSGQGLEWIGEIYPRSGNTYNNEKFKGKATLTADKSSSTVYMELRSLTSEDSAVYFCVREAYSHHYYAMDYWGQGTSVTVFS

SEQ ID NO: 127 - protein, 112 aa, Artificial Sequence
  Synthetic
DVLMTQTPLSLPVSLGGQASISCRSSQSLLHSNGNTYLEWYLQKPGQSPKLLIYKVSNRFSGVPDRLSGSGSGTDFTLKISRVEAEDLGVYYCFQGSHVPFTFGSGTKLEIN

SEQ ID NO: 128 - protein, 121 aa, Artificial Sequence
  Synthetic
QVQLQQSGAELARPGASVKLSCKASGYTFTSYGIGWVKQRTGQGLEWIGEIYPRSGNTYNNEKFKGKATLTADKSSRTVYMELRSLISEDSAVYFCAREGYSNNYYAMDYWGQGTSVTVFS

SEQ ID NO: 129 - protein, 112 aa, Artificial Sequence
  Synthetic
DVLMTQTPLSLPVSLGDQASISCISSQSLVHSNGNTYLEWYLQKPGLSPKLLIYKVSNRFSGVPDRLSGSGSGTDFTLRISRVEAEDLGVYYCFQGSHVPFTFGSGTKLEIK

SEQ ID NO: 130 - protein, 112 aa, Artificial Sequence
  Synthetic
DVLMTQTPLSLPVSLGDQASISCRSSQSLVHSNGNTYLEWYLQKPGQSPKLLIYKVSNRFSGVPDRLSGSGSGTDFTLKISRVEAEDLGVYYCFQGSHVPFTFGSGTKLEIK

SEQ ID NO: 131 - protein, 121 aa, Artificial Sequence
  Synthetic
QVQLVQSGAEVKKPGASVKVSCKASGYTFTDFYMNWVRQAPGQGLEWMGDINPNNGGTSYNQKFKGRVTMTRDTSISTAYMELSRLRSDDTAVYYCARDPIYYDYDEVAYWGQGTLVTVSS

SEQ ID NO: 132 - protein, 121 aa, Artificial Sequence
  Synthetic
QVQLVQSGAEVKKPGASVKVSCKASGYTFTDFYMNWVRQAPGQGLEWMGDINPNNGGTSYNQKFKGRVTMTVDKSISTAYMELSRLRSDDTAVYYCARDPIYYDYDEVAYWGQGTLVTVSS

SEQ ID NO: 133 - protein, 121 aa, Artificial Sequence
  Synthetic
QVQLVQSGAEVKKPGASVKVSCKASGYTFTDFYMNWVRQAPGQGLEWIGDINPNNGGTSYNQKFKGRVTLTVDKSISTAYMELSRLRSDDTAVYYCARDPIYYDYDEVAYWGQGTLVTVSS

SEQ ID NO: 134 - protein, 121 aa, Artificial Sequence
  Synthetic
QVQLVQSGAEVKKPGASVKVSCKASGYTFTDFYMNWVKQAPGKGLEWIGDINPNNGGTSYNQKFKGRVTLTVDKSISTAYMELSRLRSDDTAVYYCARDPIYYDYDEVAYWGQGTLVTVSS

SEQ ID NO: 135 - protein, 112 aa, Artificial Sequence
  Synthetic
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVPLTFGQGTKLEIK

SEQ ID NO: 136 - protein, 112 aa, Artificial Sequence
  Synthetic
DVVMTQSPLSLPVTLGQPASISCRSSQSLVHSNGNTYLHWFQQRPGQSPRLLIYKVSNRFSGVPDRFSGSGSGTDFTLKISRVEAEDVGVYYCSQSTHVPLTFGQGTKLEIK

SEQ ID NO: 137 - protein, 112 aa, Artificial Sequence
  Synthetic
DVVMTQSPLSLPVTLGQPASISCRSSQSLVHSNGNTYLHWYQQRPGQSPRLLIYKVSNRFSGVPDRFSGSGSGTDFTLKISRVEAEDVGVYYCSQSTHVPLTFGQGTKLEIK

SEQ ID NO: 138 - protein, 330 aa, Homo sapiens [sequence omitted]

SEQ ID NO: 139 - protein, 107 aa, Homo sapiens [sequence omitted]

SEQ ID NO: 140 - protein, 32 aa, Artificial Sequence
  Synthetic
  misc_feature (3)..(3): Xaa is Val or Ala.
RFXFSLDTSVSTAYLQISSLKAEDTAVYYCAR

SEQ ID NO: 141 - protein, 32 aa, Artificial Sequence
  Synthetic
  misc_feature (4)..(4): Xaa is Met or Leu.
  misc_feature (6)..(6): Xaa is Arg or Val.
  misc_feature (8)..(8): Xaa is Thr or Lys.
RVTXTXDXSISTAYMELSRLRSDDTAVYYCAR

SEQ ID NO: 142 - DNA, 363 bp, Artificial Sequence
  Synthetic
caggttcagctgcagcagtctggagctgagctggcgaggcctggggcttcagtgaagctgtcctgcaaggcttctggctactccttcacaagctctggtataggctgggtgaagcagagatctggacagggccttgagtggattggagagatttatcctagaagtggtaatacttacaacaatgagaagttcaagggcaaggccacactgactgcagacaaatcctccagcacagtgtacatggaactccgcagcctgacatctgaggactctgcggtctatttttgtgtaagagaggcctatagtcaccattactatgctatggactattggggtcaaggaacctcagtcaccgtcttctca

SEQ ID NO: 143 - DNA, 336 bp, Artificial Sequence
  Synthetic
gatgttttgatgacccaaactcctctctccctgcctgtcagtcttggaggtcaagcctccatctcttgcagatctagtcagagccttctacatagtaatggaaacacctatttagaatggtacctgcagaaaccaggccagtctccaaagctcctgatctacaaagtttccaaccgattttctggggtcccagacaggctcagtggcagtggatcagggacagattttacactcaagatcagcagagtggaggctgaggatctgggagtttattactgctttcaaggttcacatgttccattcacgttcggctcggggacaaagttggaaataaat

SEQ ID NO: 144 - DNA, 363 bp, Artificial Sequence
  Synthetic
caggttcagctgcagcagtctggagctgagctggcgaggcctggggcttcagtgaagctgtcctgcaaggcttctggctacaccttcacaagctatggtataggctgggtgaagcagagaactggacagggccttgagtggattggagagatttatcctagaagtggtaatacttacaacaatgagaagttcaagggcaaggccacactgactgcagacaaatcctccagaacagtgtacatggagctccgcagtctgatatctgaggactctgcggtctacttttgtgcaagagagggctatagtaacaattactatgctatggactactggggtcaaggaacctcagtcaccgtcttctca

SEQ ID NO: 145 - DNA, 336 bp, Artificial Sequence
  Synthetic
gatgttttgatgacccaaactccactctccctgcctgtcagtcttggagatcaagcctccatctcttgcatatctagtcagagccttgtacatagtaatggaaacacctatttagaatggtacctgcagaaaccaggcctgtctccaaaactcctgatctacaaagtttccaaccgattttctggggtcccagacaggctcagtggcagtggatcagggacagatttcacactcaggatcagcagagtggaggctgaggatctgggagtttattactgctttcaaggttcacatgttccattcacgttcggctcggggacaaagttggaaataaaa

SEQ ID NO: 146 - DNA, 363 bp, Artificial Sequence
  Synthetic
caggttcagctgcagcagtctggagctgagctggcgaggcctggggcttcagtgaagctgtcctgcaaggcttctggctacaccttcacaagttatggtataggctgggtgaagcagagaactggacagggccttgagtggattggagagatttatcctagaagtggtaatacttacaacaatgagaagttcaagggcaaggccacactgactgcggacaaatcctccagaacagtgtacatggagctccgcagtctgatatctgaggactctgcggtctacttttgtgcaagagagggctatagtaacaattactatgctatggactactggggtcaaggaacctcagtcaccgtcttctca

SEQ ID NO: 147 - DNA, 336 bp, Artificial Sequence
  Synthetic
gatgttttgatgacccaaactccactctccctgcctgtcagtcttggagatcaagcctccatctcttgcagatctagtcagagccttgtacatagtaatggaaacacctatttagaatggtacctgcagaaaccaggccagtctccaaagctcctgatctacaaagtttccaaccgattttctggggtcccagacaggctcagtggcagtggatcagggacagatttcacactcaagatcagcagagtggaggctgaggatctgggagtttattactgctttcaaggttcacatgttccattcacgttcggctcggggacaaagttggaaataaaa

SEQ ID NO: 148 - protein, 117 aa, Artificial Sequence
  Synthetic
QVQLVQSGAEVKKPGASVKVSCKASGYVFTDYYMNWVRQAPGQSLEWMGDINPNNAETLYNHKFKGRVTVTVDKSISTAYMELSRLRSDDTAVYYCVKWGDGPFAYWGQGTLVTVSS

SEQ ID NO: 149 - protein, 106 aa, Artificial Sequence
  Synthetic
DIQMTQSPSSLSASVGDRVTITCKASQNVGAAVAWYQQKPGKAPKLLIYSVSDRYTGVPSRFSGSGSGTDFTLTISSLQPEDIATYYCQQYSNYPTFGQGTKLEIK

SEQ ID NO: 150 - protein, 449 aa, Artificial Sequence
  Synthetic
QVQLVQSGAEVKKPGASVKLSCKASGYIFTTYWMHWVKQRPGQGLEWIGMIQPNSGGTKYNEKFKKKATLTVDKSISTAYMELSRLTSDDTAVYYCARGAGTVDYFDYWGQGSTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 218 aa, Artificial Sequence
  Synthetic
DIVLTQSPASLAVSVGQRATITCRASESVDIYGNSFMHWYQQKPGQPPKLLIYRASNLESGIPARFSGSGSRTDFTLTINPVEAQDTATYYCQQSTEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0294622 A1
(43) Pub. Date: Sep. 5, 2024

(54) NOVEL ANTI-GREMLIN1 ANTIBODIES

(71) Applicant: SUZHOU TRANSCENTA THERAPEUTICS CO., LTD., Suzhou, Jiangsu (CN)

(72) Inventors: Xueming QIAN, Suzhou, Jiangsu (CN); Suli CUI, Suzhou, Jiangsu (CN); Hongjun LI, Suzhou, Jiangsu (CN); Di SUN, Suzhou, Jiangsu (CN); Huanhuan GUO, Suzhou, Jiangsu (CN)

(21) Appl. No.: 18/261,834

(22) PCT Filed: Jan. 17, 2022
(86) PCT No.: PCT/CN2022/072297

§ 371 Date: Jul. 17, 2023

(30) Foreign Application Priority Data
Jan. 18, 2021 (WO) PCT/CN2021/072397
Dec. 28, 2021 (WO) PCT/CN2021/142043

(51) Int. Cl. C07K 16/22; A61K 39/00; A61K 45/06; A61P 35/00

(52) U.S. Cl. CPC C07K 16/22; A61K 45/06; A61P 35/00; A61K 2039/505; C07K 2317/24; C07K 2317/31; C07K 2317/52; C07K 2317/565

(57) ABSTRACT

The present disclosure provides herein anti-gremlin1 antibodies or antigen-binding fragments thereof, isolated polynucleotides encoding the same, pharmaceutical compositions comprising the same, and the uses thereof.

### FIELD OF THE INVENTION

The present disclosure generally relates to novel anti-gremlin1 (GREM1) antibodies that specifically bind to human gremlin1.

### BACKGROUND

Gremlin1 (GREM1) is a highly conserved secreted protein with cysteine-rich regions and cysteine knots (Wordinger et al., Exp Eye Res. 2008 August; 87(2): 78-79.). It is a member of the differential screening-selected gene aberrative in neuroblastoma (DAN) family, which serves as an antagonist of the bone morphogenetic protein (BMP) (Wordinger et al., Exp Eye Res. 2008 August; 87(2): 78-79.). GREM1 can physically bind to BMP-2, BMP-4 or BMP-7 to form heterodimers and prevent the BMP ligands from interacting with their corresponding BMP receptors, and then subsequently inhibit the activation of BMP signaling pathway.

GREM1 is closely related to fibrotic lesions of kidney, lung, liver and retina as well as several tumor types including pancreatic, colon, lung, glioma, gastric and prostate cancers (Sneddon et al., PNAS 2006 October; 103(40): 14842-14847). For example, aberrant gremlin1 upregulation endows colon cells outside of stem cell niche with tumorigenicity. It was also found that tumor stem cells highly express and secret gremlin1 to maintain their stemness in glioma (Yan, K, et al., Genes Dev 28, 1085-1100 (2014)). Accordingly, gremlin1 has been used as a therapeutic target in treating gremlin-related diseases.

However, since GREM1 is also expressed in other normal tissues, currently there is no effective treatment for GREM1-related diseases due to side effects including toxicity to normal tissues. Therefore, there is a need for novel anti-gremlin1 antibodies with reduced side effects.

### BRIEF SUMMARY OF THE INVENTION

Throughout the present disclosure, the articles “a,” “an,” and “the” are used herein to refer to one or to more than one (i.e., to at least one) of the grammatical object of the article. By way of example, “an antibody” means one antibody or more than one antibody.

The present disclosure provides, among others, novel monoclonal anti-gremlin1 (GREM1) antibodies, nucleotide sequences encoding such, and the uses thereof. The anti-GREM1 antibodies provided herein bind to a different region of the GREM1 and have differential effects on modulating the activity of GREM1 on bone morphogenetic protein (BMP) binding, when compared to existing anti-GREM1 antibodies. In particular, the anti-GREM1 antibodies provided herein are capable of reducing GREM1-mediated inhibition on BMP signaling selectively in a cancer cell over a non-cancer cell. This is unexpected and solves the long-lasting problem of side effects caused by anti-GREM1 antibodies due to universal expression of gremlin in cancer cells as well as non-cancer cells.

In one aspect, the present disclosure provides an isolated antibody against human gremlin1 (hGREM1) or an antigen-binding fragment thereof, having at least one of the following characteristics:
a) capable of reducing hGREM1-mediated inhibition on BMP signaling selectively in a cancer cell over a non-cancer cell;
b) exhibiting no more than 50% reduction of hGREM1-mediated inhibition on BMP signaling in a non-cancer cell;
c) capable of binding to a chimeric hGREM1 comprising an amino acid sequence of SEQ ID NO: 68;
d) capable of binding to hGREM1 but not specifically binding to mouse gremlin1;
e) binding to hGREM1 at an epitope comprising residue Gln27 and/or residue Asn33, wherein residue numbering is according to SEQ ID NO: 69, or binds to a hGREM1 fragment comprising residue Gln27 and/or residue Asn33, optionally the hGREM1 fragment has a length of at least 3 (e.g. 4, 5, 6, 7, 8, 9, or 10) amino acid residues;
f) capable of binding to hGREM1 at a K_{D} of no more than 1 nM as measured by Fortebio;
h) capable of blocking the binding of hGREM1 to BMP7 at a maximal blocking percentage of more than 50% as measured by ELISA;
i) capable of blocking interaction of GREM1 to FGFR; and/or
j) capable of binding to both hGREM1 and DAN.

In certain embodiments, the epitope is a linear or conformational epitope.

In another aspect, the present disclosure provides an isolated antibody against human gremlin1 (hGREM1) or an antigen-binding fragment thereof, comprising a heavy chain variable (VH) region and/or a light chain variable (VL) region, wherein the heavy chain variable region comprises:
a) a HCDR1 comprises a sequence selected from the group consisting of SEQ ID NOs: 1, 11, 21, 31, 114, 119 and 123,
b) a HCDR2 comprises a sequence selected from the group consisting of SEQ ID NOs: 2, 12, 22, 32, and 115, and
c) a HCDR3 comprises a sequence selected from the group consisting of SEQ ID NOs: 3, 13, 23, 33, 116, 120 and 124 and/or wherein the light chain variable region comprises:
d) a LCDR1 comprises a sequence selected from the group consisting of SEQ ID NOs: 4, 14, 24, 34, 117, 121, 122 and 125,
e) a LCDR2 comprises a sequence selected from the group consisting of SEQ ID NOs: 5, 15, 25 and 35, and
f) a LCDR3 comprises a sequence selected from the group consisting of SEQ ID NOs: 6, 16, 26, 36 and 118.

In certain embodiments, the heavy chain variable region of the antibody against hGREM1 or an antigen-binding fragment thereof provided herein is selected from the group consisting of:
a) a heavy chain variable region comprising a HCDR1 comprising the sequence of SEQ ID NO: 1, a HCDR2 comprising the sequence of SEQ ID NO: 2, and a HCDR3 comprising the sequence of SEQ ID NO: 3;
b) a heavy chain variable region comprising a HCDR1 comprising the sequence of SEQ ID NO: 11, a HCDR2 comprising the sequence of SEQ ID NO: 12, and a HCDR3 comprising the sequence of SEQ ID NO: 13;
c) a heavy chain variable region comprising a HCDR1 comprising the sequence of SEQ ID NO: 21, a HCDR2 comprising the sequence of SEQ ID NO: 22, and a HCDR3 comprising the sequence of SEQ ID NO: 23;
d) a heavy chain variable region comprising a HCDR1 comprising the sequence of SEQ ID NO: 31, a HCDR2 comprising the sequence of SEQ ID NO: 32, and a HCDR3 comprising the sequence of SEQ ID NO: 33;
e) a heavy chain variable region comprising a HCDR1 comprising the sequence of SEQ ID NO: 114, a HCDR2 comprising the sequence of SEQ ID NO: 115, and a HCDR3 comprising the sequence of SEQ ID NO: 116;
f) a heavy chain variable region comprising a HCDR1 comprising the sequence of SEQ ID NO: 119, a HCDR2 comprising the sequence of SEQ ID NO: 115, and a HCDR3 comprising the sequence of SEQ ID NO: 120; and
g) a heavy chain variable region comprising a HCDR1 comprising the sequence of SEQ ID NO: 123, a HCDR2 comprising the sequence of SEQ ID NO: 115, and a HCDR3 comprising the sequence of SEQ ID NO: 124.

In certain embodiments, the light chain variable region of the antibody against hGREM1 or an antigen-binding fragment thereof provided herein can be selected from the group consisting of:
a) a light chain variable region comprising a LCDR1 comprising the sequence of SEQ ID NO: 4, a LCDR2 comprising the sequence of SEQ ID NO: 5, and a LCDR3 comprising the sequence of SEQ ID NO: 6;
b) a light chain variable region comprising a LCDR1 comprising the sequence of SEQ ID NO: 14, a LCDR2 comprising the sequence of SEQ ID NO: 15, and a LCDR3 comprising the sequence of SEQ ID NO: 16;
c) a light chain variable region comprising a LCDR1 comprising the sequence of SEQ ID NO: 24, a LCDR2 comprising the sequence of SEQ ID NO: 25, and a LCDR3 comprising the sequence of SEQ ID NO: 26;
d) a light chain variable region comprising a LCDR1 comprising the sequence of SEQ ID NO: 34, a LCDR2 comprising the sequence of SEQ ID NO: 35, and a LCDR3 comprising the sequence of SEQ ID NO: 36;
e) a light chain variable region comprising a LCDR1 comprising the sequence of SEQ ID NO: 117, a LCDR2 comprising the sequence of SEQ ID NO: 35, and a LCDR3 comprising the sequence of SEQ ID NO: 118;
f) a light chain variable region comprising a LCDR1 comprising the sequence of SEQ ID NO: 121, a LCDR2 comprising the sequence of SEQ ID NO: 35, and a LCDR3 comprising the sequence of SEQ ID NO: 118;
g) a light chain variable region comprising a LCDR1 comprising the sequence of SEQ ID NO: 122, a LCDR2 comprising the sequence of SEQ ID NO: 35, and a LCDR3 comprising the sequence of SEQ ID NO: 118; and
h) a light chain variable region comprising a LCDR1 comprising the sequence of SEQ ID NO: 125, a LCDR2 comprising the sequence of SEQ ID NO: 35, and a LCDR3 comprising the sequence of SEQ ID NO: 118.

In certain embodiments, in the antibody or an antigen-binding fragment thereof provided herein:
a) the heavy chain variable region comprises a HCDR1 comprising the sequence of SEQ ID NO: 1, a HCDR2 comprising the sequence of SEQ ID NO: 2, and a HCDR3 comprising the sequence of SEQ ID NO: 3; and the light chain variable region comprises a LCDR1 comprising the sequence of SEQ ID NO: 4, a LCDR2 comprising the sequence of SEQ ID NO: 5, and a LCDR3 comprising the sequence of SEQ ID NO: 6;
b) the heavy chain variable region comprises a HCDR1 comprising the sequence of SEQ ID NO: 11, a HCDR2 comprising the sequence of SEQ ID NO: 12, and a HCDR3 comprising the sequence of SEQ ID NO: 13; and the light chain variable region comprises a LCDR1 comprising the sequence of SEQ ID NO: 14, a LCDR2 comprising the sequence of SEQ ID NO: 15, and a LCDR3 comprising the sequence of SEQ ID NO: 16;
c) the heavy chain variable region comprises a HCDR1 comprising the sequence of SEQ ID NO: 21, a HCDR2 comprising the sequence of SEQ ID NO: 22, and a HCDR3 comprising the sequence of SEQ ID NO: 23; and the light chain variable region comprises a LCDR1 comprising the sequence of SEQ ID NO: 24, a LCDR2 comprising the sequence of SEQ ID NO: 25, and a LCDR3 comprising the sequence of SEQ ID NO: 26;
d) the heavy chain variable region comprises a HCDR1 comprising the sequence of SEQ ID NO: 31, a HCDR2 comprising the sequence of SEQ ID NO: 32, and a HCDR3 comprising the sequence of SEQ ID NO: 33; and the light chain variable region comprises a LCDR1 comprising the sequence of SEQ ID NO: 34, a LCDR2 comprising the sequence of SEQ ID NO: 35, and a LCDR3 comprising the sequence of SEQ ID NO: 36;
e) the heavy chain variable region comprises a HCDR1 comprising the sequence of SEQ ID NO: 114, a HCDR2 comprising the sequence of SEQ ID NO: 115, and a HCDR3 comprising the sequence of SEQ ID NO: 116; and the light chain variable region comprises a LCDR1 comprising the sequence of SEQ ID NO: 117, a LCDR2 comprising the sequence of SEQ ID NO: 35, and a LCDR3 comprising the sequence of SEQ ID NO: 118;
f) the heavy chain variable region comprises a HCDR1 comprising the sequence of SEQ ID NO: 119, a HCDR2 comprising the sequence of SEQ ID NO: 115, and a HCDR3 comprising the sequence of SEQ ID NO: 120; and the light chain variable region comprises a LCDR1 comprising the sequence of SEQ ID NO: 121, a LCDR2 comprising the sequence of SEQ ID NO: 35, and a LCDR3 comprising the sequence of SEQ ID NO: 118;
g) the heavy chain variable region comprises a HCDR1 comprising the sequence of SEQ ID NO: 119, a HCDR2 comprising the sequence of SEQ ID NO: 115, and a HCDR3 comprising the sequence of SEQ ID NO: 120; and the light chain variable region comprises a LCDR1 comprising the sequence of SEQ ID NO: 122, a LCDR2 comprising the sequence of SEQ ID NO: 35, and a LCDR3 comprising the sequence of SEQ ID NO: 118; or
h) the heavy chain variable region comprises a HCDR1 comprising the sequence of SEQ ID NO: 123, a HCDR2 comprising the sequence of SEQ ID NO: 115, and a HCDR3 comprising the sequence of SEQ ID NO: 124; and the light chain variable region comprises a LCDR1 comprising the sequence of SEQ ID NO: 125, a LCDR2 comprising the sequence of SEQ ID NO: 35, and a LCDR3 comprising the sequence of SEQ ID NO: 118.

In certain embodiments, the heavy chain variable region of the antibody against hGREM1 or an antigen-binding fragment thereof provided herein comprises a sequence selected from the group consisting of SEQ ID NO: 7, SEQ ID NO: 17, SEQ ID NO: 27, SEQ ID NO: 37, SEQ ID NO: 41, SEQ ID NO: 43, SEQ ID NO: 45, SEQ ID NO: 51, SEQ ID NO: 53, SEQ ID NO: 55, SEQ ID NO: 57, SEQ ID NO: 126, SEQ ID NO: 128, SEQ ID NO: 131, SEQ ID NO: 132, SEQ ID NO: 133, and SEQ ID NO: 134, and a sequence thereof having at least 80% sequence identity thereof yet retaining specific binding specificity or affinity to hGREM1.

In certain embodiments, the light chain variable region of the antibody against hGREM1 or an antigen-binding fragment thereof provided herein comprises a sequence selected from the group consisting of SEQ ID NO: 8, SEQ ID NO: 18, SEQ ID NO: 28, SEQ ID NO: 38, SEQ ID NO: 47, SEQ ID NO: 49, SEQ ID NO: 59, SEQ ID NO: 61 SEQ ID NO: 127, SEQ ID NO: 129, SEQ ID NO: 130, SEQ ID NO: 135, SEQ ID NO: 136, and SEQ ID NO: 137, and a sequence having at least 80% sequence identity thereof yet retaining specific binding specificity or affinity to hGREM1.

In certain embodiments, the antibody or an antigen-binding fragment thereof provided herein comprises:
a) a heavy chain variable region comprising the sequence of SEQ ID NO: 7 and a light chain variable region comprising the sequence of SEQ ID NO: 8; or
b) a heavy chain variable region comprising a sequence of SEQ ID NO: 17 and a light chain variable region comprising a sequence of SEQ ID NO: 18; or
c) a heavy chain variable region comprising a sequence of SEQ ID NO: 27 and a light chain variable region comprising a sequence of SEQ ID NO: 28; or
d) a heavy chain variable region comprising a sequence of SEQ ID NO: 37 and a light chain variable region comprising a sequence of SEQ ID NO: 38; or
e) a heavy chain variable region comprising a sequence of SEQ ID NO: 126 and a light chain variable region comprising a sequence of SEQ ID NO: 127; or
f) a heavy chain variable region comprising a sequence of SEQ ID NO: 128 and a light chain variable region comprising a sequence of SEQ ID NO: 129; or
g) a heavy chain variable region comprising a sequence of SEQ ID NO: 128 and a light chain variable region comprising a sequence of SEQ ID NO: 130; or
h) a heavy chain variable region comprising a sequence selected from the group consisting of SEQ ID NO: 41, SEQ ID NO: 43 and SEQ ID NO: 45, and a light chain variable region comprising a sequence selected from the group consisting of SEQ ID NO: 47 and SEQ ID NO: 49; or
i) a pair of heavy chain variable region and light chain variable region sequences selected from the group consisting of: SEQ ID NOs: 41/47, 41/49, 43/47, 43/49, 45/47, and 45/49; or
j) a heavy chain variable region comprising a sequence selected from the group consisting of SEQ ID NO: 51, SEQ ID NO: 53, SEQ ID NO: 55 and SEQ ID NO: 57, and a light chain variable region comprising a sequence selected from the group consisting of SEQ ID NO: 59 and SEQ ID NO: 61; or
k) a pair of heavy chain variable region and light chain variable region sequences selected from the group consisting of: SEQ ID NOs: 51/59, 51/61, 53/59, 53/61, 55/59, 55/61, 57/59, and 57/61; or
l) a heavy chain variable region comprising a sequence selected from the group consisting of SEQ ID NO: 131, SEQ ID NO: 132, SEQ ID NO: 133 and SEQ ID NO: 134, and a light chain variable region comprising a sequence selected from the group consisting of SEQ ID NO: 135, SEQ ID NO: 136 and SEQ ID NO: 137; or
m) a pair of heavy chain variable region and light chain variable region sequences selected from the group consisting of: SEQ ID NOs: 131/135, 131/136, 131/137, 132/135, 132/136, 132/137, 133/135, 133/136, 133/137, 134/135, 134/136, and 134/137.

In certain embodiments, the antibody or antigen-binding fragment thereof provided herein further comprises one or more amino acid residue substitutions or modifications yet retains specific binding specificity or affinity to hGREM1.

In certain embodiments, the at least one of the substitutions or modifications is in one or more of the CDR sequences, and/or in one or more of the non-CDR regions of the VH or VL sequences.

In certain embodiments, the antibody or antigen-binding fragment thereof provided herein further comprises an immunoglobulin constant region, optionally a constant region of human Ig, or optionally a constant region of human IgG.

In certain embodiments, the constant region comprises a constant region of human IgG1, IgG2, IgG3, or IgG4.

In certain embodiments, the constant region comprises a heavy chain constant region comprising a sequence of SEQ ID NO: 138 and/or a light chain constant region comprising a sequence of SEQ ID NO:139.

In certain embodiments, the antibody or an antigen-binding fragment thereof provided herein is humanized.

In certain embodiments, the antibody or antigen-binding fragment thereof provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

In certain embodiments, the antibody or antigen-binding fragment thereof provided herein is bispecific.

In certain embodiments, the antibody or antigen-binding fragment thereof provided herein is capable of specifically binding to a first and a second epitope of hGREM1, or capable of specifically binding to both hGREM1 and a second antigen.

In certain embodiments, the second antigen provided herein is an immune related target.

In certain embodiments, the second antigen comprises PD-1, PD-L1, PD-L2, CTLA-4, TIM-3, LAG3, A2AR, CD160, 2B4, TGFβ, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16 or CD83.

In certain embodiments, the second antigen comprises a tumor antigen.

In certain embodiments, the tumor antigen comprises a tumor specific antigen or a tumor associated antigen.

In certain embodiments, the tumor antigen comprises prostate specific antigen (PSA), CA-125, gangliosides G(D2), G(M2) and G(D3), CD20, CD52, CD33, Ep-CAM, CEA, bombesin-like peptides, HER2/neu, epidermal growth factor receptor (EGFR), erbB2, erbB3/HER3, erbB4, CD44v6, Ki-67, cancer-associated mucin, VEGF, VEGFRs (e.g., VEGFR3), estrogen receptors, Lewis-Y antigen, TGF(31, IGF-1 receptor, EGFα, c-Kit receptor, transferrin receptor, Claudin 18.2, GPC-3, Nectin-4, ROR1, methothelin, PCMA, MAGE-1, MAGE-3, BAGE, GAGE-1, GAGE-2, p15, BCR-ABL, E2APRL, H4-RET, IGH-IGK, MYL-RAR, IL-2R, CO17-1A, TROP2, or LIV-1.

In certain embodiments, the antibody or an antigen-binding fragment thereof provided herein is not cross-reactive to mouse gremlin1.

In certain embodiments, the antibody or an antigen-binding fragment thereof provided herein is cross-reactive to mouse gremlin1.

In certain embodiments, the antibody or antigen-binding fragment thereof provided herein is linked to one or more conjugate moieties.

In certain embodiments, the conjugate moiety comprises a clearance-modifying agent, a chemotherapeutic agent, a toxin, a radioactive isotope, a lanthanide, a luminescent label, a fluorescent label, an enzyme-substrate label, a DNA-alkylator, a topoisomerase inhibitor, a tubulin-binders, or other anticancer drugs such as androgen receptor inhibitor.

In another aspect, the present disclosure provides an antibody or an antigen-binding fragment thereof, which competes for binding to hGREM1 with the antibody or antigen-binding fragment thereof provided herein.

In another aspect, the present disclosure provides a pharmaceutical composition or kit comprising the antibody or antigen-binding fragment thereof provided herein, and a pharmaceutically acceptable carrier.

In certain embodiments, the pharmaceutical composition or kit further comprises a second therapeutic agent.

In another aspect, the present disclosure provides an isolated polynucleotide encoding the antibody or an antigen-binding fragment thereof provided herein.

In another aspect, the present disclosure provides a vector comprising the isolated polynucleotide provided herein.

In another aspect, the present disclosure provides a host cell comprising the vector provided herein.

In another aspect, the present disclosure provides a method of expressing the antibody or antigen-binding fragment thereof provided herein, comprising culturing the host cell provided herein under the condition at which the vector provided herein can be expressed.

In another aspect, the present disclosure provides a method of treating a GREM1-related disease or condition in a subject, comprising administering to the subject a therapeutically effective amount of the antibody or antigen-binding fragment thereof provided herein, or the pharmaceutical composition provided herein.

In another aspect, the present disclosure provides a method of treating a GREM1-related disease or condition in a subject in need thereof, comprising administering to the subject a therapeutically effective amount of an anti-human GREM1 antibody or antigen-binding fragment thereof, which is:
a) capable of binding to hGREM1 at an epitope comprising residue Gln27 and/or residue Asn33, wherein residue number is according to SEQ ID NO: 69, and/or
b) capable of binding to a hGREM1 fragment comprising residue Gln27 and/or residue Asn33, optionally the hGREM1 fragment has a length of at least 3 (e.g. 4, 5, 6, 7, 8, 9, or 10) amino acid residues; and/or
c) capable of reducing hGREM1-mediated inhibition on BMP signaling selectively in a cancer cell over a non-cancer cell; and/or
d) exhibiting no more than 50% reduction of hGREM1-mediated inhibition on BMP signaling in a non-cancer cell; and/or
e) capable of binding to a chimeric hGREM1 comprising an amino acid sequence of SEQ ID NO: 68; and/or
f) capable of binding to hGREM1 at a K_{D} of no more than 1 nM as measured by Fortebio; and/or
h) capable of blocking the binding of hGREM1 to BMP7 at a maximal blocking percentage of more than 50% as measured by ELISA; and/or
i) capable of blocking interaction of GREM1 to FGFR.

In certain embodiments, the GREM1-related disease or condition is selected from the group consisting of cancer, fibrotic disease, angiogenesis, glaucoma or retinal disease, kidney disease, pulmonary arterial hypertension, or osteoarthritis (OA).

In certain embodiments, the cancer is a GREM1-expressing cancer. In certain embodiments, the GREM1-expressing cancer is also PD-L1-expressing. In certain embodiments, the GREM1-expressing cancer is not a PD-L1-expressing cancer. In certain embodiments, the GREM1-expressing cancer is resistant or refractory to the treatment with a PD-1/PD-L1 axis inhibitor.

In certain embodiments, the subject is identified as having a GREM1-expressing cancer cell, or having a GREM1-expressing cancer microenvironment.

In certain embodiments, the cancer is a solid tumor or hematological cancer.

In certain embodiments, the solid tumor is adrenocortical carcinoma, anal cancer, astrocytoma, childhood cerebellar or cerebral, basal-cell carcinoma, bile duct cancer, bladder cancer, bone tumor, brain cancer, cerebellar astrocytoma, cerebral astrocytoma/malignant glioma, ependymoma, medulloblastoma, supratentorial primitive neuroectodermal tumors, visual pathway and hypothalamic glioma, breast cancer, Burkitt's lymphoma, cervical cancer, colon cancer, emphysema, endometrial cancer, esophageal cancer, Ewing's sarcoma, retinoblastoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, liver cancer, lung cancer, neuroblastoma, non-Hodgkin lymphoma, ovarian cancer, pancreatic cancer, pharyngeal cancer, prostate cancer, rectal cancer, renal cell carcinoma (kidney cancer), retinoblastoma, Ewing family of tumors, skin cancer, stomach cancer, testicular cancer, throat cancer, thyroid cancer, or vaginal cancer.

In certain embodiments, the hematological cancer is leukemia (such as Acute lymphocytic leukemia (ALL), Acute myeloid leukemia (AML), Chronic lymphocytic leukemia (CLL), Chronic myeloid leukemia (CML)), lymphoma (such as Hodgkin's lymphoma, or Non-Hodgkin's lymphoma (e.g. Waldenstrom macroglobulinemia (WM))), or myeloma (such as multiple myeloma (MM)).

In certain embodiments, the cancer is prostate cancer, gastric-esophageal cancer, lung cancer (e.g., non-small cell lung cancer), liver cancer, pancreatic cancer, breast cancer, bronchial cancer, bone cancer, liver and bile duct cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, gastrointestinal cancer, skin cancer, pituitary cancer, stomach cancer, vagina cancer, thyroid cancer, glioblastoma, astrocytoma, melanoma, myelodysplastic syndrome, sarcoma, teratoma, glioma, or adenocarcinoma.

In certain embodiments, the cancer is selected from the group consisting of prostate cancer, gastric-esophageal cancer, lung cancer (e.g., non-small cell lung cancer), liver cancer, colon cancer, colorectal cancer, glioma, pancreatic cancer, bladder cancer and breast cancer. In certain embodiments, the cancer is triple negative breast cancer. In certain embodiments, the cancer is multiple myeloma.

In certain embodiments, the cancer is prostate cancer.

In certain embodiments, the fibrotic disease is a fibrotic disease in lungs, liver, kidney, eyes, skin, heart, gut or muscle.

In certain embodiments, the subject is human.

In certain embodiments, the administration is via oral, nasal, intravenous, subcutaneous, sublingual, or intramuscular administration.

In certain embodiments, the method provided herein further comprises administering a therapeutically effective amount of a second therapeutic agent.

In certain embodiments, the second therapeutic agent comprises an anti-cancer therapy, optionally the anti-cancer therapy is selected from a chemotherapeutic agent (e.g. cisplatin), an anti-cancer drug, radiation therapy, an immunotherapy agent (e.g. an immune checkpoint modulator, for example, a PD-1/PD-L1 axis inhibitor, and a TGF-beta inhibitor), anti-angiogenesis agent (e.g. antagonist of a VEGFR such as VEGFR-1, VEGFR-2, and VEGFR-3), a targeted therapy agent, a cellular therapy agent, a gene therapy agent, a hormonal therapy agent, cytokines, palliative care, surgery for the treatment of cancer (e.g., tumorectomy), one or more anti-emetics, treatments for complications arising from chemotherapy, a diet supplement for cancer patients (e.g. indole-3-carbinol), an agent that modulates tumor microenvironment (e.g. a bifunctional molecule comprising PD-L1 binding moiety and extracellular domain of TGF-beta receptor), or anti-fibrotic therapy (e.g., BMP7 treatment, ACE inhibitor (or ARB), anti-MASP2 antibody, endothelin receptor antagonist, NRF2 inhibitor steroid, CTLA4-IgG or TNF inhibitor). In certain embodiments, the second therapeutic agent comprises cisplatin. In certain embodiments, the second therapeutic agent comprises a PD-1/PD-L1 axis inhibitor.

In certain embodiments the anti-cancer therapy comprises an anti-prostate cancer drug.

In certain embodiments, the anti-prostate cancer drug comprises an androgen axis inhibitor; an androgen synthesis inhibitor; a poly ADP-ribose polymerase (PARP) inhibitor; or a combination thereof.

In certain embodiments, the androgen axis inhibitor is selected from the group consisting of Luteinizing hormone-releasing hormone (LHRH) agonists, LHRH antagonists and androgen receptor antagonist.

In certain embodiments, the androgen axis inhibitor is degarelix, bicalutamide, flutamide, nilutamide, apalutamide, darolutamide, enzalutamide, or abiraterone. In certain embodiments, the androgen synthesis inhibitor is abiraterone acetate or ketoconazole. In certain embodiments, the PARP inhibitor is olaparib, or rucaparib.

In certain embodiments, the anti-prostate cancer drug is selected from the group consisting of Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate) and Zytiga (Abiraterone Acetate).

In certain embodiments, the second therapeutic agent comprises indole-3-carbinol.

In another aspect, the present disclosure provides a kit comprising an antibody or an antigen-binding fragment provided herein.

In another aspect, the present disclosure provides a method of detecting presence or amount of GREM1 in a sample, comprising contacting the sample with the antibody or antigen-binding fragment thereof provided herein, and determining the presence or the amount of GREM1 in the sample.

In another aspect, the present disclosure provides use of the antibody or antigen-binding fragment thereof provided herein in the manufacture of a medicament for treating a GREM1-related disease or condition in a subject.

In certain embodiments the GREM1-related disease or condition is cancer.

In certain embodiments, the GREM1-related disease or condition is fibrotic disease, angiogenesis, glaucoma, retinal disease, kidney disease, pulmonary arterial hypertension, or osteoarthritis (OA).

### BRIEF DESCRIPTION OF FIGURES

FIG. 1 shows the binding of the anti-gremlin1 antibodies 69H5, 56C11, 22F1 and 14E3 to human gremlin1 (hGREM1) (FIG. 1A, 1C) and to mouse gremlin1 (FIG. 1B, 1C), and the binding of the anti-gremlin1 antibodies 42B9, 36F5, and 67G11 to human gremlin1 (hGREM1) (FIG. 1D, 1F) and to mouse gremlin1 (FIG. 1E, 1F) respectively, as measured by ELISA.

FIG. 2 shows the binding specificity of the anti-gremlin1 antibody 14E3 to gremlin-1, relative to gremlin-2, COCO, and DAN-hFc, as measured by ELISA.

FIG. 3 shows the binding affinity of gremlin1 or XM5 (gremlin 1-DAN fusion protein) to BMP2/4/7 (FIG. 3A, 3D), blocking activity of binding of gremlin1 to BMP2 (FIG. 3B) to BMP4 (FIG. 3C) by anti-gremlin1 antibodies 69H5, 56C11, 14E3 or benchmark antibody 6245P, and blocking activity of binding of gremlin1 to BMP2 (FIG. 3E, 3H), or to BMP4 (FIG. 3F, 3H), or to BMP7 (FIG. 3G, 3H) by anti-gremlin1 antibodies 42B9, 36F5, 67G11, and 14E3 HaLa or benchmark antibody 6245P as measured by ELISA.

FIG. 4 shows the blocking of gremlin mediated inhibition on BMP4 signaling by anti-gremlin1 antibodies 69H5, 56C11, 22F1, 14E3 and benchmark antibody 6245P, as measured by BMP-induced reporter assay.

FIG. 5 shows the blocking of gremlin mediated inhibition on BMP4 signaling by anti-gremlin1 antibodies 14E3 (FIG. 5A), 22F1 (FIG. 5B), 56C11 (FIG. 5C), and 69H5 (FIG. 5D), relative to benchmark antibody 6245P, as measured by BMP4-induced ATDC-5 cell differentiation.

FIG. 6 shows that gremlin1 reduces the level of BMP 4 induced smad phosphorylation in prostate cancer cells (PC-3 cells) (FIG. 6A), which is restored by the anti-gremlin1 antibodies 14E3, 22F1, 56C11, and 69H5, in prostate cancer cells (FIG. 6B), as measured by western blot.

FIG. 7 shows the blocking of gremlin mediated inhibition on BMP4 signaling by anti-gremlin1 antibody 14E3 on cancer cells and non-cancer cells.

FIG. 8 shows the binding affinity of the chimeric anti-gremlin1 antibodies 56C11-C and 14E3-C to hGREM1 as measured by ELISA.

FIG. 9 shows the binding kinetics of the chimeric anti-gremlin1 antibodies 14E3-C and 22F1-C to hGREM1 as measured by Biacore.

FIG. 10 shows results from epitope studies, wherein FIG. 10A-C shows epitope binning results of the anti-gremlin1 antibodies 14E3, 22F1, 56C11, and 69H5 as measured by competition ELISA assay (FIG. 10A), cross-competition assay results for antibodies 14E3-C, 22F1-C, and benchmark antibody 6245P (FIG. 10B), and binding of antibodies 14E3-C and benchmark antibody 6245P to gremlin-DAN fusion protein XM5 as measured by ELISA (FIG. 10C), and FIG. 10D shows epitope mapping of 14E3 as measured by Biolayer Interferometry (BLI) assay, and FIGS. 10E and 10F show binding of antibodies 42B9, 36F5, and 67G11 to human gremlin or gremlin-DAN fusion protein XM5 as measured by ELISA.

FIG. 11A and FIG. 11B show epitope analysis results of the anti-gremlin1 antibodies 14E3, 56C11, 22F1, 69H5 and benchmark antibody 6245P provided herein as measured by Fortebio. The result showed that 14E3 has completely non-overlapping epitope than that of 6245P, while 56C11 shares similar epitope to that of 6245P.

FIG. 12 shows the binding affinity of the humanized anti-hGREM 1 antibodies 14E3 and 22F1 to hGREM1 as measured by ELISA (FIG. 12 A-C) or by Fortebio (FIG. 12D), in comparison to benchmark antibody 6245P.

FIG. 13 shows the anti-GREM1 antibody 14E3 reduced the tumor volume (FIG. 13A) and tumor weight (FIG. 13B) of prostate cancer in PC3 xenograft model.

FIG. 14 shows anti-tumor effect of anti-GREM 1 antibody 56C11 on CT-26 colon cancer model.

FIG. 15A and FIG. 15B show synergistic anti-tumor effect of combination therapy with an anti-mGREM1 antibody and an immune checkpoint inhibitor (e.g., MPDL-3280A) on CT-26 model.

FIG. 16 shows the IHC staining of GREM1 or PD-L1 in E7 PDX tumor samples by either an anti-GREM1 antibody (14E3) or an anti-PD-L1 antibody (22C3).

FIG. 17A and FIG. 17B show that humanized 14E3 (hzd 14E3), alone or in combination with Cisplatin, inhibited tumor growth in a gremlin-positive esophageal PDX model. Humanized 14E3 alone achieved about 43% tumor growth inhibition (TGI). Cisplatin alone achieved about 60% TGI. Combination of humanized 14E3 and Cisplatin achieved about 64% TGI.

FIG. 18 shows the binding of Gremlin to FGFR1 (FIG. 18A), and the blocking activity of binding of gremlin to FGFR1 of huIgGI, hIgG4, the anti-gremlin1 antibodies 42B9, 36F5, 67G11, 69H5-chi, 36F5-chi, 22F1-chi, 56C11-chi and 14E3 HaLa, as well as benchmark antibody 6245P (FIG. 18B, 18C) respectively, as measured by ELISA.

FIG. 19 shows ELISA binding activity of hybridoma 36F5 (FIG. 19A) and chimeric 36F5 (36F5-chi) (FIG. 19B) to Gremlin-his and DAN-his.

FIG. 20 shows that chimeric 36F5 (36F5-chi) blocks BMP4 binding to DAN protein (FIG. 20A), and BMP2 binding to DAN protein (FIG. 20B).

FIG. 21 shows the anti-tumor activity of hybridoma 36F5 on EMT6/hPD-L1 tumor model. FIG. 21A shows that the EMT6/hPD-L1 tumor model has poor responsiveness to the anti-PD-L1 antibody AM4B6. FIG. 21B shows that the hybridoma 36F5 exhibits anti-tumor activity on the EMT6/hPD-L1 tumor model.

FIG. 22 shows the anti-tumor activity of hybridoma 14E3 or 36F5 on E7 tumor model. FIG. 22A shows that the E7 tumor model has poor responsiveness to the anti-PD-1 antibody Nivolumab and the hybridoma 14E3 exhibits anti-tumor activity on the E7 tumor model. FIG. 22B shows that the E7 tumor model has poor responsiveness to the anti-PD-1 antibody Nivolumab and the hybridoma 36F5 exhibits anti-tumor activity on the E7 tumor model.

FIG. 23 shows the anti-tumor activity of 56C11 combination therapy with anti-PDL1 antibody on MC38/hPD-L1 tumor model.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “antibody” as used herein includes any immunoglobulin, monoclonal antibody, polyclonal antibody, multivalent antibody, bivalent antibody, monovalent antibody, multispecific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain consists of a variable region (V_{H}) and a first, second, and third constant region (C_{H1}, C_{H2}, C_{H3}, respectively); mammalian light chains are classified as λ or κ, while each light chain consists of a variable region (V_{L}) and a constant region. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies and antigen-binding domains disclosed herein may be defined or identified by the conventions of Kabat, IMGT, AbM, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); N. R. Whitelegg et al, Protein Engineering, v13(12), 819-824 (2000); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs are interposed between flanking stretches known as framework regions (FRs), which are more highly conserved than the CDRs and form a scaffold to support the hypervariable loops. The constant regions of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain). In certain embodiments, the antibody provided herein encompasses any antigen-binding fragments thereof.

As used herein, the term “antigen-binding fragment” refers to an antibody fragment formed from a fragment of an antibody comprising one or more CDRs, or any other antibody portion that binds to an antigen but does not comprise an intact native antibody structure. Examples of antigen-binding fragment include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In certain embodiments, an antigen-binding fragment may comprise one or more CDRs from a particular parent antibody.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab′” refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab′)₂” refers to a dimer of Fab′ that comprises two light chains and part of two heavy chains.

“Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, a “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and VL2 coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 June 15 (2007)). “Diabodies” include small antibody fragments with two antigen-binding sites, wherein the fragments comprise a V_{H} domain connected to a V_{L} domain in a single polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more V_{H} domains are covalently joined with a peptide linker to form a bivalent or multivalent domain antibody. The two VH domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.

In certain embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In certain embodiments, a “bispecific dsFv” or “dsFv-dsFv′” comprises three peptide chains: a V_{H1}-V_{H2} moiety wherein the heavy chains are bound by a peptide linker (e.g., a long flexible linker) and paired via disulfide bridges to V_{L1} and V_{L2} moieties, respectively. Each disulfide paired heavy and light chain has a different antigen specificity.

The term “humanized” as used herein means that the antibody or antigen-binding fragment comprises CDRs derived from non-human animals, FR regions derived from human, and when applicable, constant regions derived from human. In certain embodiments, the amino acid residues of the variable region framework of the humanized gremlin antibody are substituted for sequence optimization. In certain embodiments, the variable region framework sequences of the humanized gremlin antibody chain are at least 65%, 70%, 75%, 80%, 85%, 90%, 95% or 100% identical to the corresponding human variable region framework sequences.

The term “chimeric” as used herein refers to an antibody or antigen-binding fragment that has a portion of heavy and/or light chain derived from one species, and the rest of the heavy and/or light chain derived from a different species. In an illustrative example, a chimeric antibody may comprise a constant region derived from human and a variable region derived from a non-human species, such as from mouse.

The term “germline sequence” refers to the nucleic acid sequence encoding a variable region amino acid sequence or subsequence that shares the highest determined amino acid sequence identity with a reference variable region amino acid sequence or subsequence in comparison to all other known variable region amino acid sequences encoded by germline immunoglobulin variable region sequences. The germline sequence can also refer to the variable region amino acid sequence or subsequence with the highest amino acid sequence identity with a reference variable region amino acid sequence or subsequence in comparison to all other evaluated variable region amino acid sequences. The germline sequence can be framework regions only, complementarity determining regions only, framework and complementarity determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. Germline sequences can be determined, for example, through the publicly available international ImMunoGeneTics database (IMGT) and V-base.

“Anti-human gremlin1 antibody”, “anti-hGREM1 antibody” or “an antibody against human gremlin1” as used herein interchangeably and refers to an antibody that is capable of specific binding to human gremlin1 with a sufficient specificity and/or affinity, for example, to provide for therapeutic use.

The term “affinity” as used herein refers to the strength of non-covalent interaction between an immunoglobulin molecule (i.e. antibody) or fragment thereof and an antigen.

The term “specific binding” or “specifically binds” as used herein refers to a non-random binding reaction between two molecules, such as for example between an antibody and an antigen. In certain embodiments, the antibodies or antigen-binding fragments provided herein specifically bind to human and/or non-human gremlin1 with a binding affinity (K_{D}) of ≤10⁻⁶ M (e.g., ≤5×10⁻⁷ M, ≤2×10-7 M, ≤10-7 M, ≤5×10⁻⁸ M, ≤2×10⁻⁸ M, ≤10⁻⁸M, ≤5×10-9 M, ≤4×10⁻⁹M, ≤3×10⁻⁹M, ≤2×10⁻⁹ M, or ≤10⁻⁹ M. K_{D} used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry method. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum correspondence. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm. In certain embodiments, the non-identical residue positions may differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference.

As used herein, a “homologous sequence” refers to a polynucleotide sequence (or its complementary strand) or an amino acid sequence that has sequence identity of at least 80% (e.g. at least 85%, 88%, 90%, 91%, 92%, 93%, 94%, 95%, 96%, 97%, 98%, 99%) to another sequence when optionally aligned.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An isolated “nucleic acid” or “polynucleotide” are used interchangeably and refer to the sequence of an isolated nucleic acid molecule. In certain embodiments, an “isolated antibody or antigen-binding fragment thereof” refers to the antibody or antigen-binding fragments having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, 99% as determined by electrophoretic methods (such as SDS-PAGE, isoelectric focusing, capillary electrophoresis), or chromatographic methods (such as ion exchange chromatography or reverse phase HPLC).

The term “subject” includes human and non-human animals. Non-human animals include all vertebrates, e.g., mammals and non-mammals, such as non-human primates, mouse, rat, cat, rabbit, sheep, dog, cow, chickens, amphibians, and reptiles. Except when noted, the terms “patient” or “subject” are used herein interchangeably.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a genetic element may be operably inserted so as to bring about the expression of that genetic element, such as to produce the protein, RNA or DNA encoded by the genetic element, or to replicate the genetic element. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g. expression vectors) containing the nucleic acid sequence provided herein encoding the antibody or antigen-binding fragment thereof, at least one promoter (e.g. SV40, CMV, EF-1α) operably linked to the nucleic acid sequence, and at least one selection marker.

The “host cell” as used herein refers to a cell into which an exogenous polynucleotide and/or a vector has been introduced.

The term “gremlin1” or “GREM1” refers to the variant 1 of gremlin, and encompasses gremlin1 in different species such as in human, mouse, monkey, and so on. GREM1 is evolutionarily conserved and the human gremlin1 gene (hGREM1) has been mapped to chromosome 15q13-q15 (Topol L Z et al., (1997) Mol. Cell Biol., 17: 4801-4810; Topol L Z et al., Cytogenet Cell Genet., 89: 79-84). The amino acid sequence of hGREM1 is accessible by GenBank database under the accession number NP-037504 or Uniprot Database via the accession number 060565, and is provided herein as SEQ ID NO: 66. The term “human gremlin1” and the term “hGREM1” are used interchangeably in the present disclosure.

A “gremlin1-related” or “GREM1-related” disease or condition as used herein refers to any disease or condition caused by, exacerbated by, or otherwise linked to increased expression or activities of GREM1. In some embodiments, the GREM1 related condition is, for example, glaucoma, cancer, fibrotic disease, angiogenesis, retinal disease, kidney disease, pulmonary arterial hypertension, or osteoarthritis (OA).

“Cancer” as used herein refers to any medical condition characterized by malignant cell growth or neoplasm, abnormal proliferation, infiltration or metastasis, and can be benign or malignant, and includes both solid tumors and non-solid cancers (e.g. hematologic malignancies) such as leukemia. As used herein “solid tumor” refers to a solid mass of neoplastic and/or malignant cells.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

Reference to “about” a value or parameter herein includes (and describes) embodiments that are directed to that value or parameter per se. For example, description referring to “about X” includes description of “X.” Numeric ranges are inclusive of the numbers defining the range. Generally speaking, the term “about” refers to the indicated value of the variable and to all values of the variable that are within the experimental error of the indicated value (e.g. within the 95% confidence interval for the mean) or within 10 percent of the indicated value, whichever is greater. Where the term “about” is used within the context of a time period (years, months, weeks, days etc.), the term “about” means that period of time plus or minus one amount of the next subordinate time period (e.g. about 1 year means 11-13 months; about 6 months means 6 months plus or minus 1 week; about 1 week means 6-8 days; etc.), or within 10 percent of the indicated value, whichever is greater.

### Anti-Human Gremlin1 Antibodies

The present disclosure provides anti-human gremlin1 (hGREM1) antibodies and antigen-binding fragments thereof. The anti-hGREM1 antibodies and antigen-binding fragments provided herein are uniquely differentiated from existing anti-hGREM1 antibodies in the following aspects: a) capable of reducing hGREM1-mediated inhibition on BMP signaling selectively in a cancer cell over a non-cancer cell; b) exhibiting no more than 50% reduction of hGREM1-mediated inhibition on BMP signaling in a non-cancer cell; c) capable of binding to a chimeric hGREM1 comprising an amino acid sequence of SEQ ID NO: 68; d) capable of binding to hGREM1 but not specifically binding to mouse gremlin1; e) binding to hGREM1 at an epitope comprising residue Gln27 and/or residue Asn33, wherein the residue numbering is according to SEQ ID NO: 69, or binds to a hGREM1 fragment comprising residue Gln27 and/or residue Asn33, optionally the hGREM1 fragment has a length of at least 3 (e.g. 4, 5, 6, 7, 8, 9, or 10) amino acid residues; f) capable of reducing hGREM1-mediated activation on MAPK signaling; g) capable of binding to hGREM1 at a Kd of no more than 1 nM as measured by Fortebio; h) capable of blocking the binding of hGREM1 to BMP7 at a maximal blocking percentage of at least 50% as measured by ELISA; i) capable of blocking interaction of GREM1 (e.g., hGREM1 or mGREM1) to FGFR (e.g., FGFR1, preferably human FGFR1 (hFGFR1) or mouse FGFR1 (mFGFR1)) and/or j) capable of binding to both hGREM1 and DAN.

Bone morphogenetic proteins (BMPs), such as BMP 2, BMP 4 and BMP 7, are known as glycosylated extracellular matrix (ECM) associated members of the transforming growth factor beta (TGF-β) super-family, and have a key role during morphogenesis, general organogenesis, cartilage and limb formation, cell proliferation, differentiation and apoptosis. BMP signaling is activated by the binding of BMP ligands (e.g., BMP 2, BMP 4 and BMP7) to the BMP receptors to trigger receptor phosphorylation, leading to phosphorylation of R-Smads (e.g. Smad 1/5/9) and complex formation with co-Smad4. The formed Smad complex then translocate to the nucleus to regulate expression of BMP target genes. Accordingly, activation of BMP signaling can be assayed by, for example, measuring the phosphorylation level of smads and/or the expression level of BMP target genes. The activation of BMP signaling can also be assayed by measuring the expression level of differentiation marker genes, e.g., alkaline phosphatase which is an early marker of osteoblast differentiation.

BMP signaling is known to be inhibited by BMP antagonists, such as noggin, chordin, gremlin1, and twisted gastrulation 1. GREM1 can physically bind to BMP ligands such as BMP-2, BMP-4 or BMP-7 to form heterodimers and prevent these BMP ligands from interacting with their corresponding BMP receptors, thereby inhibiting the activation of BMP signaling pathway. It is therefore expected that eliminating GREM1 and/or reducing the activity of GREM1 would reverse or reduce the inhibition on BMP signaling.

i. Binding affinity

In certain embodiments, the anti-GREM1 antibodies provided herein are capable of binding to hGREM1 at a Kd of no more than 1 nM as measured by Fortebio. Binding affinity of the anti-GREM1 antibodies and antigen-binding fragments provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g. K_{D}) can be appropriately determined using any suitable methods known in the art, including, for example, Kinetic Exclusion Assay (KinExA), Biacore, Fortebio or flow cytometry.

In certain embodiments, the “K_{D}” or “K_{D} value” according to the present disclosure is in an embodiment measured by Biacore or Fortebio assay, performed with the anti-GREM1 antibody and GREM1 as described by the following assay that measures solution binding affinity of an anti-GREM1 antibody. In general, the Biacore works by equilibrating a constant amount of one binding partner (CBP) with a varying concentration of the other binding partner (Titrant), and then capture a portion of the free CBP by fluorescence labeled secondary antibody in a short contact time which is less than the time needed for dissociation of the pre-formed CBP-Titrant complex. The fluorescence signals generated from the captured CBP are directly proportional to the concentration of free CBP in the equilibrated samples, and are used to generate a binding curve (percent free CBP vs. total Titrant concentration) when measured in a series. More details are available from Schreiber, G., Fersht, A. R. Nature Structural Biology. 1996, 3(5), 427-431. When anti-GREM1 antibody is used as CBP with a constant amount, then GREM1 protein can be used as the Titrant, or vice versa. Fortebio generally works in a similar way to Biacore, also by equilibrating a constant amount of CBP (e.g., GREM 1 protein) with a varying concentration of the Titrant (e.g., anti-GREM1 antibody). The information of binding kinetics (kₒₙ and k_{off}) between CBP and the Titrant may be obtained from the change in the interference pattern generated from a biosensor used by Fortebio. More details are available from Charles Wartchow, Frank Podlaski, Shirley Li, Karen Rowan, Xiaolei Zhang, David Mark, Kuo-Sen Huang “Biosensor-based Small Molecule Fragment Screening with Biolayer Interferometry: J Comput Aided Mol Des 2011, 25(7), 669-76.

In certain embodiments, the K_{D} of the anti-GREM1 antibodies provided herein are determined in accordance to the method as described in Example 14 in the present disclosure.

Other methods suitable for measurement of K_{D} may also be used under applicable circumstances, for example, radiolabelled antigen-binding assay (see, e.g. Chen, et al., (1999) J. Mol Biol 293:865-881), or surface plasmon resonance assays other than Biacore.

In certain embodiments, the anti-GREM1 antibodies and the antigen-binding fragments thereof provided herein specifically bind to human GREM1 at a K_{D} value of no more than 100 nM (or no more than 90, 80, 70, 60, 50, 40, 30, 29, 27, 20, 19, or 18 nM) as measured by Biacore assay. In certain embodiments, the anti-GREM1 antibodies and the antigen-binding fragments thereof provided herein specifically bind to human GREM1 at a K_{D} value of no more than 10 nM (or no more than 9, 8, 7, 6, 5, 4, 3, 2 or 1 nM) as measured by Fortebio assay.

Alternatively, binding affinity of the anti-GREM1 antibodies and antigen-binding fragments provided herein to human GREM1 can also be represented by “half maximal effective concentration” (EC₅₀) value, which refers to the concentration of an antibody where 50% of its maximal effect (e.g., binding) is observed. The EC₅₀ value can be measured by methods known in the art, for example, sandwich assay such as ELISA, Western Blot, flow cytometry assay, and other binding assay. In certain embodiments, the anti-GREM1 antibodies and the fragments thereof provided herein specifically bind to human GREM1 (e.g. a cell expressing human GREM1) at an EC50 value of no more than 120 ng/ml (or no more than 100, 90, 80, 60, 40, 30, 20, 14.5, 14, 13.5, 13, 12.5, 12, 11.5, 11, 10.5, 10, 9, 8, 7, 6, 5.5, 5, 4.5, 4, 3, 2, or 1 ng/ml) as measured by ELISA.

In certain embodiments, some of the antibodies and antigen-binding fragments thereof provided herein are capable of specifically binding to mouse GREM1 at an EC50 value of no more than 20 ng/ml as measured by ELISA. In certain embodiments, the antibodies and antigen-binding fragments thereof bind to mouse GREM1 at an EC50 of 4 ng/ml-20 ng/ml (e.g. 4 ng/ml-9 ng/ml, 5 ng/ml-8 ng/ml, 6 ng/ml-7 ng/ml, 6 ng/ml-14 ng/ml, 6 ng/ml-12 ng/ml, 4.564 ng/ml, 7.713 ng/ml, 8.512 ng/ml or 17.2 ng/ml) as measured by ELISA.

In certain embodiments, some of the antibodies and antigen-binding fragments thereof provided herein do not specifically bind to mouse GREM1.

In certain embodiments, the antibodies and antigen-binding fragments thereof provided herein do not specifically bind to GREM2.

### Antibody Sequences

In one aspect, the present disclosure provides an anti-GREM1 antibody or an antigen-binding fragment thereof provided herein, wherein the heavy chain variable region comprises:
a) a HCDR1 comprises a sequence selected from SEQ ID NOs: 1, 11, 21, 31, 114, 119 and 123,
b) a HCDR2 comprises a sequence selected from SEQ ID NOs: 2, 12, 22, 32, and 115, and
c) a HCDR3 comprises a sequence selected from SEQ ID NOs: 3, 13, 23, 33, 116, 120 and 124 and/or
a light chain variable region comprising:
d) a LCDR1 comprises a sequence of SEQ ID NOs: 4, 14, 24, 34, 117, 121, 122 and 125,
e) a LCDR2 comprises a sequence of SEQ ID NOs: 5, 15, 25 and 35, and
f) a LCDR3 comprises a sequence selected from SEQ ID NOs: 6, 16, 26, 36 and 118.

In certain embodiments, the antibody or an antigen-binding fragment thereof provided herein, wherein the heavy chain variable region is selected from the group consisting of:
a) a heavy chain variable region comprising a HCDR1 comprising the sequence of SEQ ID NO: 1, a HCDR2 comprising the sequence of SEQ ID NO: 2, and a HCDR3 comprising the sequence of SEQ ID NO: 3;
b) a heavy chain variable region comprising a HCDR1 comprising the sequence of SEQ ID NO: 11, a HCDR2 comprising the sequence of SEQ ID NO: 12, and a HCDR3 comprising the sequence of SEQ ID NO: 13;
c) a heavy chain variable region comprising a HCDR1 comprising the sequence of SEQ ID NO: 21, a HCDR2 comprising the sequence of SEQ ID NO: 22, and a HCDR3 comprising the sequence of SEQ ID NO: 23;
d) a heavy chain variable region comprising a HCDR1 comprising the sequence of SEQ ID NO: 31, a HCDR2 comprising the sequence of SEQ ID NO: 32, and a HCDR3 comprising the sequence of SEQ ID NO: 33;
e) a heavy chain variable region comprising a HCDR1 comprising the sequence of SEQ ID NO: 114, a HCDR2 comprising the sequence of SEQ ID NO: 115, and a HCDR3 comprising the sequence of SEQ ID NO: 116;
f) a heavy chain variable region comprising a HCDR1 comprising the sequence of SEQ ID NO: 119, a HCDR2 comprising the sequence of SEQ ID NO: 115, and a HCDR3 comprising the sequence of SEQ ID NO: 120; and
g) a heavy chain variable region comprising a HCDR1 comprising the sequence of SEQ ID NO: 123, a HCDR2 comprising the sequence of SEQ ID NO: 115, and a HCDR3 comprising the sequence of SEQ ID NO: 124.

In certain embodiments, the antibody or an antigen-binding fragment thereof provided herein, wherein the light chain variable region is selected from the group consisting of:
a) a light chain variable region comprising a LCDR1 comprising the sequence of SEQ ID NO: 4, a LCDR2 comprising the sequence of SEQ ID NO: 5, and a LCDR3 comprising the sequence of SEQ ID NO: 6;
b) a light chain variable region comprising a LCDR1 comprising the sequence of SEQ ID NO: 14, a LCDR2 comprising the sequence of SEQ ID NO: 15, and a LCDR3 comprising the sequence of SEQ ID NO: 16;
c) a light chain variable region comprising a LCDR1 comprising the sequence of SEQ ID NO: 24, a LCDR2 comprising the sequence of SEQ ID NO: 25, and a LCDR3 comprising the sequence of SEQ ID NO: 26;
d) a light chain variable region comprising a LCDR1 comprising the sequence of SEQ ID NO: 34, a LCDR2 comprising the sequence of SEQ ID NO: 35, and a LCDR3 comprising the sequence of SEQ ID NO: 36;
e) a light chain variable region comprising a LCDR1 comprising the sequence of SEQ ID NO: 117, a LCDR2 comprising the sequence of SEQ ID NO: 35, and a LCDR3 comprising the sequence of SEQ ID NO: 118;
f) a light chain variable region comprising a LCDR1 comprising the sequence of SEQ ID NO: 121, a LCDR2 comprising the sequence of SEQ ID NO: 35, and a LCDR3 comprising the sequence of SEQ ID NO: 118;
g) a light chain variable region comprising a LCDR1 comprising the sequence of SEQ ID NO: 122, a LCDR2 comprising the sequence of SEQ ID NO: 35, and a LCDR3 comprising the sequence of SEQ ID NO: 118; and
h) a light chain variable region comprising a LCDR1 comprising the sequence of SEQ ID NO: 125, a LCDR2 comprising the sequence of SEQ ID NO: 35, and a LCDR3 comprising the sequence of SEQ ID NO: 118.

In certain embodiments, the antibody or an antigen-binding fragment thereof provided herein, wherein:
a) the heavy chain variable region comprises a HCDR1 comprising the sequence of SEQ ID NO: 1, a HCDR2 comprising the sequence of SEQ ID NO: 2, and a HCDR3 comprising the sequence of SEQ ID NO: 3; and the light chain variable region comprises a LCDR1 comprising the sequence of SEQ ID NO: 4, a LCDR2 comprising the sequence of SEQ ID NO: 5, and a LCDR3 comprising the sequence of SEQ ID NO: 6;
b) the heavy chain variable region comprises a HCDR1 comprising the sequence of SEQ ID NO: 11, a HCDR2 comprising the sequence of SEQ ID NO: 12, and a HCDR3 comprising the sequence of SEQ ID NO: 13; and the light chain variable region comprises a LCDR1 comprising the sequence of SEQ ID NO: 14, a LCDR2 comprising the sequence of SEQ ID NO: 15, and a LCDR3 comprising the sequence of SEQ ID NO: 16;
c) the heavy chain variable region comprises a HCDR1 comprising the sequence of SEQ ID NO: 21, a HCDR2 comprising the sequence of SEQ ID NO: 22, and a HCDR3 comprising the sequence of SEQ ID NO: 23; and the light chain variable region comprises a LCDR1 comprising the sequence of SEQ ID NO: 24, a LCDR2 comprising the sequence of SEQ ID NO: 25, and a LCDR3 comprising the sequence of SEQ ID NO: 26;
d) the heavy chain variable region comprises a HCDR1 comprising the sequence of SEQ ID NO: 31, a HCDR2 comprising the sequence of SEQ ID NO: 32, and a HCDR3 comprising the sequence of SEQ ID NO: 33; and the light chain variable region comprises a LCDR1 comprising the sequence of SEQ ID NO: 34, a LCDR2 comprising the sequence of SEQ ID NO: 35, and a LCDR3 comprising the sequence of SEQ ID NO: 36;
e) the heavy chain variable region comprises a HCDR1 comprising the sequence of SEQ ID NO: 114, a HCDR2 comprising the sequence of SEQ ID NO: 115, and a HCDR3 comprising the sequence of SEQ ID NO: 116; and the light chain variable region comprises a LCDR1 comprising the sequence of SEQ ID NO: 117, a LCDR2 comprising the sequence of SEQ ID NO: 35, and a LCDR3 comprising the sequence of SEQ ID NO: 118;
f) the heavy chain variable region comprises a HCDR1 comprising the sequence of SEQ ID NO: 119, a HCDR2 comprising the sequence of SEQ ID NO: 115, and a HCDR3 comprising the sequence of SEQ ID NO: 120; and the light chain variable region comprises a LCDR1 comprising the sequence of SEQ ID NO: 121, a LCDR2 comprising the sequence of SEQ ID NO: 35, and a LCDR3 comprising the sequence of SEQ ID NO: 118;
g) the heavy chain variable region comprises a HCDR1 comprising the sequence of SEQ ID NO: 119, a HCDR2 comprising the sequence of SEQ ID NO: 115, and a HCDR3 comprising the sequence of SEQ ID NO: 120; and the light chain variable region comprises a LCDR1 comprising the sequence of SEQ ID NO: 122, a LCDR2 comprising the sequence of SEQ ID NO: 35, and a LCDR3 comprising the sequence of SEQ ID NO: 118; and
h) the heavy chain variable region comprises a HCDR1 comprising the sequence of SEQ ID NO: 123, a HCDR2 comprising the sequence of SEQ ID NO: 115, and a HCDR3 comprising the sequence of SEQ ID NO: 124; and the light chain variable region comprises a LCDR1 comprising the sequence of SEQ ID NO: 125, a LCDR2 comprising the sequence of SEQ ID NO: 35, and a LCDR3 comprising the sequence of SEQ ID NO: 118.

In certain embodiments, the antibodies provided herein comprise one or more (e.g. 1, 2, 3, 4, 5, or 6) CDR sequences of anti-hGREM1 antibodies 14E3, 69H5, 22F1, 56C11, 36F5, 42B9 and 67G11.

“14E3” as used herein refers to a mouse antibody having a heavy chain variable region of SEQ ID NO: 7, and a light chain variable region of SEQ ID NO: 8.

“69H5” as used herein refers to a mouse antibody having a heavy chain variable region of SEQ ID NO: 27, and a light chain variable region of SEQ ID NO: 28.

“22F1” as used herein refers to a mouse antibody having a heavy chain variable region of SEQ ID NO: 17, and a light chain variable region of SEQ ID NO: 18.

“56C11” as used herein refers to a mouse antibody having a heavy chain variable region of SEQ ID NO: 37, and a light chain variable region of SEQ ID NO: 38.

“36F5” as used herein refers to a mouse antibody having a heavy chain variable region of SEQ ID NO: 126, and a light chain variable region of SEQ ID NO: 127.

“42B9” as used herein refers to a mouse antibody having a heavy chain variable region of SEQ ID NO: 128, and a light chain variable region of SEQ ID NO: 129.

“67G11” as used herein refers to a mouse antibody having a heavy chain variable region of SEQ ID NO: 128, and a light chain variable region of SEQ ID NO: 130.

Table 1 shows the CDR sequences of these anti-hGREM1 antibodies. The CDRs are determined according to Kabat numbering. A skilled person would understand that other known methods for CDR determination can also be applied to the antibodies provided herein, and those CDR sequences are also encompassed within the present disclosure. The heavy chain and light chain variable region sequences are also provided below in Table 2.

TABLE 1
Sequences of anti-hGREMI antibodies' CDR region

| Antibody | Region | CDR1 | CDR2 | CDR3 |
|---|---|---|---|---|
| 14E3 | HCDR | SEQ ID NO: 1 | SEQ ID NO: 2 | SEQ ID NO: 3 |
| | | TYGMA | WINTLSGEPTYADDF | EPMDY |
| | | | KG | |
| | LCDR | SEQ ID NO: 4 | SEQ ID NO: 5 | SEQ ID NO: 6 |
| | | KSSQSLLDSDGKTYL | LVSKLDS | WQGAHFPLT |
| | | S | | |
| 22F1 | HCDR | SEQ ID NO: 11 | SEQ ID NO: 12 | SEQ ID NO: 13 |
| | | DYYMN | DINPKDGDSGYSHKF | GFTTVVARGDY |
| | | | KG | |
| | LCDR | SEQ ID NO: 14 | SEQ ID NO: 15 | SEQ ID NO: 16 |
| | | KSSQSLLDSDGKTYL | LVSKLDS | WQGTHFPYT |
| | | N | | |
| 69H5 | HCDR | SEQ ID NO: 21 | SEQ ID NO: 22 | SEQ ID NO: 23 |
| | | DDYMH | WIDPENGDTEYASKF | WATVPDFDY |
| | | | QG | |
| | LCDR | SEQ ID NO: 24 | SEQ ID NO: 25 | SEQ ID NO: 26 |
| | | KSSQSLLNRSNQKNY | FTSTRES | QQHYSTPFT |
| | | LA | | |
| 56C11 | HCDR | SEQ ID NO: 31 | SEQ ID NO: 32 | DPIYYDYDEVA |
| | | DFYMN | DINPNNGGTSYNQKF | SEQ ID NO: 33 |
| | | | KG | Y |
| | LCDR | SEQ ID NO: 34 | SEQ ID NO: 35 | SEQ ID NO: 36 |
| | | RSSQSLVHSNGNTYL | KVSNRFS | SQSTHVPLT |
| | | H | | |
| 36F5 | HCDR | SEQ ID NO: 114 | SEQ ID NO: 115 | SEQ ID NO: 116 |
| | | SSGIG | EIYPRSGNTYNNEKF | EAYSHHYYAM |
| | | | KG | DY |
| | LCDR | SEQ ID NO: 117 | SEQ ID NO: 35 | SEQ ID NO: 118 |
| | | RSSQSLLHSNGNTYL | KVSNRFS | FQGSHVPFT |
| | | E | | |
| 42B9 | HCDR | SEQ ID NO: 119 | SEQ ID NO: 115 | EGYSNNYYAM |
| | | SYGIG | EIYPRSGNTYNNEKF | SEQ ID NO: 120 |
| | | | KG | DY |
| | LCDR | SEQ ID NO: 121 | SEQ ID NO: 35 | SEQ ID NO: 118 |
| | | ISSQSLVHSNGNTYL | KVSNRFS | FQGSHVPFT |
| | | E | | |
| 67G11 | HCDR | SEQ ID NO: 119 | SEQ ID NO: 115 | SEQ ID NO: 120 |
| | | SYGIG | EIYPRSGNTYNNEKF | EGYSNNYYAM |
| | | | KG | DY |
| | LCDR | SEQ ID NO: 122 | SEQ ID NO: 35 | SEQ ID NO: 118 |
| | | RSSQSLVHSNGNTYL | KVSNRFS | FQGSHVPFT |
| | | E | | |
| 36F5/ | HCDR | SEQ ID NO: 123 | | SEQ ID NO: 124 |
| 42B9/ | | SX₃₂GIG | | EX₂₇YSX₂₈X₂₉YY |
| 67G11 | | | | AMDY |
| | LCDR | SEQ ID NO: 125 | | |
| | | X₃₀SSQSLX₃₁HSNGNT | | |
| | | YLE | | |

Wherein X₃₂ is S or Y; X₂₇ is A or G; X₂₈ is H or N; X₂₉ is H or N; X₃₀ is R or I; X₃₁ is L or V.

TABLE 2
Sequences of mouse antibody VH/VL

| | VH | VL |
|---|---|---|
| 14E3 | SEQ ID NO: 7 | SEQ ID NO: 8 |
| | QIQLVQSGPELKKPGETVKISCKTS | DVVMTQTPLTLSITIGQPASISCKS |
| | GSTFTTYGMAWMKQAPGKGLTW | SQSLLDSDGKTYLSWLLQRPDQS |
| | MGWINTLSGEPTYADDFKGRFAFS | PKRLISLVSKLDSGVPDRITGSGS |
| | LKTSANTAYLQINNLKNEDAATYF | GTDFTLKISRVEAEDLGIYYCWQ |
| | CAREPMDYWGQGTSVIVSS | GAHFPLTFGAGTKLELK |
| 22F1 | SEQ ID NO: 17 | SEQ ID NO: 18 |
| | EAQLQQSGPELVKPGASVKISCKA | DVVMTQTPLTLSVTIGQPASISCK |
| | SGYSFTDYYMNWLKQSHGKSLE | SSQSLLDSDGKTYLNWLLQRPG |
| | WIGDINPKDGDSGYSHKFKGKAT | QSPKRLIYLVSKLDSGFPDRFTGS |
| | LTVDKSSSTAYMELRSLTSEDSAV | GSGTDFTLKISRVEAEDLGVYYC |
| | YYCASGFTTVVARGDYWGQGTTL | WQGTHFPYTFGGGTKLEIK |
| | TVSS | |
| 69H5 | SEQ ID NO: 27 | SEQ ID NO: 28 |
| | EVQLQQSGAELVRPGASVKLSCT | DIVMTQSPSSLAMSVGQKVTMS |
| | ASGFNIKDDYMHWVKRRPEQGL | CKSSQSLLNRSNQKNYLAWYQQ |
| | EWIGWIDPENGDTEYASKFQGKA | KPGQSPKLLVHFTSTRESGVPDR |
| | TITADTSSNTAYLQLSSLTSEDTAV | FIGSGSGTDFTLTISNLQAEDLAD |
| | YYCTTWATVPDFDYWGQGTTLTV | YFCQQHYSTPFTFGSGTKLEIK |
| | SS | |
| 56C11 | SEQ ID NO: 37 | SEQ ID NO: 38 |
| | EVQLQQSGPELVKPGASVKISCKA | DVVMTQTPLSLPVSLGDQASISC |
| | SGYTFTDFYMNWVKQSHGKSLE | RSSQSLVHSNGNTYLHWYLQKP |
| | WIGDINPNNGGTSYNQKFKGKAT | GQSPKLLIYKVSNRFSGVPDRFS |
| | LTVDKSSSTAYMELRSLTSEDSAV | GSGSGTDFTLKISRVEAEDLGVY |
| | YYCARDPIYYDYDEVAYWGQGTL | FCSQSTHVPLTFGAGTKLELK |
| | VTVSA | |
| 36F5 | SEQ ID NO: 126 | SEQ ID NO: 127 |
| | QVQLQQSGAELARPGASVKLSCK | DVLMTQTPLSLPVSLGGQASISC |
| | ASGYSFTSSGIGWVKQRSGQGLE | RSSQSLLHSNGNTYLEWYLQKP |
| | WIGEIYPRSGNTYNNEKFKGKATL | GQSPKLLIYKVSNRFSGVPDRLS |
| | TADKSSSTVYMELRSLTSEDSAVY | GSGSGTDFTLKISRVEAEDLGVY |
| | FCVREAYSHHYYAMDYWGQGTS | YCFQGSHVPFTFGSGTKLEIN |
| | VTVFS | |
| 42B9 | SEQ ID NO: 128 | SEQ ID NO: 129 |
| | QVQLQQSGAELARPGASVKLSCK | DVLMTQTPLSLPVSLGDQASISCI |
| | ASGYTFTSYGIGWVKQRTGQGLE | SSQSLVHSNGNTYLEWYLQKPG |
| | WIGEIYPRSGNTYNNEKFKGKATL | LSPKLLIYKVSNRFSGVPDRLSGS |
| | TADKSSRTVYMELRSLISEDSAVY | GSGTDFTLRISRVEAEDLGVYYC |
| | FCAREGYSNNYYAMDYWGQGTS | FQGSHVPFTFGSGTKLEIK |
| | VTVFS | |
| 67G11 | SEQ ID NO: 128 | SEQ ID NO: 130 |
| | QVQLQQSGAELARPGASVKLSCK | DVLMTQTPLSLPVSLGDQASISC |
| | ASGYTFTSYGIGWVKQRTGQGLE | RSSQSLVHSNGNTYLEWYLQKP |
| | WIGEIYPRSGNTYNNEKFKGKATL | GQSPKLLIYKVSNRFSGVPDRLS |
| | TADKSSRTVYMELRSLISEDSAVY | GSGSGTDFTLKISRVEAEDLGVY |
| | FCAREGYSNNYYAMDYWGQGTS | YCFQGSHVPFTFGSGTKLEIK |
| | VTVFS | |

The anti-hGREM1 antibodies or antigen-binding fragments thereof provided herein can be a monoclonal antibody, polyclonal antibody, humanized antibody, chimeric antibody, recombinant antibody, bispecific antibody, labeled antibody, bivalent antibody, or anti-idiotypic antibody. A recombinant antibody is an antibody prepared in vitro using recombinant methods rather than in animals.

CDRs are known to be responsible for antigen binding, however, it has been found that not all of the 6 CDRs are necessarily indispensable or unchangeable. In other words, it is possible to replace or change or modify 1, 2, or 3 CDRs in anti-hGREM1 antibodies 14E3, 69H5, 22F1, 56C11, 36F5, 42B9 or 67G11 (corresponding to any one of SEQ ID NOs: 1-36 and 114-125), yet substantially retain the specific binding affinity to hGREM1.

In certain embodiments, the anti-hGREM1 antibodies and the antigen-binding fragments provided herein comprise a heavy chain CDR3 sequence of one of the anti-hGREM1 antibodies 14E3, 69H5, 22F1, 56C11, 36F5, 42B9 or 67G11. In certain embodiments, the anti-hGREM1 antibodies and the antigen-binding fragments provided herein comprise a heavy chain CDR3 sequence of SEQ ID NOs: 3, 13, 23, 33, 116, 120 and 124. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, etc. J Mol Biol. 263:551-67).

In some embodiments, the anti-hGREM1 antibodies and the antigen-binding fragments provided herein comprise all or a portion of the heavy chain variable domain and/or all or a portion of the light chain variable domain. In one embodiment, the anti-hGREM1 antibodies and the antigen-binding fragments provided herein is a single domain antibody which consists of all or a portion of the heavy chain variable domain provided herein. More information of such a single domain antibody is available in the art (see, e.g., U.S. Pat. No. 6,248,516).

In certain embodiments, the antibodies and antigen-binding fragments thereof provided herein comprise suitable framework region (FR) sequences, as long as the antibodies and antigen-binding fragments thereof can specifically bind to GREM1. The CDR sequences provided in Table 1 are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are chimeric. The VH/VL are grafted to human IgG1 and Human Kappa sequences.

In certain embodiments, the antibodies and antigen-binding fragments thereof provided herein are humanized. A humanized antibody or antigen-binding fragment is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al. (1986) Nature 321:522-525; Riechmann et al. (1988) Nature 332:323-327; Verhoeyen et al. (1988) Science 239:1534-1536). Simulation of the three dimensional structure of variable regions or domains of the parent non-human antibody can be performed before or after this.

Suitable human heavy chain and light chain variable domains can be selected to achieve CDR grafting using methods known in the art. In an illustrative example, “best-fit” approach can be used, where a non-human (e.g., rodent) antibody variable domain sequence is screened or BLASTed against a database (e.g., Protein Data Bank, http://www.rcsb.org/) of known human variable domain germline sequences, and the human sequence closest to the non-human query sequence is identified and used as the human scaffold for grafting the non-human CDR sequences (see, for example, Sims et al, (1993) J. Immunol. 151:2296; Chothia et al. (1987) J. Mot. Biol. 196:901). Alternatively, a framework derived from the consensus sequence of all human antibodies may be used for the grafting of the non-human CDRs (see, for example, Carter et al. (1992) Proc. Natl. Acad. Sci. USA, 89:4285; Presta et al. (1993) J. Immunol., 151:2623).

In certain embodiments, the humanized antibodies or antigen-binding fragments provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment comprise human heavy/light chain FR1-4.

Table 3 below shows the HFR amino acid sequences of the humanized antibodies for 22F1, 14E3 and 56C11.

| Antibody | | FR1 | FR2 | FR3 | FR4 |
|---|---|---|---|---|---|
| hu22F1 | Ha | SEQ ID NO: 73 | SEQ ID NO: | SEQ ID NO: 75 | SEQ ID NO: |
| | | QVQLVQSGAEVKKPGAS | 74 | RVTMTRDtSTSTVY | 76 |
| | | VKVSCKASGYSFT | WVRQAPGQG | MELSSLRSEDTAVYY | WGQGTTVTVSS |
| | | | LEWMG | CAS | |
| | Hb | SEQ ID NO: 73 | SEQ ID NO: | SEQ ID NO: 78 | SEQ ID NO: |
| | | QVQLVQSGAEVKKPGAS | 74 | RVTMTVDKSTSTVY | 76 |
| | | VKVSCKASGYSFT | WVRQAPGQG | MELSSLRSEDTAVYY | WGQGTTVTVSS |
| | | | LEWMG | CAS | |
| | Hc | SEQ ID NO: 77 | SEQ ID NO: | SEQ ID NO: 79 | SEQ ID NO: |
| | | QAQLVQSGAEVKKPGAS | 74 | RVTLTVDKSTSTVY | 76 |
| | | VKVSCKASGYSFT | WVRQAPGQG | MELRSLRSEDTAVYY | WGQGTTVTVSS |
| | | | LEWMG | CAS | |
| | Hd | SEQ ID NO: 77 | SEQ ID NO: | SEQ ID NO: 81 | SEQ ID NO: |
| | | QAQLVQSGAEVKKPGAS | 80 | RATLTVDKSTSTVY | 76 |
| | | VKVSCKASGYSFT | WLRQAPGQG | MELRSLRSEDTAVYY | WGQGTTVTVSS |
| | | | LEWIG | CAS | |
| hu14E3 | Ha | SEQ ID NO: 82 | SEQ ID NO: | SEQ ID NO: 84 | SEQ ID NO: |
| | | QVQLVQSGSELKKPGAS | 83 | RFVFSLDTSVSTAYL | 85 |
| | | VKVSCKASGYTFT | WMRQAPGQ | QISSLKAEDTAVYYC | WGQGTMVTVS |
| | | | GLEWMG | AR | S |
| | Hb | SEQ ID NO: 86 | SEQ ID NO: | SEQ ID NO: 87 | SEQ ID NO: |
| | | QIQLVQSGSELKKPGASV | 83 | RFAFSLDTSVSTAYL | 85 |
| | | KVSCKASGYTFT | WMRQAPGQ | QISSLKAEDTAVYYC | WGQGTMVTVS |
| | | | GLEWMG | AR | S |
| | Hc | SEQ ID NO: 88 | SEQ ID NO: | SEQ ID NO: 87 | SEQ ID NO: |
| | | QIQLVQSGSELKKPGASV | 89 | RFAFSLDTSVSTAYL | 85 |
| | | KVSCKASGSTFT | WMKQAPGQ | QISSLKAEDTAVYYC | WGQGTMVTVS |
| | | | GLTWMG | AR | S |
| hu56C11 | H0 | SEQ ID NO: 90 | SEQ ID NO: | SEQ ID NO: 91 | SEQ ID NO: |
| | | QVQLVQSGAEVKKPGAS | 74 | RVTMTRDTSISTAY | 92 |
| | | VKVSCKASGYTFT | WVRQAPGQG | MELSRLRSDDTAVY | WGQGTLVTVSS |
| | | | LEWMG | YCAR | |
| | Ha | SEQ ID NO: 90 | SEQ ID NO: | SEQ ID NO: 93 | SEQ ID NO: |
| | | QVQLVQSGAEVKKPGAS | 74 | RVTMTVDKSISTAY | 92 |
| | | VKVSCKASGYTFT | WVRQAPGQG | MELSRLRSDDTAVY | WGQGTLVTVSS |
| | | | LEWMG | YCAR | |
| | Hb | SEQ ID NO: 90 | SEQ ID NO: | SEQ ID NO: 94 | SEQ ID NO: |
| | | QVQLVQSGAEVKKPGAS | 95 | RVTLTVDKSISTAYM | 92 |
| | | VKVSCKASGYTFT | WVRQAPGQG | ELSRLRSDDTAVYYC | WGQGTLVTVSS |
| | | | LEWIG | AR | |
| | Hc | SEQ ID NO: 90 | SEQ ID NO: | SEQ ID NO: 94 | SEQ ID NO: |
| | | QVQLVQSGAEVKKPGAS | 96 | RVTLTVDKSISTAYM | 92 |
| | | VKVSCKASGYTFT | WVKQAPGKG | ELSRLRSDDTAVYYC | WGQGTLVTVSS |
| | | | LEWIG | AR | |
| | | SEQ ID NO: 97 | SEQ ID NO: | hu22F1 (SEQ ID | SEQ ID NO: |
| | | QX₁QLVQSGX₂EX₃KKPG | 98 | NO: 99) | 100 |
| | | ASVKVSCKASGX₄X₅FT | WX₆X₇QAPGX₈ | RX₁₁TX₁₂TX₁₃DX₁₄S | WGQGTX₂₀VTV |
| | | | GLX₉WX₁₀G | TSTVYMELX₁₅SLRS | SS |
| | | | | EDTAVYYCAS | |
| | | | | hu14E3 (SEQ ID | |
| | | | | NO: 140) | |
| | | | | RFX₁₆FSLDTSVSTAY | |
| | | | | LQISSLKAEDTAVYY | |
| | | | | CAR | |
| | | | | hu56C11 (SEQ ID | |
| | | | | NO: 141) | |
| | | | | RVTX₁₇TX₁₈DX₁₉SIS | |
| | | | | TAYMELSRLRSDDTA | |
| | | | | VYYCAR | |

Table 4 below shows the LFR amino acid sequences of the humaniized antibodies for 22F1, 14E3 and 56C11

| | | | | | |
|---|---|---|---|---|---|
| hu22F1 | La | SEQ ID NO: 101 | SEQ ID NO: | SEQ ID NO: 103 | SEQ ID NO: |
| | | DVVMTQSPLSLPVTLGQ | 102 | GVPDRFSGSGSGTD | 104 |
| | | PASISC | WLQQRPGQS | FTLKISRVEAEDVGV | FGGGTKVEIK |
| | | | PRRLIY | YYC | |
| | Lb | SEQ ID NO: 101 | SEQ ID NO: | SEQ ID NO: 105 | SEQ ID NO: |
| | | DVVMTQSPLSLPVTLGQ | 102 | GFPDRFSGSGSGTD | 104 |
| | | PASISC | WLQQRPGQS | FTLKISRVEAEDVGV | FGGGTKVEIK |
| | | | PRRLIY | YYC | |
| hu14E3 | La | SEQ ID NO: 101 | SEQ ID NO: | SEQ ID NO: 103 | SEQ ID NO: |
| | | DVVMTQSPLSLPVTLGQ | 102 | GVPDRFSGSGSGTD | 106 |
| | | PASISC | WLQQRPGQS | FTLKISRVEAEDVGV | FGQGTKLEIK |
| | | | PRRLIY | YYC | |
| | Lb | SEQ ID NO: 101 | SEQ ID NO: | SEQ ID NO: 103 | SEQ ID NO: |
| | | DVVMTQSPLSLPVTLGQ | 107 | GVPDRFSGSGSGTD | 106 |
| | | PASISC | WLQQRPGQS | FTLKISRVEAEDVGV | FGQGTKLEIK |
| | | | PRRLIS | YYC | |
| hu56C11 | L0 | SEQ ID NO: 101 | SEQ ID NO: | SEQ ID NO: 103 | SEQ ID NO: |
| | | DVVMTQSPLSLPVTLGQ | 108 | GVPDRFSGSGSGTD | 106 |
| | | PASISC | WFQQRPGQS | FTLKISRVEAEDVG | FGQGTKLEIK |
| | | | PRRLIY | VYYC | |
| | La | SEQ ID NO: 101 | SEQ ID NO: | SEQ ID NO: 103 | SEQ ID NO: |
| | | DVVMTQSPLSLPVTLGQ | 109 | GVPDRFSGSGSGTD | 106 |
| | | PASISC | WFQQRPGQS | FTLKISRVEAEDVGV | FGQGTKLEIK |
| | | | PRLLIY | YYC | |
| | Lb | SEQ ID NO: 101 | SEQ ID NO: | SEQ ID NO: 103 | SEQ ID NO: |
| | | DVVMTQSPLSLPVTLGQ | 110 | GVPDRFSGSGSGTD | 106 |
| | | PASISC | WYQQRPGQS | FTLKISRVEAEDVGV | FGQGTKLEIK |
| | | | PRLLIY | YYC | |
| | | | SEQ ID NO: | SEQ ID NO: 112 | SEQ ID NO: |
| | | | 111 | GX₂₄PDRFSGSGSGT | 113 |
| | | | WX₂₁QQRPG | DFTLKISRVEAEDVG | FGX₂₅GTKX₂₆E |
| | | | QSPRX₂₂LIX₂₃ | VYYC | IK |

Wherein X₁ is V, I or A; X₂ is A or S; X₃ is V or L; X₄ is Y or S; X₅ is T or S; X₆ is V L, or M; X₇ is R or K; X₈ is Q or K; X₉ is E or T; X₁₀ is M, or I; X₁₁ is V or A; X₁₂ is M or L; X₁₃ is R or V; X₁₄ is T or K; X₁₅ is S or R; X₁₆ is V or A; X₁₇ is M or L; X₁₈ is R or V; X₁₉ is T or K; X₂₀ is T, M or L; X₂₁ is L, F or Y; X₂₂ is R or L; X₂₃ is Y or S; X₂₄ is V or F; X₂₅ is G or Q; X₂₆ is V or L.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure to reduce or avoid immunogenicity and/or improve or retain the binding activity or binding affinity.

In certain embodiments, the humanized antibody or antigen-binding fragment provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FRs of a heavy or a light chain variable domain. In some embodiments, such change in amino acid residue could be present in heavy chain FR regions only, in light chain FR regions only, or in both chains. In certain embodiments, the one or more amino acid residues are mutated, for example, back-mutated to the corresponding residue found in the non-human parent antibody (e.g. in the mouse framework region) from which the CDR sequences are derived. Suitable positions for mutations can be selected by a skilled person following principles known in the art. For example, a position for mutation can be selected where: 1) the residue in the framework of the human germline sequence is rare (e.g. in less than 20% or less than 10% in human variable region sequence); 2) the position is immediately adjacent to one or more of the 3 CDR's in the primary sequence of the human germline chain, as it is likely to interact with residues in the CDRs; or 3) the position is close to CDRs in a 3-dimensional model, and therefore can have a good probability of interacting with amino acids in the CDR. The residue at the selected position can be mutated back to the corresponding residue in the parent antibody, or to a residue which is neither the corresponding residue in human germline sequence nor in parent antibody, but to a residue typical of human sequences, i.e. that occurs more frequently at that position in the known human sequences belonging to the same subgroup as the human germline sequence (see U.S. Pat. No. 5,693,762). In certain embodiments, for hul4E3, the heavy chain variants Ha, Hb and He were obtained by direct grafting the three CDRs to their germline sequence with the back mutation of V37M for heavy chain variant Ha, back mutations of V37M, V68A, V2I for heavy chain variant Hb, and back mutations of V37M, V68A, V2I, Y27S, R38K, E46T for heavy chain variant He, respectively, all based on Kabat numbering; the light chain variants were obtained by direct grafting the three CDRs to their germline sequence with back mutation of F36L for light chain variant La and back mutations of F36L, Y49S, for light chain variant Lb, respectively, all based on Kabat numbering. In certain embodiments, for hu22F1, the heavy chain variants Ha, Hb, He and Hd were obtained by direct grafting the three CDRs to their germline sequence without back mutation for heavy chain variant Ha, back mutations of R72V, T74K, T28S, R98S for heavy chain variant Hb, back mutations of R71V, T73K, T28S, R94S, S82AR, M69L, V2A for heavy chain variant Hc, and back mutations of R71V, T73K, T28S, R94S, S82AR, M69L, V2A, V67A, M48I, V37L for heavy chain variant Hd, respectively, all based on Kabat numbering; the light chain variants were obtained by direct grafting the three CDRs to their germline sequence with back mutation of F36L for light chain variant La and back mutations of F36L, V58F for light chain variant Lb, respectively, all based on Kabat numbering. In certain embodiments, for hu56C11, heavy chain (HC) variants 1, 2, 3 and 4 were obtained by direct grafting the three CDRs to the germline sequence without back mutation for heavy chain variant H0, back mutation of R71V, T73K for heavy chain variant Ha, back mutation of R71V, T73K, M69L, M48I for heavy chain variant Hb, and back mutation of R71V, T73K, M69L, M48I, Q43K, R38K, for heavy chain variant Hc, respectively, all based on Kabat numbering; light chain (LC) vairant 1, 2 and 3 were obtained by direct grafting the three CDRs to germline sequence without back mutation for light chain variant L0, R46L for light chain variant La, and F36Y, R46L, for light chain variant Lb, respectively, all based on Kabat numbering.

In certain embodiments, the humanized light and heavy chains of the present disclosure are substantially non-immunogenic in humans and retain substantially the same affinity as or even higher affinity than the parent antibody to hGREM1. Specifically, humanized antibodies provided herein (e.g., Hu22F1 and Hu56C11) and the humanized anti-hGREM1 antibodies derived from 69H5, 36F5, 42B9 and 67G11 are expected to show similar properties (e.g., high binding affinity to human gremlin 1 and/or mouse gremlin 1, high blocking effect on gremlin mediated inhibition on BMP4 signaling, high blocking activity of binding of gremlin 1 to FGFR1, and/or high anti-tumor effect) or even better than, the parent antibodies thereof.

In certain embodiments, the humanized antibodies and antigen-binding fragment thereof provided herein comprise one or more light chain FR sequences of human germline framework sequence IGKV/2-30, and/or one or more heavy chain FR sequences of human germline framework sequence IGHV/7-4 or IGHV/1-46 or IGHV1-2, with or without back mutations. Back mutations can be introduced in to the human germline framework sequence, if needed.

In certain embodiments, the humanized antibody or antigen-binding fragment provided herein comprises a heavy chain variable region comprising a sequence selected from the group consisting of SEQ ID NO: 41, SEQ ID NO: 43, SEQ ID NO: 45, SEQ ID NO: 51, SEQ ID NO: 53, SEQ ID NO: 55, SEQ ID NO: 57, SEQ ID NO: 131, SEQ ID NO: 132, SEQ ID NO: 133 and SEQ ID NO: 134, and a homologous sequence thereof having at least 80% (e.g. at least 85%, 90%, 95%, 96%, 97%, 98%, or 99%) sequence identity yet retaining specific binding specificity or affinity to hGREM1.

In certain embodiments, the humanized antibody or an antigen-binding fragment thereof provided herein further comprises a light chain variable region comprising a sequence selected from the group consisting of SEQ ID NO: 47, SEQ ID NO: 49, SEQ ID NO: 59, SEQ ID NO: 61, SEQ ID NO: 135, SEQ ID NO: 136 and SEQ ID NO: 137 and a homologous sequence thereof having at least 80% (e.g. at least 85%, 90%, 95%, 96%, 97%, 98%, or 99%) sequence identity yet retaining specific binding specificity or affinity to hGREM1.

In certain embodiments, the humanized antibody or antigen-binding fragment provided herein comprises a heavy chain variable region comprising a sequence selected from the group consisting of SEQ ID NO: 41, SEQ ID NO: 43, and SEQ ID NO: 45, and a homologous sequence thereof having at least 80% (e.g. at least 85%, 90%, 95%, 96%, 97%, 98%, or 99%) sequence identity yet retaining specific binding specificity or affinity to hGREM1, and a light chain variable region comprising a sequence selected from the group consisting of SEQ ID NO: 47, and SEQ ID NO: 49, and a homologous sequence thereof having at least 80% (e.g. at least 85%, 90%, 95%, 96%, 97%, 98%, or 99%) sequence identity yet retaining specific binding specificity or affinity to hGREM1.

In certain embodiments, the humanized antibody or an antigen-binding fragment thereof provided herein further comprises a pair of heavy chain variable region and light chain variable region sequences selected from the group consisting of: SEQ ID NOs: 41/47, 41/49, 43/47, 43/49, 45/47, and 45/49, or a pair of sequences having at least 80% (e.g. at least 85%, 90%, 95%, 96%, 97%, 98%, or 99%) sequence identity thereof yet retaining specific binding specificity or affinity to hGREM1.

In certain embodiments, the humanized antibody or antigen-binding fragment provided herein comprises a heavy chain variable region comprising a sequence selected from the group consisting of SEQ ID NO: 51, SEQ ID NO: 53, SEQ ID NO: 55 and SEQ ID NO: 57, and a homologous sequence thereof having at least 80% (e.g. at least 85%, 90%, 95%, 96%, 97%, 98%, or 99%) sequence identity yet retaining specific binding specificity or affinity to hGREM1, and a light chain variable region comprising a sequence selected from the group consisting of SEQ ID NO: 59 and SEQ ID NO: 61, and a homologous sequence thereof having at least 80% (e.g. at least 85%, 90%, 95%, 96%, 97%, 98%, or 99%) sequence identity yet retaining specific binding specificity or affinity to hGREM1.

In certain embodiments, the humanized antibody or an antigen-binding fragment thereof provided herein further comprises a pair of heavy chain variable region and light chain variable region sequences selected from the group consisting of: SEQ ID NOs: 51/59, 51/61, 53/59, 53/61, 55/59, 55/61, 57/59, and 57/61, or a pair of sequences having at least 80% (e.g. at least 85%, 90%, 95%, 96%, 97%, 98%, or 99%) sequence identity thereof yet retaining specific binding specificity or affinity to hGREM1.

In certain embodiments, the humanized antibody or antigen-binding fragment provided herein comprises a heavy chain variable region comprising a sequence selected from the group consisting of SEQ ID NO: 131, SEQ ID NO: 132, SEQ ID NO: 133 and SEQ ID NO: 134, and a homologous sequence thereof having at least 80% (e.g. at least 85%, 90%, 95%, 96%, 97%, 98%, or 99%) sequence identity yet retaining specific binding specificity or affinity to hGREM1, and a light chain variable region comprising a sequence selected from the group consisting of SEQ ID NO: 135, SEQ ID NO: 136 and SEQ ID NO: 137, and a homologous sequence thereof having at least 80% (e.g. at least 85%, 90%, 95%, 96%, 97%, 98%, or 99%) sequence identity yet retaining specific binding specificity or affinity to hGREM1.

In certain embodiments, the humanized antibody or an antigen-binding fragment thereof provided herein further comprises a pair of heavy chain variable region and light chain variable region sequences selected from the group consisting of: SEQ ID NOs: 131/135, 131/136, 131/137, 132/135, 132/136, 132/137, 133/135, 133/136, 133/137, 134/135, 134/136, and 134/137, or a pair of sequences having at least 80% (e.g. at least 85%, 90%, 95%, 96%, 97%, 98%, or 99%) sequence identity thereof yet retaining specific binding specificity or affinity to hGREM1.

TABLE 5
Sequences of humanized 14E3 (Hu14E3), humanized
22F1 (Hu22F1) and humanized 56C11 (Hu56C11)

| Antibody chain | Sequences |
|---|---|
| Hu14E3-Ha VH | QVQLVQSGSELKKPGASVKVSCKASGYTFTTYGMAWMR |
| | QAPGQGLEWMGWINTLSGEPTYADDFKGRFVFSLDTSVS |
| | TAYLQISSLKAEDTAVYYCAREPMDYWGQGTMVTVSS |
| | (SEQ ID NO: 41) |
| Hu14E3-Hb VH | QIQLVQSGSELKKPGASVKVSCKASGYTFTTYGMAWMRQ |
| | APGQGLEWMGWINTLSGEPTYADDFKGRFAFSLDTSVST |
| | AYLQISSLKAEDTAVYYCAREPMDYWGQGTMVTVSS |
| | (SEQ ID NO: 43) |
| Hu14E3-Hc VH | QIQLVQSGSELKKPGASVKVSCKASGSTFTTYGMAWMKQ |
| | APGQGLTWMGWINTLSGEPTYADDFKGRFAFSLDTSVST |
| | AYLQISSLKAEDTAVYYCAREPMDYWGQGTMVTVSS |
| | (SEQ ID NO: 45) |
| Hu14E3-La VL | DVVMTQSPLSLPVTLGQPASISCKSSQSLLDSDGKTYLSW |
| | LQQRPGQSPRRLIYLVSKLDSGVPDRFSGSGSGTDFTLKIS |
| | RVEAEDVGVYYCWQGAHFPLTFGQGTKLEIK (SEQ ID |
| | NO: 47) |
| Hu14E3-Lb VL | DVVMTQSPLSLPVTLGQPASISCKSSQSLLDSDGKTYLSW |
| | LQQRPGQSPRRLISLVSKLDSGVPDRFSGSGSGTDFTLKISR |
| | VEAEDVGVYYCWQGAHFPLTFGQGTKLEIK (SEQ ID NO: |
| | 49) |
| Hu22F1-Ha VH | QVQLVQSGAEVKKPGASVKVSCKASGYSFTDYYMNWVR |
| | QAPGQGLEWMGDINPKDGDSGYSHKFKGRVTMTRDTSTS |
| | TVYMELSSLRSEDTAVYYCASGFTTVVARGDYWGQGTT |
| | VTVSS (SEQ ID NO: 51) |
| Hu22F1-Hb VH | QVQLVQSGAEVKKPGASVKVSCKASGYSFTDYYMNWVR |
| | QAPGQGLEWMGDINPKDGDSGYSHKFKGRVTMTVDKST |
| | STVYMELSSLRSEDTAVYYCASGFTTVVARGDYWGQGTT |
| | VTVSS (SEQ ID NO: 53) |
| Hu22F1-Hc VH | QAQLVQSGAEVKKPGASVKVSCKASGYSFTDYYMNWVR |
| | QAPGQGLEWMGDINPKDGDSGYSHKFKGRVTLTVDKSTS |
| | TVYMELRSLRSEDTAVYYCASGFTTVVARGDYWGQGTT |
| | VTVSS (SEQ ID NO: 55) |
| Hu22F1-Hd VH | QAQLVQSGAEVKKPGASVKVSCKASGYSFTDYYMNWLR |
| | QAPGQGLEWIGDINPKDGDSGYSHKFKGRATLTVDKSTST |
| | VYMELRSLRSEDTAVYYCASGFTTVVARGDYWGQGTTV |
| | TVSS (SEQ ID NO: 57) |
| Hu22F1-La VL | DVVMTQSPLSLPVTLGQPASISCKSSQSLLDSDGKTYLNW |
| | LQQRPGQSPRRLIYLVSKLDSGVPDRFSGSGSGTDFTLKIS |
| | RVEAEDVGVYYCWQGTHFPYTFGGGTKVEIK (SEQ ID |
| | NO: 59) |
| Hu22F1-Lb VL | DVVMTQSPLSLPVTLGQPASISCKSSQSLLDSDGKTYLNW |
| | LQQRPGQSPRRLIYLVSKLDSGFPDRFSGSGSGTDFTLKISR |
| | VEAEDVGVYYCWQGTHFPYTFGGGTKVEIK (SEQ ID NO: |
| | 61) |
| Hu56C11-H0 VH | QVQLVQSGAEVKKPGASVKVSCKASGYTFTDFYMNWVR |
| | QAPGQGLEWMGDINPNNGGTSYNQKFKGRVTMTRDTSIS |
| | TAYMELSRLRSDDTAVYYCARDPIYYDYDEVAYWGQGTL |
| | VTVSS (SEQ ID NO: 131) |
| Hu56C11-Ha VH | QVQLVQSGAEVKKPGASVKVSCKASGYTFTDFYMNWVR |
| | QAPGQGLEWMGDINPNNGGTSYNQKFKGRVTMTVDKSIS |
| | TAYMELSRLRSDDTAVYYCARDPIYYDYDEVAYWGQGTL |
| | VTVSS (SEQ ID NO: 132) |
| Hu56C11-Hb VH | QVQLVQSGAEVKKPGASVKVSCKASGYTFTDFYMNWVR |
| | QAPGQGLEWIGDINPNNGGTSYNQKFKGRVTLTVDKSIST |
| | AYMELSRLRSDDTAVYYCARDPIYYDYDEVAYWGQGTLV |
| | TVSS (SEQ ID NO: 133) |
| Hu56C11-Hc VH | QVQLVQSGAEVKKPGASVKVSCKASGYTFTDFYMNWVK |
| | QAPGKGLEWIGDINPNNGGTSYNQKFKGRVTLTVDKSIST |
| | AYMELSRLRSDDTAVYYCARDPIYYDYDEVAYWGQGTLV |
| | TVSS (SEQ ID NO: 134) |
| Hu56C11-L0 VL | DVVMTQSPLSLPVTLGQPASISCRSSQSLVHSNGNTYLHW |
| | FQQRPGQSPRRLIYKVSNRFSGVPDRFSGSGSGTDFTLKIS |
| | RVEAEDVGVYYCSQSTHVPLTFGQGTKLEIK (SEQ ID NO: |
| | 135) |
| Hu56C11-La VL | DVVMTQSPLSLPVTLGQPASISCRSSQSLVHSNGNTYLHWF |
| | QQRPGQSPRLLIYKVSNRFSGVPDRFSGSGSGTDFTLKISR |
| | VEAEDVGVYYCSQSTHVPLTFGQGTKLEIK (SEQ ID NO: |
| | 136) |
| Hu56C11-Lb VL | DVVMTQSPLSLPVTLGQPASISCRSSQSLVHSNGNTYLHW |
| | YQQRPGQSPRLLIYKVSNRFSGVPDRFSGSGSGTDFTLKIS |
| | RVEAEDVGVYYCSQSTHVPLTFGQGTKLEIK (SEQ ID NO: |
| | 137) |

The humanized anti-hGREM1 antibodies provided herein retained the specific binding affinity to hGREM1, and are at least comparable to, or even better than, the parent antibodies in that aspect. In certain embodiments, the anti-hGREM1 antibodies and the fragments thereof provided herein further comprise an immunoglobulin constant region, optionally a constant region of human Ig, or optionally a constant region of human IgG. In some embodiments, an immunoglobulin constant region comprises a heavy chain and/or a light chain constant region. The heavy chain constant region comprises CH1, hinge, and/or CH2-CH3 regions. In certain embodiments, the heavy chain constant region comprises an Fc region. In certain embodiments, the light chain constant region comprises Cx or Ck.

In certain embodiments, the anti-hGREM1 antibodies and the fragments thereof provided herein further comprise a constant region of human IgG1, IgG2, IgG3, or IgG4. In certain embodiments, the anti-hGREM1 antibodies and antigen-binding fragments thereof provided herein comprises a constant region of IgG1 isotype. In certain embodiments, the anti-hGREM1 antibodies and antigen-binding fragments thereof provided herein comprises a constant region of IgG2b isotype.

In certain embodiments, the humanized antibodies provided herein may comprise the heavy chain variable region fused to the constant region of human IgG1 isotype and the light chain variable region fused to the constant region of human kappa chain.

### Antibody Variants

The anti-hGREM1 antibodies and antigen-binding fragments thereof provided herein also encompass various types of variants of the antibody sequences provided herein.

In certain embodiments, the variants comprise one or more modification(s) or substitution(s) in 1, 2, or 3 CDR sequences as provided in Table 1, in one or more FR sequences, in the heavy or light chain variable region sequences provided herein, and/or in the constant region (e.g., Fc region). Such antibody variants retain specific binding affinity to hGREM1 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, reduced or increased effector function(s), improved FcRn receptor binding, increased pharmacokinetic half-life, pH sensitivity, and/or compatibility to conjugation (e.g., one or more introduced cysteine residues), to name a few.

A parent antibody sequence may be screened to identify suitable or preferred residues to be modified or substituted, using methods known in the art, for example “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

i. Affinity Variant

An affinity variant retain specific binding affinity to hGREM1 of the parent antibody, or even have improved hGREM1 specific binding affinity over the parent antibody. Various methods known in the art can be used to achieve this purpose. For example, a library of antibody variants (such as Fab or scFv variants) can be generated and expressed with phage display technology, and then screened for the binding affinity to human GREM1. For another example, computer software can be used to virtually simulate the binding of the antibodies to human GREM1, and identify the amino acid residues on the antibodies which form the binding interface. Such residues may be either avoided in the substitution so as to prevent reduction in binding affinity, or targeted for substitution to provide for a stronger binding.

In certain embodiments, at least one (or all) of the substitution(s) in the CDR sequences, FR sequences, or variable region sequences comprises a conservative substitution.

In certain embodiments, the anti-hGREM1 antibodies or antigen-binding fragments provided herein comprise one or more amino acid residue substitutions in one or more CDR sequences, and/or one or more FR sequences. In certain embodiments, an affinity variant comprises no more than 10, 9, 8, 7, 6, 5, 4, 3, 2, or 1 substitutions in one or more of the CDR sequences and/or FR sequences in total.

In certain embodiments, the anti-hGREM1 antibodies and antigen-binding fragments thereof comprise 1, 2, or 3 CDR sequences having at least 80% (e.g., at least 85%, 88%, 90%, 91%, 92%, 93%, 94%, 95%, 96%, 97%, 98%, 99%) sequence identity to that (or those) listed in Table 1, and in the meantime retain the binding affinity to hGREM1 at a level similar to or even higher than its parental antibody.

ii. Glycosylation Variant

The anti-hGREM1 antibodies and antigen-binding fragments provided herein also encompass a glycosylation variant, which can be obtained to either increase or decrease the extent of glycosylation of the antibody or antigen binding fragment. The term “glycosylation” as used herein, refers to enzymatic process that attaches glycans such as fucose, xylose, mannose, or GlcNAc phosphoserine glycan to proteins, lipids, or other organic molecules. Depending on the carbon linked to the glycan, glycosylation can be divided into five classes including: N-linked glycosylation, O-linked glycosylation, phospho-glycosylation, C-linked glycosylation, and glypiation.

Glycosylation of antibodies is typically N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine.

In certain embodiments, the antibody glycosylation variants can be obtained by, for example, removal of a native glycosylation site (e.g. by N297A substitution), such that tripeptide sequences for N-linked glycosylation sites or serine or threonine residues for O-linked glycosylation sites no longer present in the antibody or Fc sequence. Alternatively, in certain embodiments, antibody glycosylation variants can be obtained by producing the antibody in a host cell line that is defective in adding the selected sugar group(s) to the mature core carbohydrate structure in the antibody.

iii. Cysteine-Engineered Variant

The anti-hGREM1 antibodies and antigen-binding fragments provided herein also encompass a cysteine-engineered variant, which comprises one or more introduced free cysteine amino acid residues.

A free cysteine residue is one which is not part of a disulfide bridge. A cysteine-engineered variant is useful for conjugation with, for example a cytotoxic and/or imaging compound, a label, or a radioisoptype among others, at the site of the engineered cysteine, through for example a maleimide or haloacetyl. Methods for engineering antibodies or antigen-binding fragments to introduce free cysteine residues are known in the art, see, for example, WO2006/034488.

### Antigen-Binding Fragments

Provided herein are also anti-hGREM1 antigen-binding fragments. Various types of antigen-binding fragments are known in the art and can be developed based on the anti-hGREM1 antibodies provided herein, including for example, the exemplary antibodies whose CDR sequences are shown in Tables 1, and their different variants (such as affinity variants, glycosylation variants, Fe variants, cysteine-engineered variants and so on).

In certain embodiments, an anti-hGREM1 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. Coli (e.g., for Fab, Fv and ScFv antibody fragments), screening from a phage display library as discussed above (e.g., for ScFv), and chemical coupling of two Fab′-SH fragments to form F(ab′)₂ fragments (Carter et al., Bio/Technology 10:163-167 (1992)). Other techniques for the production of antibody fragments will be apparent to a skilled practitioner.

In certain embodiments, the antigen-binding fragment is a scFv. Generation of scFv is described in, for example, WO 93/16185; U.S. Pat. Nos. 5,571,894; and 5,587,458. scFv may be fused to an effector protein at either the amino or the carboxyl terminus to provide for a fusion protein (see, for example, Antibody Engineering, ed. Borrebaeck).

In certain embodiments, the anti-hGREM1 antibodies and antigen-binding fragments thereof provided herein are bivalent, tetravalent, hexavalent, or multivalent. The term “valent” as used herein refers to the presence of a specified number of antigen binding sites in a given molecule. As such, the terms “bivalent”, “tetravalent”, and “hexavalent” denote the presence of two binding site, four binding sites, and six binding sites, respectively, in an antigen-binding molecule. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e. having the same sequences), or structurally different (i.e. having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Epitope

In another aspect, the present disclosure provides antibodies that bind to the same epitope to which the antibody or antigen-binding fragment thereof provided herein binds. In another aspect, the present disclosure provides antibodies that competes for binding to hGREM1 with the antibody or antigen-binding fragment thereof provided herein.

The term “epitope” as used herein refers to the specific group of atoms or amino acids on an antigen to which an antibody binds. An epitope can include specific amino acids, sugar side chains, phosphoryl or sulfonyl groups that directly contact an antibody. Those skilled in the art will recognize that it is possible to determine, without undue experimentation, if an antibody binds to the same or overlapping or adjacent epitope as the antibody of present disclosure (e.g., hybridoma/chimeric or humanized antibodies 14E3, 69H5, 22F1, 56C11, 36F5, 42B9 and 67G11 and any of the chimeric and humanized variant thereof provided herein) by ascertaining whether the two competes for binding to a GREM1 antigen polypeptide.

The term “compete for binding” as used herein with respect to two antigen-binding proteins (e.g. antibodies), means that one antigen-binding protein blocks or reduces binding of the other to the antigen (e.g., human/mouse GREM1), as determined by a competitive binding assay. Competitive binding assays are well known in the art, include, for example, direct or indirect radioimmunoassay (RIA), direct or indirect enzyme immunoassay (EIA), Fortebio, competition ELISA assay, and sandwich competition assay (see, e.g., Stahli et al., 1983, Methods in Enzymology 9:242-253). Typically, such an assay involves the use of purified antigen bound to a solid surface or cells bearing the antigen, an unlabelled test antibody and a labeled reference antibody. Competitive inhibition is measured by determining the amount of label bound to the solid surface or cells in the presence of the test antibody. Usually the test antibody is present in excess. If two antibodies compete for binding to the hGREM1, then the two antibodies bind to the same or overlapping epitope, or an adjacent epitope sufficiently proximal to the epitope bound by the other antibody for steric hindrance to occur. Usually, when a competing antibody is present in excess, it will inhibit (e.g., reduce) specific binding of a test antibody to a common antigen by at least 50-55%, 55-60%, 60-65%, 65-70%, 70-75% 75-80%, 80-85%, 85-90% or more.

### Groups of Antibodies Categorized by Biological Properties

The antibodies provided herein have certain unique biological properties. Antibodies share certain unique biological properties can thus be categorized into multiple groups.

i) Antibodies with cancer cell-selective reduction of hGREM1-mediated inhibition on BMP signaling

In certain embodiments, the antibodies provided herein are capable of reducing hGREM1-mediated inhibition on BMP signaling selectively in a cancer cell over a non-cancer cell, and comprise the heavy chain CDR1 (HCDR1), HCDR2 and HCDR3 and light chain CDR1 (LCDR1), LCDR2, and LCDR3 of an antibody selected from the group consisting of 14E3, 22F1, 56C11, 69H5, 42B9, 36F5, and 67G11. These antibodies are particularly useful for methods of treating cancer.

The present disclosure unexpectedly found that the neutralization of GREM1 using certain anti-GREM1 antibodies provided herein selectively inhibit GREM1-mediated inhibition on BMP signaling in cancer cells, but does not show such inhibition or only show very limited inhibition in non-cancer cells.

Eliminating GREM1 and/or reducing the activity of GREM1 in cancer cells are preferred, as this will have an inhibitory impact on cancer cell proliferation and sphere formation, and also induce cancer cell apoptosis. However, inhibition of GREM1 in general could be undesirable, as conventional knockout of GREM1 in mice causes abnormal development of the intestinal tract and disorder of the hematopoietic system (Rowan, S. C. et al. Gremlin 1 depletion in vivo causes severe enteropathy and bone marrow failure. J Pathol 251, 117-122). This suggests that conventional elimination of GREM1 and/or reduction in its activity would inevitably cause detrimental effects on other normal tissues. In this regard, the unexpected cancer-specific regulation of GREM1-mediated inhibition on BMP signaling by the anti-GREM1 antibodies provided herein is advantageous by avoiding the undesired side effects on normal tissues.

In certain embodiments, the anti-GREM1 antibodies provided herein exhibits no more than 50%, 45%, 40%, 35%, 30%, 25%, 20%, 15%, 10% or 5% reduction of GREM1-mediated inhibition on BMP signaling in a non-cancer cell.

The phrase “BMP signaling” as used herein means signaling of one or more BMP ligands that can be inhibited by GREM1. In certain embodiments, BMP signaling is BMP-2 signaling and/or BMP-4 signaling.

The phrase “non-cancer cell” as used herein refers to cells that are not cancer cell. A non-cancer cell can be a cell line, a primary cell isolated from a subject.

GREM1-mediated inhibition on BMP signaling can be determined by measuring BMP signaling of the BMP ligand(s) in the presence and absence of GREM1, respectively, where the difference is indicative of the GREM1-mediated inhibition. An anti-GREM1 antibody, however, can reduce the GREM1-mediated inhibition on BMP signaling, or in other words, restore the BMP signaling. GREM1-mediated inhibition reduction or restoration of BMP signaling can be calculated as increase in BMP signaling in the presence of anti-GREM1 antibody relative to that in the absence thereof. Percentage of such reduction or such restoration can be calculated as the ratio of the reduction in the GREM1-mediated inhibition to the total GREM1-mediated inhibition. 100% reduction of GREM1-mediated inhibition on BMP signaling would mean the BMP signaling is restored to a level substantially the same as the level in the absence of GREM1, and 0% reduction would mean the BMP signaling is not restored.

ii) Antibodies that bind to a chimeric hGREM1 (i.e. XM5)

In certain embodiments, certain anti-GREM1 antibodies provided herein bind to GREM1 at an epitope outside of BMP-binding loop. In certain embodiments, the BMP-binding loop comprises an amino acid sequence of SEQ ID NO: 63.

In certain embodiments, anti-GREM1 antibodies provided herein are capable of binding to a chimeric GREM1 comprising an amino acid sequence of SEQ ID NO: 68 (also referred to herein as “XM5”). The chimeric GREM1 XM5 comprises a mutated version of hGREM1 in which the binding loop of BMPs (i.e. the 123^{rd}-143^{rd} amino acid residues of hGREM1 (NSFYIPRHIRKEEGSFQSCSF, SEQ ID NO: 63)) was replaced by the 63^{rd}-83^{rd} amino acids of DAN (FSYSVPNTFPQSTESLVHCDS, SEQ ID NO: 64), which does not bind to BMPs. Accordingly, this chimeric hGREM1 does not bind to BMPs. Certain existing anti-GREM1 antibodies cannot bind to this chimeric hGREM1, suggesting that their binding to hGREM1 requires the BMP-binding loop in hGREM1. In contrast, the anti-GREM1 antibodies provided herein are capable of binding to this chimeric hGREM1, indicating that they bind to hGREM1 at an epitope that is outside of this BMP-binding loop.

In certain embodiments, the antibodies provided herein are capable of binding to a chimeric hGREM1 comprising an amino acid sequence of SEQ ID NO: 68, and comprise the heavy chain CDR1 (HCDR1), HCDR2 and HCDR3 and light chain CDR1 (LCDR1), LCDR2, and LCDR3 of an antibody selected from the group consisting of 14E3, 42B9, 67G11, 36F5, 56C11, 22F1 and 69H5.

The benchmark antibody 6245P is not capable of binding to X5.

iii) Antibodies that have cross-reactivity or having no cross-reactivity to mouse GREM1

In certain embodiments, some of the anti-GREM1 antibodies provided herein are capable of binding to hGREM1 but not specifically binding to mouse GREM1.

hGREM1 and mouse GREM1 share a sequence identity of 98%, and the different amino acid residues are found only in the N-terminal portion of the hGREM1, including Gln27 and Asn33 of hGREM1, in which the residue numbering is according to SEQ ID NO: 69. Accordingly, for the anti-GREM1 antibodies that do not cross-react with mouse GREM1, it is expected that they could bind to hGREM1 at an epitope comprising Gln27 and/or Asn33 of hGREM1, in which the residue numbering is according to SEQ ID NO: 69, or binds to a hGREM1 fragment comprising residue Gln27 and/or residue Asn33, optionally the hGREM1 fragment has a length of at least 3 (e.g. 4, 5, 6, 7, 8, 9, or 10) amino acid residues. The epitope can be a conformation epitope or a linear epitope.

In certain embodiments, the antibodies provided herein are capable of binding to hGREM1 but not specifically binding to mouse gremlin1, and comprise the heavy chain CDR1 (HCDR1), HCDR2 and HCDR3 and light chain CDR1 (LCDR1), LCDR2, and LCDR3 of an antibody selected from the group consisting of 69H5, 22F1, and 14E3.

In certain embodiments, the anti-hGREM1 antibodies provided herein are cross-reactive to mouse GREM1, and comprise the heavy chain CDR1 (HCDR1), HCDR2 and HCDR3 and light chain CDR1 (LCDR1), LCDR2, and LCDR3 of an antibody selected from the group consisting of 56C11, 42B9, 36F5, and 67G11.

The benchmark antibody 6245P is cross-reactive to mouse GREM1.

iv) Antibodies that block the binding of hGREM1 to BMP7

In certain embodiments, the antibodies provided herein are capable of blocking the binding of hGREM1 to BMP7 at a maximal blocking percentage of at least 50% as measured by ELISA.

As used herein, the term “blocking percentage” refers to the percentage of reduced interaction between two proteins (e.g., reduced binding of human gremlin 1 to BMP7) in the presence of a blocker (e.g., anti-gremlin 1 antibody), relative to the interaction between the two proteins in absence of the blocker.

As used herein, the term “maximal blocking percentage” refers to the highest (i.e. plateau of) blocking percentage achievable by a blocker (e.g., anti-gremlin 1 antibody) to block the interaction between two proteins (e.g., human gremlin 1 and BMP7). In general, the percentage of blocking increases with the increase in concentration of the blocker (e.g., anti-gremlin 1 antibody), however, it could reach a plateau where no more blocking can be achieved despite of further increase in the concentration of the blocker (e.g., anti-gremlin 1 antibody). The higher the maximal blocking percentage, the more effective of the blocking. The maximal blocking percentage may vary to some degree depending on different assays, such as competitive ELISA assay and competitive FACS assay.

In certain embodiments, certain anti-gremlin 1 antibodies provided herein have maximal blocking percentage of at least 50% for hGREM1-BMP7 interaction, as measured by competitive ELISA assay. The assay conditions can be similar to those provide in Example 6 of the present disclosure (the concentration of human gremlin 1 is 1 μg/ml, and the concentration of BMP7 is 0.5 g/ml). Exemplary anti-gremlin 1 antibodies having the above-mentioned blocking activity for hGREM1-BMP7 interaction include 14E3 (e.g., 14E3HaLa), 42B9, 36F5 and 67G11.

In certain embodiments, certain anti-gremlin 1 antibodies provided herein have maximal blocking percentage of at least 60%, at least 70%, or at least 75% for hGREM1-BMP7 interaction, as measured by competitive ELISA assay. The assay conditions can be similar to those provide in Example 6 of the present disclosure (the concentration of human gremlin 1 is 1 μg/ml, and the concentration of BMP7 is 0.5 μg/ml). Exemplary anti-gremlin 1 antibodies having the above-mentioned blocking activity for hGREM1-BMP7 interaction include 42B9, 36F5 and 67G11.

BMP-7 is a homodimeric protein with cysteine-knot, which only selectively expressed in several adult organs including the kidney (Rui et al., Role of bone morphogenetic protein-7 in renal fibrosis, Front. Physiol., 23 Apr. 2015). The expression of BMP-7 in normal kidney is the highest in adult organs, and is downregulated in kidneys of patients with ischemia-reperfusion injury, diabetic nephropathy and hypertensive nephrosclerosis (Dudley et al., A requirement for bone morphogenetic protein-7 during development of the mammalian kidney and eye. Genes Dev. 9, 2795-2807, 1995; Luo et al., BMP-7 is an inducer of nephrogenesis, and is also required for eye development and skeletal patterning. Genes Dev. 9, 2808-2820. 1995; Simon et al., Expression of bone morphogenetic protein-7 mRNA in normal and ischemic adult rat kidney. Am. J. Physiol. 276, F382-F389, 1999; Wang et al., Loss of tubular bone morphogenetic protein-7 in diabetic nephropathy. J. Am. Soc. Nephrol. 12, 2392-2399, 2001; Bramlage et al., Bone morphogenetic protein (BMP)-7 expression is decreased in human hypertensive nephrosclerosis. BMC Nephrol. 11:31., 2010; Vukicevic et al., Osteogenic protein-1 (bone morphogenetic protein-7) reduces severity of injury after ischemic acute renal failure in rat. J. Clin. Invest. 102, 202-214., 1998; Simon et al., Expression of bone morphogenetic protein-7 mRNA in normal and ischemic adult rat kidney. Am. J. Physiol. 276, F382-F389., 1999).

BMP7, as one of the key cytokines in the TGFβ superfamily, plays an anti-fibrotic role in chronic kidney disease by counterbalancing the TGF-β signaling pathways, which mediates renal fibrogenesis by increasing extracellular matrix (ECM) production and reducing its degradation (Rui et al., Role of bone morphogenetic protein-7 in renal fibrosis, Front. Physiol., 23 Apr. 2015). BMP7 treatment in several animal models of kidney injury was reported to reverse renal fibrosis, and to restore renal function (Hruska et al., Osteogenic protein-1 prevents renal fibrogenesis associated with ureteral obstruction. Am. J Physiol. Renal Physiol. 279, F130-F143. (2000); Jeremiah et al., Bone morphogenetic protein-7 improves renal fibrosis and accelerates the return of renal function, J Am Soc Nephrol. 2002 January;).

However, the activity of BMP7 in the kidney is not only determined by availability of BMP7 itself, but also by a balance of agonists and antagonists (e.g., gremlin). When BMP7 is used to treat renal fibrosis and other kidney diseases (e.g., acute and chronic kidney injury), presence of BMP7 antagonist (e.g., gremlin) has to be considered for its treatment efficacy (Michael et al., Reversal of experimental renal fibrosis by BMP7 provides insights into novel therapeutic strategies for chronic kidney disease, Pediatr Nephrol. 2008 September; 23(9): 1395-8).

The present disclosure shows that gremlin 1 binds to BMP7, and that the anti-gremlin 1 antibodies provided herein (e.g., 42B9, 36F5, 67G11 and 14E3HaLa) have more potent blocking activity for gremlin 1 binding to BMP7, as compared to a benchmark antibody. As used herein, the term “benchmark antibody” refers to any existing anti-GREM1 antibody, such as 6245P, which was produced according to the sequence of H4H6245P disclosed in WO2014159010, disclosure of which is incorporated by reference in its entirety. In other words, the anti-gremlin 1 antibodies provided herein (e.g., 42B9, 36F5, 67G11 and 14E3HaLa) are capable of restoring BMP7 activity in BMP7-expressing organ (e.g., kidney) function. Accordingly, it can be reasonably expected that the anti-gremlin 1 antibodies provided herein (e.g., 42B9, 36F5, 67G11 and 14E3HaLa) can improve the therapeutic efficacy of treatment for fibrotic diseases and kidney diseases (e.g., renal fibrosis).

In certain embodiments, the antibodies provided herein are capable of capable of blocking the binding of hGREM1 to BMP7 at a maximal blocking percentage of at least 50% as measured by ELISA, and comprise the heavy chain CDR1 (HCDR1), HCDR2 and HCDR3 and light chain CDR1 (LCDR1), LCDR2, and LCDR3 of an antibody selected from the group consisting of 42B9, 36F5, and 67G11. In certain embodiments, the antibodies provided herein are capable of capable of blocking the binding of hGREM1 to BMP7 at a maximal blocking percentage of 30% to 50% as measured by ELISA, and comprise the heavy chain CDR1 (HCDR1), HCDR2 and HCDR3 and light chain CDR1 (LCDR1), LCDR2, and LCDR3 of antibody 14E3.

v) Antibodies that block the binding of FGFR to GREM1

In certain embodiments, the antibodies provided herein are capable of blocking the binding of blocking interaction of GREM1 (e.g., hGREM1 or mGREM1) to FGFR (e.g., FGFR1, preferably human FGFR1 (hFGFR1) or mouse FGFR1 (mFGFR1)), and comprise the heavy chain CDR1 (HCDR1), HCDR2 and HCDR3 and light chain CDR1 (LCDR1), LCDR2, and LCDR3 of an antibody selected from the group consisting of 14E3, 42B9, 67G11 and 36F5. These antibodies are particularly useful for methods of treating conditions or disease associated with GREM1 binding to FGFR or FGFR activity.

In certain embodiments, the antibodies provided herein does not block the binding of blocking interaction of GREM1 (e.g., hGREM1 or mGREM1) to FGFR (e.g., FGFR1, preferably human FGFR1 (hFGFR1) or mouse FGFR1 (mFGFR1)), and comprise the heavy chain CDR1 (HCDR1), HCDR2 and HCDR3 and light chain CDR1 (LCDR1), LCDR2, and LCDR3 of an antibody selected from the group consisting of 56C11.

In certain embodiments, the antibodies provided herein partially block (with an IC50 of at least 2 nM, at least 3 nM, at least 4 nM, at least 5 nM, at least 6 nM, or at least 7 nM) the binding of blocking interaction of GREM1 (e.g., hGREM1 or mGREM1) to FGFR (e.g., FGFR1, preferably human FGFR1 (hFGFR1) or mouse FGFR1 (mFGFR1)), and comprise the heavy chain CDR1 (HCDR1), HCDR2 and HCDR3 and light chain CDR1 (LCDR1), LCDR2, and LCDR3 of an antibody 22F1 or 69H5.

vi) Antibodies that reduce GREM1-mediated activation on MAPK signaling

In certain embodiments, the anti-GREM1 antibodies provided herein are capable of reducing GREM1-mediated activation on MAPK signaling. It is well known in the art that MAPK signaling is a critical signal pathway to maintain tumor cell proliferation, migration, angiogenesis and epithelial-mesenchymal transition (EMT). It is known in the art that MAPK signaling can be activated either via an epidermal growth factor (EGF) receptor by its ligand called EGF, or via a fibroblast growth factor (FGF) receptor by its ligand called FGF. The present disclosure surprisingly found that GREM1 appears to play a role in the activation of MAPK signaling, and possibly acts as a novel ligand of FGFR. The present disclosure further found that the anti-GREM1 antibodies provided herein are capable of reducing GREM1-mediated activation on MAPK signaling, especially capable of blocking interaction of GREM1 to FGFR.

vii) Antibodies that Bind to Both hGREM1 and DAN

The antibodies provided in the present disclosure may specifically bind to one or more (e.g., 1, 2, 3 or more) DAN family members including GREM1. In certain embodiments, the antibodies provided herein are capable of binding to both hGREM1 and DAN. As used herein, the term “DAN” refers to the founding member (also named as NbI1 and DAND1) of the DAN family, which is a moderate antagonist for modulating BMP signaling. DAN initially acted as tumor suppressor gene in neuroblastoma. Mis-regulation of the balance between BMP signaling and DAN inhibition can lead to numerous disease states, including cancer, kidney nephropathy, and pulmonary arterial hypertension. Gremlin acts as strong antagonists, and DAN functions as a modest antagonist. Although they could both antagonizes BMP2, BMP4 and BMP7, but they only share ˜20% identities.

In certain embodiments, the anti-hGREM1 antibodies provided herein are capable of binding to both hGREM1 and DAN, and comprise the heavy chain CDR1 (HCDR1), HCDR2 and HCDR3 and light chain CDR1 (LCDR1), LCDR2, and LCDR3 of an antibody selected from the group consisting of 36F5, 42B9 and 67G11. These antibodies are particularly useful for methods of treating conditions or disease associated with both GREM1 and DAN.

In certain embodiments, the anti-hGREM1 antibodies provided herein are capable of binding to hGREM1 but not to DAN, and comprise the heavy chain CDR1 (HCDR1), HCDR2 and HCDR3 and light chain CDR1 (LCDR1), LCDR2, and LCDR3 of antibody 14E3, 22F1, 56C11 or 69H5.

### Bispecific Antibodies

In certain embodiments, the antibodies and antigen-binding fragments thereof provided herein are bispecific. The term “bispecific” as used herein encompasses molecules having more than two specificity and molecules having more than two specificity, i.e. multispecific. In certain embodiments, the bispecific antibodies and antigen-binding fragments thereof provided herein is capable of specifically binding to a first and a second epitopes of hGREM1, or capable of specifically binding to hGREM1 and a second antigen. In certain embodiments, the first epitope and the second epitopes of hGREM1 are distinct from each other or non-overlapping. In certain embodiments, the bispecific antibodies and antigen-binding fragments thereof can bind to both the first epitope and the second epitope at the same time. In certain embodiments, the second antigen is different from hGREM1.

In certain embodiments, the second antigen is an immune related target. An immune related target as used herein, encompasses a biological molecule that is involved in the generation, inhibition or modulation of an immune response, optionally, cellular immune responses. An example of the immune related target is immune checkpoint molecule.

Immune checkpoint molecule can mediate co-stimulatory signal to augment immune response, or can mediate co-inhibitory signals to suppress immune response. Examples of an immune checkpoint molecule include, for example, PD-L1, PD-L2, PD-1, CTLA-4, TIM-3, LAG3, A2AR, CD160, 2B4, TGF R, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16 and CD83. In certain embodiments, the second antigen comprises PD-1, PD-L1, CTLA-4, or LAG-3.

In certain embodiments, the second antigen comprises a tumor antigen. “Tumor antigen” as used herein refers to tumor specific antigens (e.g. those unique to tumor cells and normally not found on non-tumor cells), and tumor-associated antigens (e.g. found in both tumor and non-tumor cells but expressed differently in tumor cells, or found in tumor microenvironment). Tumor specific antigens can also include tumor neo-antigens (e.g. that are expressed in cancer cells because of somatic mutations that change the protein sequence or create fusion proteins between two unrelated sequences).

Examples of tumor antigens include, without limitation, prostate specific antigen (PSA), CA-125, gangliosides G(D2), G(M2) and G(D3), CD20, CD52, CD33, Ep-CAM, CEA, bombesin-like peptides, HER2/neu, epidermal growth factor receptor (EGFR), erbB2, erbB3/HER3, erbB4, CD44v6, Ki-67, cancer-associated mucin, VEGF, VEGFRs (e.g., VEGFR3), estrogen receptors, Lewis-Y antigen, TGF(31, IGF-1 receptor, EGFua, c-Kit receptor, transferrin receptor, Claudin 18.2, GPC-3, Nectin-4, ROR1, methothelin, PCMA, MAGE-1, MAGE-3, BAGE, GAGE-1, GAGE-2, p15, BCR-ABL, E2APRL, H4-RET, IGH-IGK, MYL-RAR, IL-2R, CO17-1A, TROP2, or LIV-1.

In certain embodiments, the tumor antigen comprises prostate specific antigen (PSA), CA-125, gangliosides G(D2), G(M2) and G(D3), CD20, CD52, CD33, Ep-CAM, CEA, bombesin-like peptides, HER2/neu, epidermal growth factor receptor (EGFR), erbB2, erbB3/HER3, erbB4, CD44v6, Ki-67, cancer-associated mucin, VEGF, VEGFRs (e.g., VEGFR3), estrogen receptors, Lewis-Y antigen, TGF(31, IGF-1 receptor, EGFua, c-Kit receptor, transferrin receptor, Claudin 18.2, GPC-3, Nectin-4, ROR1, methothelin, PCMA, MAGE-1, MAGE-3, BAGE, GAGE-1, GAGE-2, p15, BCR-ABL, E2APRL, H4-RET, IGH-IGK, MYL-RAR, IL-2R, CO17-1A, TROP2, or LIV-1.

Bispecific antibodies and antigen-binding fragments thereof provided herein can be in a suitable format known in the art. For example, an exemplary bispecific format can be, bispecific diabodies, scFv-based bispecific formats, IgG-scFv fusions, dual variable domain (DVD)-Ig, Quadroma, knobs-into-holes, common light chain (e.g., common light chain with knobs-into-holes, etc.), BiTE, CrossMab, CrossFab, Duobody, SEEDbody, leucine zipper, dual acting Fab (DAF)-IgG, and Mab² bispecific formats (see, e.g., Brinkmann et al. 2017, Mabs, 9(2): 182-212). The bispecific molecules can be in symmetric or asymmetric architecture.

The bispecific antibodies and antigen-binding fragments provided herein can be made with any suitable methods known in the art.

In one embodiment, two immunoglobulin heavy chain-light chain pairs having different antigenic specificities are co-expressed in a host cell to produce bispecific antibodies in a recombinant way (see, for example, Milstein and Cuello, Nature, 305: 537 (1983)), followed by purification by affinity chromatography.

In another embodiment, sequences encoding the antibody heavy chain variable domains for the two specificities are respectively fused to immunoglobulin constant domain sequences, followed by insertion to one or more expression vector(s) which is/are co-transfected with an expression vector for the light chain sequences to a suitable host cell for recombinant expression of the bispecific antibody (see, for example, WO 94/04690; Suresh et al., Methods in Enzymology, 121:210 (1986)). Similarly, scFv dimers can also be recombinantly constructed and expressed from a host cell (see, e.g., Gruber et al., J. Immunol., 152:5368 (1994).)

In another method, leucine zipper peptides from the Fos and June proteins can be linked to the Fab′ portions of two different antibodies by gene fusion. The linked antibodies are reduced at the hinge region to four half antibodies (i.e. monomers) and then re-oxidized to form heterodimers (Kostelny et al., J. Immunol., 148(5):1547-1553 (1992)).

The two antigen-binding domains may also be conjugated or cross-linked to form a bispecific antibody or antigen-binding fragment. For example, one antibody can be coupled to biotin while the other antibody to avidin, and the strong association between biotin and avidin would complex the two antibodies together to form a bispecific antibody (see, for example, U.S. Pat. No. 4,676,980; WO 91/00360, WO 92/00373, and EP 03089). For another example, the two antibodies or antigen-binding fragments can be cross-linked by conventional methods known in the art, for example, as disclosed in U.S. Pat. No. 4,676,980.

Bispecific antigen-binding fragments may be generated from a bispecific antibody, for example, by proteolytic cleavage, or by chemical linking. For example, an antigen-binding fragment (e.g., Fab′) of an antibody may be prepared and converted to Fab′-thiol derivative and then mixed and reacted with another converted Fab′ derivative having a different antigenic specificity to form a bispecific antigen-binding fragment (see, for example, Brennan et al., Science, 229: 81 (1985)).

In certain embodiments, the bispecific antibody or antigen-binding fragments thereof provided herein may be engineered at the interface so that a knob-into-hole association can be formed to promote heterodimerization of the two different antigen-binding sites. This can maximize the percentage of heterodimers which are recovered from recombinant cell culture. “Knob-into-hole” as used herein, refers to an interaction between two polypeptides (such as Fc), where one polypeptide has a protuberance (i.e. “knob”) due to presence of an amino acid residue having a bulky side chain (e.g., tyrosine or tryptophan), and the other polypeptide has a cavity (i.e. “hole”) where a small side chain amino acid residue resides (e.g., alanine or threonine), and the protuberance is positionable in the cavity so as to promote interaction of the two polypeptides to form a heterodimer or a complex. Methods of generating polypeptides with knobs-into-holes are known in the art, e.g., as described in U.S. Pat. No. 5,731,168.

### Conjugates

In some embodiments, the anti-hGREM1 antibodies and antigen-binding fragments thereof are linked to one or more conjugate moieties. A conjugate is a moiety that can be attached to the antibody or antigen-binding fragment thereof. It is contemplated that a variety of conjugates may be linked to the antibodies or antigen-binding fragments provided herein (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In certain embodiments, the antibodies or antigen binding fragments thereof are linked to one or more conjugates via a linker. In certain embodiments, the linker is a hydrazone linker, a disulfide linker, a bifunctional linker, dipeptide linker, glucuronide linker, a thioether linker.

In certain embodiments, the anti-hGREM1 antibodies and antigen-binding fragments disclosed herein may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate.

The conjugate can be a clearance-modifying agent, therapeutic agent (e.g., a chemotherapeutic agent), a toxin, a radioactive isotope, a detectable label (e.g., a lanthanide, a luminescent label, a fluorescent label, or an enzyme-substrate label), a pharmacokinetic modifying moiety, a DNA-alkylator, a topoisomerase inhibitor, a tubulin-binders, other anticancer drugs called such as androgen receptor inhibitor.

Examples of detectable label may include a fluorescent labels (e.g., fluorescein, rhodamine, dansyl, phycoerythrin, or Texas Red), enzyme-substrate labels (e.g., horseradish peroxidase, alkaline phosphatase, luceriferases, glucoamylase, lysozyme, saccharide oxidases or β-D-galactosidase), radioisotuopes, other lanthanides, luminescent labels, chromophoric moiety, digoxigenin, biotin/avidin, a DNA molecule or gold for detection.

Examples of radioisotopes may include ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P. Radioisotope labelled antibodies are useful in receptor targeted imaging experiments.

In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like.

In certain embodiments, the conjugate can be a purification moiety such as a magnetic bead or a nanoparticle.

### Pharmaceutical Composition

The present disclosure provides pharmaceutical compositions comprising the anti-hGREM1 antibodies or antigen-binding fragments thereof and one or more pharmaceutically acceptable carriers.

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment and conjugates as provided herein decreases oxidation of the antibody or antigen-binding fragment. This reduction in oxidation prevents or reduces loss of binding affinity, thereby improving antibody stability and maximizing shelf-life. Therefore, in certain embodiments compositions are provided that comprise one or more antibodies or antigen-binding fragments as disclosed herein and one or more antioxidants such as methionine. Further provided are methods for preventing oxidation of, extending the shelf-life of, and/or improving the efficacy of an antibody or antigen-binding fragment as provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-hGREM1 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

In certain embodiments, the pharmaceutical composition further comprises a second therapeutic agent.

In certain embodiments, the second therapeutic agent can be an agent for treating cancer, for example, a chemotherapeutic agent, an anti-cancer drug, radiation therapy, an immunotherapy, anti-angiogenesis agent (e.g. antagonist of a VEGFR such as VEGFR-1, VEGFR-2, and VEGFR-3), a targeted therapy, a cellular therapy, a gene therapy agent, a hormonal therapy agent, cytokines, palliative care, surgery for the treatment of cancer (e.g., tumorectomy), or one or more anti-emetics or other treatments for complications arising from chemotherapy.

In certain embodiments, the second therapeutic agent comprises anti-angiogenesis agent, for example, antagonist of VEGFR or VEGF. In certain embodiments, the second therapeutic agent comprises an anti-VEGFR antibody or an anti-VEGF antibody. In certain embodiments, the second therapeutic agent comprises an anti-VEGFR-2 antibody.

In certain embodiments, the second therapeutic agent can be an agent for treating fibrotic disease.

In certain embodiments, the second therapeutic agent manages or treats at least one complication associated with fibrosis or cancer.

### Polynucleotides and Recombinant Methods

The present disclosure provides isolated polynucleotides that encode the anti-hGREM1 antibodies and antigen-binding fragments thereof. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In certain embodiments, the isolated polynucleotides comprise one or more nucleotide sequences as shown in SEQ ID NOs: 9, 10, 19, 20, 29, 30, 39, 40, 42, 44, 46, 48, 50, 52, 54, 56, 58, 60, 62, and 142-147, and/or a sequence having at least 80% (e.g. at least 85%, 88%, 90%, 92%, 93%, 94%, 95%, 96%, 97%, 98%, or 99%) sequence identity thereof, and/or a variant thereof having only degenerate substitutions, and encodes the variable region of the exemplary antibodies provided herein.

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

The present disclosure provides vectors (e.g. expression vectors) comprising the isolated polynucleotide provided herein. In certain embodiments, the expression vector provided herein comprises the polynucleotide encoding the antibodies or antigen-binding fragments thereof provided herein, at least one promoter (e.g. SV40, CMV, EF-1α) operably linked to the polynucleotide sequence, and at least one selection marker. Examples of vectors include, but are not limited to, retrovirus (including lentivirus), adenovirus, adeno-associated virus, herpesvirus (e.g. herpes simplex virus), poxvirus, baculovirus, papillomavirus, papovavirus (e.g. SV40), lambda phage, and M13 phage, plasmids such as pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pProl8, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof can be introduced to a host cell for cloning or gene expression. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g. E. coli, Enterobacter, Erwinia, Klebsiella, Proteus, Salmonella, e.g. Salmonella typhimurium, Serratia, e.g. Serratia marcescans, and Shigella, as well as Bacilli such as B. subtilis and B. licheriformis, Pseudomonas such as P. aeruginosa, and Streptomyces.

In addition to prokaryotes, eukaryotic microbes such as filamentous fungi or yeast are suitable cloning or expression hosts for anti-hGREM1 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

Suitable host cells for the expression of glycosylated antibodies or antigen-fragment provided herein are derived from multicellular organisms such as invertebrate cells, for example plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g. the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some preferable embodiments, the host cell is a mammalian cultured cell line, such as CHO, BHK, NS0, 293 and their derivatives.

Host cells are transformed with the above-described expression or cloning vectors for anti-hGREM1 antibody production and cultured in conventional nutrient media modified as appropriate for inducing promoters, selecting transformants, or amplifying the genes encoding the desired sequences. In another embodiment, the antibody may be produced by homologous recombination known in the art.

The host cells used to produce the antibodies or antigen-binding fragments provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-hGREM1 antibodies and antigen-binding fragments thereof prepared from the cells can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Methods of Use

In one aspect, the present disclosure provides therapeutic uses of the antibodies provided herein.

In certain embodiments, the present disclosure provides methods of treating or preventing a GREM1-related disease or condition in a subject in need thereof, comprising: administering a therapeutically effective amount of the antibody or antigen-binding fragment as provided herein and/or the pharmaceutical composition provided herein, thereby treating or preventing the GREM1-related disease or condition.

In another aspect, the present disclosure provides methods of treating a GREM1-related disease or condition in a subject in need thereof, comprising administering to the subject a therapeutically effective amount of an anti-human GREM1 antibody or antigen-binding fragment thereof, which is:
a) capable of binding to hGREM1 at an epitope comprising residue Gln27 and/or residue Asn33, wherein residue number is according to SEQ ID NO: 69, and/or
b) capable of binding to a hGREM1 fragment comprising residue Gln27 and/or residue Asn33, optionally the hGREM1 fragment has a length of at least 3 (e.g. 4, 5, 6, 7, 8, 9, or 10) amino acid residues; and/or
c) capable of reducing hGREM1-mediated inhibition on BMP signaling selectively in a cancer cell over a non-cancer cell; and/or
d) exhibiting no more than 50% reduction of hGREM1-mediated inhibition on BMP signaling in a non-cancer cell; and/or
e) capable of binding to a chimeric hGREM1 comprising an amino acid sequence of SEQ ID NO: 68; and/or
g) capable of binding to hGREM1 at a K_{D} of no more than 1 nM as measured by Fortebio; and/or
h) capable of blocking the binding of hGREM1 to BMP7 at a maximal blocking percentage of more than 50% as measured by ELISA; and/or
i) capable of blocking interaction of GREM1 (e.g., hGREM1 or mGREM1) to FGFR (e.g., FGFR1, preferably human FGFR1 (hFGFR1) or mouse FGFR1 (mFGFR1)).

In another aspect, the present disclosure provides a method of inhibiting FGFR1 activation in a subject in need thereof, or a method of treating a disease or condition associated with FGFR1 activation mediated by GREM1, comprising administering to the subject a therapeutically effective amount of an anti-human GREM1 antibody or antigen-binding fragment thereof, wherein the anti-human GREM1 antibody or antigen-binding fragment thereof comprises:
a) a HCDR1 comprising the sequence of SEQ ID NO: 1, a HCDR2 comprising the sequence of SEQ ID NO: 2, and a HCDR3 comprising the sequence of SEQ ID NO: 3; a LCDR1 comprising the sequence of SEQ ID NO: 4, a LCDR2 comprising the sequence of SEQ ID NO: 5, and a LCDR3 comprising the sequence of SEQ ID NO: 6;
b) a HCDR1 comprising the sequence of SEQ ID NO: 11, a HCDR2 comprising the sequence of SEQ ID NO: 12, and a HCDR3 comprising the sequence of SEQ ID NO: 13; a LCDR1 comprising the sequence of SEQ ID NO: 14, a LCDR2 comprising the sequence of SEQ ID NO: 15, and a LCDR3 comprising the sequence of SEQ ID NO: 16;
c) a HCDR1 comprising the sequence of SEQ ID NO: 21, a HCDR2 comprising the sequence of SEQ ID NO: 22, and a HCDR3 comprising the sequence of SEQ ID NO: 23; a LCDR1 comprising the sequence of SEQ ID NO: 24, a LCDR2 comprising the sequence of SEQ ID NO: 25, and a LCDR3 comprising the sequence of SEQ ID NO: 26;
d) a HCDR1 comprising the sequence of SEQ ID NO: 31, a HCDR2 comprising the sequence of SEQ ID NO: 32, a HCDR3 comprising the sequence of SEQ ID NO: 33; a LCDR1 comprising the sequence of SEQ ID NO: 34, a LCDR2 comprising the sequence of SEQ ID NO: 35, and a LCDR3 comprising the sequence of SEQ ID NO: 36;
e) a HCDR1 comprising the sequence of SEQ ID NO: 114, a HCDR2 comprising the sequence of SEQ ID NO: 115, and a HCDR3 comprising the sequence of SEQ ID NO: 116; a LCDR1 comprising the sequence of SEQ ID NO: 117, a LCDR2 comprising the sequence of SEQ ID NO: 35, and a LCDR3 comprising the sequence of SEQ ID NO: 118;
f) a HCDR1 comprising the sequence of SEQ ID NO: 119, a HCDR2 comprising the sequence of SEQ ID NO: 115, and a HCDR3 comprising the sequence of SEQ ID NO: 120; a LCDR1 comprising the sequence of SEQ ID NO: 121, a LCDR2 comprising the sequence of SEQ ID NO: 35, and a LCDR3 comprising the sequence of SEQ ID NO: 118; or
g) a HCDR1 comprising the sequence of SEQ ID NO: 119, a HCDR2 comprising the sequence of SEQ ID NO: 115, and a HCDR3 comprising the sequence of SEQ ID NO: 120; a LCDR1 comprising the sequence of SEQ ID NO: 122, a LCDR2 comprising the sequence of SEQ ID NO: 35, and a LCDR3 comprising the sequence of SEQ ID NO: 118.

In another aspect, the present disclosure provides a method of inhibiting FGFR1 activation in a subject in need thereof, or a method of treating a disease or condition associated with FGFR1 activation mediated by GREM1, comprising administering to the subject a therapeutically effective amount of an anti-human GREM1 antibody or antigen-binding fragment thereof, wherein the anti-human GREM1 antibody or antigen-binding fragment thereof comprises:
a) a HCDR1 comprising the sequence of SEQ ID NO: 123, a HCDR2 comprising the sequence of SEQ ID NO: 115, a HCDR3 comprising the sequence of SEQ ID NO: 124; a LCDR1 comprising the sequence of SEQ ID NO: 125, a LCDR2 comprising the sequence of SEQ ID NO: 35, and a LCDR3 comprising the sequence of SEQ ID NO: 118,
b) a HCDR1 comprising the sequence of SEQ ID NO: 114, a HCDR2 comprising the sequence of SEQ ID NO: 115, a HCDR3 comprising the sequence of SEQ ID NO: 116; a LCDR1 comprising the sequence of SEQ ID NO: 117, a LCDR2 comprising the sequence of SEQ ID NO: 35, and a LCDR3 comprising the sequence of SEQ ID NO: 118;
c) a HCDR1 comprising the sequence of SEQ ID NO: 119, a HCDR2 comprising the sequence of SEQ ID NO: 115, a HCDR3 comprising the sequence of SEQ ID NO: 120; a LCDR1 comprising the sequence of SEQ ID NO: 121, a LCDR2 comprising the sequence of SEQ ID NO: 35, and a LCDR3 comprising the sequence of SEQ ID NO: 118; or
d) a HCDR1 comprising the sequence of SEQ ID NO: 119, a HCDR2 comprising the sequence of SEQ ID NO: 115, a HCDR3 comprising the sequence of SEQ ID NO: 120; a LCDR1 comprising the sequence of SEQ ID NO: 122, a LCDR2 comprising the sequence of SEQ ID NO: 35, and a LCDR3 comprising the sequence of SEQ ID NO: 118.

A GREM1-related disease or condition can be a disease or condition that would benefit from modulation of GREM1 activity (e.g. reduction in GREM1 activity). In some embodiment, the GREM1 related disease or condition is characterized in GREM1 expression or overexpression.

The term “overexpression” with respect to GREM1 as used herein refers to an increased expression level relative to a reference level. The reference level can be the level of GREM1 expression found in normal cells of the same tissue type, optionally normalized to expression level of another gene (e.g. a house keeping gene). Alternatively, the reference level can be the level of GREM1 expression found in healthy subjects. In some embodiments, the GREM1-expressing cancer has a GREM1 expression level at least 10% higher (e.g. at least 15%, 20%, 30%, 35%, 40%, 50% or 1-fold, 2-fold, 3-fold or even higher) than a reference level.

Expression of GREM1 can be determined based on nucleic acid level or protein level. Expression level of GREM1 can be measured at nucleic acid level by any methods known in the art, for example, without limitation, an amplification assay (such as polymerase chain reaction, quantitative real-time PCR, rolling circle replication, isothermal amplification, and so on), a hybridization assay (e.g. Northern blotting, microarrays, Fluorescence in situ hybridization (FISH), etc.), or a sequencing assay (e.g. RNA sequencing). Alternatively, expression level of GREM1 can be measured at protein level by any methods known in the art, for example, without limitation, immunoassays (such as Western blotting, enzyme-linked immunosorbent assay (ELISA), enzyme immunoassay (EIA), radioimmunoassay (RIA), sandwich assays, competitive assays, immunofluorescent staining and imaging, immunohistochemistry (IHC), and fluorescent activating cell sorting (FACS)).

In certain embodiments, the subject is human. In certain embodiments, the subject is identified as having a GREM1-expression or overexpression, optionally in a biological sample obtained from the subject.

In some embodiment, the GREM1-related disease or condition is selected from the group consisting of cancer, fibrotic disease, angiogenesis, glaucoma or retinal disease, kidney disease, pulmonary arterial hypertension, and osteoarthritis (OA). Increased levels of GREM1 have been associated with many of these diseases and conditions, such as scleroderma, diabetic nephropathy, glioma, head and neck cancer, prostate cancer and colorectal cancer.

i. Cancer Treatment

In certain embodiments, the present disclosure provides methods of treating or preventing cancer using the antibodies provided herein.

In some embodiment, the cancer is a GREM1-expressing cancer. The phrase “GREM1-expressing cancer” as used herein refers to a cancer characterized in having a GREM1-expressing cancer cell, and/or having GREM1 expression in a cancer microenvironment. In some embodiment, the GREM1-expressing cancer has GREM1 overexpression in a cancer cell and/or in cancer microenvironment.

GREM1 can act via an autocrine way to promote growth of tumor cells expressing GREM1. GREM1 can also be secreted by non-cancer cells residing within or surrounding the cancer microenvironment to create a niche suitable for the growth or survival of cancer cells, even if the cancer cells themselves may not necessarily express GREM1.

Cancer microenvironment as used herein, refers to the tissue, cell and environment that surround the cancer cell. Cancer microenvironment can comprise stromal cells such as fibroblasts, pericytes, endothelial cells, adipose cells, and bone marrow mesenchymal stromal cells (MSCs). Cancer microenvironment can also comprise extracellular matrix associated with the cancer cells or associated with the stromal cells surrounding the cancer cells. Extracellular matrix is primarily composed of ground substance—a porous, hydrated gel, made mainly from proteoglycan aggregates—and connective tissue fibers. Expression of GREM1 in cancer microenvironment can be observed in, for example, stromal cells or extracellular matrix. In certain embodiments, the GREM1-expressing cancer has GREM1 expression or overexpression in stroma (such as a desmoplastic stroma) or stromal cells.

In certain embodiments, the subject is identified as having a GREM1-expressing cancer cell, or having GREM1 expression in cancer microenvironment. The presence and/or expression level of GREM1 on a cancer cell or in a cancer microenvironment can be determined by various methods known in the art or provided herein, using a biological sample obtained from the subject. A biological sample containing or suspected of containing a cancer cell or from a cancer microenvironment can be obtained or derived from the subject, for example, formalin fixed paraffin embedded (FFPE) tissue, fresh biopsy, blood (suspected of containing circulating tumor cells), or other body fluid. In some embodiments, the cancer cell, stromal cell and/or extracellular matrix may be isolated from the biological sample. In certain embodiments, the biological sample may be further processed to, for example, isolate the analyte such as the nucleic acids or proteins.

GREM1-expressing cancer can be any type of cancers. In certain embodiments, the cancer is selected from solid tumors or hematological tumors. In certain embodiments, the solid tumor is adrenocortical carcinoma, anal cancer, astrocytoma, childhood cerebellar or cerebral, basal-cell carcinoma, bile duct cancer, bladder cancer, bone tumor, brain cancer, cerebellar astrocytoma, cerebral astrocytoma/malignant glioma, ependymoma, medulloblastoma, supratentorial primitive neuroectodermal tumors, visual pathway and hypothalamic glioma, breast cancer, Burkitt's lymphoma, cervical cancer, colon cancer, emphysema, endometrial cancer, esophageal cancer, Ewing's sarcoma, retinoblastoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, liver cancer, lung cancer, neuroblastoma, non-Hodgkin lymphoma, ovarian cancer, pancreatic cancer, pharyngeal cancer, prostate cancer, rectal cancer, renal cell carcinoma (kidney cancer), retinoblastoma, Ewing family of tumors, skin cancer, stomach cancer, testicular cancer, throat cancer, thyroid cancer, or vaginal cancer.

In certain embodiments, the GREM1-expressing cancer is also PD-L1-expressing. In certain embodiments, the GREM1-expressing cancer is not a PD-L1 expressing cancer. The term “PD-L1 expressing” with respect to a cancer as used herein refers to a cancer positive for PD-L1 expression using any detection method known in the art, e.g., immunohistochemistry (IHC), flow cytometry (such as FACS), etc. For example, a PD-L1-expressing cancer can refer to a cancer positive for PD-L1 expression using a simple binary positive/negative characterization approach based on IHC data, with a positive result defined if the percentage of cancer cells in a tumor tissue section that exhibited cell-surface membrane staining of PD-L1 is at least 1%, 2%, 3%, 4% or 5% of total cancer cells. Detailed description can be found in Thompson, R. H, et al., PNAS 101 (49); 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4, 127ra37 (2012); and Top/ian, S. L. et al., New Eng. J Med. 366 (26): 2443-2454 (2012). A PD-L1-expressing cancer can also refer to a cancer positive for PD-L1 expressing using the scoring process described in WO2014165422A1. In certain embodiment, the GREM1-expressing cancer is resistant or refractory to the treatment with a PD-1/PD-L1 axis inhibitor.

In certain embodiments, the hematological tumor is leukemia (such as Acute lymphocytic leukemia (ALL), Acute myeloid leukemia (AML), Chronic lymphocytic leukemia (CLL), Chronic myeloid leukemia (CML)), lymphoma (such as Hodgkin's lymphoma, or Non-Hodgkin's lymphoma (e.g. Waldenstrom macroglobulinemia (WM))), or myeloma (such as multiple myeloma (MM)).

In certain embodiments, the cancer is prostate cancer, gastric-esophageal cancer, lung cancer (e.g., non-small cell lung cancer), liver cancer, pancreatic cancer, breast cancer, bronchial cancer, bone cancer, liver and bile duct cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, gastrointestinal cancer, skin cancer, pituitary cancer, stomach cancer, vagina cancer, thyroid cancer, glioblastoma, astrocytoma, melanoma, myelodysplastic syndrome, sarcoma, teratoma, glioma, adenocarcinoma, leukemia (such as Acute lymphocytic leukemia (ALL), Acute myeloid leukemia (AML), Chronic lymphocytic leukemia (CLL), Chronic myeloid leukemia (CML)), lymphoma (such as Hodgkin's lymphoma, or Non-Hodgkin's lymphoma (e.g. Waldenstrom macroglobulinemia (WM))), or myeloma (such as multiple myeloma (MM)).

In certain embodiments, the cancer is selected from the group consisting of prostate cancer, gastric-esophageal cancer, lung cancer (e.g., non-small cell lung cancer), liver cancer, colon cancer, colorectal cancer, glioma, pancreatic cancer, bladder cancer and breast cancer. In certain embodiments, the cancer is triple negative breast cancer. In certain embodiments, the cancer is multiple myeloma.

In certain embodiments, the cancer is metastatic. In certain embodiments, the present disclosure further provides methods of treating or preventing cancer metastasis using the antibodies provided herein. Cancer metastasis is the process during which cancer cells spread from its original site to another site within the body.

In certain embodiments, the cancer is prostate cancer, breast cancer or liver cancer.

In certain embodiments, the breast cancer is triple negative breast cancer. The term “triple-negative breast cancer” or “TNBC” refers to a breast cancer that is tested negative for estrogen receptors, progesterone receptors, and excess HER2 protein. TNBC can be non-responsive to hormone therapies or drugs targeting HER2.

In certain embodiments, the cancer is multiple myeloma (MM). GREM1 is found to be abundantly secreted by a subset of bone marrow (BM) mesenchymal stromal cells, and is considered to play a critical role in MM disease development. Analysis of human and mouse BM stromal samples by quantitative PCR showed that GREM1/Grem1 expression was significantly higher in the MM tumor-bearing cohorts compared to healthy control. Anti-GREM1 antibodies have been shown to decrease MM tumor burden in mice (K. Clark et al., Cancers 2020, 12, 2149).

ii. Treating Fibrotic Diseases

In some embodiment, the GREM1-related disease or condition is a fibrotic disease. Fibrotic disease is a disease or condition that involves fibrosis. Fibrosis is a scarring process that is a common feature of chronic organ injury, for example in lungs, liver, kidney, skin, heart, gut or muscle. Fibrosis is characterized by elevated activity of transforming growth factor-beta (TGF-β) resulting in increased and altered deposition of extracellular matrix and other fibrosis-associated proteins. Elevated GREM1 expression has been found in many fibrotic diseases, suggesting that GREM1 may be an important marker of fibrosis (Costello, et al., 2010, Am. J. Respir. Cell. Mol. Biol. 42: 517-523; Lappin, et al., 2002, Nephrol. Dial. Transplant. 17: 65-67; Boers et al., 2006, J. Biol. Chem. 281: 16289-16295).

Fibrotic disease can include fibrotic disease in lungs, liver, kidney, eyes, skin, heart, gut or muscle. Examples of fibrotic disease in lungs include pulmonary fibrosis, cystic fibrosis, pulmonary hypertension, progressive massive fibrosis, bronchiolitis obliterans, airway remodeling associated with chronic asthma or idiopathic pulmonary. Examples of fibrotic disease in liver include cirrhosis or non-alcoholic steatohepatitis. Examples of fibrotic disease in kidney include such as renal fibrosis, ischemic renal injury, tubulointerstitial fibrosis, diabetic nephropathy, nephrosclerosis, or nephrotoxicity. Examples of fibrotic disease in eyes include such as corneal fibrosis, subretinal fibrosis. Examples of fibrotic disease in skin include such as nephrogenic systemic fibrosis, keloid or scleroderma. Examples of fibrotic disease in heart include endomyocardial fibrosis or old myocardial infarction.

In another aspect, the present disclosure also provides a method of improving the efficacy of BMP7 treatment in treating fibrotic diseases (e.g., renal fibrosis) in a subject in need thereof, comprising administering to the subject a therapeutically effective amount of the anti-gremlin 1 antibodies provided herein. In certain embodiments, the subject is being subject to BMP7 treatment. In another aspect, the present disclosure also provides a method of treating fibrotic diseases (e.g., renal fibrosis) in a subject in need thereof, comprising administering to the subject a therapeutically effective amount of the anti-gremlin 1 antibodies provided herein in combination with BMP7 treatment. The term “BMP7 treatment” as used herein can be any treatment that involves increasing the level of BMP7 in a subject in need thereof. For example, the BMP7 treatment can be administration of a recombinant BMP7 or peptide mimics of BMP7. The BMP7 treatment can also involve restoring the endogenous BMP7, such as by reducing the level and/or activity of the antagonists (e.g., noggin, or uterine sensitization-associated gene-1 (USAG-1)) of BMP7 or by increasing the level and/or activity of the agonists of (e.g., Kielin/chordin-like protein (KCP) or BMP receptors) BMP7 (Michael et al., Reversal of experimental renal fibrosis by BMP7 provides insights into novel therapeutic strategies for chronic kidney disease, Pediatr Nephrol. 2008 September; 23(9): 1395-8). In certain embodiments,

In another aspect, the present disclosure also provides a method of treating a disease that can benefit from increasing BMP7 activity or reducing gremlin-mediated inhibition on BMP7 activity, comprising administering to the subject a therapeutically effective amount of the anti-gremlin 1 antibodies provided herein. In certain embodiments, the disease is a fibrotic disease and/or kidney disease. In certain embodiments, the disease is selected from the group consisting of ischemia-reperfusion injury, ischemic acute renal failure, diabetic nephropathy and hypertensive nephrosclerosis, renal fibrosis, chronic kidney disease, acute kidney disease, hypertensive nephrosclerosis, Immunoglobulin A Nephropathy (IgAN) and other autoimmune disease such as lupus nephritis or systemic lupus erythematous (SLE).

iii. Treatment of other diseases

In some embodiment, the GREM1-related disease or condition is pulmonary artery hypertension (PAH). The term “pulmonary arterial hypertension” (“PAH”) refers to a progressive lung disorder which is characterized by sustained elevation of pulmonary artery pressure. GREM1 has been found to be elevated in the wall of small intrapulmonary vessels of mice during hypoxia. Anti-GREM1 antibodies have been found to alleviate or ameliorate one or more symptoms associated with PAH, for example, inhibits thickening of the pulmonary artery, increases stroke volume and/or stroke volume to end systolic volume ratio (“SV/ESV”), increases right ventricle cardiac output and/or cardiac index (CI), improve other hemodynamic measurements in a subject having PAH, such as, for example, right atrium pressure, pulmonary artery pressure, pulmonary capillary wedge pressure in the presence of end expiratory pressure, systemic artery pressure, heart beat, pulmonary vascular resistance, and/or systemic vascular resistance (see, for details, U.S. patent application US20180057580A1).

In some embodiment, the GREM1-related disease or condition is osteoarthritis (OA). GREM1 is reported as a mechanical loading-inducible factor in chondrocytes, and is detected at high levels in middle and deep layers of cartilage after cyclic strain or hydrostatic pressure loading. GREM1 is reported to be up-regulated in osteoarthritis, and GREM1 concentrations in serum and in synovial fluid are correlated with the onset and severity of knee OA (J. Yi, et al., Med Sci Monit, 2016; 22: 4062-4065). GREM1 activates nuclear factor-xB signalling, leading to subsequent induction of catabolic enzymes. Intra-articular administration of GREM1 antibody or chondrocyte-specific deletion of GREM1 in mice was reported to decelerate osteoarthritis development (see, S. H. Chang et al., Nature Communications, (2019) 10: 1442).

In some embodiment, the GREM1-related disease or condition is angiogenesis. GREM1 is an agonist of the major proangiogenic receptor vascular endothelial growth factor receptor-2 (VEGFR-2). Heparan sulfate (HS) and heparin, glycosaminoglycans (GAGs) known for their anticoagulant effects, have been shown to bind to GREM1. GREM1 binds to heparin and activates VEGFR-2 in a BMP-independent manner (Chiodelli et al 2011; Arterioscler. Thromb. Vasc. Biol. 31: el16-e127). Anti-GREM1 antibodies have been found to alleviate or ameliorate one or more symptoms associated with angiogenesis or heparin-mediated angiogenesis (see, for details, U.S. patent application US20200157194).

In some embodiment, the GREM1-related disease or condition is glaucoma. Glaucoma may be caused by altered expression of one or more BMP family genes in the eye, which leads to elevated increased intraocular pressure and/or glaucomatous optic neuropathy. GREM1 has been found to have an increased expression in glaucomatous trabecular meshwork cells. GREM1 antagonists have been found to alleviate or ameliorate one or more symptoms associated with angiogenesis or glaucoma (see, for details, U.S. patent U.S. Pat. No. 7,744,873).

In some embodiment, the GREM1-related disease or condition is retinal disease. In some embodiment, the GREM1-related disease or condition is kidney disease.

### Administration Route and Dosage Regime

The antibody or antigen-binding fragment as provided herein may be administered at a therapeutically effective dosage. The therapeutically effective amount of an antibody or antigen-binding fragment as provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by one of ordinary skill in the art (e.g., physician or veterinarian) as indicated by these and other circumstances or requirements.

In certain embodiments, the antibody or antigen-binding fragment as provided herein may be administered at a therapeutically effective dosage of about 0.01 mg/kg to about 100 mg/kg. In certain embodiments, the administration dosage may change over the course of treatment. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies and antigen-binding fragments disclosed herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

### Combination Therapies

In some embodiments, the antibodies or antigen-binding fragments disclosed herein may be administered alone or in combination with one or more additional therapeutic means or agents, which can be selected based on the disease or condition to be treated.

In some embodiments, the antibodies or antigen-binding fragments disclosed herein may be administered for treating cancer in combination with a second anti-cancer drug, for example, a chemotherapeutic agent, an anti-cancer drug, radiation therapy, an immunotherapy, anti-angiogenesis agent, a targeted therapy, a cellular therapy, a gene therapy agent, a hormonal therapy agent, cytokines, palliative care, surgery for the treatment of cancer (e.g., tumorectomy), one or more anti-emetics, treatments for complications arising from chemotherapy, or a diet supplement for cancer patients, or an agent that modulates tumor microenvironment.

The term “chemotherapeutic drug” is a biological (macromolecule) or chemical (small molecule) compound that can be used to treat cancer. The types of chemotherapeutic drugs include, but are not limited to, histone deacetylase inhibitor (HDACI), alkylating agents, antimetabolites, alkaloids, cytotoxic/anti-cancer antibiotics, topoisomerase inhibitors, tubulin inhibitors, proteins, antibodies, kinase inhibitors, and the like. Examples of chemotherapeutic drugs include, erlotinib, afatinib, docetaxel, adriamycin, 5-FU (5-fluorouracil), panobinostat, gemcitabine, cisplatin, carboplatin, paclitaxel, bevacizumab, trastuzumab, pertuzumab, metformin, temozolomide, tamoxifen, doxorubicin, rapamycin, lapatinib, hydroxycamptothecin, trimetinib. In certain embodiments, the chemotherapeutic drug is cisplatin.

The term “immunotherapy” as used herein, refers to a type of that stimulates immune system to fight against disease such as cancer or that boosts immune system in a general way. Immunotherapy includes passive immunotherapy by delivering agents with established tumor-immune reactivity (such as effector cells) that can directly or indirectly mediate anti-tumor effects and does not necessarily depend on an intact host immune system (such as an antibody therapy or CAR-T cell therapy). Immunotherapy can further include active immunotherapy, in which treatment relies on the in vivo stimulation of the endogenous host immune system to react against diseased cells with the administration of immune response-modifying agents.

Examples of immunotherapy include, without limitation, checkpoint modulators, adoptive cell transfer, cytokines, oncolytic virus and therapeutic vaccines.

Checkpoint modulators can interfere with the ability of cancer cells to avoid immune system attack, and help the immune system respond more strongly to a tumor. Immune checkpoint molecule can mediate co-stimulatory signal to augment immune response, or can mediate co-inhibitory signals to suppress immune response. Examples of checkpoint modulators include, without limitation, modulators of PD-1, PD-L1, PD-L2, CTLA-4, TIM-3, LAG3, A2AR, CD160, 2B4, TGF β, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16 and CD83. In certain embodiments, the immune checkpoint modulator comprises a PD-1/PD-L1 axis inhibitor.

Adoptive cell transfer, which is a treatment that attempts to boost the natural ability of the T cells to fight cancer. In this treatment, T cells are taken from the patient, and are expanded and activated in vitro. In certain embodiments, the T cells are modified in vitro to CAR-T cells. T cells or CAR-T cells that are most active against the cancer are cultured in large batches in vitro for 2 to 8 weeks. During this period, the patients will receive treatments such as chemotherapy and radiation therapy to reduce the body's immunity. After these treatments, the in vitro cultured T cells or CAR-T cells will be given back to the patient. In certain embodiments, the immunotherapy is CAR-T therapy.

Cytokine therapy can also be used to enhance tumor antigen presentation to the immune system. The two main types of cytokines used to treat cancer are interferons and interleukins. Examples of cytokine therapy include, without limitation, interferons such as interferon-α, -β, and -γ, colony stimulating factors such as macrophage-CSF, granulocyte macrophage CSF, and granulocyte-CSF, insulin growth factor (IGF-1), vascular endothelial growth factor (VEGF), transforming growth factor (TGF), fibroblast growth factor (FGF), interleukins such as IL-1, IL-1α, IL-2, IL-3, IL-4, IL-5, IL-6, IL-7, IL-8, IL-9, IL-10, IL-11, and IL-12, tumor necrosis factors such as TNF-α and TNF-β or any combination thereof.

Oncolytic virus are genetically modified virus that can kill cancer cells. Oncolytic virus can specifically infect tumor cells, thereby leading to tumor cell lysis followed by release of large amount of tumor antigens that trigger the immune system to target and eliminate cancer cells having such tumor antigens. Examples of oncolytic virus include, without limitation, talimogene laherparepvec.

Therapeutic vaccines work against cancer by boosting the immune system's response to cancer cells. Therapeutic vaccines can comprise non-pathogenic microorganism (e.g. Mycobacterium bovis Bacillus Calmette-Guérin, BCG), genetically modified virus targeting a tumor cell, or one or more immunogenic components. For example, BCG can be inserted directly into the bladder with a catheter and can cause an immune response against bladder cancer cells.

Anti-angiogenesis agent can block the growth of blood vessels that support tumor growth. Some of the anti-angiogenesis agent target VEGF or its receptor VEGFR. Examples of anti-angiogenesis agent include, without limitation, Axitinib, Bevacizumab, Cabozantinib, Everolimus, Lenalidomide, Lenvatinib mesylate, Pazopanib, Ramucirumab, Regorafenib, Sorafenib, Sunitinib, Thalidomide, Vandetanib, and Ziv-aflibercept.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

Targeted therapy can target, for example, tyrosine kinase receptors and nuclear receptors. Examples of such receptors include, erbB1 (EGFR or HER1), erbB2 (HER2), erbB3, erbB4, FGFR, platelet-derived growth factor receptor (PDGFR), and insulin-like growth factor-1 receptor (IGF-1R), estrogen receptors (ERs), nuclear receptors (NR) and PRs.

Targeted therapy can target molecules in tyrosine kinase or nuclear receptors signaling cascade, such as, Erk and PI3K/Akt, AP-2a, AP-20, AP-27, mitogen-activated protein kinase (MAPK), PTEN, p53, p19ARF, Rb, Apaf-1, CD-95/Fas, TRAIL-R1/R2, Caspase-8, Forkhead, Box 03A, MDM2, IAPs, NF-kB, Myc, P13K, Ras, FLIP, heregulin (HRG) (also known as gp30), Bcl-2, Bcl-xL, Bax, Bak, Bad, Bok, Bik, Blk, Hrk, BNIP3, BimL, Bid, and EGL-1.

Targeted therapy can also target tumor-associated ligands such estrogen, estradiol (E2), progesterone, oestrogen, androgen, glucocorticoid, prolactin, thyroid hormone, insulin, P70 S6 kinase protein (PS6), Survivin, fibroblast growth factors (FGFs), EGF, Neu Differentiation Factor (NDF), transforming growth factor alpha (TGF-α), IL-1A, TGF-beta, IGF-1, IGF-II, IGFBPs, IGFBP proteases, and IL-10.

In certain embodiments, the second therapeutic agent modulates tumor microenvironment. In certain embodiments, the second therapeutic agent is a bifunctional molecule comprising PD-L1 binding moiety and extracellular domain of TGF-beta receptor.

In some embodiments, the antibodies or antigen-binding fragments disclosed herein may be administered for treating prostate cancer in combination with a second anti-cancer drug. In certain embodiments, the anti-cancer drug comprises an anti-prostate cancer drug. In some embodiments, the anti-prostate cancer drug comprises an androgen axis inhibitor; an androgen synthesis inhibitor; a ADP-ribose polymerase (PARP) inhibitor; or a combination thereof.

In certain embodiments, the androgen axis inhibitor is selected from the group consisting of Luteinizing hormone-releasing hormone (LHRH) agonists, LHRH antagonists and androgen receptor antagonist.

In certain embodiments, the androgen axis inhibitor is degarelix, bicalutamide, flutamide, nilutamide, apalutamide, darolutamide, enzalutamide, or abiraterone.

In certain embodiments, the androgen synthesis inhibitor is abiraterone acetate or ketoconazole.

In certain embodiments, the PARP inhibitor is olaparib, or rucaparib.

In certain embodiments, the anti-prostate cancer drug is selected from the group consisting of Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynparza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate) and Zytiga (Abiraterone Acetate).

In certain embodiments, the diet supplement for cancer patients can be a suitable supplement that has a protective effect against cancer. In certain embodiments, the diet supplement comprises indole-3-carbinol or comprises a derivative thereof that gives rise to indole-3-carbinol after ingestion. Indole-3-carbinol is believed to have protective effects against cancer and also may be preventative against precancerous conditions.

In certain embodiments, the antibodies or antigen-binding fragments disclosed herein may be administered in combination with indole-3-carbinol or a derivative thereof that gives rise to indole-3-carbinol after ingestion. In certain embodiments, such combination is useful for treating gremlin-related diseases. In certain embodiments, such combination is useful for treating cancer, for example, breast cancer, hepatocellular carcinoma, and colorectal cancer. In certain embodiments, such combination is useful for treating breast cancer, for example, triple negative breast cancer.

In some embodiments, the antibodies or antigen-binding fragments disclosed herein may be administered for treating fibrotic disease in combination with a second therapeutic agent, for example, a second anti-fibrotic agent (e.g., a recombinant BMP7 or peptide mimics of BMP7). In certain embodiments, the second anti-fibrotic agent is ACE inhibitor (or ARB), anti-MASP2 antibody, endothelin receptor antagonist, NRF2 inhibitor steroid, CTLA4-IgG or TNF inhibitor.

In another embodiment, the second therapeutic agent is selected from the group consisting of an anti-fibrotic agent such as pirfenidone, an anti-inflammatory drug, a NSAID, a corticosteroid such as prednisone, a nutritional supplement, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab)], an antibody to a cytokine such as IL-1, IL-6, IL-13, IL-4, IL-17, IL-25, IL-33 or TGF-β, negative regulators of TGF-3/Smad signaling pathway (a recombinant BMP7 or peptide mimics of BMP7), and any other palliative therapy useful for ameliorating at least one symptom associated with a fibrosis-associated condition or cancer. In certain embodiment, the second therapeutic agent is anti-integrin inhibitor.

In some embodiments, the second therapeutic agent may be administered to manage or treat at least one complication associated with fibrosis or cancer.

In certain of these embodiments, an antibody or antigen-binding fragment as disclosed herein that is administered in combination with one or more additional therapeutic agents may be administered simultaneously with the one or more additional therapeutic agents, and in certain of these embodiments the antibody or antigen-binding fragment and the additional therapeutic agent(s) may be administered as part of the same pharmaceutical composition. However, an antibody or antigen-binding fragment administered “in combination” with another therapeutic agent does not have to be administered simultaneously with or in the same composition as the agent. An antibody or antigen-binding fragment administered prior to or after another agent is considered to be administered “in combination” with that agent as the phrase is used herein, even if the antibody or antigen-binding fragment and second agent are administered via different routes. Where possible, additional therapeutic agents administered in combination with the antibodies or antigen-binding fragments disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

In another aspect, the present disclosure provides kits or pharmaceutical compositions comprising the antibody or antigen-binding fragment thereof provided herein and the second therapeutic agent, which may be formulated in one composition, or in different compositions. Instructions for use or indications can be further included to provide information on how combined therapy are to be carried out.

Methods of Detection and/or Diagnosis

In some embodiments, the present disclosure provides methods of detecting presence or amount of GREM1 in a sample derived from a subject, comprising contacting the sample with the antibody or antigen-binding fragment thereof, and determining the presence or the amount of GREM1 in the sample.

The presence or amount of GREM1 in a sample can also be detected by measuring the mRNA level of GREM1 using techniques including, without limitation, RNA sequencing (RNA-seq) and RNAscope (Wang, Z., Gerstein, M., & Snyder, M. (2009). RNA-seq: a revolutionary tool for transcriptomics. Nature Reviews Genetics, 10(1), 57-63; Wang et al., RNAscope: a novel in situ RNA analysis platform for formalin-fixed, paraffin-embedded tissues, J Mol Diagn. 2012 January; 14(1): 22-9.). Briefly, the RNA-seq comprises reverse transcribing a target mRNA into a cDNA, fragmenting and sequencing the cDNA and analyzing the sequence data for mRNA quantification; the RNAscope comprises in situ hybridizing a target mRNA with one or more oligonucleotides conjugated with a fluorescent probe and detecting the level of mRNA by measuring the fluorescence intensity.

In certain embodiments, the biological sample comprises a cancer cell or a sample from tumor microenvironment (e.g. stromal cells or stroma).

In some embodiments, the present disclosure provides methods of detecting presence or amount of GREM1 in a sample, or diagnosing a GREM1 related disease or condition in a subject, comprising: a) contacting a sample obtained from the subject with the antibody or antigen-binding fragment thereof provided herein; b) determining the presence or the amount of GREM1 in the sample; and optionally c) correlating the presence or the amount of GREM1 to existence or status of the GREM1 related disease or condition in the subject. In certain embodiments, the biological sample comprises a cancer cell, stromal cell, stroma or a fibrotic cell.

In some embodiments, the present disclosure provides kits comprising the antibody or antigen-binding fragment thereof provided herein, optionally conjugated with a detectable moiety. The kits may be useful in detection of presence or amount of GREM1 in a biological sample, or may be useful in the methods of diagnosis provided herein.

In some embodiments, the present disclosure provides kits comprising the antibody or antigen-binding fragment thereof provided herein and a second therapeutic agent. The kits may be useful in treatment, prevention, and/or amelioration of GREM1-related disease.

In some embodiments, the present disclosure also provides use of the antibody or antigen-binding fragment thereof provided herein in the manufacture of a medicament for treating or diagnosing a GREM1-related disease or condition in a subject.

### EXAMPLES

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1: Preparation of Antigens

HGREM1-His (R&D): Recombinant HGREM1 Protein (Accession #060565) was expressed in NS-0 cells. Briefly the coding region of the hGREM1 gene from Lys25-Asp184 with 10×his tag at C-terminus was used for transfection. The supernatant was purified using His-tag affinity column. The resulting purified protein was characterized using SDS PAGE gel. The protein was purchased from R&D systems (Cat #5190-GR).

Mouse Gremlin-His (R&D): Recombinant mouse Gremlin (Accession #070326) Lys25-Asp184 was fused with 10×His tag at the C-terminal and produced in NS-0 cells. The transfection supernatant was purified using His-tag affinity column. The resulting purified protein was characterized using SDS PAGE gel. The protein was purchased from R&D systems (Cat #956-GR).

HGREM1-His (ACRO): Recombinant hGREM1 Protein Lys25-Asp184 (Accession #NP_037504) was fused with polyhistidine tag at C-terminus and produced in human 293 cells (HEK293). The transfection supernatant from HEK293 cells was purified using His-tag affinity column. The resulting purified protein was characterized using SDS page gel. This protein was purchased from ACRO Biosystems (Cat #GR1-H52H3).

HGREM1-Fc (ACRO): Recombinant hGREM1 Protein Lys25-Asp184 (Accession #NP_037504) was fused with hIgG1 Fc tag at C-terminus and produced in human 293 cells (HEK293). The resulting purified protein was characterized using SDS PAGE gel. This protein was purchased from ACRO Biosystems (Cat #GR1-H5254).

The above Gremlin proteins were used in the following experiments.

### Example 2: Antibody Generation

### 1. Antigen Conjugation and Immunization

For immunization, the recombinant hGremlin-His protein was conjugated with various MabSpace immune-enhancing peptides. Briefly 2-8 fold molar excess of the peptide was mixed with Sulfo-SMCC (sulfosuccinimidyl 4-[N-maleimidomethyl]cyclohexane-1-carboxylate, Pierce, Cat #22322)-activated hGremlin protein and incubated for one hour at room temperature. The reaction was stopped and the conjugated protein was analyzed and QCed using SDS-PAGE gel.

Above conjugated hGremlin-His protein was emulsified in a 1:1 ratio using Complete Freund's Adjuvant (Pierce, Cat #77140), respectively, and then immunized sub-cutaneously and intraperitoneally into C57B/L6 mice. Additional immunizations were carried out using CpG and Alum to preserve native conformation of the protein. Immunization occurred at least every 2 weeks and antiserum from the mice was taken after the 1st immunizations for anti-hGremlin titer analysis by ELISA assay.

For determining the serum titer, 20 μl of mouse serum was prepared from each immunized mouse. High-binding clear polystyrene 96 well plates (Nunc) were coated with 100 μl/well of a 1 μg/ml solution consisting of hGREM1-His in high pH coating buffer (0.16% Na₂CO₃, 0.3% NaHCO₃, pH9.8). The plates were incubated overnight at 4° C., and then washed once on an automatic plate washer using washing buffer PBS+0.1% Tween 20 (Sigma). 200p1 of blocking buffer (PBS+1% BSA+1% Goat serum+0.05% Tween 20) was added to each well and incubated for 2 hours at room temperature. The blocking buffer was then aspirated and 100 μl of serially diluted serum in dilution buffer (PBS+1% BSA+1% Goat serum+0.01% Tween 20) was transferred to each well of the ELISA plate and allowed to incubate for 60 min at room temperature. The plates were then washed 3 times using the method described above. 100p/well of solution of HRP conjugated goat anti-mouse Fc antibody (Abcam, Cat #Ab98808) diluted in dilution buffer was then added to each well of the plate. After that the ELISA plates were allowed to incubate for 60 min at RT, the plates were washed 3 times with 250 μl/well washing buffer. Finally, 100 μl/well of TMB was added to each well and the reaction was terminated using 0.64M H₂SO₄. The plates were read on a Thermo Multiscan FC at 450 nM.

### 2. Fusions

Four days prior to fusion, each mouse was boosted intraperitoneally with unconjugated hGremlin-His protein in PBS. On the fusion day, the spleens were removed aseptically and the organs were processed into a single cell suspension. The red blood cells were lysed and the splenocytes were washed with DMEM (Gibco). Viable, log-phase growth myeloma cells (SP2/0) were mixed with the murine splenocytes in a 1:4 ratio. The cells were then washed 2 times before the fusion with PEG. The post fusion cells were washed with DMEM and suspended in cell growth media supplemented with 10% FBS+HFCS+OPI+1×HAT. 200p1 per well of this cell suspension was plated into 96-well cell culture plates and incubated overnight in a 37° C. humidified 10% CO₂ incubator. The cultures were incubated for 7 days and then the growth media was aspirated out of the wells and exchanged for fresh growth media. Screening of hybridoma supernatants commenced 2-3 days after the media change.

### 3. Antibody Screening by ELISA Assay

Same protocol of determining serum titer above was used. Briefly, l μg/ml hGremlin-His were coated overnight at 4° C. After wash, 100 μl of hybridoma supernatant was added and allowed completely binding. HRP conjugated goat anti-mouse Fc antibody was then added to detect bound Gremlin antibody. Finally, the plates were read on a Thermo Multiscan FC at 450 nM after TMB reaction and H₂SO₄ termination. Cells from the ELISA positive hybridoma wells were subsequently expanded in cell culture for further characterization studies.

### Example 3: Subcloning of Positive Hybridoma Clones and Small-Scale Antibody Production

### 1. Subcloning of Positive Hybridoma Clones

Cells from the ELISA positive hybridoma wells with the desired binding profile and blocking activity were selected and each plated using limited dilution in 96 well plates. These cells were allowed to grow for 7 days. Once the adequate cell mass was reached, supernatant from each well was collected and re-screened for antigen binding ability (see screening in Example 2).

From each 96 well plate, clones with highest antigen binding activity were identified and expanded with limited dilution further into 96-well plates with 200 μl of hybridoma growth medium per well. After 7 days, cells from 96-well plates were tested for antigen binding. The subcloning was done more than 2 times. When more than 90 of the wells display positive binding signal, two clones with the highest antigen binding activity were identified and transferred to 24-well plates with medium and was allowed to grow for 2 additional days. Once 24 well plates were confluent, cells were transferred to 6-well plates. After 5 days of incubation, a portion of the cells were frozen down. The remainder of the cells were transferred into a flask and allowed to expand. Once the flasks were confluent, half of cells were frozen down (3 vials per clone) for additional backup. The other half was allowed to expand further in flasks with medium for antibody production. Isotypes were determined using standard methodologies.

### 2. Small Scale Antibody Production

Hybridoma cells were inoculated to roller bottle and cultured for 14 days with 200-300 ml of hybridoma culture medium (Invitrogen). Gremlin monoclonal antibodies (mAbs) were purified from hybridoma cell culture as follows. All purification processes were carried out at room temperature. One purification scheme was used to purify various mAbs and used affinity chromatography.

The host cell culture fluid (CCF) was centrifuged to remove cell debris. The CCF supernatant was then filtered, diluted and then loaded onto Protein G chromatography media in the form of a column, Protein G High Performance (Bio-Rad) and equilibrated.

After loading, the Protein G column was washed until the absorbance at 280 nm of the flow-through returned to baseline. The Gremlin mAb was then eluted from the column using glycine, pH 2.5 and immediately neutralized by adding 50 μL of a stock solution of 1 M Tris Base per mL of elution volume. The absorbance at 280 nm of the eluate was monitored and fractions containing protein were collected to make the Protein A pool.

Following purification, the Gremlin mAbs were formulated in PBS by dialysis using 10,000 MWCO membranes (Pierce Slide-A-Lyzer or dialysis tubing). Following formulation, the Gremlin mAbs were filtered.

### Example 4: Binding Analysis of Purified Hybridoma Anti-Gremlin1 Antibodies to Captured Human and Mouse Gremlin by ELISA

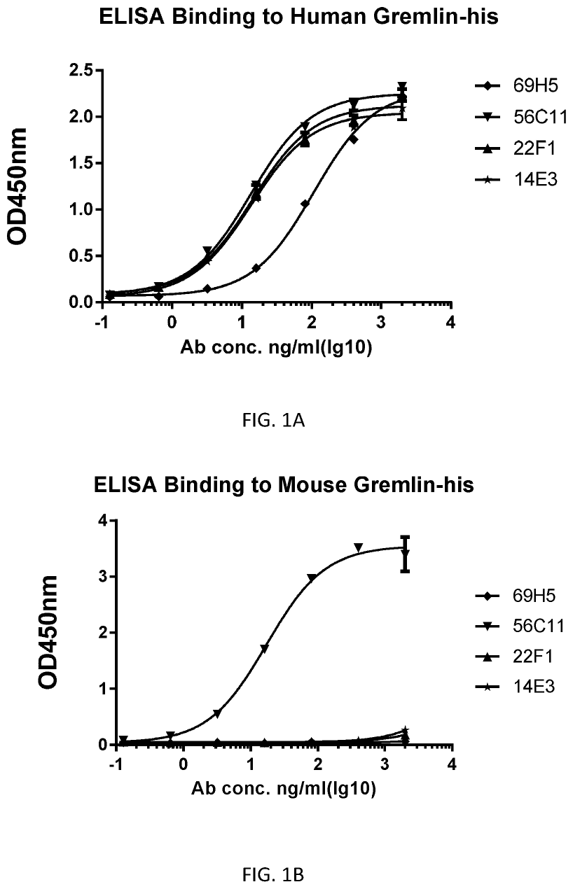

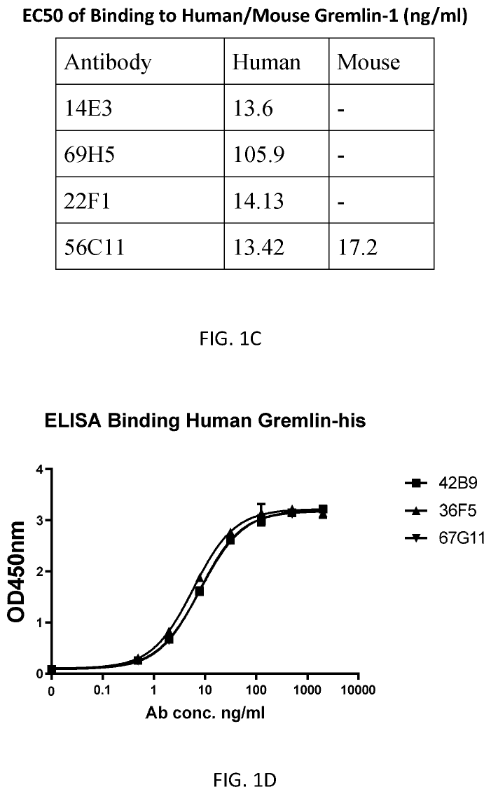

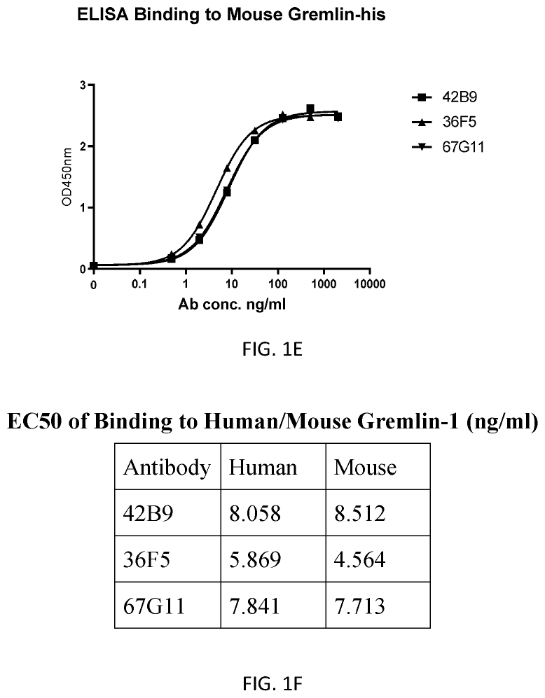

Clear polystyrene plates (BEAVER) were coated with 100 μl/well of a 0.5 g/ml hGREM1 (ACRO) and mouse gremlin (R&D) in high pH coating buffer overnight at 4° C. Then the plates were washed once on an automatic plate washer using PBS+0.1% Tween 20 (Sigma). 100 μl of block solution consisting of PBS+1% BSA+1% normal goat serum+0.5% Tween 20 (Sigma) was added to each well and incubated at room temperature for 2 hours. Then 100 μl of antibodies in antibody dilution buffer containing PBS+1% BSA+1% normal goat serum+0.01% Tween 20 starting from 2 μg/ml and then serial dilutions were added to each well of the plate and incubated for 1 hours at room temperature. Afterward the plates were washed three times with 200 μl of PBS+0.1% Tween20 followed by adding 100 μl/well 1:10000 Goat anti mouse IgG-HRP (abcam), and incubate for 1 hour at room temperature. They were then washed 3× with PBS+0.1% Tween20. Finally, 100 l/well of TMB (Pierce) was added to each well and after several minutes add 50 μl of stop solution to each well. The plates were read on a Multiscan FC microplate reader (Thermo Scientific) at 450 nM. As shown in FIG. 1, 56C11, 42B9, 36F5 and 67G11 showed high binding affinity to both hGREM1 and mouse gremlin (with a EC50 value of 13.42 ng/ml and 17.2 ng/ml respectively for 56C11, a EC50 value of 8.058 ng/ml and 8.512 ng/ml respectively for 42B9, a EC50 value of 5.869 ng/ml and 4.564 ng/ml respectively for 36F5, and a EC50 value of 7.841 ng/ml and 7.713 ng/ml respectively for 67G11), whereas 69H5, 22F1 and 14E3 showed binding affinity selectively to hGREM1 (with a EC50 value of 105.9 ng/ml, 14.13 ng/ml and 13.6 ng/ml respectively) over mouse gremlin.

Humanized antibodies provided herein (e.g., Hu14E3, Hu22F1 and Hu56C11) showed similar high binding affinity to hGREM1 and/or mGREM1.

### Example 5: Characterization of Binding Specificity of Hybridoma Antibodies Binding to Captured hGREM1 or Related Family Protein by ELISA

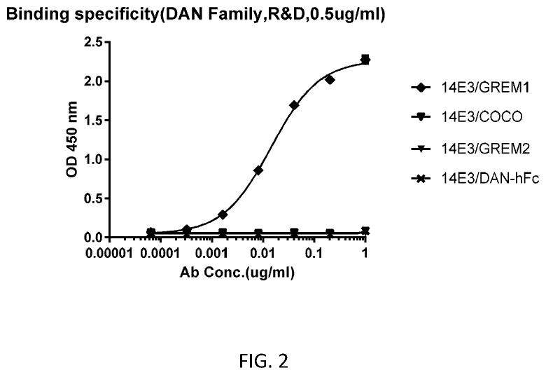

In addition, the specificity of the binding of 14E3 and a benchmark antibody was assessed with ELISA. Briefly, clear polystyrene plates (BEAVER) were coated with 100 μl/well of a 0.5 g/ml hGREM1 (ACRO), or human gremlin-2 (R&D), human COCO (R&D) and human DAN protein (R&D) in high pH coating buffer overnight at 4° C. Then the plates were washed once on an automatic plate washer using PBS+0.1% Tween 20 (Sigma). 100 μl of block solution consisting of PBS+1% BSA+1% normal goat serum+0.5% Tween 20 (Sigma) was added to each well and incubated at room temperature for 2 hours. Then 100 ul of antibodies in antibody dilution buffer containing PBS+1% BSA+1% normal goat serum+0.01% Tween 20 starting from 2 μg/ml and then 5 fold serial dilution were added to each well of the plate and incubated for 1 hours at room temperature. Afterward the plates were washed three times with 200 ul of PBS+0.1% Tween20 followed by adding 100 μl/well 1:10000 Goat anti mouse IgG-HRP (Abcam), and incubate for 1 hour at room temperature. They were then washed 3× with PBS+0.1% Tween20. Finally, 100 l/well of TMB (Pierce) was added to each well and after 5 minutes add 50 ul of stop solution to each well. The plates were read on a Multiscan FC microplate reader (Thermo Scientific) at 450 nM. The result showed that 14E3 binds specifically to hGREM1 but not human gremlin-2, COCO and DAN protein which share high homology structurally (FIG. 2).

### Example 6: Characterization of Antibody Activity in Blocking of Gremlin Binding to Captured BMP 2/4/7

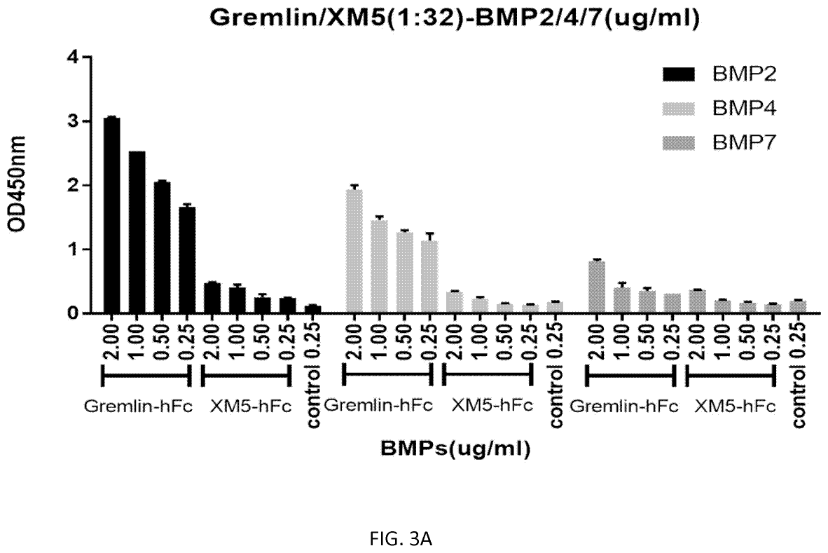

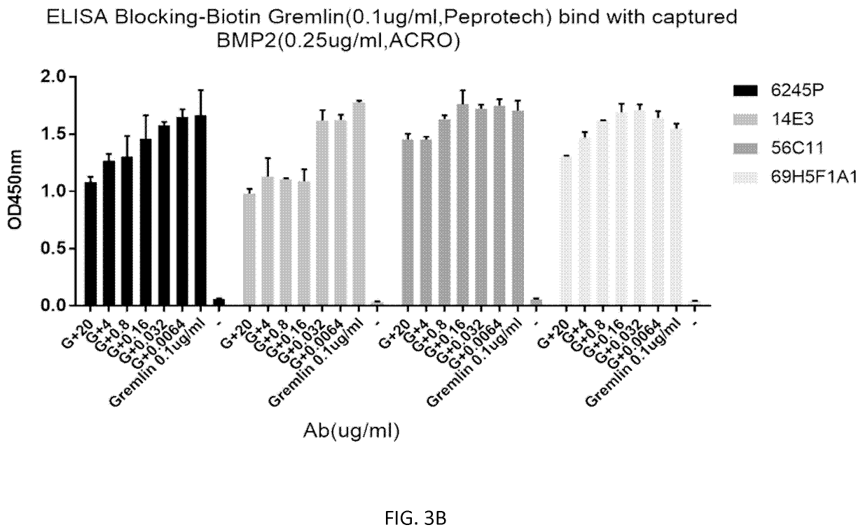

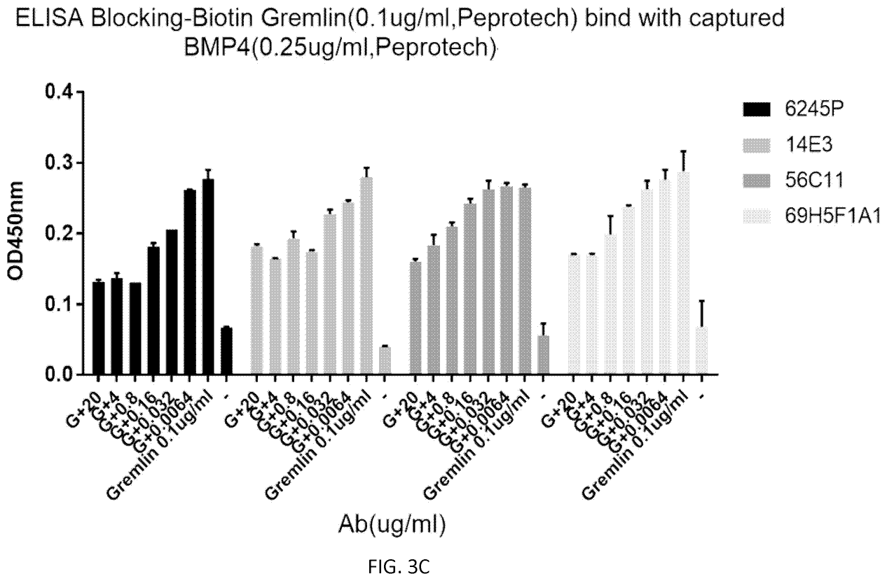

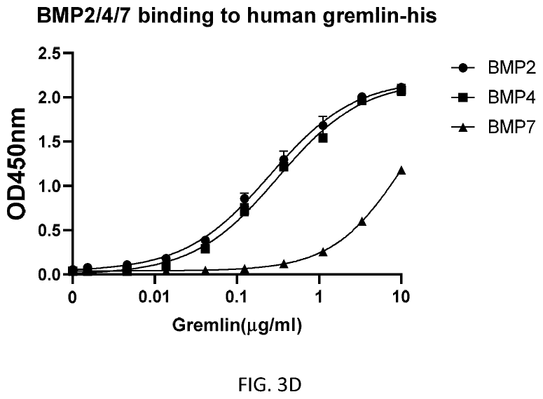

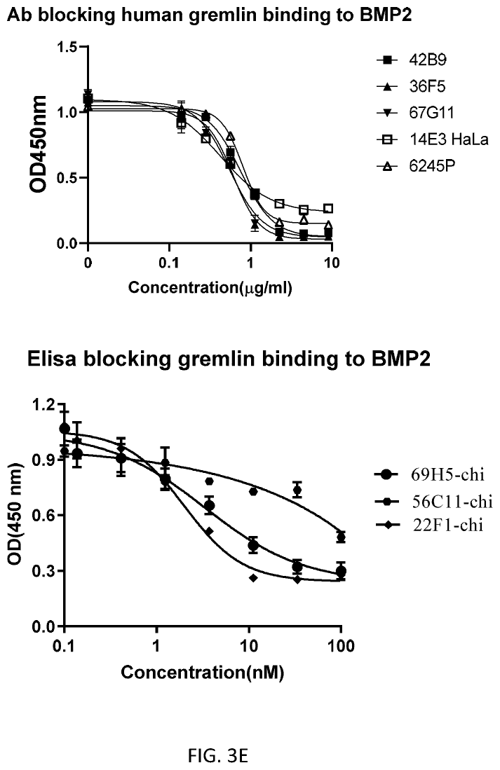

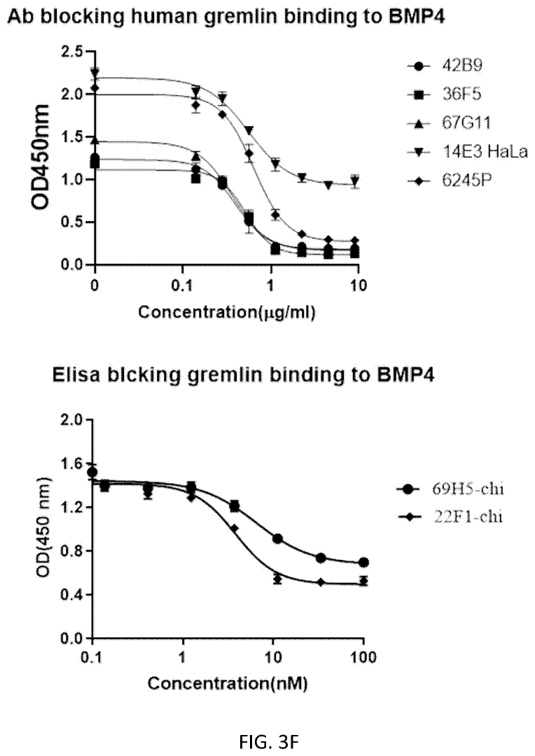

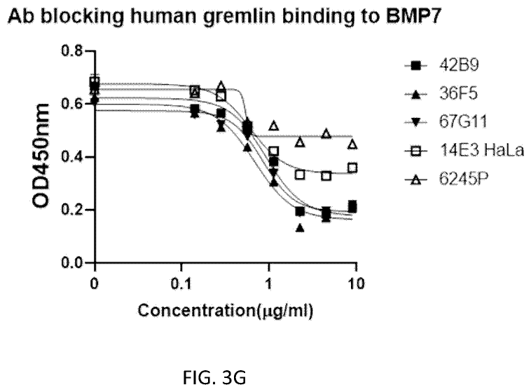

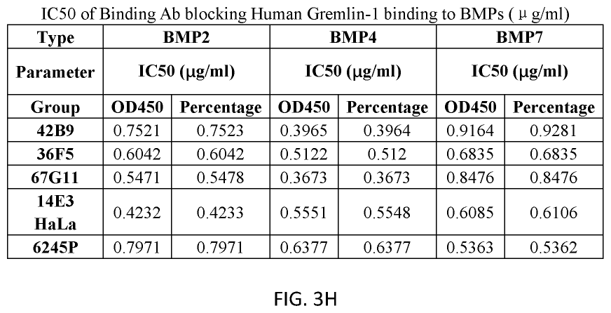

Gremlin is known to be able to bind to BMP proteins and is validated by the present disclosure. Briefly, gremlin-Fc was tested for its binding ability to BMP 2/4/7 immobilized on plate. Briefly, plates were coated with 0.5 g/ml of recombinant human BMP2, BMP4) (hBMP4, Peprotech) or human BMP7 (R&D) overnight and wild-type gremlin-1 was added to coated plates and allowed to incubate for an additional hour at RT. The plates were then washed and plate bound biotin-hGremlin was detected with neutravidin conjugated with HRP (thermo). Plates were then developed with a TMB solution and stopped by adding stop solution. The plates were read on a plate reader at 450 nm. FIG. 3A and FIG. 3D shows that wild-type gremlin could bind to BMP 2, BMP 4 and BMP 7, and the binding affinity to BMP 2 and 4 was stronger than that to BMP7.

A mutated version of gremlin XM5 was also tested for its binding ability to BMP 2/4/7 head to head with gremlin. XM5 was constructed and expressed by Mabspace, wherein the 123-143 amino acids of gremlin (NSFYIPRHIRKEEGSFQSCSF, SEQ ID NO: 63), which is known as the binding loop of BMPs, was replaced by the 63-83 amino acids of DAN (FSYSVPNTFPQSTESLVHCDS, SEQ ID NO: 64), which does not bind to BMPs. His-tag or Fc-tag were constructed at C-terminal of protein. For stickiness of XM5 and gremlin, unpurified supernatant was used for this assay. Briefly, anti-human Fc antibody (1 g/ml) was coated and supernatant of XM5-Fc or Gremlin-Fc (1:32 diluted) were added. After incubation, serial diluted BMPs (BMP2/4/7) with his-tag were added and then secondary antibody anti-his-HRP were used for detection of binding.

As shown in FIG. 3A, contrary to gremlin, BMPs had no binding to XM5, which was expected and validated that at least one amino acid in the binding loop of BMP comprising the SEQ ID NO: 63 on gremlin is essential for its binding to BMP. The ability of antibodies to block Gremlin binding to human bone morphogenetic protein 2 and 4 (hBMP2, hBMP4) was examined via ELISA. Plates were coated with recombinant human BMP 2 and BMP 4 (0.25 μg/ml) (hBMP2, hBMP4, Peprotech) overnight and then serial dilutions of antibodies were incubated with 0.1 μg/ml of hGREM1 (Peprotech) modified with a biotin tag for 1 h at RT before this complex was added to coated plates and allowed to incubate for an additional hour at RT. The plates were then washed and plate bound biotin-hGremlin was detected with neutravidin conjugated with HRP (thermo). Plates were then developed with a TMB solution and stopped by adding stop solution. The plates were read on a plate reader at 450 nm. The results were shown in FIG. 3B and FIG. 3C, indicating that the anti-gremlin 1 antibodies provided herein (e.g, 14E3, 56C11 and 69H5 as well as a benchmark antibody 6245P, which was produced according to the sequence of H4H6245P disclosed in WO2014159010, disclosure of which is incorporated by reference in its entirety) can inhibit the binding of gremlin to BMP2 and BMP4 in a dose-dependent manner, though to different degree.

In addition, the ability of serial dilutions of antibodies provided herein to block Gremlin binding to human BMP2/4/7 was also examined via ELISA. Plates were coated with recombinant human BMP2/4/7 (0.5 μg/ml) overnight and then serial dilutions of antibodies were incubated with 1 μg/ml of human Gremlin-his modified for 1 h at RT before this complex was added to coated plates and allowed to incubate for an additional hour at RT. The plates were then washed and added with anti-his HRP (GenScript). Plates were then developed with a TMB solution and stopped by adding stop solution. The plates were read on a plate reader at 450 nm. The results were shown in FIG. 3E-FIG. 3H, indicating that the anti-gremlin 1 antibodies provided herein (e.g., 42B9, 36F5, 67G11, 14E3 HaLa, chimeric antibody 69H5 (69H5-chi), chimeric antibody 56C11(56C11-chi) and chimeric antibody 22F1(22F1-chi)) as well as a benchmark antibody 6245P can inhibit the binding of gremlin to BMP2 (see, FIG. 3E), BMP4 (see, FIG. 3F) and BMP 7 (see, FIG. 3G). The chimeric anti-gremlin 1 antibodies (69H5-chi) and chimeric anti-gremlin 1 antibodies (22F1-chi) also can inhibit the binding of gremlin to BMP2 and BMP4 (see, FIG. 3E and FIG. 3F). The chimeric anti-gremlin 1 antibodies (56C11-chi) can inhibit the binding of gremlin to BMP2 (see, FIG. 3E).

In particular, as shown in FIG. 3G, the anti-gremlin 1 antibodies provided herein (42B9, 36F5, 67G11 and 14E3 HaLa) can significantly block the binding of gremlin to BMP7, and showed a maximal blocking percentage of at least 50% or even at least 70%. In contrast, the benchmark antibody 6245P only showed less than 30% of maximal blocking on the binding of human gremlin to BMP7, suggesting 6245P was much less effective in blocking BMP7.

### Example 7: Characterization of Antibody Activity in Blocking of Gremlin Mediated BMP Signaling Using BMP Responsive Reporter Assay

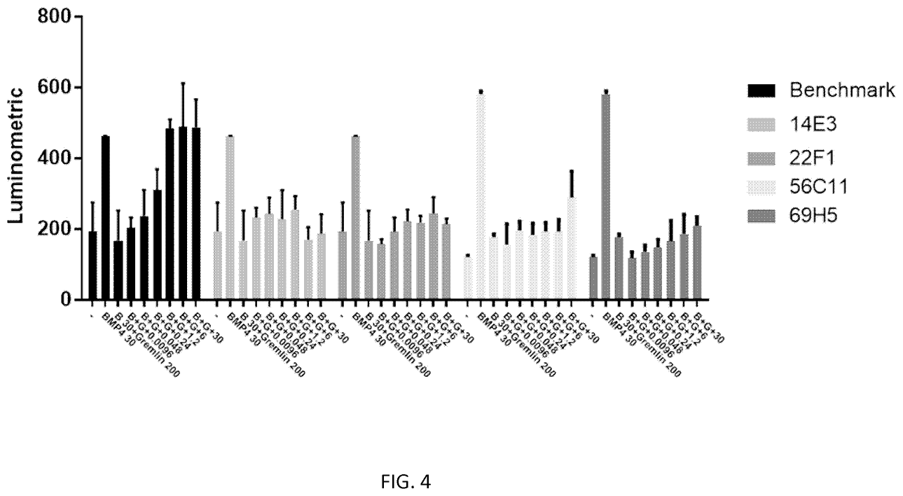

These purified hybridoma antibodies were also tested for their ability in reducing gremlin-mediated inhibition of BMP signaling using BMP4-induced luciferase reporter assay in BRITER (a BMP responsive osteoblast reporter cell line, Abmgood, T3105). Briefly, the BRITER cells were plated in 96-well plates at 10000 cells/well and incubated at 37° C. and 5% CO₂ overnight. The next day, the cells were stimulated with either control medium, or medium with 30 ng/ml BMP 4 (BMP4 30) or 30 ng/ml BMP 4 plus Gremlin 200 ng/ml (B 30+Gremlin 200); or 30 ng/ml BMP 4 plus gremlin 200 ng/ml with different concentrations of antibodies provided herein (B+G+0.096/0.048/0.24/1.2/6/30 μg/ml) as shown in the x axis of FIG. 4. The luciferase activity of the cells in each well was measured after 3 hours of incubation in 37° C. and 5% CO₂ using a plate reader (Thermo Scientific Varioskan Flash).

As shown in FIG. 4, in contrast to the benchmark antibody, the antibodies showed no activity in reducing gremlin-mediated inhibition of BMP signaling in the reporter cells which is not cancer cell origin while the benchmark antibody can reduce or reverse gremlin-mediated inhibition of BMP signaling.

### Example 8: Characterization of Antibody Activity in Reducing Gremlin-Mediated Inhibition of BMP Signaling and Cell Differentiation

The purified hybridoma antibodies were also tested for their ability in reducing gremlin-mediated inhibition on BMP signaling using BMP4-induced ATDC-5 cell differentiation. ATDC-5 is a chondrogenic cell line and can differentiate in response to BMP4 signaling. The differentiation of the chondrogenic cell line can be blocked by Gremlin, a known BMIP inhibitor. Blocking of Gremlin results in a reversal of BMP4 inhibition in this assay. Differentiation can be measured colorimetrically by using a substrate to detect endogenous expression of alkaline phosphatase (ALP), an early marker of osteoblast differentiation. The differentiation level can positively reflect the activity of BMP4 signaling.

ATDC-5 cells were plated in 96-well plates at 3000 cells/well and grown in DMEM/1F12, 10% FBS+1% PS at a volume of 100 ul/well and incubated at 37° C. and 5% CO₂ overnight. The next day, hGREM1 was mixed with serially diluted antibodies in serum free medium and incubated at 37° C. for 30 mins. Human BMP4(Peprotech) diluted in serum free medium was added to the Gremlin/antibody mixtures and then incubated at 37° C. for additional 30 mins. After incubation, 100 ul of the mixtures was added to ATDC-5 cells plated in 100 ul of complete medium. The final concentration of hBMP4 and hGremlin on cells in each well was 100 ng/ml and 400 ng/ml, respectively. After 3 days of growth at 37° C. and 5% CO₂, medium was aspirated and wash twice with cold PBS. The cells were lysed with M-PER buffer (Thermo)+protein inhibitor (Roche). ALP was measured using p-nitrophenyl phosphate (PNPP) (Sigma). OD405 was measured on a Multiscan FC microplate reader (Thermo Scientific).

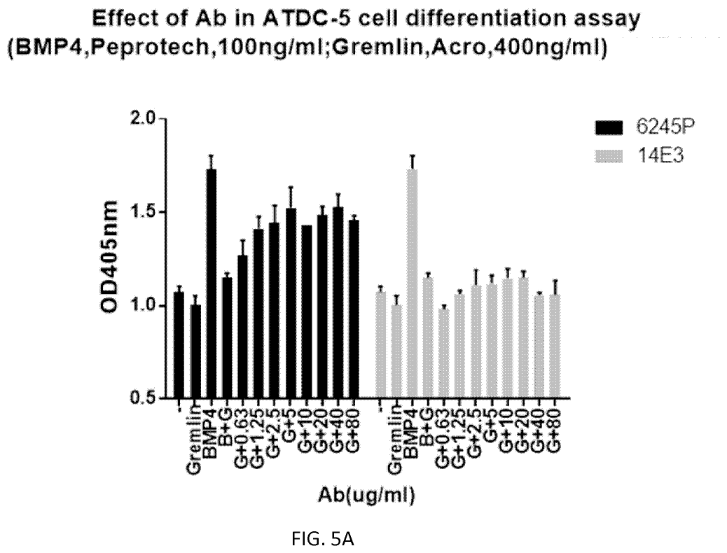

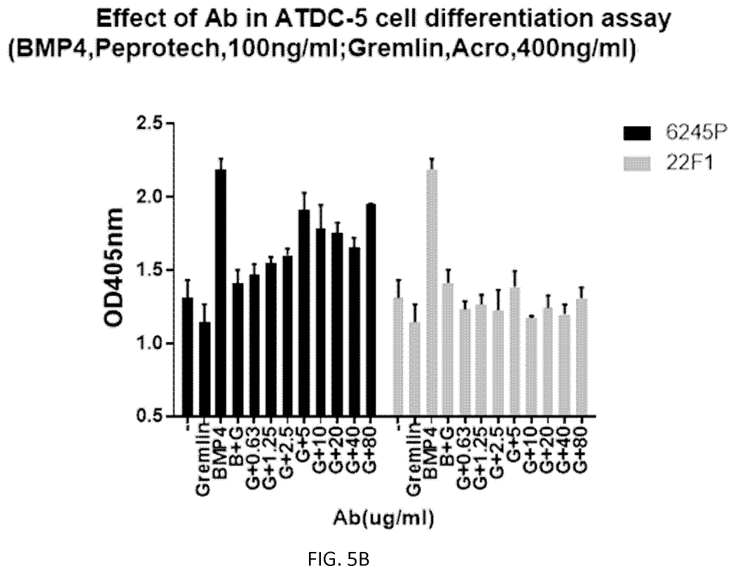

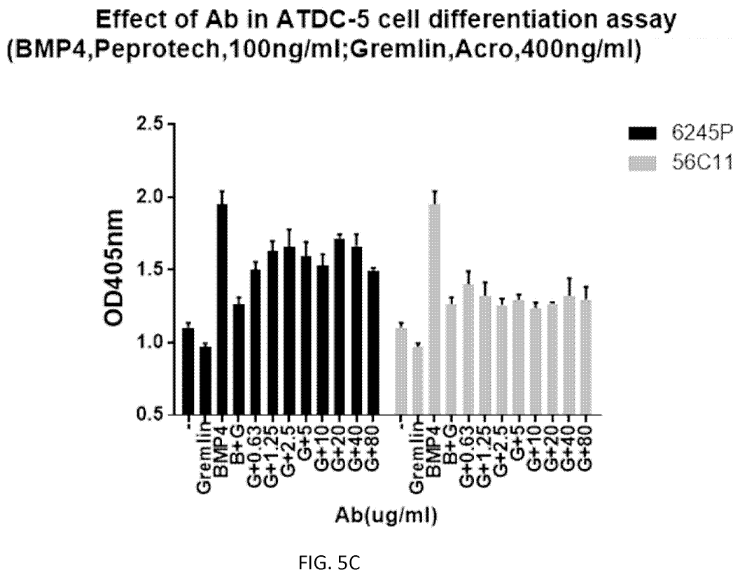

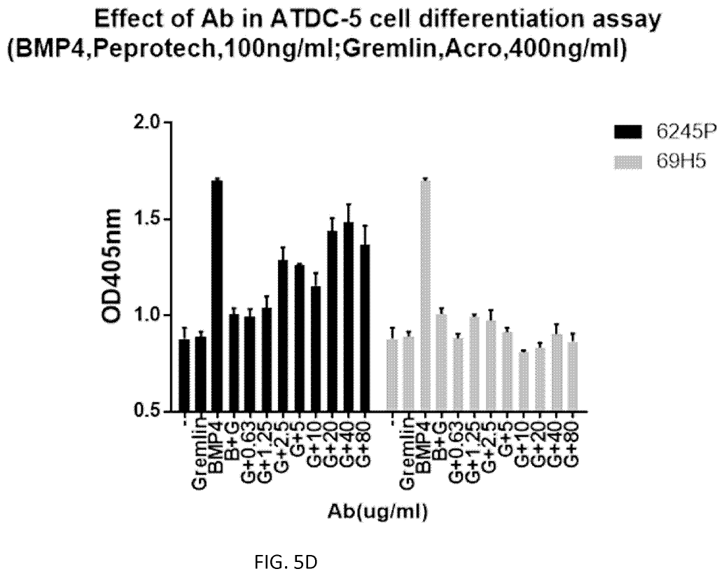

As shown in FIG. 5, while the benchmark antibody 6245P could reduce the gremlin-mediated inhibition on BMP signaling in cell differentiation, the antibodies 14E3 (FIG. 5A), 22F1 (FIG. 5B), 56C11 (FIG. 5C), and 69H5 (FIG. 5D) showed no such effect. In other words, the antibodies provided herein were not able to restore the gremlin inhibition on BMP signaling involved in cell differentiation, which is not cancer origin either.

### Example 9: Characterization of Gremlin Mediated Inhibition of BMP Signaling in PC3 Prostate Cancer Cells and Evaluation of the Antibodies Provided Herein in Reversing Gremlin-Mediated Inhibition on BMP Signaling in PC3 Cells

PC3 prostate cancer cells were plated in 12-well plates at 100000/well in DMEM/10% FBS, 1% PS (complete media) and grown to 90% confluency at 37° C. and 5% CO₂. After starved with serum free medium overnight, the PC3 cells were stimulated with BMP4 with or without gremlin for 30 min. The cells were lysed in RIPA buffer (CST) for a Western blot analysis. The cell lysates were separated using a 4%-12% SDS-PAGE (Genscript) and transferred to a PVDF membrane (Millipore). The membranes were incubated with an antibody specific for smad phosphorylation (p-smad1/5/9) (1:1000, CST) that positively reflects the activity of BMP signaling and an antibody specific for β-actin (1:5000, Abbkine) as a control respectively, at 4° C. overnight, followed by incubation with the corresponding secondary antibodies. Color development was carried out using Pierce ECL Western Blotting Substrate (Thermo Scientific) and visualized using a Cheniluminescent Imager (MiniChemi, Sagecreation).

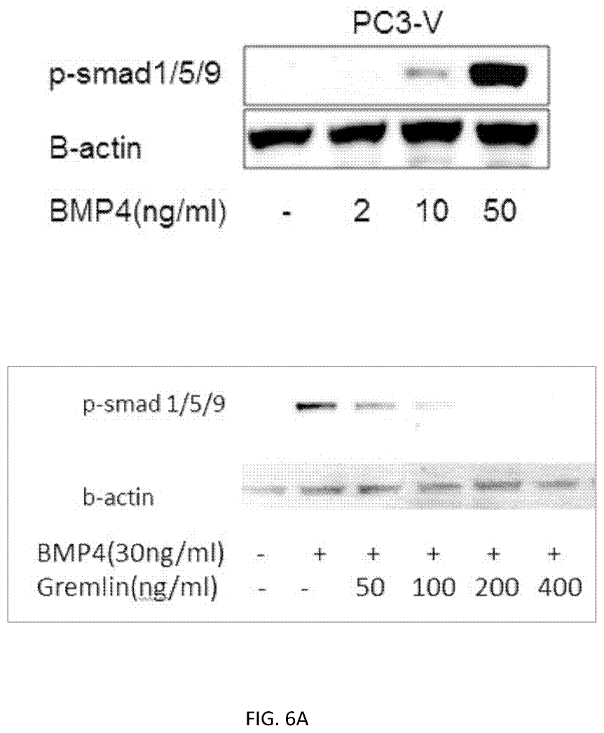

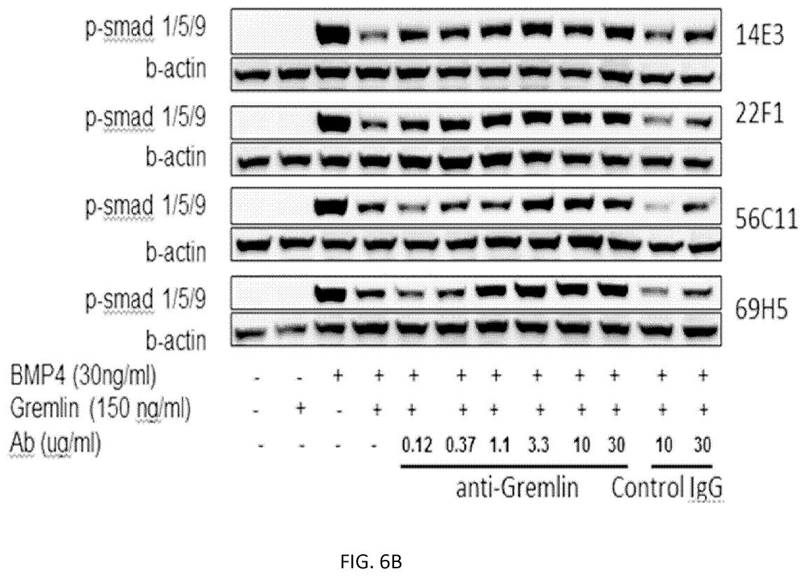

Results showed that gremlin reduced the level of BMP induced p-smad1/5/9 in a dose dependent manner, suggesting that gremlin could indeed inhibit BMP signaling (FIG. 6A).

The ability of different antibodies provided herein in blocking gremlin-mediated inhibition on BMP induced p-smad1/5/9 was further evaluated. Gremlin was incubated with the antibodies provided herein of different concentrations for 30 min, 37° C., and the mixture of gremlin and antibodies was incubated with BMP4 for 30 min, 37° C., before being added into the plated PC3 cells. After 30 mins, the cells were lysed in RIPA buffer (CST) for Western blot analysis using the above mentioned protocol.

As shown in FIG. 6B, the intensity of p-smad 1/5/9 or pSMAD1/5/9 recovered from the GREM1-mediated inhibition increased as the concentration of the anti-hGREM1 antibodies provided herein (e.g., 14E3, 22F1, 56C11, 69H5) increased. This experiment showed that these antibodies provided herein can modulate (e.g., reduce) gremlin-mediated inhibition on BMP signaling in a dose dependent manner.

### Example 10: Characterization of the Differential Reversal of Gremlin-Mediated Inhibition on BMP Signaling by Anti-Gremlin1 Antibodies Provided Herein in Different Cell Types

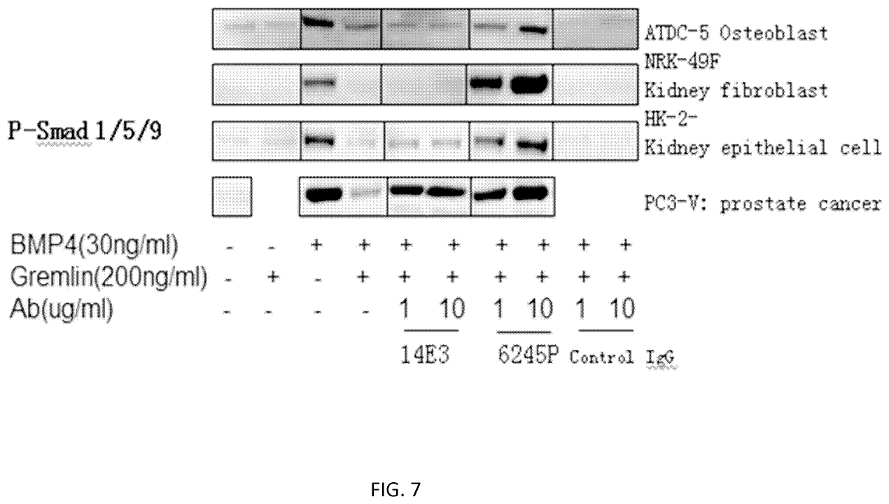

As gremlin is expressed in multiple physiological tissues and has impact on multiple cell types, we evaluated whether the anti-gremlin1 antibodies provided herein had similar effect on different cell types that are responsive to gremlin-mediated inhibition on BMP signaling. Briefly, multiple cell types of different origins, including osteoblast cell ATDC-5, kidney fibroblast NRK49F, kidney epithelial cells HK2 cells as well as tumor cell PC3 were stimulated with BMP4, or BMP4 with gremlin. The cells stimulated with both BMP4 and gremlin were then supplied with 1 or 10 μg/ml gremlin antibodies or control IgG. The antibodies tested here include 14E3 and benchmark antibody 6245P. As shown in FIG. 7, whereas gremlin can potently inhibit BMP4 induced pSMAD1/5/9, and benchmark antibody 6245P can reverse the gremlin-mediated inhibition of BMP4 induced pSMAD1/5/9 in all cell types tested, 14E3 only reverse gremlin-mediated inhibition on BMP4 signaling in tumor cells such as PC3 cells but not in other cells types (e.g., ATDC-5 osteoblast, NRK-49F kidney fibroblast, HK-2-kidney epithelial cells). This unexpected result suggests that the anti-gremlin1 antibodies provided herein (14E3, 22F1, 56C11, 42B9, 36F5, 67G11 and 69H5, Hu14E3, Hu22F1 and Hu56C11) are very different from the benchmark antibody in their biological activity, which is consistent with their differential activity using ALP assay in ATDC-5 cell as described in Example 8. Such selectivity for BMP signaling reversion in cancer cells over non-cancer cells indicates that the anti-hGREM1 antibodies (e.g., 14E3, 22F1, 56C11, 42B9, 36F5, 67G11 and 69H5, Hu14E3, Hu22F1 and Hu56C11) provided herein can selectively affect cancer cells while have little toxicity to non-cancer cells.

### Example 11: Cloning and Sequencing of Hybridoma Antibodies

Four of the lead antibodies that displayed desired profile were selected for gene cloning. The sequences of the murine anti-hGREM1 light chain and heavy chain variable regions were obtained by the polymerase chain reaction (PCR) amplification technique known as 5′ RACE (rapid amplification of cDNA ends). Total RNA from gremlin antibody producing hybridoma cell were isolated using Trizol (Invitrogen) and cDNA was synthesized using Superscript first strand synthesis system (Invitrogen) with Oligo (dT)12-18 primer. The variable regions of mouse IgG gene were cloned by PCR with MuIgG VH3′-2 and MuIg-5′leader primers for heavy chain variable region and MuIgK VL3′-1 and MuIg-5″ leader primer for light chain variable region (NOVAGEN). The resulting band was cloned into TOPO TA cloning vector and DNAs from more than 10 clones were submitted for sequencing and determined using ABI DNA sequencing instruments (Perkin Elmer). Consensus sequences were determined using Vector NTI Advance 10 software (Invitrogen). After sequencing analysis and confirmation, the variable region of the gremlin gene was cloned into a recombinant expression vector (VL into pCP-mCK; VH into pCP-mCg2a) for antibody production and purification. The sequence of these hybridoma antibodies are shown in Table 10. Table 10 shows all sequences used in the present application.

The antibody was isotyped as mouse IgG2b. To facilitate the secretion of the antibody, a signal peptide (MGWSCIILFLVATGVHS (SEQ ID NO: 65)) was fused at N-terminal of the antibody.

### Example 12: Expression and Purification of Recombinant Antibody Protein in 293E6 Cells

The expression and purification of a recombinant antibody protein were conducted by following methods: HEK293E cells cultured in Freestyle 293 Expression Medium with 10% of Pluronic F-68 at 1×10⁶ cell/ml were transfected with equal amount of heavy chain vector and light chain vector DNA at final concentration of 0.5 μg/ml and PEI (Polyethylenimine-linear, Polyscience) at 1.0 μg/ml. DNA to PEI ratio was 1:2. DNA and PEI complexes formed period with Optimal MEM should be 15 minutes at the room temperature. Transfected cells were cultured in the flasks with 5% CO₂, 37° C. and 125 rpm shaking speed. 1% Peptone medium was added at 22 to 26 hours post transfection. Conditioned medium was harvested on day 6 and supernatant was centrifuged at 3,000 rpm for 30 minutes. The clarified conditioned medium were then loaded onto nProteinA column (G.E. Healthcare), washed with PBS plus 0.1% triton-X100 and finally the bound IgG was eluted with a solution containing 0.1M glycine at pH 3.5. The eluted antibody protein was dialyzed to PBS and stored at −80° C. To remove endotoxin, the purified protein was further processed by passing through Hitrap DEAE Sepharose F.F. column and the resulting antibody was analyzed to determine the level of purity using size exclusion chromatography (Superdex 200 5/150 GL, G.E. Healthcare).

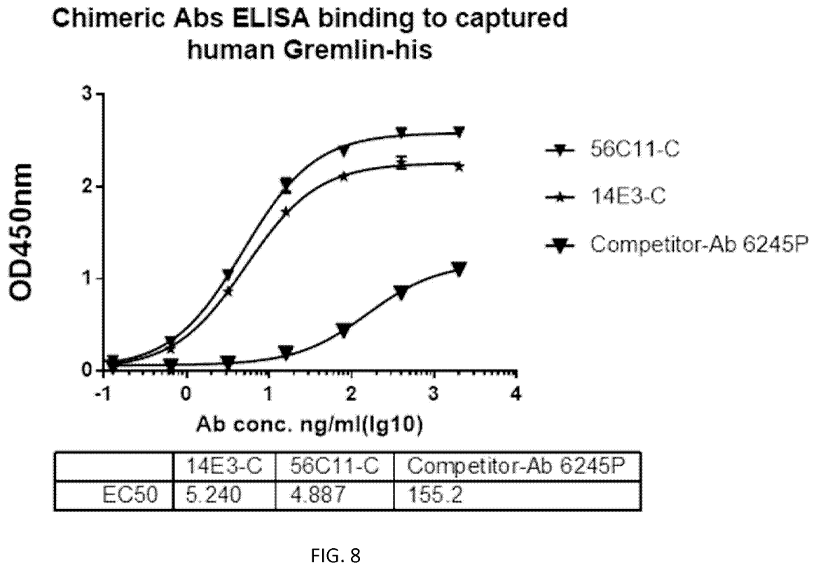

Binding analysis of recombinant chimeric anti-gremlin1 antibodies prepared according to the method described above is shown in FIG. 8, wherein the antibodies 56C11-C (i.e. chimeric antibody of 56C11) and 14E3-C (i.e. chimeric antibody of 14E3) showed significantly higher binding affinity with a lower EC50 value (5.240 ng/ml for 14E3-C and 4.887 ng/ml for 56C11-C) as compared to the benchmark antibody 6245P (115.2 ng/ml).

### Example 13: Large Scale Production of Selected Antibodies for In Vivo Studies

Each of these cloned antibodies was scaled up to produce large quantity of antibody for in vivo testing in renal failure models.

The hybridoma cells were cultured in roller bottle with in vitro production medium, then the monoclonal antibodies produced in the conditioned medium will be processed and purified by Protein A affinity column by low endotoxin procedure.

Briefly, the hybridoma cells were recovered and expanded and the cells were adapted to grow in hybridoma production medium (DMEM+2% Low IgG FBS) and inoculated into roller bottles with 300 ml of culture medium each. Then culture the cells in roller bottle for 2-3 weeks and hHarvest and clarify the culture medium before purification. Then the produced mAb from culture medium was purified by Protein A affinity column, dialyzed against PBS, pH 7.4, and concentrated to 1.0 mg/ml or higher if needed. The following parameters were measured for quality control: antibody product purity, endotoxin level, aggregation level as well as binding to target antigen.

### Expected Product Specification:

a. Buffer: Phosphate buffer saline (PBS), pH 7.2-7.4, sterile filtered and no preservatives.
b. Concentration: 1.0 mg/ml or higher
c. purity: 90% or better by SDS-PAGE and HPLC
d. aggregation: Less than 10% by HPLC
e. endotoxin: 3 EU/mg or less

### Example 14: Characterization of Binding Affinities Measurement of Selected Gremlin Antibodies to Captured hGREM1

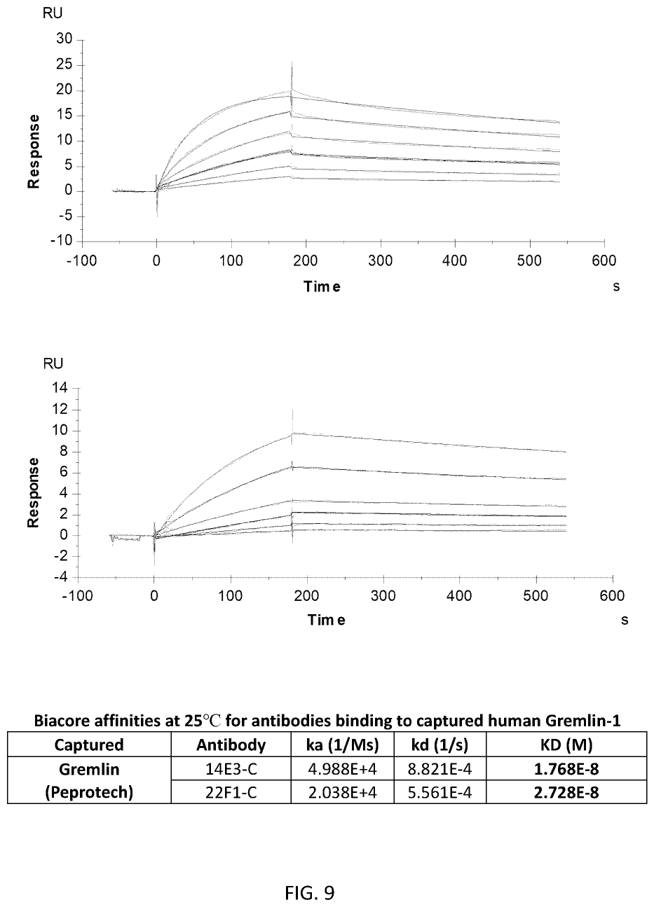

The affinity of the antibodies were measured by Biacore T200 at 25° C. HGREM1 (Peprotech) was immobilized on the Biacore sensor chip. Kinetic experiments were carried out using HBS-EP+ as both the running buffer and the sample buffer. Antibody-antigen association rate were measured by injecting various concentrations (ranging from 12.5 to 400 nM, 2-fold dilutions) of antibodies over the captured hGREM1 surface. Antibody-antigen association was monitored for 180 s while dissociation in buffer was monitored for 360 s. Kinetic analysis was performed using Biacore T200 evaluation software to determine Ka and Kd values. KD was calculated from the experimentally determined Ka and Kd values as KD=Kd/Ka. As shown in FIG. 9, the recombinant chimeric anti-gremlin1 antibody 14E3 (14E3-C) has a KD value of 17.68 nM and the chimeric anti-gremlin1 antibody 22F1 (22F1-C) has a KD value of 27.28 nM.

### Example 15: Epitope Studies

### Epitope Binning Analysis by Competition ELISA Assay

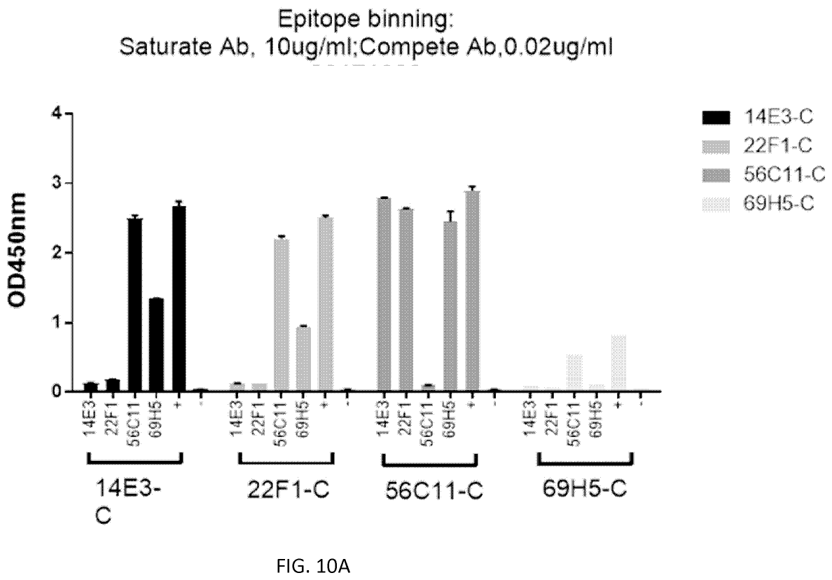

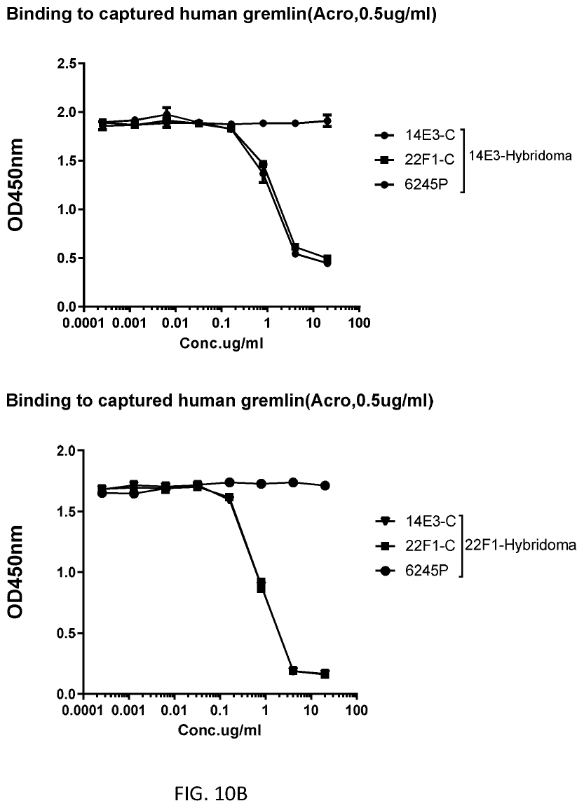

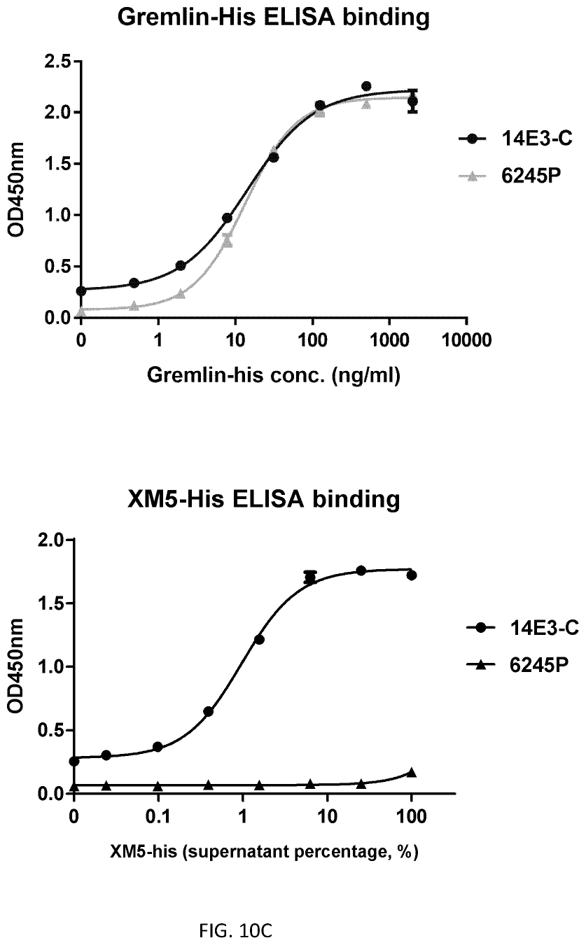

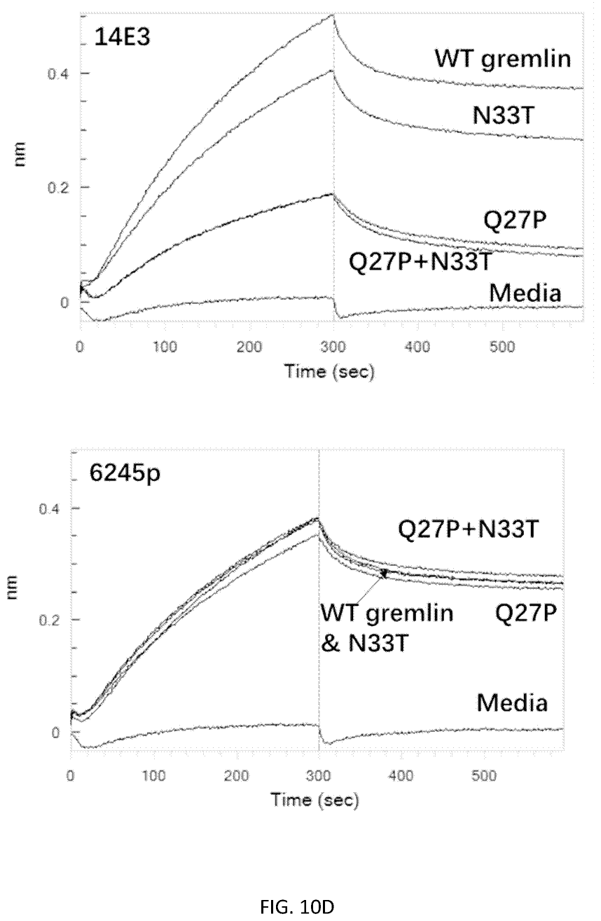

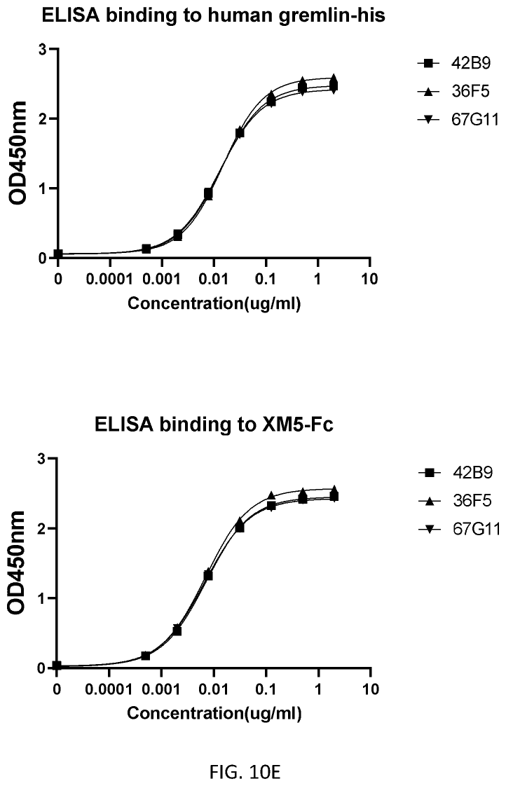

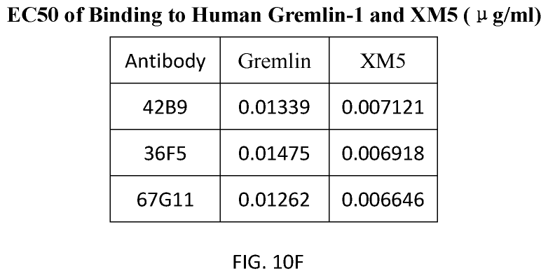

Epitope binning of anti-gremlin hybridoma antibodies were performed using a competition ELISA assay. Clear polystyrene plates (BEAVER) were coated with 100 μl/well of a 0.5 μg/ml hGREM1 (ACRO) in high pH coating buffer overnight at 4° C. Then the plates were washed once on an automatic plate washer using PBS+0.1% Tween 20 (Sigma). 100 μl of block solution consisting of PBS+1% BSA+1% normal goat serum+0.05% Tween 20 (Sigma) was added to each well and incubated at room temperature for 2 hours. Then 50 μl/well of 20 μg/ml saturated antibodies (hybridoma antibodies) in antibody dilution buffer (PBS+1% BSA+1% normal goat serum+0.01% Tween 20) were then added to each well of the plate and incubate for 1 hour. Then add 50 μl/well of 40 ng/ml competitive antibodies (chimeric antibodies, e.g., 14E3-C, 22F1-C, 56C11-C, and 69H5-C) to each well of the plate and incubate for another 1 hour at room temperature. They were then washed 3× with PBS+0.1% Tween20 followed by adding 100 μl 1:10000 goat anti-human IgG-HRP (abcam), and incubate for 1 hour at room temperature. Finally, 100 μl/well of TMB (Pierce) was added to each well after 5 minutes add 50 μl of stop solution to each well. The plates were read on a plate reader at 450 nm. The failure of the second antibody to bind in the presence of saturating quantities of the first antibody indicates the two antibodies were in the same epitope bin; the success of the second antibody to bind in the presence of the saturating quantities of the first antibody indicates the two antibodies were in different epitope bins. Based on complete set of data, there are multiple antibody bins. Examples of the data are shown in FIG. 10A.

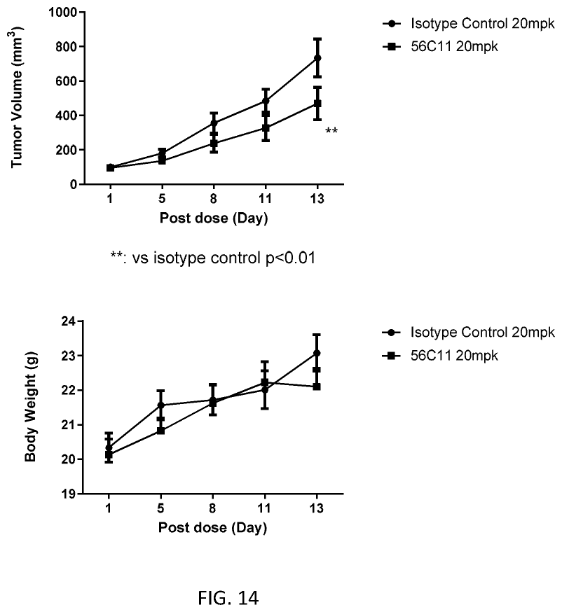

As shown in FIGS. 10A, 14E3 and 22F1 competed with each other and they also could block 69H5-C binding, indicating these three antibodies were in one epitope bin. On the other hand, the binding of 56C11 to gremlin was not competed out by 14E3, 22F1 or 69H5, indicating that 56C11 was in a different epitope bin from the epitope bin of 14E3, 22F1 and 69H5. This result echoes with the data shown in FIG. 1 that 56C11 was able to bind to mouse Gremlin whereas 14E3, 22F1 and 69H5 could not.

To analyze whether 14E3/22F1 bind to a different epitope from that of the benchmark antibody 6245P, cross-competition experiment were also conducted. In brief, fixed amount of either chimeric antibody 14E3-C/22F1-C or 6245P were added followed by adding increasing amount of hybridoma antibodies 14E3 and 22F1. The amount of chimeric antibody bound to gremlin were determined. As shown in FIGS. 10B, 14E3 and 22F1 competed with each other but cannot compete with 6245P binding even at 100×fold excess, indicating 14E3-C and 22F1-C bind to a different epitope from that of 6245P.

### Epitope Mapping of 14E3

The sequences of human Gremlin and mouse Gremlin were aligned and only 2 different amino acids were observed: Q27 (human)-P27 (mouse) and N33 (human)-T33 (mouse), where the numbering is with reference to SEQ ID NO: 69 and SEQ ID NO: 70, respectively. 14E3 binds to human Gremlin but not to mouse Gremlin, so we speculate that these two different amino acids may be the key amino acids that affect antibody binding. To verify the two key residues for 14E3 binding, 4 variants, including human Gremlin_WT, human Gremlin_Q27P, human Gremlin N33T and human Gremlin_Q27P/N33T, with signal peptide (SEQ ID NO: 71) and C-terminal His-tag were cloned into pcDNA3.1(+) vector, and all of the constructs were confirmed by DNA sequencing. The plasmids of four constucts, purified by using QIAGEN Plasmid Midi Kit, were transfected into ExpiCHO cell with 3 ml-scale by using ExpiCHO transfection kit. The transfected cells were cultured in shake flasks at 125 rpm in 8% CO₂ and 37° C. incubator. Cell Culture was harvested on day 4, and centrifuged at 8000 rpm for 30 minutes, then the supernatants were used for antibody binding assay.

Huam Gremlin1 Sequence without Signal Peptide:

(SEQ ID NO: 69)
KKKGSQGAIPPPDKAQHNDSEQTQSPQQPGSRNRGRGQGRGTAMPGEEV
LESSQEALHVTERKYLKRDWCKTQPLKQTIHEEGCNSRTIINRFCYGQC
NSFYIPRHIRKEEGSFQSCSFCKPKKFTTMMVTLNCPELQPPTKKKRVT
RVKQCRCISIDLD

Mouse Gremlin1 Sequence without Signal Peptide:

(SEQ ID NO: 70)
KKKGSQGAIPPPDKAQHNDSEQTQSPPQPGSRTRGRGQGRGTAMPGEEV
LESSQEALHVTERKYLKRDWCKTQPLKQTIHEEGCNSRTIINRFCYGQC
NSFYIPRHIRKEEGSFQSCSFCKPKKFTTMMVTLNCPELQPPTKKKRVT
RVKQCRCISIDLD

Note: Two different amino acids between human Gremlin and mouse Gremlin were bolded and underlined. The signal peptide sequence of human Gremlin1 is set forth in SEQ ID NO: 71, and the signal peptide sequence of mouse Gremlin1 is set forth in SEQ ID NO: 72.

### Biolayer Interferometry (BLI) Assay

Human gremlin Ab, 14E3 and 6245p was diluted with kinetics buffer (PBS pH 7.4, 0.1% BSA+0.2% Tween-20) to get a concentration of 100 nM in the Loading Column of 96-well half-area Microplate (Greiner Bio-one), 100 ul per well. The supernatant for Gremlin WT and mutants to be tested were added in the Association Column of the plate, 100 ul per well. Media or KD buffer was used as a reference control. Put AHC sensors in the 1st Baseline Column for 60 s to get the 1st baseline, then in the Loading Column for 300 s to capture the Gremlin antibody. After that, put the sensors in the 2nd Baseline Column for 60 s to get the 2nd baseline. Then put them in the Association Column for 300 s to let Gremlin/Gremlin Ab associate completely, put the sensors in the dissociation Column for 300 s. Analyze the group data by ForteBio (Octet96)

FIG. 10D shows that 14E3 can still bind to human gremlin 1 variant having substitution of Asn33 with Thr (i.e., N33T), although the binding showed partially reduction. However, substitution of Gln27 with Pro (i.e., Q27P) in human gremlin 1 significantly reduces 14E3 binding, which reduction was also observed in human gremlin 1 variant having substitution of both N33T and Q27P. In contrast, neither N33T nor Q27P single mutation in human gremlin 1 significantly reduced binding of 6245P, indicating that 6245P binds to an epitope that does not contain N33 or Q27 in human gremlin 1. This was consistent with the binding results observed with human gremlin 1 variant having both N33T and Q27P mutations. These data suggests that 14E3 binds to an epitope comprising Q27 of SEQ ID NO. 69. The epitope to which 14E3 binds may include, to a lesser extent, N33 of SEQ ID NO. 69.

### Example 16: Binding to Gremlin-DAN Fusion Protein XM5

To further demonstrate that 14E3 binds to different epitope from that of 6245P, we evaluated the binding of these antibodies to either wild type gremlin protein or XM5 as described in Example 6.

Using a similar ELISA protocol mentioned above, binding of chimeric antibody 14E3 to XM5 or Gremlin were tested. Briefly, 14E3-C or 6245P were coated (1 g/ml) and serial diluted XM5-his supernatant or Gremlin-his (from 2 g/ml to 0.49 g/ml) were added for binding. The secondary antibody anti-his-HRP was used for detection. As shown in FIG. 10C, both 14E3-C and 6245P could bind to Gremlin. However, only 14E3-C but not 6245P could bind to XM5. This indicates that 14E3-C and 6245P bind to different epitopes, consistent with epitope binning results mentioned above.

Binding affinities to hGREM1 and XM5 were also compared. Briefly, XM5-Fc or Gremlin-his (1 g/ml) was coated. 100 μl of block solution consisting of PBS+1% BSA+1% normal goat serum+0.5% Tween 20 (Sigma) was added to each well and incubated at room temperature for 2 hours. 4-fold serial antibody dilution from 2 μg/ml were added. Afterward the plates were washed three times with 200 ul of PBS+0.1% Tween20 followed by adding 100 μl/well 1:10000 Goat anti mouse IgG-HRP (abeam), and incubate for 1 hour at room temperature. They were then washed 3× with PBS+0.1% Tween20. Finally, 100 μl/well of TMB (InnoReagents) was added to each well and after 2 minutes add 50 ul of stop solution to each well. The plates were read on a Multiscan FC microplate reader (Thermo Scientific) at 450 nM. The EC50 values were show in table below.

As shown in FIG. 10E and FIG. 10F, 42B9, 36F5 and 67G11 bind to hGREM1 with an EC50 value from 0.012 ng/ml to 0.015 ng/ml, and bind to XM5 with an EC50 value from 0.006 ng/ml to 0.008 ng/ml.

### Example 17: Epitope Analysis by Fortebio

The epitope analysis of the anti-gremlin1 antibodies provided herein was further performed by Fortebio. In brief, a first anti-gremlin1 antibody (1st Ab) was diluted in kinetics buffer (PBS) in the Loading Column of Microplate (Greiner Bio-one), 250 μl/well. The hGremlin-his was in kinetics buffer in the Association Column of the plate, 250 μl/well; AHC sensors were put in the 1st Baseline Column for 60 s to get the 1st baseline, and then in the Loading Column for 300 s to capture the first anti-gremlin1 antibody. After that, the sensors were put in the 2^{nd} Baseline Column for 180 s to get the 2^{nd} baseline, and then in the Association Column for 300 s to associate gremlin with the first anti-gremlin1 antibody completely. The sensors were put in the second anti-gremlin1 antibody (2^{nd} Ab) Column for 300 s to let the second anti-gremlin1 antibody compete or non-compete with the first antibody. Data were analyzed by ForteBio (Octet96).

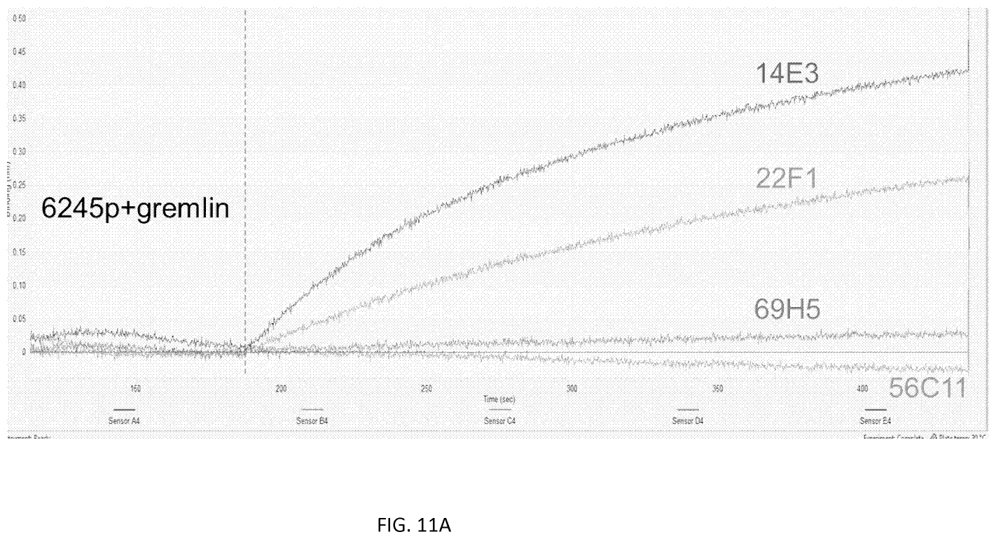

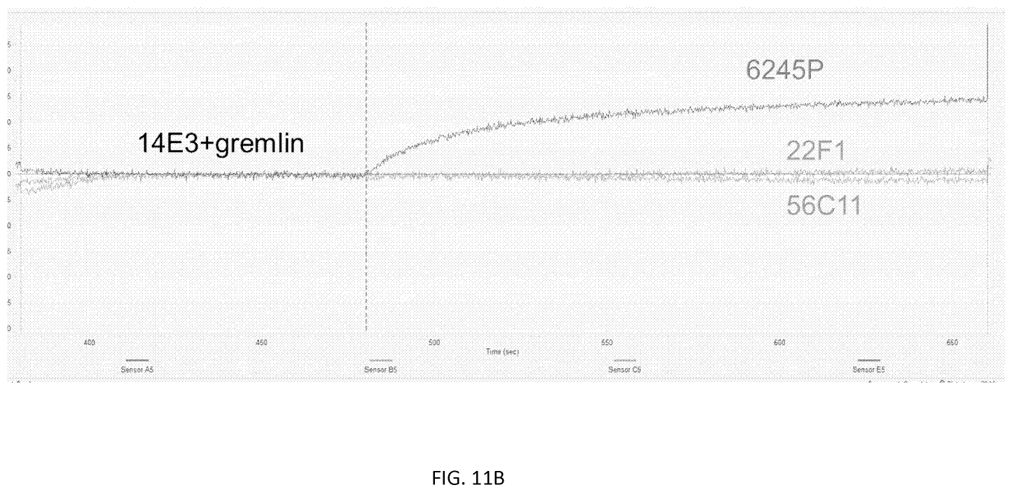

If the second anti-gremlin1 antibody cannot bind to gremlin, it indicates that it binds to a similar epitope with the first anti-gremlin1 antibody; if the second anti-gremlin1 antibody can bind without any effect by the first anti-gremlin1 antibody, it indicates that their epitopes are different. As shown in FIG. 11A, 6245P, as the second anti-gremlin1 antibody, still could bind to gremlin, indicating it could not compete with 14E3 and binds to a different epitope; whereas 22F1/69H5 could not bind to gremlin in presence of 14E3, indicating that 22F1/69H5 and 14E3 may bind to a similar epitope site. Conversely, 6245P, as the first anti-gremlin1 antibody, could not block 14E3/22F1 binding to antigen but completely block that of 56C11/69H5 (FIG. 11B). So according to the type of epitopes, there are probably 3 groups: one group includes 14E3 and 22F1, one group includes 56C11 and 69H5, epitopes of which may overlap with that of 6245P (Table 6).

TABLE 6

| | | 1st Ab on AHC sensor | | | | |
|---|---|---|---|---|---|---|
| | | 14E3 | 22F1 | 56C11 | 69H5 | 6245P |
| 2^{nd} | 14E3 | + | + | − | partial | − |
| Ab | 22F1 | + | + | − | partial | − |
| in | 56C11 | + | + | + | partial | + |
| solution | 69H5 | + | + | + | + | + |
| | 6245P | − | − | + | − | + |

Note:
+: competitor;
−: non-competitor;
partial: partial competitor

### Example 18: Humanization of Anti-GREM 1 Antibodies

### Humanized 14E3

The humanized antibodies of 14E3 were designed in the following protocol using three dimensional structure simulation and humanization by CDR-grafting.

The first step of antibody humanization is simulation of the three dimensional structure of variable domains of 14E3. The sequence of each variable domain (Vk and Vh) of murine antibody was blasted in the PDB database (Protein Data Bank, http://www.rcsb.org/) to identify the most homologous antibody sequence with known high resolution structure. Selected structure templates for modeling 14E3 had the best similarity to the target antibodies. We changed each residue of the structure to meet the target sequence manually. Certain side chain conformation was adjusted while the main chain conformations were retained. In the position where the parent structure and the simulated structure have the same residue, the side chain conformation remains unchanged; if the residue is different between the template structure and modeled structure in some positions, the side chain conformation is mutated and refined according to the template structure and packaging considerations.

We also simulated the structure for CDR-grafted 14E3 in order to guide the back mutation design and evaluation of developablity and stability of the humanized antibody. The structure simulation was performed in a similar way.

Humanization was carried out by CDR-grafting. After blasting the sequence of murine 14E3 in the human immunoglobulin gene database in IMGT, human germline framework sequence IGHV/7-4 for heavy chain and IGKV/2-30 for light chain was used for CDR grafting, respectively, and the humanized 14E3 without back mutation was obtained. To further keep the activity of the humanized 14E3, we aligned the framework sequence of the humanized antibody and that of its corresponding murine antibody. Different residues were double checked in the murine antibody structure model: if any of them were in the position which might interact with and influence the CDR residues, it should be back mutated to murine residue. The present disclosure obtained three humanized heavy chain variable regions with different back mutations, labeled as Hu14E3_Ha VH, Hu14E3_Hb VH and Hu14E3_Hc VH, respectively, and two humanized light chain variable regions with different back mutations, labeled as Hu14E3-La VL and Hu14E3-Lb VL (see Table 5). The cDNAs of these humanized heavy chain and light chain variable regions were fused to hIgG1 and hKappa constant regions and were inserted into a mammalian vector. Each humanized heavy chain was co-expressed with a humanized light chain to obtain 6 versions humanized antibodies, i.e., Hu14E3_HaLa, Hu14E3_HaLb, Hu14E3_HbLa, Hu14E3_HbLb, Hu14E3_HcLa and Hu14E3_HcLb. The expression and purification procedure are same as the chimeric antibodies.

### Humanized 22F1

The humanized antibodies of 22F1 were designed in the similar protocol. Briefly, human germline framework sequence IGHV/1-46 for heavy chain and IGKV/2-30 for light chain was used for CDR grafting, respectively, then computer-modeling was used to design humanized variants with CDR grafting and back mutations. The present disclosure obtained four humanized heavy chain variable regions with back mutations, i.e., Hu22F1_Ha VH, Hu22F1_Hb VH, Hu22F1_Hc VH and Hu22F1_Hd VH and two light chain variable regions with different back mutations, i.e., Hu22F1-La VL and Hu22F1-Lb VL (See Table 5). Each humanized heavy chain was co-expressed with a humanized light chain to obtain 8 versions humanized antibodies for 22F1, i.e., Hu22F1_HaLa, Hu22F1_HaLb, Hu22F1_HbLa, Hu22F1_HbLb, Hu22F1_HcLa, Hu22F1_HcLb, Hu22F1_HdLa and Hu22F1_HdLb. The expression and purification procedure are same as the chimeric antibodies.

The expression and purification of a recombinant antibody protein were conducted by following steps: ExpiCHO cells were seeded in ExpiCHO Expression Medium at 5-6×10⁶ cells/ml. Subsequently, ExpiCHO cells were transfected using ExpiCHO transfection kit with equal amount of heavy chain vector and light chain vector DNA at a final concentration of 1.0 μg/ml. The transfected cells were cultured in shake flasks at 125 rpm in a 37° C. incubator supplemented with 8% CO₂. ExpiCHO feed was added after 18 to 22 hours post transfection. The cell culture was harvested on day 10. Harvest Cell Culture Fluid (HCCF) was obtained by centrifugation. The HCCF was then loaded onto rProteinA column (G.E. Healthcare) and washed with PBS. The final IgG antibody was eluted with a solution containing 20 mM citrate acid at pH3.2. Finally, the eluted antibody protein was neutralized and stored at −80° C. for long-term usage. The resulting antibody was analyzed to determine the level of purity using SDS-PAGE and size exclusion chromatography (TSKgel G3000SWXL, TOSOH).

### Humanized 56C11

The humanized antibodies of 56C11 were designed in the similar protocol. Briefly, human germline framework sequence IGHV1-2*02 for heavy chain and IGKV2-30*02 for light chain were used for CDR grafting, respectively.

Heavy chain (HC) variants 1, 2, 3 and 4 were obtained by direct grafting the three CDRs to the germline sequence. The combination of the above heavy chain variable regions and light chain variable regions generate the following humanized 56C11 antibodies: 56C11-H0L0, 56C11-HaL0, 56C11-HbL0, 56C11-HcL0, 56C11-H0La, 56C11-HaLa, 56C11-HbLa, 56C11-HcLa, 56C11-H0Lb, 56C11-HaLb, 56C11-HbLb, 56C11-HcLb.

The humanized variants of the heavy chain and light chain of 56C11 are linked to human IgG1 heavy chain constant region and kappa light chain constant region as shown below:

Human IgG1 heavy chain constant region (SEQ ID
NO: 138):
ASTKGPSVFPLAPSSKSTSGGTAALGCLVKDYFPEPVTVSW
NSGALTSGVHTFPAVLQSSGLYSLSSVVTVPSSSLGTQTYICNVNHKPS
NTKVDKKVEPKSCDKTHTCPPCPAPELLGGPSVFLFPPKPKDTLMISRT
PEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
Human Kappa light chain constant region (SEQ ID
NO: 139):
RTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The variable regions of the above heavy chain and light chain cDNAs were synthesized and fused with the constant region of human IgG1 and human kappa. The heavy chain and light chain of the selected antibody genes were cloned into an expression vector and the large-scale DNA was prepared using Plasmid Maxiprep System from Qiagen. Transfection was carried out using the ExpiFectamine™ CHO Reagent from Invitrogen according to the manufacturer's protocol. Supernatants were harvested when the cell viability was around 60%. The cell culture supernatant was filtered through 0.22 m filtration capsule to remove the cell debris. Load the supernatant onto a pre-equilibrated Protein-A affinity column. Then Protein A resin inside the column was washed with equilibration buffer (PBS), and 25 mM citrate (pH 3.5) was used to elute the antibody. The pH was adjusted to about 6.0-7.0 with 1M Tris-base (pH 9.0). The endotoxin was controlled below 1 EU/mg. The purified antibody was then characterized by SDS-PAGE and SEC-HPLC.

### Example 19: Binding of Humanized Antibodies to hGremlin in ELISA and Fortebio

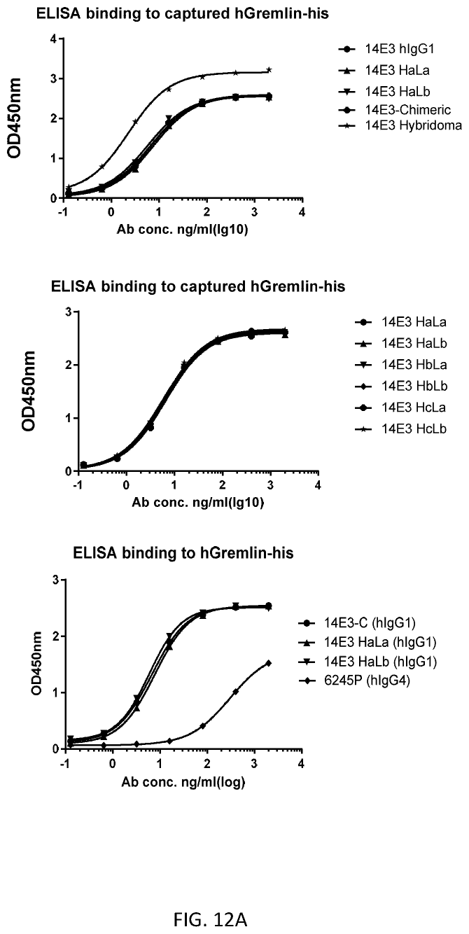

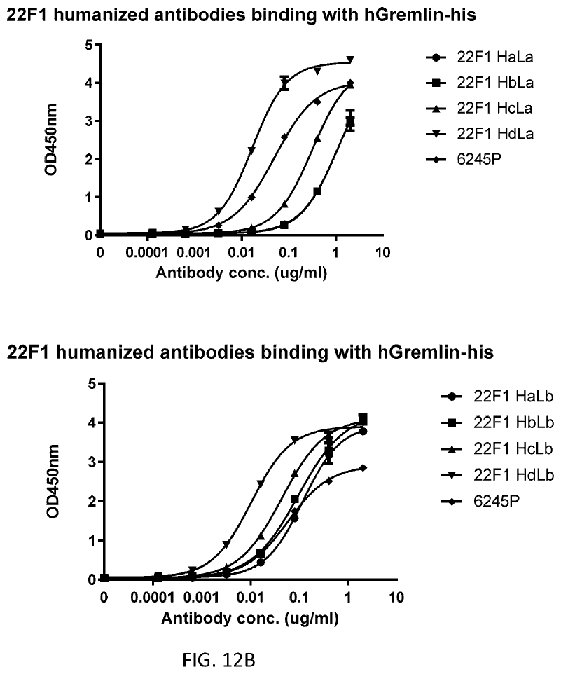

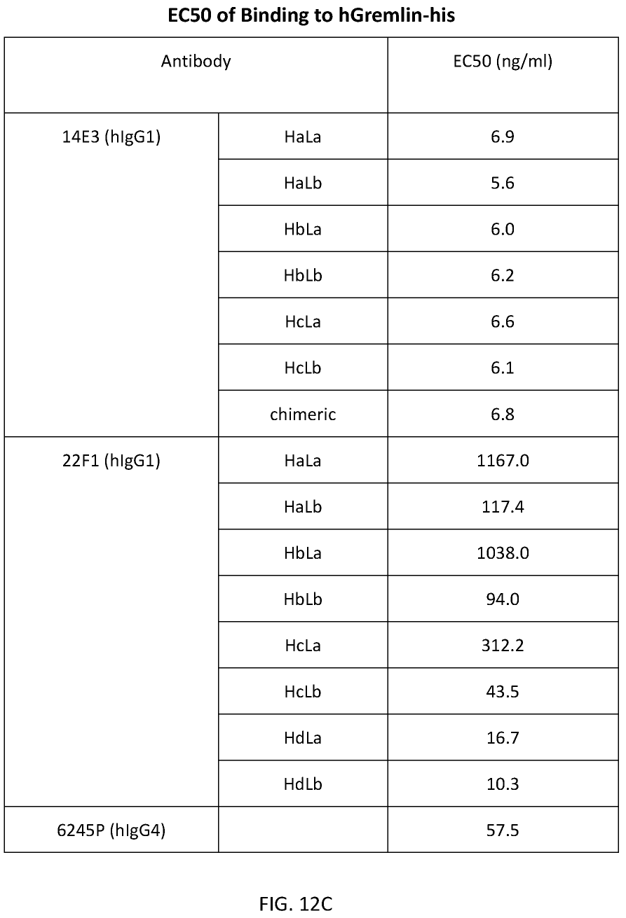

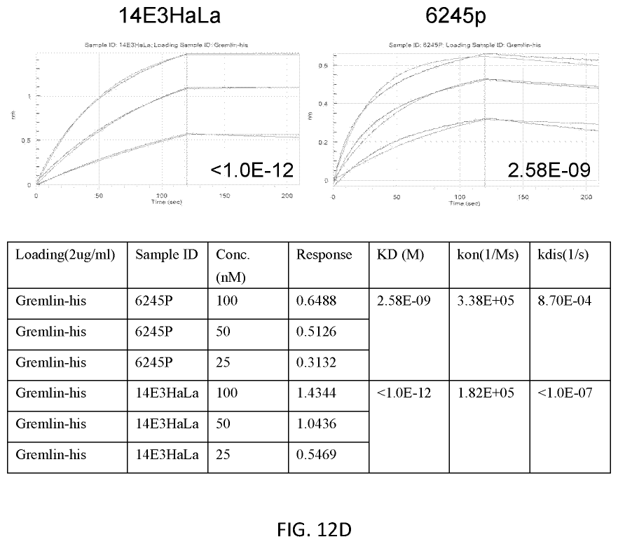

Same protocol of Example 4. As shown in FIG. 12A, humanized variants of 14E3 retained similar binding activity as the chimeric 14E3 (e.g., 14E3 hIgG1 or 14E3-C), indicating that the biological activity was probably unaffected by humanization. However, most humanized variants of 22F1 lost binding significantly and only 22F1-HdLa and 22F1-HdLb still had good affinity (FIGS. 12B and C).

The affinity of the humanized antibodies were also measured by Fortebio. Human gremlin protein was diluted with kinetics buffer to get a concentration of 2 μg/ml. 0 nM was used as a reference control. Antibodies to be tested were diluted with ForteBio kinetics buffer (PBS pH 7.4, 0.1% BSA+0.002% Tween-20) to a concentration of 100 nM, 50 nM, and 25 nM. Human gremlin-his was immobilized onto NTA biosensor. The baseline was detected for 60 seconds, and then anti-gremlin antibody association was detected for 120 seconds to get the Kₒₙ factor data. Followed by dissociation in kinetic buffer for 90 seconds to get the K_{off} factor data. As shown in FIG. 12D, the humanized anti-gremlin1 antibody 14E3 has a KD value of less than 1 nM, much lower than that of the benchmark antibody.

### Example 20: Tumor Growth Inhibition Activity of Humanized Antibody 14E3 in PC-3 Xenograft Tumor Model

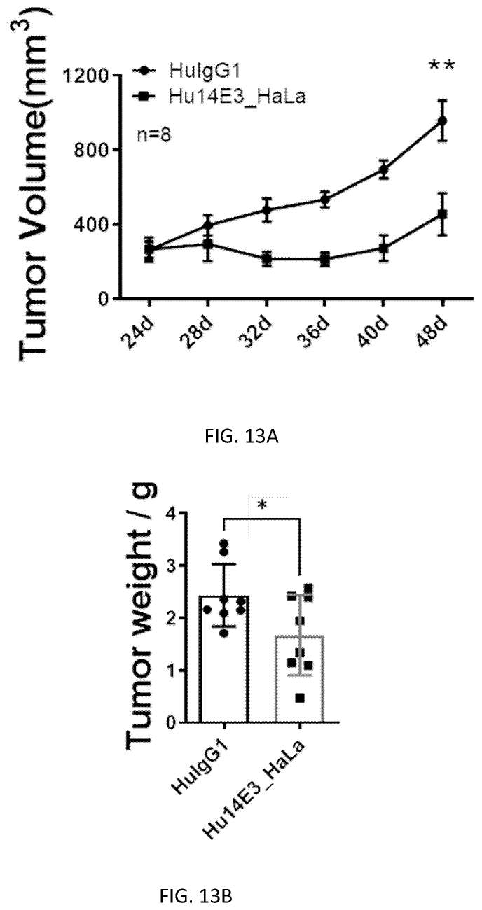

Briefly, Human prostate cancer PC3 cells were maintained in vitro as a monolayer culture in RPMI1640 medium (Thermo Fisher) supplemented with 10% heat inactivated fetal bovine serum (ExCell Biology), 100 U/ml penicillin, 100 ug/ml streptomycin (Hyclone) and 1 μg/mL puromycin (Gibco) at 37° C. in an atmosphere with 5% CO₂ in air. The tumor cells were routinely sub-culture twice weekly by trypsin-EDTA treatment (Hyclone). The cells growing in an exponential growth phase were harvested and counted for tumor inoculation. SPF grade male nude mice were subcutaneously inoculated with mixed 1*10\hat{ }6 PC3 cells with 50% matri-gel. 10 days later, the inoculated mice were castrated by orchiectomy. When the tumor size reached around 200 mm\hat{ }3, tumor bearing mice were selected and randomized to 2 groups (n=8). Animals were intraperitoneally injected with 10 mg/kg hIgG1 control and 10 mg/kg Hu14E3_HaLa, every 4 days for 3 weeks. Tumor size was measured every 4 days in two dimensions using a caliper (INSIZE) and the volume was expressed in mm\hat{ }3 using the formula: V=0.5 a*b\hat{ }2 where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01. As a result, Hu14E3_HaLa could effectively inhibit the tumor growth either from the volume or weight evaluation (FIGS. 13A and 13B).

### Example 21: Efficacy of 56C11 on CT-26 Tumor Model on BALB/c (MSB-Pharm2018025)

As gremlin1 can be expressed in both tumor cells and stromal fibroblast cells, we evaluated the contribution of gremlin1 in modulating the tumor microenvironment and its potential in modulating tumor growth either alone or in combination with checkpoint inhibitor. Therefore we evaluated the antitumor activity of 56C11 on CT-26 model, a syngeneic tumor model. Briefly, 24 female Balb/c mice of 5-6 weeks were inoculated with 2×10⁶ CT26 tumor cells. When tumor volume reached around 100 mm³, animals were randomized and grouped as below: 1) Isotype control 20 mg/kg, and 2) 56C11 at 20 mg/kg. The antibodies were injected intraperitoneally (IP) twice a week for 2 weeks. The results shown in FIG. 14 demonstrated that anti-GREM1 antibody 56C11 (which is cross reactive to mouse GREM1) alone can have significant anti-tumor activity without significant effect on total body weight. Humanized anti-GREM1 antibodies provided herein (e.g., Hu14E3, Hu14E3, Hu22F1 and Hu56C11) are expected to show the similar technical effect in human as their chimeric counterparts in exhibiting significant anti-tumor activity without significant effect on total body weight.

### Example 22: Efficacy of MPDL-3820A and Anti-GREM 1 Antibody Combination Therapy on CT26 Tumor Model (MSB-Pharm2018004)

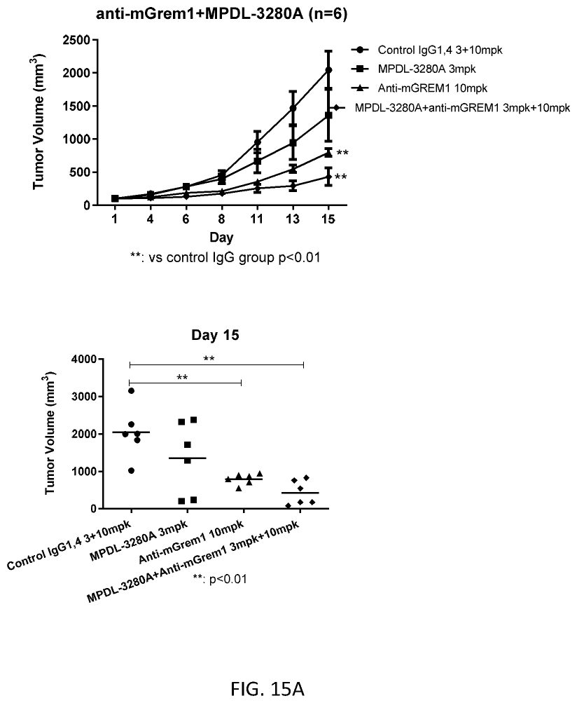

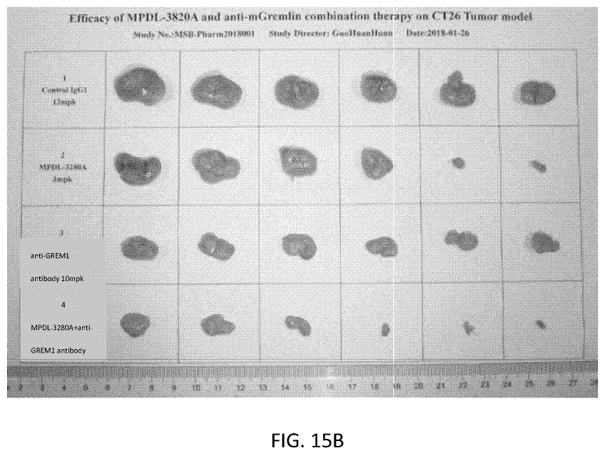

To further test the anti-tumor efficacy of combination of anti-GREM 1 antibody with an immune checkpoint inhibitor, we evaluated the anti-tumor activity of combining MPDL-3280A and surrogate anti-mouse GREM 1 antibody (anti-mGREM 1 antibody) in CT26 tumor model. Briefly, female Balb/c mice of 5-6 weeks age were inoculated with 5×10⁵/mouse CT26 cells. The animal were randomized. When the tumor volume reaches to around 100 mm³, the mice divided into four groups treated with: 1) control IgG1 alone at 10 mpk, 2) MPDL3280A alone at 3 mpk, 3) anti-mGREM 1 antibody alone at 10 mpk, or 4) combination of MPDL3280a at 3 mpk and anti-mGREM 1 antibody at 10 mpk, by IP twice a week for 2 weeks. As shown in FIG. 15A and FIG. 15B, the combination of anti-mGREM 1 antibody with immune checkpoint inhibitor (e.g., an antibody against PD-L1) resulted in significantly higher anti-tumor activity (in terms of either tumor volume or tumor weight) as compared to anti-mGREM 1 antibody alone or the immune checkpoint inhibitor alone. This suggests that anti-mGREM 1 antibody can enhance the anti-tumor activity of immune checkpoint inhibitor.

### Example 23: Efficacy of Humanized 14E3 Combined with Cisplatin on Esophageal Cancer PDX Model

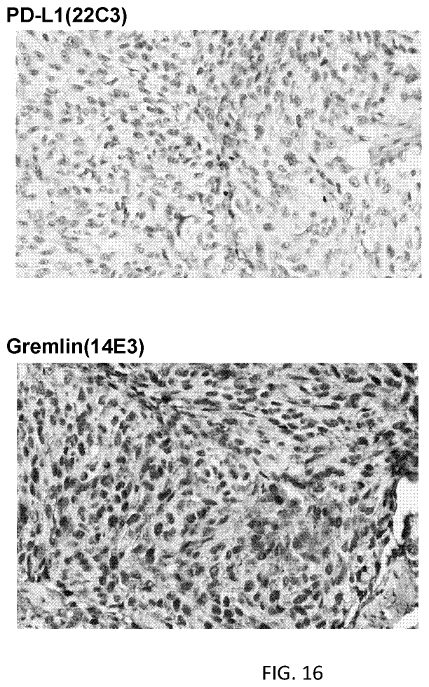

Human gremlin IHC specifically positive esophageal tumor tissue (E7) was obtained from Beijing Cancer Hospital passage in NOD/SCID mice and established PDX bank. We tested the GREM1 expression and PD-L1 expression in the E7 esophageal PDX model by immunohistochemistry using either an anti-GREM1 antibody (14E3) or an anti-PD-L1 antibody (22C3). FIG. 16 shows the esophageal cancer PDX model E7 was positive in GREM1 expression but did not have PD-L1 expression.

Each mouse was subcutaneously inoculated with a small tumor tissue block approximately 3 mm in diameter which sheared from integrated tumor decollement form a tumor bearing mouse. 18 days after inoculation animals with tumor size at about 70 mm³ were selected and randomly divided into 4 groups, each group consisting of 8 mice. Then the mice were treated with isotype control+PBS, humanized 14E3 (hzd14E3) at dose of 20 mg/kg, Cisplatin at dose of 3 mg/kg and combination of hzd14E3 and Cisplatin. Isotype control and hzd14E3 administrated twice a week for 4 weeks by i.p. injection and PBS, while Cisplatin was administrated once a week for 4 weeks by i.v. injection. Animals were sacrificed at the end of the study with CO₂ inhalation. Tumor size was measured twice or triple times a week in two dimensions using a caliper (INSIZE) and the volume was expressed in mm\hat{ }3 using the formula: V=0.5 a*b2 where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01.

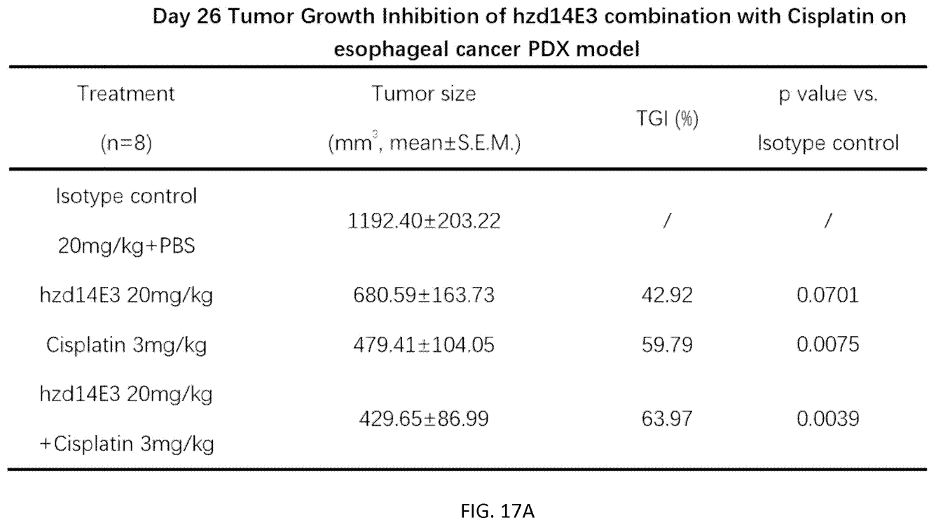

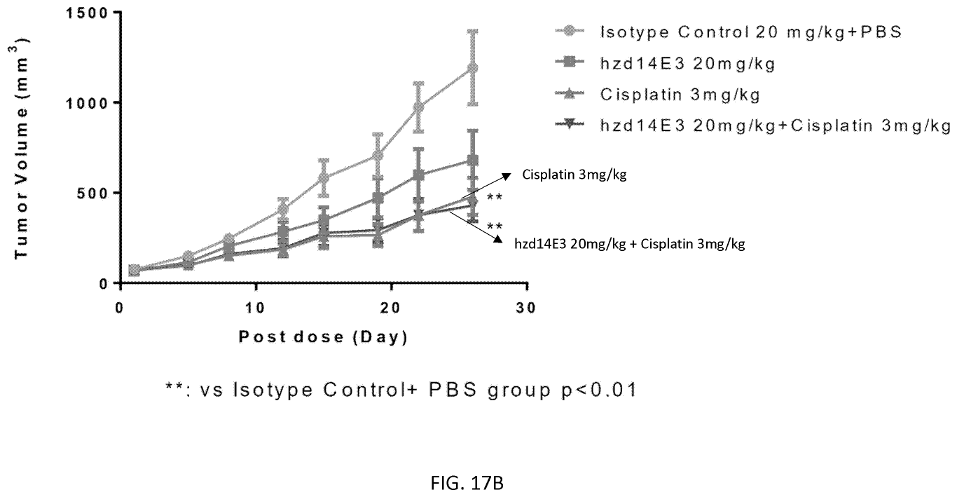

FIG. 17A and FIG. 17B show significantly enhanced tumor growth inhibition when humanized 14E3 alone was used in this experiment as compared to Isotype control with 42.92% TGI. Combination of humanized 14E3 and Cisplatin further inhibited tumor growth when compared to either humanized 14E3 alone (63.97% TGI vs 42.92% TGI) or Cisplatin alone (63.97% TGI vs 59.79% TGI), suggesting synergistic effect of the combination treatment with humanized 14E3 and Cisplatin on esophageal cancer.

To date, first-line therapy for esophageal cancer generally includes esophagectomy, chemotherapy, targeted therapy, immunotherapy (e.g., targeting PD-1 or PD-L1), and/or a combination thereof. Second-line and subsequent therapy for esophageal cancer may involve targeted therapy such as ramucirumab to target vascular endothelial growth factor (VEGF) receptor or trastuzumab for metastatic adenocarcinoma that overexpresses HER2 (NCCN Clinical Practice Guidelines in Oncology. Esophageal and Esophagogastric Junction Cancers. National Comprehensive Cancer Network. V1. 2020). Our data described above show that the anti-GREM1 antibodies provided herein can effectively treat tumors that do not express PD-L1, e.g., esophageal cancer that do not overexpress PD-L1, and can further achieve synergistic effect when in combination with chemotherapy, e.g., Cisplatin. This suggests that the anti-GREM1 antibodies provided herein can serve as a new option for either first-line therapy or second-line therapy for esophageal cancer.

### Example 24: Efficacy of hzd14E3 (Combination with DC101) and 6245P on Esophageal Cancer PDX Model

Human gremlin IHC specifically positive esophageal tumor tissue (E7) was obtained from Beijing Cancer Hospital passage in NOD/SCID mice and established PDX bank. Each mouse was subcutaneously inoculated with a small tumor tissue block approximately 3 mm in diameter which sheared from integrated tumor decollement form a tumor bearing mouse. 18 days after inoculation animals with tumor size at about 70 mm³ were selected and randomly divided into 4 groups, each group consisting of 8 mice. Then the mice were treated with isotype control, hzd14E3 and 6245P at dose of 20 mg/kg, DC101 at dose of 10 mg/kg and combination of hzd14E3 and DC101. DC101 is a monoclonal antibody that reacts with mouse VEGFR-2 and is commercially available (e.g. under catalog #BE0060 from BioXell). Control and test articles were administrated twice a week for 4 weeks by i.p. injection. Animals were sacrificed at the end of the study with CO₂ inhalation. Tumor size was measured twice or triple times a week in two dimensions using a caliper (INSIZE) and the volume was expressed in mm\hat{ }3 using the formula: V=0.5 a*b² where a and b are the long and short diameters of the tumor, respectively. Results were analyzed using Prism GraphPad and expressed as mean±S.E.M. Comparisons between two groups were made by T-test, and the difference is considered significant if p is *<0.05 and **<0.01. Other humanized anti-GREM1 antibodies provided herein (e.g., Hu22F1 and Hu56C11) are expected to show the similar technical effect.

### Example 25: Characterization of Antibody Activity in Blocking of Gremlin Binding to Captured FGFR1

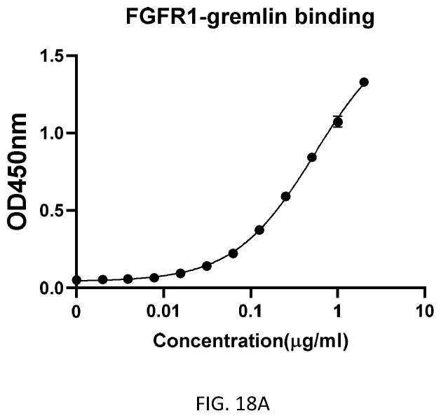

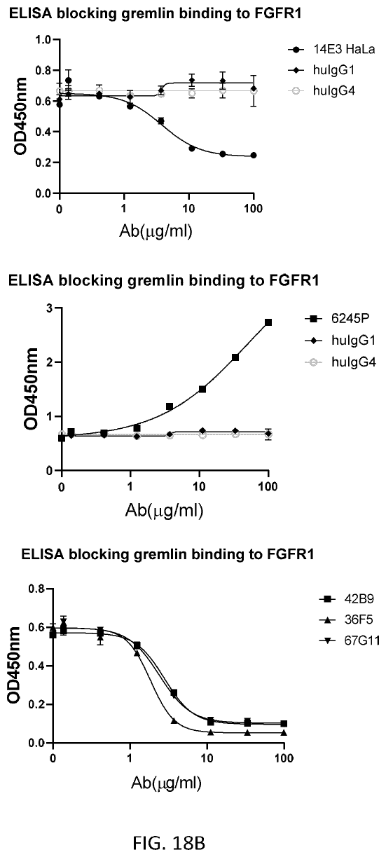

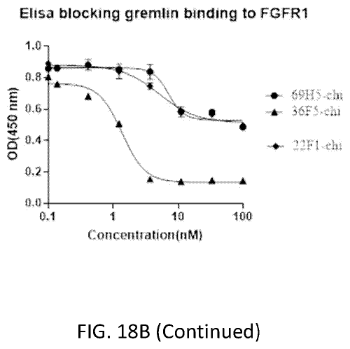

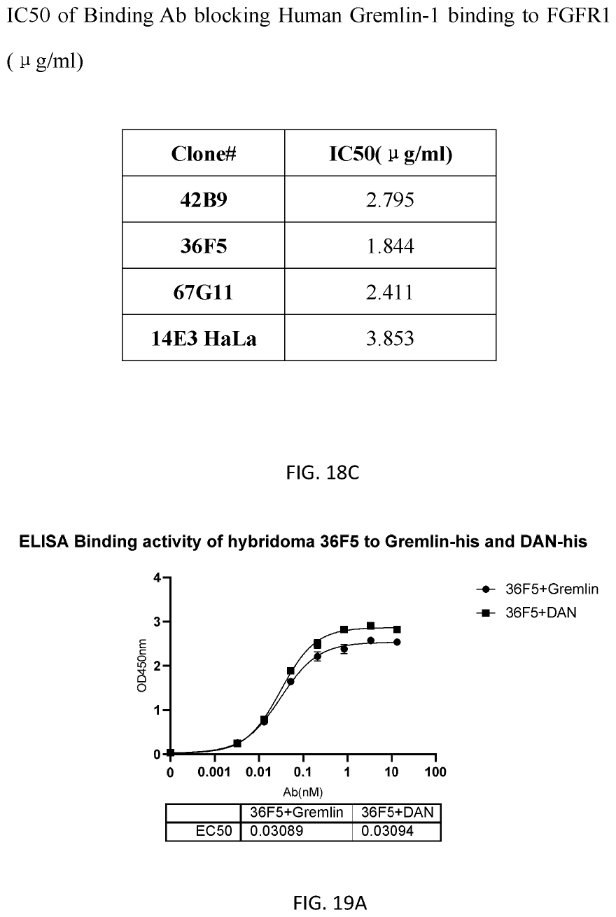

Gremlin-his was tested for its binding ability to FGFR1 immobilized on plate. Briefly, plates were coated with recombinant human 2 μg/ml of FGFR1-Fc (Sino-Biological) overnight and then 2-fold serial dilutions of gremlin-his (ACRO) from 2 μg/ml were added to coated plates and incubated for 1 h at RT (Room Temperature). The plates were then washed and plate bound Gremlin-his was detected with anti-his HRP (GenScript). Plates were then developed with a TMB solution and stopped by adding stop solution. The plates were read on a plate reader at 450 nm. The incubation time was about 20 mins. As shown is FIG. 18A, hGREM1 can bind to FGFR1.

Next, 0.25 μg/ml of gremlin was selected to test the blocking activity. The ability of antibodies to block Gremlin binding to human FGFR1-Fc was examined via ELISA. Plates were coated with recombinant FGFR1-Fc (2 μg/ml) overnight and then serial dilutions of antibodies were incubated with 0.25 μg/ml of human Gremlin-his modified for 1 h at RT before this complex was added to coated plates and allowed to incubate for an additional hour at RT. The plates were then washed and added with anti-his HRP (GenScript). Plates were then developed with a TMB solution and stopped by adding stop solution. The plates were read on a plate reader at 450 nm. As shown in FIGS. 18B and 18C, the anti-gremlin 1 antibodies provided herein (e.g, 42B9, 36F5, 67G11 and 14E3 HaLa, chimeric antibody 69H5 (69H5-chi), chimeric antibody 36F5 (36F5-chi), chimeric antibody 22F1 (22F1-chi)) can inhibit or block the binding of hGREM1 to FGFR1, whereas the benchmark antibody 6245P does not block the binding of hGREM1 to FGFR1. 36F5-chi could block the binding of hGREM1 to FGFR1 with an IC50 of 1.368 nM. 69H5-chi and 22F1-chi had a partial blocking activity, wherein 69H5-chi could block the binding of hGREM1 to FGFR1 with an IC50 of 7.138 nM and 22F1-chi could block the binding of hGREM1 to FGFR1 with an IC50 of 5.117 nM. Humanized anti-GREM1 antibodies provided herein (e.g., Hu14E3, Hu22F1) are expected to show the similar technical effect as their chimeric counterparts in blocking the binding of hGREM1 to FGFR1.

### Example 26: Binding Analysis of Purified Hybridoma or Chimeric Anti-Gremlin Antibody to Captured Human Gremlin and DAN Protein by ELISA

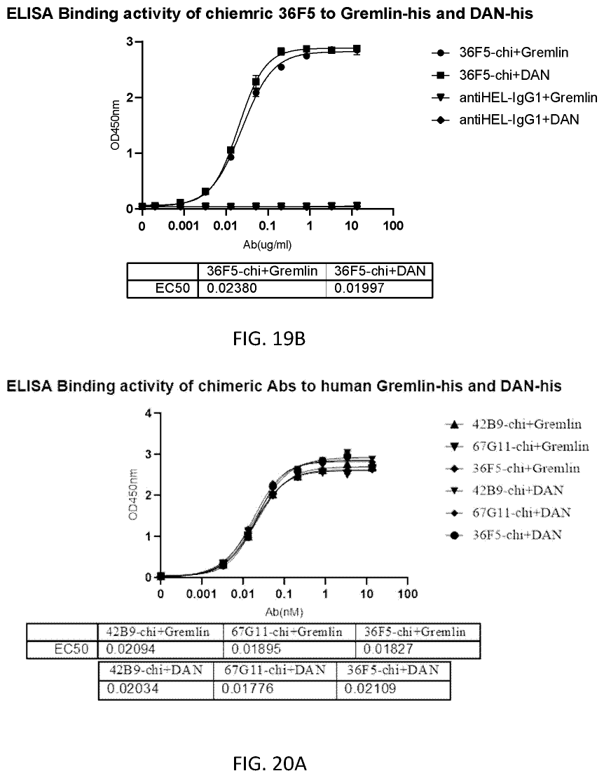

Clear polystyrene plates (BEAVER) were coated with 100 μl/well of a 0.5 μg/ml human gremlin (ACRO) and DAN (Sinobiological) in high pH coating buffer overnight at 4° C. Then the plates were washed once on an automatic plate washer using PBS+0.1% Tween 20 (Sigma). 100 μl of block solution consisting of PBS+1% BSA+1% normal goat serum+0.5% Tween 20 (Sigma) was added to each well and incubated at room temperature for 2 hours. Then 100 ul of antibodies (hybridoma or chimeric antibody 36F5, chimeric antibody 67G11, chimeric antibody 42B9) in antibody dilution buffer containing PBS+1% BSA+1% normal goat serum+0.01% Tween 20 starting from 2 μg/ml (13.33 nM) and then 4 fold serial dilution were added to each well of the plate and incubated for 1 hours at room temperature. Afterward the plates were washed three times with 200 ul of PBS+0.1% Tween20 followed by adding 100 μl/well 1:10000 Goat anti mouse IgG-HRP or mouse anti-human IgG-HRP (abcam), and incubate for 1 hour at room temperature. They were then washed 3× with PBS+0.1% Tween20. Finally, 100 μl/well of TMB (InnoReagents) was added to each well and after 2 minutes add 50 ul of stop solution to each well. The plates were read on a Multiscan FC microplate reader (Thermo Scientific) at 450 nm. The EC50 values were also shown in FIGS. 19A, 19B and 19C, below. As a result, hybridoma or chimeric antibody 36F5, chimeric antibody 67G11 and chimeric antibody 42B9 had a similar binding activity of gremlin with DAN protein.

### Example 27: Characterization of Antibody Activity in Blocking of DAN Protein Binding to Captured BMP2/4

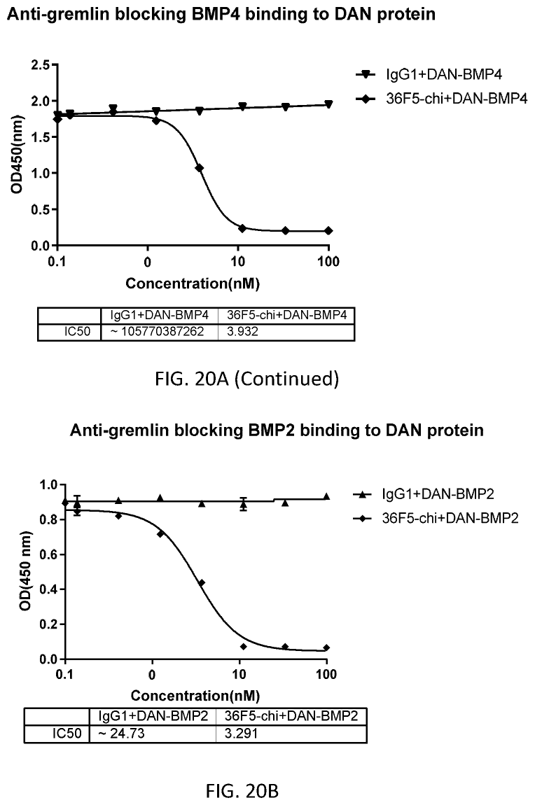

Plates were coated with recombinant human BMP2/4 (0.5 μg/ml) overnight. Then the plates were washed once on an automatic plate washer using PBS+0.1% Tween 20 (Sigma). 100 μl of block solution consisting of PBS+1% BSA+1% normal goat serum+0.5% Tween 20 (Sigma) was added to each well and incubated at room temperature for 2 hours. Then the plates were washed three 3 times. Then 55 ul serial dilutions of chimeric antibodies 36F5 and 55 ul 0.5 g/ml of human DAN-his in dilution buffer containing PBS+1% BSA+1% normal goat serum+0.01% Tween 20 separately were mixed and incubated for 1 h at RT before 100 ul of this complex was added to coated plates and allowed to incubate for an additional hour at room temperature. The plates were then washed 3 times and added with 100 ul of anti-his HRP (GenScript) in dilution buffer. Plates were then developed with a TMB solution and stopped by adding stop solution. After wash three times with wash buffer, the plates were read on a plate reader at 450 nm. Also, consistent with previous result, besides blocking gremlin activity, 36F5 could block BMP2/4 binding to DAN protein. (FIGS. 20A and 20B).

### Example 28: Efficacy of Hybridoma 36F5 on EMT6/hPD-L1 Tumor Model

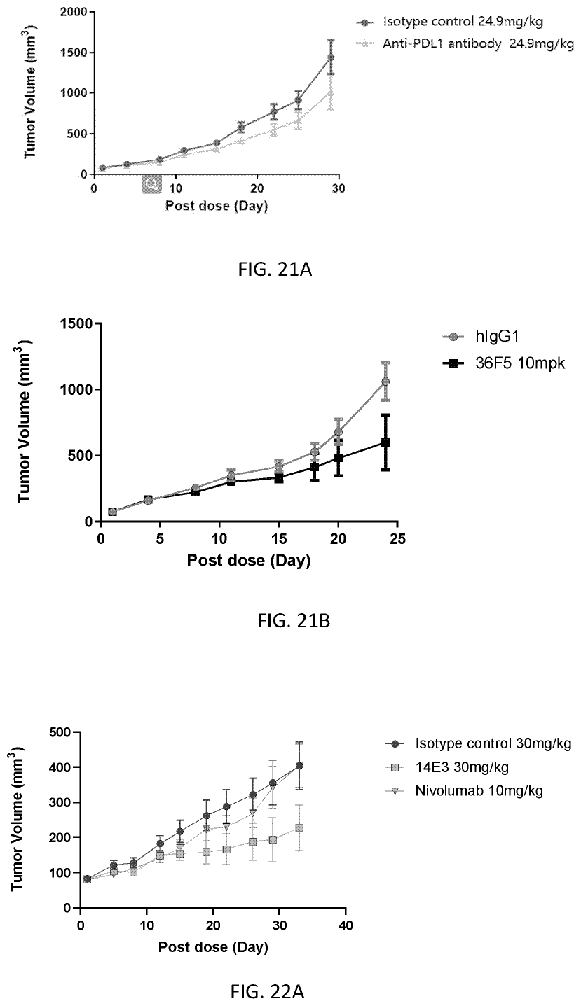

Mouse breast cancer cell line EMT6 was transfected with human PD-L1 gene screened stable expressing human PD-L1 is named EMT6/hPD-L1. EMT6/hPD-L1 cells were maintained in vitro as a monolayer culture in DMEM medium (Hyclone) supplemented with 10% heat inactivated fetal bovine serum (ExCell Biology), 100 U/ml penicillin, 100 ug/ml streptomycin (Hyclone) at 37° C. in an atmosphere with 5% C02 in air. The tumor cells were routinely sub-culture twice weekly by trypsin-EDTA treatment (Hyclone). The cells growing in an exponential growth phase were harvested and counted for tumor inoculation. Female SPF grade BABL/c mice were inoculated with mixed 2*10\hat{ }6 EMT6/hPD-L1 cells with 50% matri-gel. In the first study, When the tumor size around 80 mm\hat{ }3, tumor bearing mice were selected and randomized to 2 groups (n=10). Animals were treated with 24.9 mg/kg hIgG1 control and 24.9 mg/kg AM4B6 twice a week for 4 weeks by i.p. injection. In the second study, When the tumor size around 70 mm\hat{ }3, tumor bearing mice were selected and randomized to 2 groups (n=8). Animals were treated with 10 mg/kg hIgG1 control and 10 mg/kg 36F5 twice a week for 3 weeks by i.p. injection. Tumor size was measured twice times a week in two dimensions using a caliper (INSIZE) and the volume was expressed in mm\hat{ }3 using the formula: V=0.5 a*b\hat{ }2 where a and b ate the long and shirt diameters of the tumor, respectively. Results were analyzed using Prism GraphPad and expressed as mean±S.E.M. Comparisons between two groups were made by T-test, and the difference is considered significant if p is *<0.05 and **<0.01. Table 7 and FIG. 21A were the result of the first study. The results show the anti-PD-L1 antibody (AM4B6) did not have antitumor activity on EMT6/hPD-L1 tumor model. The EMT6/hPD-L1 tumor model exhibits poor response to PD-L1 antibody. Table 8 and FIG. 21B were the result of the second study. The result shows the anti-Gemlin1 antibody (36F5) has promising antitumor activity on EMT6/hPD-L1 tumor model that exhibits poor response to PD-L1 antibody.

TABLE 7
Efficacy of AM4B6 on EMT6/hPD-L1 tumor model on Day 29

| Treatment | Tumor size | | p value vs Isotype |
|---|---|---|---|
| (n = 10) | (mm\hat{ }3, mean ± S.E.M.) | TGI (%) | control |
| Isotype control | 1445.07 ± 208.18 | / | / |
| AM4B6 | 1016.29 ± 214.42 | 29.67 | 0.1685 |

TABLE 8
Efficacy of 36F5 on EMT6/hPD-L1 tumor model on Day 24

| Treatment | Tumor size | | p value vs Isotype |
|---|---|---|---|
| (n = 8) | (mm\hat{ }3, mean ± S.E.M.) | TGI (%) | control |
| Isotype control | 1060.76 ± 141.45 | / | / |
| 36F5 | 599.72 ± 208.49 | 43.46 | 0.0886 |

### Example 29: Efficacy of Hybridoma 14E3, Hybridoma 36F5 or Nivolumab on E7 Tumor Model in PBMC Humanized Mice

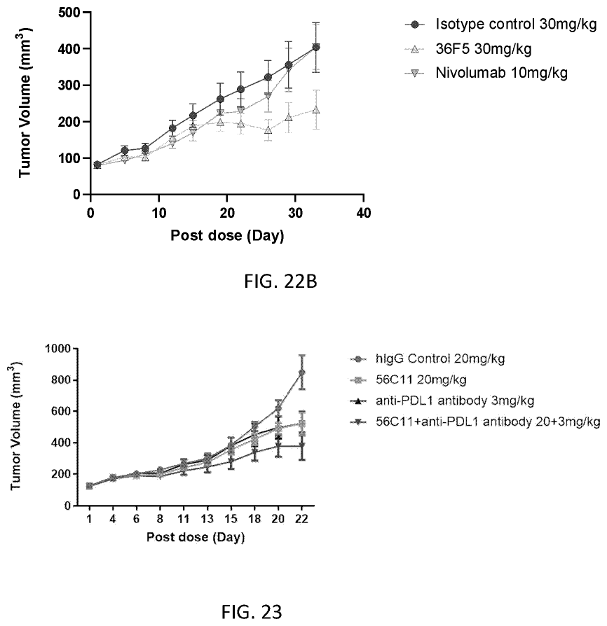

E7 is esophageal cancer PDX with human Gremlin high expression obtained from Beijing Cancer Hospital passage in NOD-SCID mice and established PDX bank. NOG mice were severe immunodeficiency purchased from Vital River. Each mouse was subcutaneously inoculated with a small tumor tissue block approximately 3 mm in diameter which sheared from integrated tumor decollement from a tumor bearing moue. 27 days after inoculation animals with tumor size at about 50 mm\hat{ }3 were selected and intravenously injected with 5*10\hat{ }6/mouse human PBMC. A week later, animals were screened for reconstruction profile and randomly divided into 6 groups, each group consisting of 8 mice. Animals were treated with 30 mg/kg isotype control, 30 mg/kg 14E3, 30 mg/kg 36F5, 10 mg/kg Nivolumab, twice a week for 5 weeks by i.p. injection. Tumor size was measured twice times a week in two dimensions using a caliper (INSIZE) and the volume was expressed in mm\hat{ }3 using the formula: V=0.5 a*b\hat{ }2 where a and b ate the long and shirt diameters of the tumor, respectively. Results were analyzed using Prism GraphPad and expressed as mean±S.E.M. Comparisons between two groups were made by T-test, and the difference is considered significant if p is *<0.05 and **<0.01. Table 9, FIG. 22A and FIG. 22B were the result of the study. The results show the anti-PD-1 antibody (nivolumab) did not have antitumor activity on E7 tumor model. The anti-Gemlin1 antibody 36F5 and 14E3 have promising antitumor activity on E7 tumor model that exhibits poor response to PD-1 antibody.

TABLE 9
Efficacy of 14E3 or 36F5 compared with Nivolumab on
E7 Tumor model in PBMC humanized mice on Day 35

| | Tumor size | | p value vs |
|---|---|---|---|
| Treatment | (mm\hat{ }3, | | Isotype |
| (n = 8) | mean ± S.E.M.) | TGI (%) | control |
| 30 mg/kg Isotype control | 527.84 ± 129.07 | / | / |
| 30 mg/kg 14E3 | 279.96 ± 92.85 | 46.96 | 0.1606 |
| 30 mg/kg 36F5 | 264.61 ± 63.69 | 49.87 | 0.0888 |
| 10 mg/kg Nivolumab | 440.71 ± 84.33 | 16.51 | 0.5809 |

### Example 30: Efficacy of 56C11 Combination Therapy with Anti-PDL1 Antibody on MC38/hPD-L1 Tumor Model

Mouse colon cancer cell line MC38 was transfected with human PD-L1 gene screened stable expressing human PD-L1 is named MC38/hPD-L1. MC38/hPD-L1 cells were maintained in vitro as a monolayer culture in 1640 medium (Hyclone) supplemented with 10% heat inactivated fetal bovine serum (ExCell Biology), 100 U/ml penicillin, 100 ug/ml streptomycin (Hyclone) at 37° C. in an atmosphere with 5% CO2 in air. The tumor cells were routinely sub-culture twice weekly by trypsin-EDTA treatment (Hyclone). The cells growing in an exponential growth phase were harvested and counted for tumor inoculation. Female SPF grade C57BL/6 mice were inoculated with mixed 2*10\hat{ }6 MC38/hPD-L1 cells with 50% matri-gel. When the tumor size around 120 mm\hat{ }3, tumor bearing mice were selected and randomized to 4 groups (n=8). Animals were treated with 3 mg/kg hIgG1 control, 20 mg/kg 56C11, 3 mg/kg 23F11 (anti-PDL1 antibody) and combination of 20 mg/kg 56C11 and 3 mg/kg 23F11, twice a week for 3 weeks by i.p. injection. Tumor size was measured twice a week in two dimensions using a caliper (INSIZE) and the volume was expressed in mm\hat{ }3 using the formula: V=0.5 a*b\hat{ }2 where a and b are the long and shirt diameters of the tumor, respectively. Results were analyzed using Prism GraphPad and expressed as mean±S.E.M. Comparisons between two groups were made by T-test, and the difference is considered significant if p is *<0.05 and **<0.01. 56C11 combination with anti-PDL1 antibody potentiates antitumor activity on MC38/hPD-L1 tumor model (Table 10 and FIG. 23)

TABLE 10
Efficacy of 56C11 combination on
MC38/hPD-L1 tumor model on Day 22

| | Tumor size | | p value vs |
|---|---|---|---|
| Treatment | (mm\hat{ }3, | | Isotype |
| (n = 8) | mean ± S.E.M.) | TGI (%) | control |
| hIgG1 control | 849.22 ± 109.77 | / | / |
| 56C11 | 521.52 ± 69.89 | 38.59 | 0.0246 |
| anti-PDL1 antibody | 522.04 ± 74.92 | 38.53 | 0.0274 |
| 56C11+ anti-PDL1 antibody | 378.94 ± 87.31 | 55.38 | 0.0047 |

TABLE 10
Sequences mentioned or used in the present application

| SEQ | | |
|---|---|---|
| ID | | |
| NO | Sequence | Annotation |
| 1 | TYGMA | 14E3/Hu14E3-Ha/ |
| | | Hu14E3-Hb/ |
| | | Hu14E3-Hc HCDR1 |
| 2 | WINTLSGEPTYADDFKG | 14E3/Hu14E3-Ha/ |
| | | Hu14E3-Hb/ |
| | | Hu14E3-Hc HCDR2 |
| 3 | EPMDY | 14E3/Hu14E3-Ha/ |
| | | Hu14E3-Hb/ |
| | | Hu14E3-Hc HCDR3 |
| 4 | KSSQSLLDSDGKTYLS | 14E3/Hu14E3-La/ |
| | | Hu14E3-Lb LCDR1 |
| 5 | LVSKLDS | 14E3/Hu14E3-La/ |
| | | Hu14E3-Lb LCDR2 |
| 6 | WQGAHFPLT | 14E3/Hu14E3-La/ |
| | | Hu14E3-Lb LCDR3 |
| 7 | QIQLVQSGPELKKPGETVKISCKTSGSTFTTY | 14E3 VH |
| | GMAWMKQAPGKGLTWMGWINTLSGEPTY | |
| | ADDFKGRFAFSLKTSANTAYLQINNLKNED | |
| | AATYFCAREPMDYWGQGTSVIVSS | |
| 8 | DVVMTQTPLTLSITIGQPASISCKSSQSLLDS | 14E3 VL |
| | DGKTYLSWLLQRPDQSPKRLISLVSKLDSGV | |
| | PDRITGSGSGTDFTLKISRVEAEDLGIYYCW | |
| | QGAHFPLTFGAGTKLELK | |
| 9 | CAGATCCAGTTGGTACAGTCTGGACCTGA | 14E3 VHnu |
| | ACTGAAGAAGCCTGGAGAGACAGTCAAGA | |
| | TCTCCTGCAAGACTTCTGGATCTACGTTCA | |
| | CAACCTATGGAATGGCCTGGATGAAGCAG | |
| | GCTCCAGGAAAGGGTTTAACGTGGATGGG | |
| | CTGGATAAACACCCTCTCTGGAGAGCCAA | |
| | CATATGCTGATGACTTCAAGGGACGGTTT | |
| | GCCTTCTCTTTGAAAACCTCTGCCAACACT | |
| | GCCTATTTGCAGATCAACAACCTCAAAAA | |
| | TGAGGACGCGGCTACATATTTCTGTGCAC | |
| | GAGAACCAATGGACTACTGGGGTCAAGGA | |
| | ACCTCAGTCATCGTCTCCTCA | |
| 10 | GATGTTGTGATGACCCAGACTCCACTCACT | 14E3 VLnu |
| | TTGTCGATTACCATTGGACAACCAGCCTCC | |
| | ATCTCTTGCAAATCAAGTCAGAGCCTCTTA | |
| | GATAGTGATGGAAAGACATATTTGAGTTG | |
| | GTTGTTACAGAGGCCAGACCAGTCTCCAA | |
| | AGCGCCTAATCTCTCTGGTGTCCAAACTGG | |
| | ACTCTGGAGTCCCTGACAGGATCACTGGC | |
| | AGTGGATCAGGGACAGATTTCACACTGAA | |
| | AATCAGCAGAGTGGAGGCTGAAGATTTGG | |
| | GCATCTATTATTGCTGGCAAGGTGCACATT | |
| | TTCCGCTCACGTTCGGTGCTGGGACCAAGC | |
| | TGGAGCTGAAA | |
| 11 | DYYMN | 22F1/Hu22F1-Ha/ |
| | | Hu22F1-Hb/ |
| | | Hu22F1-Hc/Hu22F1- |
| | | Hd HCDR1 |
| 12 | DINPKDGDSGYSHKFKG | 22F1/Hu22F1-Ha/ |
| | | Hu22F1-Hb/ |
| | | Hu22F1-Hc/Hu22F1- |
| | | Hd HCDR2 |
| 13 | GFTTVVARGDY | 22F1/Hu22F1-Ha/ |
| | | Hu22F1-Hb/ |
| | | Hu22F1-Hc/Hu22F1- |
| | | Hd HCDR3 |
| 14 | KSSQSLLDSDGKTYLN | 22F1/Hu22F1-La/ |
| | | Hu22F1-Lb LCDR1 |
| 15 | LVSKLDS | 22F1/Hu22F1-La/ |
| | | Hu22F1-Lb LCDR2 |
| 16 | WQGTHFPYT | 22F1/Hu22F1-La/ |
| | | Hu22F1-Lb LCDR3 |
| 17 | EAQLQQSGPELVKPGASVKISCKASGYSFTD | 22F1 VH |
| | YYMNWLKQSHGKSLEWIGDINPKDGDSGYS | |
| | HKFKGKATLTVDKSSSTAYMELRSLTSEDSA | |
| | VYYCASGFTTVVARGDYWGQGTTLTVSS | |
| 18 | DVVMTQTPLTLSVTIGQPASISCKSSQSLLDS | 22F1 VL |
| | DGKTYLNWLLQRPGQSPKRLIYLVSKLDSGF | |
| | PDRFTGSGSGTDFTLKISRVEAEDLGVYYCW | |
| | QGTHFPYTFGGGTKLEIK | |
| 19 | GAGGCCCAGCTGCAACAATCTGGACCTGA | 22F1 VHnu |
| | ACTGGTGAAGCCTGGGGCTTCAGTGAAGA | |
| | TATCCTGTAAGGCTTCTGGATACTCGTTCA | |
| | CTGACTACTACATGAACTGGCTGAAGCAG | |
| | AGCCATGGAAAGAGCCTTGAGTGGATTGG | |
| | AGATATTAATCCTAAAGATGGTGATAGTG | |
| | GTTACAGCCATAAGTTCAAGGGCAAGGCC | |
| | ACATTGACTGTAGACAAGTCCTCCAGCAC | |
| | AGCCTACATGGAGCTCCGCAGCCTGACAT | |
| | CTGAGGACTCTGCAGTCTATTACTGTGCAA | |
| | GCGGATTTACCACGGTAGTAGCTAGGGGG | |
| | GACTACTGGGGCCAAGGCACCACTCTCAC | |
| | AGTCTCCTCA | |
| 20 | GATGTTGTGATGACCCAGACTCCACTCACT | 22F1 VLnu |
| | TTGTCGGTTACCATTGGACAACCAGCCTCC | |
| | ATCTCTTGCAAGTCAAGTCAGAGCCTCTTA | |
| | GATAGTGATGGAAAGACATATTTGAATTG | |
| | GTTGTTACAGAGGCCAGGCCAGTCTCCAA | |
| | AGCGCCTAATCTATTTGGTGTCTAAACTGG | |
| | ACTCTGGATTCCCTGACAGGTTCACTGGCA | |
| | GTGGATCAGGGACAGATTTCACACTGAAA | |
| | ATCAGCAGAGTGGAGGCTGAGGATTTGGG | |
| | AGTTTATTATTGCTGGCAAGGTACACATTT | |
| | TCCGTACACGTTCGGAGGGGGGACCAAGC | |
| | TGGAAATAAAA | |
| 21 | DDYMH | 69H5 HCDR1 |
| 22 | WIDPENGDTEYASKFQG | 69H5 HCDR2 |
| 23 | WATVPDFDY | 69H5 HCDR3 |
| 24 | KSSQSLLNRSNQKNYLA | 69H5 LCDR1 |
| 25 | FTSTRES | 69H5 LCDR2 |
| 26 | QQHYSTPFT | 69H5 LCDR3 |
| 27 | EVQLQQSGAELVRPGASVKLSCTASGFNIKD | 69H5 VH |
| | DYMHWVKRRPEQGLEWIGWIDPENGDTEY | |
| | ASKFQGKATITADTSSNTAYLQLSSLTSEDT | |
| | AVYYCTTWATVPDFDYWGQGTTLTVSS | |
| 28 | DIVMTQSPSSLAMSVGQKVTMSCKSSQSLL | 69H5 VL |
| | NRSNQKNYLAWYQQKPGQSPKLLVHFTSTR | |
| | ESGVPDRFIGSGSGTDFTLTISNLQAEDLADY | |
| | FCQQHYSTPFTFGSGTKLEIK | |
| 29 | GAGGTGCAGCTGCAACAGTCCGGCGCTGA | 69H5 VHnu |
| | ACTGGTGAGGCCTGGAGCCTCCGTGAAGC | |
| | TGTCCTGCACCGCCAGCGGCTTCAACATCA | |
| | AGGACGACTACATGCACTGGGTGAAGAGG | |
| | AGGCCTGAGCAGGGCCTGGAGTGGATCGG | |
| | CTGGATCGACCCCGAGAACGGCGACACCG | |
| | AGTACGCCTCCAAGTTCCAGGGCAAGGCC | |
| | ACCATCACCGCCGACACCTCCTCCAACAC | |
| | CGCCTACCTGCAGCTGAGCTCCCTGACCTC | |
| | CGAGGACACCGCCGTGTACTATTGCACCA | |
| | CCTGGGCCACCGTGCCCGACTTCGACTACT | |
| | GGGGACAGGGCACCACCCTGACCGTGTCC | |
| | AGC | |
| 30 | GATATCGTGATGACCCAGTCTCCTTCCTCT | 69H5 VLnu |
| | CTGGCTATGTCAGTGGGACAGAAAGTGAC | |
| | CATGTCTTGCAAGTCCTCTCAGTCTCTGCT | |
| | GAACAGGTCCAACCAGAAGAACTACCTGG | |
| | CTTGGTACCAGCAGAAACCAGGACAGTCT | |
| | CCTAAGCTGCTGGTGCATTTTACCTCTACC | |
| | AGGGAATCCGGAGTGCCAGATAGATTTAT | |
| | CGGCTCTGGCTCCGGCACAGATTTTACACT | |
| | GACCATCTCCAATCTGCAGGCAGAAGATC | |
| | TGGCTGACTACTTTTGCCAGCAGCACTACT | |
| | CCACCCCTTTTACCTTTGGCTCCGGCACCA | |
| | AGCTGGAGATCAAG | |
| 31 | DFYMN | 56C11 HCDR1 |
| 32 | DINPNNGGTSYNQKFKG | 56C11 HCDR2 |
| 33 | DPIYYDYDEVAY | 56C11 HCDR3 |
| 34 | RSSQSLVHSNGNTYLH | 56C11 LCDR1 |
| 35 | KVSNRFS | 56C11/36F5/42B9/ |
| | | 67G11 LCDR2 |
| 36 | SQSTHVPLT | 56C11 LCDR3 |
| 37 | EVQLQQSGPELVKPGASVKISCKASGYTFTD | 56C11 VH |
| | FYMNWVKQSHGKSLEWIGDINPNNGGTSYN | |
| | QKFKGKATLTVDKSSSTAYMELRSLTSEDSA | |
| | VYYCARDPIYYDYDEVAYWGQGTLVTVSA | |
| 38 | DVVMTQTPLSLPVSLGDQASISCRSSQSLVH | 56C11 VL |
| | SNGNTYLHWYLQKPGQSPKLLIYKVSNRFS | |
| | GVPDRFSGSGSGTDFTLKISRVEAEDLGVYF | |
| | CSQSTHVPLTFGAGTKLELK | |
| 39 | GAGGTGCAGCTGCAGCAGTCCGGCCCTGA | 56C11 VHnu |
| | GCTGGTGAAGCCTGGAGCCTCCGTGAAGA | |
| | TCTCCTGTAAGGCCTCCGGCTACACCTTCA | |
| | CCGACTTCTACATGAACTGGGTGAAGCAG | |
| | TCCCACGGCAAGTCCCTGGAGTGGATCGG | |
| | CGACATCAATCCCAACAACGGCGGCACCT | |
| | CCTACAACCAGAAGTTCAAGGGCAAGGCC | |
| | ACCCTGACAGTGGACAAGTCCTCCAGCAC | |
| | CGCCTACATGGAGCTGAGGTCCCTGACCT | |
| | CCGAGGACTCCGCCGTGTACTACTGCGCC | |
| | AGGGACCCCATCTACTACGACTACGACGA | |
| | GGTGGCCTACTGGGGCCAGGGAACCCTGG | |
| | TGACAGTGTCCGCC | |
| 40 | GATGTGGTGATGACACAGACACCTCTGTC | 56C11 VLnu |
| | TCTGCCAGTGTCTCTCGGAGATCAGGCTTC | |
| | TATCTCTTGCAGATCCTCTCAGTCTCTGGT | |
| | GCATTCCAACGGAAACACCTACCTGCATT | |
| | GGTACCTGCAGAAACCAGGACAGTCTCCT | |
| | AAGCTGCTGATCTACAAGGTGTCCAACAG | |
| | GTTCTCCGGAGTGCCAGATAGATTTTCCGG | |
| | ATCTGGATCTGGCACCGATTTTACCCTGAA | |
| | GATCTCTAGAGTGGAAGCAGAGGATCTGG | |
| | GAGTGTACTTTTGTAGCCAGTCTACCCACG | |
| | TGCCTCTGACATTTGGAGCAGGAACAAAG | |
| | CTGGAGCTGAAG | |
| 41 | QVQLVQSGSELKKPGASVKVSCKASGYTFT | Hu14E3-Ha VH |
| | TYGMAWMRQAPGQGLEWMGWINTLSGEP | |
| | TYADDFKGRFVFSLDTSVSTAYLQISSLKAE | |
| | DTAVYYCAREPMDYWGQGTMVTVSS | |
| 42 | CAGGTGCAGCTGGTGCAGTCCGGCTCCGA | Hu14E3-Ha VHnu |
| | GCTGAAGAAGCCTGGCGCCTCCGTGAAGG | |
| | TGTCCTGCAAGGCCTCCGGCTACACCTTCA | |
| | CCACCTACGGCATGGCCTGGATGAGGCAG | |
| | GCTCCTGGCCAGGGACTGGAGTGGATGGG | |
| | CTGGATCAACACCCTGTCCGGCGAACCCA | |
| | CCTACGCCGACGACTTCAAGGGCAGGTTC | |
| | GTGTTCTCCCTGGACACCAGCGTGTCCACC | |
| | GCCTACCTGCAGATCTCCTCCCTGAAGGCC | |
| | GAGGACACCGCCGTGTACTACTGCGCCAG | |
| | GGAGCCCATGGACTACTGGGGCCAGGGCA | |
| | CCATGGTGACCGTGTCCTCC | |
| 43 | QIQLVQSGSELKKPGASVKVSCKASGYTFTT | Hu14E3-Hb VH |
| | YGMAWMRQAPGQGLEWMGWINTLSGEPT | |
| | YADDFKGRFAFSLDTSVSTAYLQISSLKAED | |
| | TAVYYCAREPMDYWGQGTMVTVSS | |
| 44 | CAGATCCAGCTGGTGCAGAGCGGCAGCGA | Hu14E3-Hb VHnu |
| | GCTGAAGAAGCCCGGCGCTAGCGTGAAGG | |
| | TGTCCTGCAAGGCCAGCGGCTACACCTTC | |
| | ACCACCTACGGCATGGCCTGGATGAGGCA | |
| | GGCTCCTGGACAGGGCCTGGAGTGGATGG | |
| | GCTGGATCAACACCCTGTCCGGCGAGCCT | |
| | ACCTACGCCGACGACTTCAAGGGCAGGTT | |
| | CGCCTTCTCCCTGGACACCTCCGTGAGCAC | |
| | CGCCTACCTGCAGATCTCCAGCCTGAAGG | |
| | CCGAGGACACCGCCGTGTACTACTGCGCC | |
| | AGGGAGCCTATGGACTACTGGGGCCAGGG | |
| | CACCATGGTGACCGTGTCCAGC | |
| 45 | QIQLVQSGSELKKPGASVKVSCKASGSTFTT | Hu14E3-Hc VH |
| | YGMAWMKQAPGQGLTWMGWINTLSGEPT | |
| | YADDFKGRFAFSLDTSVSTAYLQISSLKAED | |
| | TAVYYCAREPMDYWGQGTMVTVSS | |
| 46 | CAGATCCAGCTGGTGCAGTCCGGCAGCGA | Hu14E3-Hc VHnu |
| | GCTCAAGAAGCCCGGAGCCAGCGTGAAGG | |
| | TGTCCTGCAAGGCCAGCGGCTCCACCTTCA | |
| | CCACATACGGCATGGCCTGGATGAAGCAG | |
| | GCTCCTGGCCAGGGCCTGACCTGGATGGG | |
| | ATGGATCAACACCCTGTCCGGCGAGCCTA | |
| | CCTACGCCGATGACTTCAAGGGCAGGTTC | |
| | GCCTTCTCCCTGGACACCTCCGTGTCCACC | |
| | GCTTACCTGCAGATCTCCTCCCTGAAGGCC | |
| | GAGGACACCGCCGTGTACTACTGCGCCAG | |
| | GGAGCCCATGGACTACTGGGGCCAGGGCA | |
| | CCATGGTGACCGTGTCCTCC | |
| 47 | DVVMTQSPLSLPVTLGQPASISCKSSQSLLDS | Hu14E3-La VL |
| | DGKTYLSWLQQRPGQSPRRLIYLVSKLDSG | |
| | VPDRFSGSGSGTDFTLKISRVEAEDVGVYYC | |
| | WQGAHFPLTFGQGTKLEIK | |
| 48 | GATGTGGTGATGACACAGTCTCCTCTGTCT | Hu14E3-La VLnu |
| | CTGCCAGTGACACTGGGACAGCCAGCTTC | |
| | TATCTCTTGCAAGTCCTCTCAGTCTCTGCT | |
| | GGATTCCGACGGAAAGACCTATCTGTCTT | |
| | GGCTGCAGCAGAGACCAGGACAGTCTCCT | |
| | AGAAGACTGATCTACCTGGTGTCCAAGCT | |
| | GGATTCTGGAGTGCCAGATAGATTTTCCG | |
| | GCTCCGGCTCTGGCACAGATTTCACCCTGA | |
| | AGATCTCTAGAGTGGAGGCAGAAGACGTG | |
| | GGAGTGTACTATTGTTGGCAGGGAGCTCA | |
| | CTTCCCTCTGACATTTGGACAGGGAACAA | |
| | AGCTGGAGATCAAG | |
| 49 | DVVMTQSPLSLPVTLGQPASISCKSSQSLLDS | Hu14E3-Lb VL |
| | DGKTYLSWLQQRPGQSPRRLISLVSKLDSGV | |
| | PDRFSGSGSGTDFTLKISRVEAEDVGVYYCW | |
| | QGAHFPLTFGQGTKLEIK | |
| 50 | GATGTGGTGATGACACAGTCTCCTCTGTCT | Hu14E3-Lb VLnu |
| | CTGCCAGTGACACTGGGACAGCCAGCTTC | |
| | TATCTCTTGCAAGTCCTCTCAGTCTCTGCT | |
| | GGATTCCGACGGAAAGACCTATCTGTCTT | |
| | GGCTGCAGCAGAGACCAGGACAGTCTCCT | |
| | AGAAGACTGATCTCCCTGGTGTCTAAGCT | |
| | GGATTCCGGAGTGCCAGATAGATTTTCCG | |
| | GATCTGGATCTGGCACCGATTTTACCCTGA | |
| | AGATCTCTAGAGTGGAGGCAGAAGACGTG | |
| | GGAGTGTACTATTGTTGGCAGGGAGCTCA | |
| | CTTCCCTCTGACATTTGGACAGGGAACAA | |
| | AGCTGGAGATCAAG | |
| 51 | QVQLVQSGAEVKKPGASVKVSCKASGYSFT | Hu22F1-Ha VH |
| | DYYMNWVRQAPGQGLEWMGDINPKDGDS | |
| | GYSHKFKGRVTMTRDTSTSTVYMELSSLRS | |
| | EDTAVYYCASGFTTVVARGDYWGQGTTVT | |
| | VSS | |
| 52 | CAAGTTCAGCTGGTGCAGTCCGGAGCCGA | Hu22F1-Ha VHnu |
| | GGTGAAGAAGCCCGGCGCTTCCGTGAAGG | |
| | TGTCTTGTAAGGCCTCCGGCTACTCCTTCA | |
| | CCGATTACTACATGAACTGGGTGAGGCAA | |
| | GCTCCCGGTCAAGGTCTGGAGTGGATGGG | |
| | CGACATCAACCCCAAGGACGGCGACTCCG | |
| | GCTATTCCCACAAGTTCAAGGGTCGTGTG | |
| | ACCATGACCAGGGACACGTCCACCAGCAC | |
| | CGTGTACATGGAGCTGTCCTCTTTAAGGTC | |
| | CGAGGACACCGCCGTGTACTACTGCGCCA | |
| | GCGGATTCACCACCGTGGTGGCTAGGGGC | |
| | GACTATTGGGGCCAAGGTACCACCGTGAC | |
| | AGTGTCCAGC | |
| 53 | QVQLVQSGAEVKKPGASVKVSCKASGYSFT | Hu22F1-Hb VH |
| | DYYMNWVRQAPGQGLEWMGDINPKDGDS | |
| | GYSHKFKGRVTMTVDKSTSTVYMELSSLRS | |
| | EDTAVYYCASGFTTVVARGDYWGQGTTVT | |
| | VSS | |
| 54 | CAAGTTCAGCTGGTGCAGTCCGGAGCCGA | Hu22F1-Hb VHnu |
| | GGTGAAGAAGCCCGGCGCTTCCGTGAAGG | |
| | TGTCTTGTAAGGCCTCCGGCTACTCCTTCA | |
| | CCGATTACTACATGAACTGGGTGAGGCAA | |
| | GCTCCCGGTCAAGGTCTGGAGTGGATGGG | |
| | CGACATCAACCCCAAGGACGGCGACTCCG | |
| | GCTATTCCCACAAGTTCAAGGGTCGTGTG | |
| | ACCATGACCGTGGACAAGTCCACCAGCAC | |
| | CGTGTACATGGAGCTGTCCTCTTTAAGGTC | |
| | CGAGGACACCGCCGTGTACTACTGCGCCA | |
| | GCGGATTCACCACCGTGGTGGCTAGGGGC | |
| | GACTATTGGGGCCAAGGTACCACCGTGAC | |
| | AGTGTCCAGC | |
| 55 | QAQLVQSGAEVKKPGASVKVSCKASGYSFT | Hu22F1-Hc VH |
| | DYYMNWVRQAPGQGLEWMGDINPKDGDS | |
| | GYSHKFKGRVTLTVDKSTSTVYMELRSLRS | |
| | EDTAVYYCASGFTTVVARGDYWGQGTTVT | |
| | VSS | |
| 56 | CAAGCTCAGCTGGTGCAGTCCGGCGCTGA | Hu22F1-Hc VHnu |
| | GGTGAAAAAGCCCGGCGCCAGCGTGAAGG | |
| | TGTCTTGTAAGGCCTCCGGCTACTCCTTCA | |
| | CCGACTACTACATGAACTGGGTGAGGCAA | |
| | GCTCCCGGTCAAGGTCTGGAGTGGATGGG | |
| | CGACATCAACCCCAAGGACGGCGACAGCG | |
| | GCTACTCCCACAAGTTCAAGGGTCGTGTG | |
| | ACTTTAACCGTGGACAAGTCCACCTCCACC | |
| | GTCTACATGGAGCTGAGGTCTTTAAGGTCC | |
| | GAGGATACCGCCGTGTACTACTGCGCTAG | |
| | CGGCTTCACCACCGTGGTGGCTCGTGGCG | |
| | ATTACTGGGGACAAGGTACCACCGTGACC | |
| | GTGTCCTCC | |
| 57 | QAQLVQSGAEVKKPGASVKVSCKASGYSFT | Hu22F1-Hd VH |
| | DYYMNWLRQAPGQGLEWIGDINPKDGDSG | |
| | YSHKFKGRATLTVDKSTSTVYMELRSLRSE | |
| | DTAVYYCASGFTTVVARGDYWGQGTTVTV | |
| | SS | |
| 58 | CAAGCTCAACTGGTGCAGTCCGGCGCCGA | Hu22F1-Hd VHnu |
| | GGTGAAAAAGCCCGGTGCCTCCGTGAAGG | |
| | TGAGCTGCAAGGCCTCCGGCTACTCCTTTA | |
| | CCGACTACTACATGAACTGGCTGAGGCAA | |
| | GCTCCCGGTCAAGGTCTGGAGTGGATCGG | |
| | CGATATCAACCCCAAGGACGGCGACTCCG | |
| | GCTACAGCCATAAGTTCAAGGGTCGTGCC | |
| | ACTTTAACCGTGGACAAGTCCACCAGCAC | |
| | CGTGTACATGGAGCTGAGGTCTTTAAGGT | |
| | CCGAGGACACCGCCGTGTACTACTGCGCC | |
| | TCCGGCTTCACCACAGTGGTGGCTCGTGGC | |
| | GACTATTGGGGCCAAGGTACCACCGTGAC | |
| | CGTGAGCTCC | |
| 59 | DVVMTQSPLSLPVTLGQPASISCKSSQSLLDS | Hu22F1-La VL |
| | DGKTYLNWLQQRPGQSPRRLIYLVSKLDSG | |
| | VPDRFSGSGSGTDFTLKISRVEAEDVGVYYC | |
| | WQGTHFPYTFGGGTKVEIK | |
| 60 | GATGTGGTGATGACACAGTCTCCTCTGTCT | Hu22F1-La VLnu |
| | CTGCCAGTGACACTGGGACAGCCAGCTTC | |
| | TATCTCTTGCAAGTCCTCTCAGTCTCTGCT | |
| | GGATTCCGACGGAAAGACCTACCTGAATT | |
| | GGCTGCAGCAGAGACCAGGACAGTCTCCT | |
| | AGAAGACTGATCTACCTGGTGTCCAAGCT | |
| | GGATTCTGGAGTGCCAGATAGATTTTCCG | |
| | GCTCCGGCTCTGGCACAGATTTCACCCTGA | |
| | AGATCTCTAGAGTGGAGGCAGAAGACGTG | |
| | GGAGTGTACTATTGTTGGCAGGGAACCCA | |
| | CTTCCCTTACACATTTGGAGGAGGCACAA | |
| | AGGTGGAGATCAAG | |
| 61 | DVVMTQSPLSLPVTLGQPASISCKSSQSLLDS | Hu22F1-Lb VL |
| | DGKTYLNWLQQRPGQSPRRLIYLVSKLDSG | |
| | FPDRFSGSGSGTDFTLKISRVEAEDVGVYYC | |
| | WQGTHFPYTFGGGTKVEIK | |
| 62 | GATGTGGTGATGACACAGTCTCCTCTGTCT | Hu22F1-Lb VLnu |
| | CTGCCAGTGACACTGGGACAGCCAGCTTC | |
| | TATCTCTTGCAAGTCCTCTCAGTCTCTGCT | |
| | GGATTCCGACGGAAAGACCTACCTGAATT | |
| | GGCTGCAGCAGAGACCAGGACAGTCTCCT | |
| | AGAAGACTGATCTACCTGGTGTCCAAGCT | |
| | GGATTCTGGATTCCCAGATAGATTTTCCGG | |
| | CTCCGGCTCTGGCACAGATTTCACCCTGAA | |
| | GATCTCTAGAGTGGAGGCAGAAGACGTGG | |
| | GAGTGTACTATTGTTGGCAGGGAACCCAC | |
| | TTCCCTTACACATTTGGAGGAGGCACAAA | |
| | GGTGGAGATCAAG | |
| 63 | NSFYIPRHIRKEEGSFQSCSF | BMP-binding loop |
| 64 | FSYSVPNTFPQSTESLVHCDS | the 63-83 amino |
| | | acids of DAN |
| 65 | MGWSCIILFLVATGVHS | signal peptide |
| 66 | MSRTAYTVGALLLLLGTLLPAAEGKKKGSQ | Human gremlin1 |
| | GAIPPPDKAQHNDSEQTQSPQQPGSRNRGRG | (hGREM1) |
| | QGRGTAMPGEEVLESSQEALHVTERKYLKR | |
| | DWCKTQPLKQTIHEEGCNSRTIINRFCYGQC | |
| | NSFYIPRHIRKEEGSFQSCSFCKPKKFTTMMV | |
| | TLNCPELQPPTKKKRVTRVKQCRCISIDLD | |
| 67 | MNRTAYTVGALLLLLGTLLPTAEGKKKGSQ | Mouse gremlin1 |
| | GAIPPPDKAQHNDSEQTQSPPQPGSRTRGRG | (mGREM1) |
| | QGRGTAMPGEEVLESSQEALHVTERKYLKR | |
| | DWCKTQPLKQTIHEEGCNSRTIINRFCYGQC | |
| | NSFYIPRHIRKEEGSFQSCSFCKPKKFTTMMV | |
| | TLNCPELQPPTKKKRVTRVKQCRCISIDLD | |
| 68 | MSRTAYTVGALLLLLGTLLPAAEGKKKGSQ | chimeric hGREM1 |
| | GAIPPPDKAQHNDSEQTQSPQQPGSRNRGRG | |
| | QGRGTAMPGEEVLESSQEALHVTERKYLKR | |
| | DWCKTQPLKQTIHEEGCNSRTIINRFCYGQC | |
| | FSYSVPNTFPQSTESLVHCDSCKPKKFTTMM | |
| | VTLNCPELQPPTKKKRVTRVKQCRCISIDLD | |
| 69 | KKKGSQGAIPPPDKAQHNDSEQTQSPQQPGS | Human Gremlin 1 |
| | RNRGRGQGRGTAMPGEEVLESSQEALHVTE | sequence without |
| | RKYLKRDWCKTQPLKQTIHEEGCNSRTIINR | signal peptide |
| | FCYGQCNSFYIPRHIRKEEGSFQSCSFCKPKK | |
| | FTTMMVTLNCPELQPPTKKKRVTRVKQCRC | |
| | ISIDLD | |
| 70 | KKKGSQGAIPPPDKAQHNDSEQTQSPPQPGS | Mouse Gremlin 1 |
| | RTRGRGQGRGTAMPGEEVLESSQEALHVTE | sequence without |
| | RKYLKRDWCKTQPLKQTIHEEGCNSRTIINR | signal peptide |
| | FCYGQCNSFYIPRHIRKEEGSFQSCSFCKPKK | |
| | FTTMMVTLNCPELQPPTKKKRVTRVKQCRC | |
| | ISIDLD | |
| 71 | MSRTAYTVGALLLLLGTLLPAAEG | Human Gremlin 1 |
| | | signal peptide |
| 72 | MNRTAYTVGALLLLLGTLLPTAEG | Mouse Gremlin 1 |
| | | signal peptide |
| 73 | QVQLVQSGAEVKKPGASVKVSCKASGYSFT | Hu22F1-Ha/Hb FR1 |
| 74 | WVRQAPGQGLEWMG | Hu22F1-Ha/Hb/Hc |
| | | FR2 |
| | | Hu56C11-H0/Ha |
| | | FR2 |
| 75 | RVTMTRDTSTSTVYMELSSLRSEDTAVYYC | Hu22F1-Ha FR3 |
| | AS | |
| 76 | WGQGTTVTVSS | Hu22F1-Ha/Hb/Hc/ |
| | | Hd FR4 |
| 77 | QAQLVQSGAEVKKPGASVKVSCKASGYSFT | Hu22F1-Hc/Hd FR1 |
| 78 | RVTMTVDKSTSTVYMELSSLRSEDTAVYYC | Hu22F1-Hb FR3 |
| | AS | |
| 79 | RVTLTVDKSTSTVYMELRSLRSEDTAVYYC | Hu22F1-Hc FR3 |
| | AS | |
| 80 | WLRQAPGQGLEWIG | Hu22F1-Hd FR2 |
| 81 | RATLTVDKSTSTVYMELRSLRSEDTAVYYC | Hu22F1-Hd FR3 |
| | AS | |
| 82 | QVQLVQSGSELKKPGASVKVSCKASGYTFT | Hu14E3-Ha FR1 |
| 83 | WMRQAPGQGLEWMG | Hu14E3-Ha/Hb FR2 |
| 84 | RFVFSLDTSVSTAYLQISSLKAEDTAVYYCA | Hu14E3-Ha FR3 |
| | R | |
| 85 | WGQGTMVTVSS | Hu14E3-Ha/Hb/Hc |
| | | FR4 |
| 86 | QIQLVQSGSELKKPGASVKVSCKASGYTFT | Hu14E3-Hb FR1 |
| 87 | RFAFSLDTSVSTAYLQISSLKAEDTAVYYCA | Hu14E3-Hb/Hc FR3 |
| | R | |
| 88 | QIQLVQSGSELKKPGASVKVSCKASGSTFT | Hu14E3-Hc FR1 |
| 89 | WMKQAPGQGLTWMG | Hu14E3-Hc FR2 |
| 90 | QVQLVQSGAEVKKPGASVKVSCKASGYTFT | Hu56C11-H0/Ha/Hb/ |
| | | Hc FR1 |
| 91 | RVTMTRDTSISTAYMELSRLRSDDTAVYYC | Hu56C11-H0 FR3 |
| | AR | |
| 92 | WGQGTLVTVSS | Hu56C11-H0/Ha/Hb/ |
| | | Hc FR4 |
| 93 | RVTMTVDKSISTAYMELSRLRSDDTAVYYC | Hu56C11-Ha FR3 |
| | AR | |
| 94 | RVTLTVDKSISTAYMELSRLRSDDTAVYYC | Hu56C11-Hb/Hc |
| | AR | FR3 |
| 95 | WVRQAPGQGLEWIG | Hu56C11-Hb FR2 |
| 96 | WVKQAPGKGLEWIG | Hu56C11-Hc FR2 |
| 97 | QX₁QLVQSGX₂EX₃KKPGASVKVSCKASGX₄ | Hu22F1/Hu14E3/Hu |
| | X₅FT | 56C11 HFR1 |
| 98 | WB₆X₇QAPGX₈GLB₉WX₁₀G | Hu22F1/Hu14E3/Hu |
| | | 56C11 HFR2 |
| 99 | RX₁₁TX₁₂TX₁₃DX₁₄STSTVYMELX₁₅SLRSEDTA | Hu22F1 HFR3 |
| | VYYCAS | |
| 100 | WGQGTX₂₀VTVSS | Hu22F1/Hu14E3/Hu |
| | | 56C11 HFR4 |
| 101 | DVVMTQSPLSLPVTLGQPASISC | Hu22F1-La/Lb FR1 |
| | | Hu14E3-La/Lb FR1 |
| | | Hu56C11-L0/La/Lb |
| | | FR1 |
| 102 | WLQQRPGQSPRRLIY | Hu22F1-La/Lb FR2 |
| | | Hu14E3-La FR2 |
| 103 | GVPDRFSGSGSGTDFTLKISRVEAEDVGVYY | Hu22F1-La FR3 |
| | C | Hu14E3-La/Lb FR3 |
| | | Hu56C11-L0/La/Lb |
| | | FR3 |
| 104 | FGGGTKVEIK | Hu22F1-La/Lb FR4 |
| 105 | GFPDRFSGSGSGTDFTLKISRVEAEDVGVYY | Hu22F1-Lb FR3 |
| | C | |
| 106 | FGQGTKLEIK | Hu14E3-La/Lb FR4 |
| | | Hu56C11-L0/La/Lb |
| | | FR4 |
| 107 | WLQQRPGQSPRRLIS | Hu14E3-Lb FR2 |
| 108 | WFQQRPGQSPRRLIY | Hu56C11-L0 FR2 |
| 109 | WFQQRPGQSPRLLIY | Hu56C11-La FR2 |
| 110 | WYQQRPGQSPRLLIY | Hu56C11-Lb FR2 |
| 111 | WX₂₁QQRPGQSPRX₂₂LIX₂₃ | Hu22F1/Hu14E3/Hu |
| | | 56C11-LFR2 |
| 112 | GX₂₄PDRFSGSGSGTDFTLKISRVEAEDVGVY | Hu22F1/Hu14E3/Hu |
| | YC | 56C11-LFR3 |
| 113 | FGX₂₅GTKX₂₆EIK | Hu22F1/Hu14E3/Hu |
| | | 56C11-LFR4 |
| 114 | SSGIG | 36F5 HCDR1 |
| 115 | EIYPRSGNTYNNEKFKG | 36F5/42B9/67G11 |
| | | HCDR2 |
| 116 | EAYSHHYYAMDY | 36F5 HCDR3 |
| 117 | RSSQSLLHSNGNTYLE | 36F5 LCDR1 |
| 118 | FQGSHVPFT | 36F5/42B9/67G11 |
| | | LCDR3 |
| 119 | SYGIG | 42B9/67G11 |
| | | HCDR1 |
| 120 | EGYSNNYYAMDY | 42B9/67G11 |
| | | HCDR3 |
| 121 | ISSQSLVHSNGNTYLE | 42B9 LCDR1 |
| 122 | RSSQSLVHSNGNTYLE | 67G11 LCDR1 |
| 123 | SX₃₂GIG | 36F5/42B9/67G11 |
| | | HCDR1 |
| 124 | EX₂₇YSX₂₈X₂₉YYAMDY | 36F5/42B9/67G11 |
| | | HCDR3 |
| 125 | X₃₀SSQSLX₃₁HSNGNTYLE | 36F5/42B9/67G11 |
| | | LCDR1 |
| 126 | QVQLQQSGAELARPGASVKLSCKASGYSFTSSGI | 36F5 VH |
| | GWVKQRSGQGLEWIGEIYPRSGNTYNNEKFKGK | |
| | ATLTADKSSSTVYMELRSLTSEDSAVYFCVREAY | |
| | SHHYYAMDYWGQGTSVTVFS | |
| 127 | DVLMTQTPLSLPVSLGGQASISCRSSQSLLHSNG | 36F5 VL |
| | NTYLEWYLQKPGQSPKLLIYKVSNRFSGVPDRLS | |
| | GSGSGTDFTLKISRVEAEDLGVYYCFQGSHVPFT | |
| | FGSGTKLEIN | |
| 128 | QVQLQQSGAELARPGASVKLSCKASGYTFTSYG | 42B9/67G11 VH |
| | IGWVKQRTGQGLEWIGEIYPRSGNTYNNEKFKG | |
| | KATLTADKSSRTVYMELRSLISEDSAVYFCAREG | |
| | YSNNYYAMDYWGQGTSVTVFS | |
| 129 | DVLMTQTPLSLPVSLGDQASISCISSQSLVHSNGN | 42B9 VL |
| | TYLEWYLQKPGLSPKLLIYKVSNRFSGVPDRLSG | |
| | SGSGTDFTLRISRVEAEDLGVYYCFQGSHVPFTF | |
| | GSGTKLEIK | |
| 130 | DVLMTQTPLSLPVSLGDQASISCRSSQSLVHSNG | 67G11 VL |
| | NTYLEWYLQKPGQSPKLLIYKVSNRFSGVPDRLS | |
| | GSGSGTDFTLKISRVEAEDLGVYYCFQGSHVPFT | |
| | FGSGTKLEIK | |
| 131 | QVQLVQSGAEVKKPGASVKVSCKASGYTFTDFY | Hu56C11-H0 VH |
| | MNWVRQAPGQGLEWMGDINPNNGGTSYNQKF | |
| | KGRVTMTRDTSISTAYMELSRLRSDDTAVYYCAR | |
| | DPIYYDYDEVAYWGQGTLVTVSS | |
| 132 | QVQLVQSGAEVKKPGASVKVSCKASGYTFTDFY | Hu56C11-Ha VH |
| | MNWVRQAPGQGLEWMGDINPNNGGTSYNQKF | |
| | KGRVTMTVDKSISTAYMELSRLRSDDTAVYYCA | |
| | RDPIYYDYDEVAYWGQGTLVTVSS | |
| 133 | QVQLVQSGAEVKKPGASVKVSCKASGYTFTDFY | Hu56C11-Hb VH |
| | MNWVRQAPGQGLEWIGDINPNNGGTSYNQKFK | |
| | GRVTLTVDKSISTAYMELSRLRSDDTAVYYCARD | |
| | PIYYDYDEVAYWGQGTLVTVSS | |
| 134 | QVQLVQSGAEVKKPGASVKVSCKASGYTFTDFY | Hu56C11-Hc VH |
| | MNWVKQAPGKGLEWIGDINPNNGGTSYNQKFK | |
| | GRVTLTVDKSISTAYMELSRLRSDDTAVYYCARD | |
| | PIYYDYDEVAYWGQGTLVTVSS | |
| 135 | DVVMTQSPLSLPVTLGQPASISCRSSQSLVHSNG | Hu56C11-L0 VL |
| | NTYLHWFQQRPGQSPRRLIYKVSNRFSGVPDRFS | |
| | GSGSGTDFTLKISRVEAEDVGVYYCSQSTHVPLT | |
| | FGQGTKLEIK | |
| 136 | DVVMTQSPLSLPVTLGQPASISCRSSQSLVHSNG | Hu56C11-La VL |
| | NTYLHWFQQRPGQSPRLLIYKVSNRFSGVPDRFS | |
| | GSGSGTDFTLKISRVEAEDVGVYYCSQSTHVPLT | |
| | FGQGTKLEIK | |
| 137 | DVVMTQSPLSLPVTLGQPASISCRSSQSLVHSNG | Hu56C11-Lb VL |
| | NTYLHWYQQRPGQSPRLLIYKVSNRFSGVPDRF | |
| | SGSGSGTDFTLKISRVEAEDVGVYYCSQSTHVPL | |
| | TFGQGTKLEIK | |
| 138 | ASTKGPSVFPLAPSSKSTSGGTAALGCLVKDYFP | Human IgG1 heavy |
| | EPVTVSWNSGALTSGVHTFPAVLQSSGLYSLSSV | chain constant |
| | VTVPSSSLGTQTYICNVNHKPSNTKVDKKVEPKS | region |
| | CDKTHTCPPCPAPELLGGPSVFLFPPKPKDTLMIS | |
| | RTPEVTCVVVDVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYTLP | |
| | PSRDELTKNQVSLTCLVKGFYPSDIAVEWESNGQ | |
| | PENNYKTTPPVLDSDGSFFLYSKLTVDKSRWQQG | |
| | NVFSCSVMHEALHNHYTQKSLSLSPGK | |
| 139 | RTVAAPSVFIFPPSDEQLKSGTASVVCLLNNF | Human Kappa light |
| | YPREAKVQWKVDNALQSGNSQESVTEQDS | chain constant |
| | KDSTYSLSSTLTLSKADYEKHKVYACEVTH | region |
| | QGLSSPVTKSFNRGEC | |
| 140 | RFX₁₆FSLDTSVSTAYLQISSLKAEDTAVYYCA | Hu14E3 HFR3 |
| | R | |
| 141 | RVTX₁₇TX₁₈DX₁₉SISTAYMELSRLRSDDTAVY | Hu56C11 HFR3 |
| | YCAR | |
| 142 | CAGGTTCAGCTGCAGCAGTCTGGAGCTGA | 36F5 VHnu |
| | GCTGGCGAGGCCTGGGGCTTCAGTGAAGC | |
| | TGTCCTGCAAGGCTTCTGGCTACTCCTTCA | |
| | CAAGCTCTGGTATAGGCTGGGTGAAGCAG | |
| | AGATCTGGACAGGGCCTTGAGTGGATTGG | |
| | AGAGATTTATCCTAGAAGTGGTAATACTTAC | |
| | AACAATGAGAAGTTCAAGGGCAAGGCCAC | |
| | ACTGACTGCAGACAAATCCTCCAGCACAG | |
| | TGTACATGGAACTCCGCAGCCTGACATCTG | |
| | AGGACTCTGCGGTCTATTTTTGTGTAAGAG | |
| | AGGCCTATAGTCACCATTACTATGCTATGGA | |
| | CTATTGGGGTCAAGGAACCTCAGTCACCGT | |
| | CTTCTCA | |
| 143 | GATGTTTTGATGACCCAAACTCCTCTCTCC | 36F5 VLnu |
| | CTGCCTGTCAGTCTTGGAGGTCAAGCCTCC | |
| | ATCTCTTGCAGATCTAGTCAGAGCCTTCTA | |
| | CATAGTAATGGAAACACCTATTTAGAATGGT | |
| | ACCTGCAGAAACCAGGCCAGTCTCCAAAG | |
| | CTCCTGATCTACAAAGTTTCCAACCGATTT | |
| | TCTGGGGTCCCAGACAGGCTCAGTGGCAG | |
| | TGGATCAGGGACAGATTTTACACTCAAGAT | |
| | CAGCAGAGTGGAGGCTGAGGATCTGGGAG | |
| | TTTATTACTGCTTTCAAGGTTCACATGTTCC | |
| | ATTCACGTTCGGCTCGGGGACAAAGTTGG | |
| | AAATAAAT | |
| 144 | CAGGTTCAGCTGCAGCAGTCTGGAGCTGA | 42B9 VHnu |
| | GCTGGCGAGGCCTGGGGCTTCAGTGAAGC | |
| | TGTCCTGCAAGGCTTCTGGCTACACCTTCA | |
| | CAAGCTATGGTATAGGCTGGGTGAAGCAGA | |
| | GAACTGGACAGGGCCTTGAGTGGATTGGA | |
| | GAGATTTATCCTAGAAGTGGTAATACTTACA | |
| | ACAATGAGAAGTTCAAGGGCAAGGCCACA | |
| | CTGACTGCAGACAAATCCTCCAGAACAGT | |
| | GTACATGGAGCTCCGCAGTCTGATATCTGA | |
| | GGACTCTGCGGTCTACTTTTGTGCAAGAGA | |
| | GGGCTATAGTAACAATTACTATGCTATGGAC | |
| | TACTGGGGTCAAGGAACCTCAGTCACCGT | |
| | CTTCTCA | |
| 145 | GATGTTTTGATGACCCAAACTCCACTCTCC | 42B9 VLnu |
| | CTGCCTGTCAGTCTTGGAGATCAAGCCTCC | |
| | ATCTCTTGCATATCTAGTCAGAGCCTTGTAC | |
| | ATAGTAATGGAAACACCTATTTAGAATGGT | |
| | ACCTGCAGAAACCAGGCCTGTCTCCAAAA | |
| | CTCCTGATCTACAAAGTTTCCAACCGATTT | |
| | TCTGGGGTCCCAGACAGGCTCAGTGGCAG | |
| | TGGATCAGGGACAGATTTCACACTCAGGAT | |
| | CAGCAGAGTGGAGGCTGAGGATCTGGGAG | |
| | TTTATTACTGCTTTCAAGGTTCACATGTTCC | |
| | ATTCACGTTCGGCTCGGGGACAAAGTTGG | |
| | AAATAAAA | |
| 146 | CAGGTTCAGCTGCAGCAGTCTGGAGCTGA | 67G11 VHnu |
| | GCTGGCGAGGCCTGGGGCTTCAGTGAAGC | |
| | TGTCCTGCAAGGCTTCTGGCTACACCTTCA | |
| | CAAGTTATGGTATAGGCTGGGTGAAGCAGA | |
| | GAACTGGACAGGGCCTTGAGTGGATTGGA | |
| | GAGATTTATCCTAGAAGTGGTAATACTTACA | |
| | ACAATGAGAAGTTCAAGGGCAAGGCCACA | |
| | CTGACTGCGGACAAATCCTCCAGAACAGT | |
| | GTACATGGAGCTCCGCAGTCTGATATCTGA | |
| | GGACTCTGCGGTCTACTTTTGTGCAAGAGA | |
| | GGGCTATAGTAACAATTACTATGCTATGGAC | |
| | TACTGGGGTCAAGGAACCTCAGTCACCGT | |
| | CTTCTCA | |
| 147 | GATGTTTTGATGACCCAAACTCCACTCTCC | 67G11 VLnu |
| | CTGCCTGTCAGTCTTGGAGATCAAGCCTCC | |
| | ATCTCTTGCAGATCTAGTCAGAGCCTTGTA | |
| | CATAGTAATGGAAACACCTATTTAGAATGGT | |
| | ACCTGCAGAAACCAGGCCAGTCTCCAAAG | |
| | CTCCTGATCTACAAAGTTTCCAACCGATTT | |
| | TCTGGGGTCCCAGACAGGCTCAGTGGCAG | |
| | TGGATCAGGGACAGATTTCACACTCAAGAT | |
| | CAGCAGAGTGGAGGCTGAGGATCTGGGAG | |
| | TTTATTACTGCTTTCAAGGTTCACATGTTCC | |
| | ATTCACGTTCGGCTCGGGGACAAAGTTGG | |
| | AAATAAAA | |
| 148 | QVQLVQSGAEVKKPGASVKVSCKASGYVFT | AM4B6 VH |
| | DYYMNWVRQAPGQSLEWMGDINPNNAETL | |
| | YNHKFKGRVTVTVDKSISTAYMELSRLRSD | |
| | DTAVYYCVKWGDGPFAYWGQGTLVTVSS | |
| 149 | DIQMTQSPSSLSASVGDRVTITCKASQNVGA | AM4B6 VL |
| | AVAWYQQKPGKAPKLLIYSVSDRYTGVPSR | |
| | FSGSGSGTDFTLTISSLQPEDIATYYCQQYSN | |
| | YPTFGQGTKLEIK | |
| 150 | QVQLVQSGAEVKKPGASVKLSCKASGYIFT | 23F11 heavy chain |
| | TYWMHWVKQRPGQGLEWIGMIQPNSGGTK | (HC) |
| | YNEKFKKKATLTVDKSISTAYMELSRLTSDD | |
| | TAVYYCARGAGTVDYFDYWGQGSTLTVSS | |
| | ASTKGPSVFPLAPSSKSTSGGTAALGCLVKD | |
| | YFPEPVTVSWNSGALTSGVHTFPAVLQSSGL | |
| | YSLSSVVTVPSSSLGTQTYICNVNHKPSNTK | |
| | VDKKVEPKSCDKTHTCPPCPAPELLGGPSVF | |
| | LFPPKPKDTLMISRTPEVTCVVVDVSHEDPE | |
| | VKFNWYVDGVEVHNAKTKPREEQYASTYR | |
| | VVSVLTVLHQDWLNGKEYKCKVSNKALPA | |
| | PIEKTISKAKGQPREPQVYTLPPSRDELTKNQ | |
| | VSLTCLVKGFYPSDIAVEWESNGQPENNYK | |
| | TTPPVLDSDGSFFLYSKLTVDKSRWQQGNV | |
| | FSCSVMHEALHNHYTQKSLSLSPGK | |
| 151 | DIVLTQSPASLAVSVGQRATITCRASESVDIY | 23F11 light chain |
| | GNSFMHWYQQKPGQPPKLLIYRASNLESGIP | (LC) |
| | ARFSGSGSRTDFTLTINPVEAQDTATYYCQQ | |
| | STEDPYTFGGGTKLEIKRTVAAPSVFIFPPSD | |
| | EQLKSGTASVVCLLNNFYPREAKVQWKVD | |
| | NALQSGNSQESVTEQDSKDSTYSLSSTLTLS | |
| | KADYEKHKVYACEVTHQGLSSPVTKSFNRG | |
| | EC | |

1-5. (canceled)

6. An isolated antibody against human gremlin1 (hGREM1) or an antigen-binding fragment thereof, comprising a heavy chain variable (VH) region and/or a light chain variable (VL) region, wherein:
a) the heavy chain variable region comprises a HCDR1 comprising the sequence of SEQ ID NO: 1, a HCDR2 comprising the sequence of SEQ ID NO: 2, and a HCDR3 comprising the sequence of SEQ ID NO: 3; and the light chain variable region comprises a LCDR1 comprising the sequence of SEQ ID NO: 4, a LCDR2 comprising the sequence of SEQ ID NO: 5, and a LCDR3 comprising the sequence of SEQ ID NO: 6;
b) the heavy chain variable region comprises a HCDR1 comprising the sequence of SEQ ID NO: 11, a HCDR2 comprising the sequence of SEQ ID NO: 12, and a HCDR3 comprising the sequence of SEQ ID NO: 13; and the light chain variable region comprises a LCDR1 comprising the sequence of SEQ ID NO: 14, a LCDR2 comprising the sequence of SEQ ID NO: 15, and a LCDR3 comprising the sequence of SEQ ID NO: 16;
c) the heavy chain variable region comprises a HCDR1 comprising the sequence of SEQ ID NO: 21, a HCDR2 comprising the sequence of SEQ ID NO: 22, and a HCDR3 comprising the sequence of SEQ ID NO: 23; and the light chain variable region comprises a LCDR1 comprising the sequence of SEQ ID NO: 24, a LCDR2 comprising the sequence of SEQ ID NO: 25, and a LCDR3 comprising the sequence of SEQ ID NO: 26;
d) the heavy chain variable region comprises a HCDR1 comprising the sequence of SEQ ID NO: 31, a HCDR2 comprising the sequence of SEQ ID NO: 32, and a HCDR3 comprising the sequence of SEQ ID NO: 33; and the light chain variable region comprises a LCDR1 comprising the sequence of SEQ ID NO: 34, a LCDR2 comprising the sequence of SEQ ID NO: 35, and a LCDR3 comprising the sequence of SEQ ID NO: 36;
e) the heavy chain variable region comprises a HCDR1 comprising the sequence of SEQ ID NO: 114, a HCDR2 comprising the sequence of SEQ ID NO: 115, and a HCDR3 comprising the sequence of SEQ ID NO: 116; and
the light chain variable region comprises a LCDR1 comprising the sequence of SEQ ID NO: 117, a LCDR2 comprising the sequence of SEQ ID NO: 35, and a LCDR3 comprising the sequence of SEQ ID NO: 118;
f) the heavy chain variable region comprises a HCDR1 comprising the sequence of SEQ ID NO: 119, a HCDR2 comprising the sequence of SEQ ID NO: 115, and a HCDR3 comprising the sequence of SEQ ID NO: 120; and
the light chain variable region comprises a LCDR1 comprising the sequence of SEQ ID NO: 121, a LCDR2 comprising the sequence of SEQ ID NO: 35, and a LCDR3 comprising the sequence of SEQ ID NO: 118;
g) the heavy chain variable region comprises a HCDR1 comprising the sequence of SEQ ID NO: 119, a HCDR2 comprising the sequence of SEQ ID NO: 115, and a HCDR3 comprising the sequence of SEQ ID NO: 120; and
the light chain variable region comprises a LCDR1 comprising the sequence of SEQ ID NO: 122, a LCDR2 comprising the sequence of SEQ ID NO: 35, and a LCDR3 comprising the sequence of SEQ ID NO: 118;
h) the heavy chain variable region comprises a HCDR1 comprising the sequence of SEQ ID NO: 123, a HCDR2 comprising the sequence of SEQ ID NO: 115, and a HCDR3 comprising the sequence of SEQ ID NO: 124; and
the light chain variable region comprises a LCDR1 comprising the sequence of SEQ ID NO: 125, a LCDR2 comprising the sequence of SEQ ID NO: 35, and a LCDR3 comprising the sequence of SEQ ID NO: 118;
i) the heavy chain variable region comprises a HCDR1 comprising the sequence of SEQ ID NO: 114, a HCDR2 comprising the sequence of SEQ ID NO: 115, and a HCDR3 comprising the sequence of SEQ ID NO: 116; and
the light chain variable region comprises a LCDR1 comprising the sequence of SEQ ID NO: 117, a LCDR2 comprising the sequence of SEQ ID NO: 35, and a LCDR3 comprising the sequence of SEQ ID NO: 118;
j) the heavy chain variable region comprises a HCDR1 comprising the sequence of SEQ ID NO: 119, a HCDR2 comprising the sequence of SEQ ID NO: 115, and a HCDR3 comprising the sequence of SEQ ID NO: 120; and the light chain variable region comprises a LCDR1 comprising the sequence of SEQ ID NO: 121, a LCDR2 comprising the sequence of SEQ ID NO: 35, and a LCDR3 comprising the sequence of SEQ ID NO: 118; or
k) the heavy chain variable region comprises a HCDR1 comprising the sequence of SEQ ID NO: 119, a HCDR2 comprising the sequence of SEQ ID NO: 115, and a HCDR3 comprising the sequence of SEQ ID NO: 120; and the light chain variable region comprises a LCDR1 comprising the sequence of SEQ ID NO: 122, a LCDR2 comprising the sequence of SEQ ID NO: 35, and a LCDR3 comprising the sequence of SEQ ID NO: 118.

7-8. (canceled)

9. The antibody or an antigen-binding fragment thereof of claim 6, comprising:
a) a heavy chain variable region comprising the sequence of SEQ ID NO: 7 and a light chain variable region comprising the sequence of SEQ ID NO: 8; or
b) a heavy chain variable region comprising a sequence of SEQ ID NO: 17 and a light chain variable region comprising a sequence of SEQ ID NO: 18; or
c) a heavy chain variable region comprising a sequence of SEQ ID NO: 27 and a light chain variable region comprising a sequence of SEQ ID NO: 28; or
d) a heavy chain variable region comprising a sequence of SEQ ID NO: 37 and a light chain variable region comprising a sequence of SEQ ID NO: 38; or
e) a heavy chain variable region comprising a sequence of SEQ ID NO: 126 and a light chain variable region comprising a sequence of SEQ ID NO: 127; or
f) a heavy chain variable region comprising a sequence of SEQ ID NO: 128 and a light chain variable region comprising a sequence of SEQ ID NO: 129; or
g) a heavy chain variable region comprising a sequence of SEQ ID NO: 128 and a light chain variable region comprising a sequence of SEQ ID NO: 130; or
h) a heavy chain variable region comprising a sequence selected from the group consisting of SEQ ID NO: 41, SEQ ID NO: 43 and SEQ ID NO: 45, and a light chain variable region comprising a sequence selected from the group consisting of SEQ ID NO: 47 and SEQ ID NO: 49; or
i) a pair of heavy chain variable region and light chain variable region sequences selected from the group consisting of: SEQ ID NOs: 41/47, 41/49, 43/47, 43/49, 45/47, and 45/49; or
j) a heavy chain variable region comprising a sequence selected from the group consisting of SEQ ID NO: 51, SEQ ID NO: 53, SEQ ID NO: 55 and SEQ ID NO: 57, and a light chain variable region comprising a sequence selected from the group consisting of SEQ ID NO: 59 and SEQ ID NO: 61; or
k) a pair of heavy chain variable region and light chain variable region sequences selected from the group consisting of: SEQ ID NOs: 51/59, 51/61, 53/59, 53/61, 55/59, 55/61, 57/59, and 57/61; or
l) a heavy chain variable region comprising a sequence selected from the group consisting of SEQ ID NO: 131, SEQ ID NO: 132, SEQ ID NO: 133 and SEQ ID NO: 134, and a light chain variable region comprising a sequence selected from the group consisting of SEQ ID NO: 135, SEQ ID NO: 136 and SEQ ID NO: 137; or
m) a pair of heavy chain variable region and light chain variable region sequences selected from the group consisting of: SEQ ID NOs: 131/135, 131/136, 131/137, 132/135, 132/136, 132/137, 133/135, 133/136, 133/137, 134/135, 134/136, and 134/137.

10-11. (canceled)

12. The antibody or antigen-binding fragment thereof of claim 6, further comprising an immunoglobulin constant region, or further comprising a constant region of-human IgG.

13. (canceled)

14. The antibody or an antigen-binding fragment thereof of claim 6, which is humanized or chimeric.

15. The antibody or antigen-binding fragment thereof of claim 6, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody.

16. The antibody or antigen-binding fragment thereof of claim 6, which is bispecific, capable of specifically binding to a first and a second epitope of gremlin, or capable of specifically binding to both hGREM1 and a second antigen.

17-18. (canceled)

19. The antibody or antigen-binding fragment thereof of claim 16, wherein the second antigen is selected fro the group consisting of: PD-1, PD-L1, PD-L2, CTLA-4, TIM-3, LAG3, A2AR, CD160, 2B4, TGF β, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16, CD83, prostate specific antigen (PSA_, CA-125, gangliosides G(D2), G(M2) and (D3), CD20, CD52, C1D33, Ep-CAM, CEA, bombesin-like peptides, HER2/neu, epidermal growth factor receptor (EGFR), erbB2, erbB3/HER3, erbB4, CD44v6, Ki-67, cancer-associated mucin, VEGF, VEGFRs (e.g., VEGFR-1, VEGFR-2, VEGFR-3), estrogen receptors, Lewis-Y antigen, TGFfβ1, IGF-1 receptor, EGFα, c-Kit receptor, transferrin receptor, Claudin 18.2, GPC-3, Nectin-4, ROR1, methothelin, PCMA, MAGE-1, MAGE-3, BAGE, GAGE-1, GAGE-2, p15, BCR-ABL, E2APRL, 1H4-RET, IGH-1GK, MYL-RAR, IL-2R, CO17-1 A, TROP2 and LIV-1.

20-24. (canceled)

25. The antibody or antigen-binding fragment thereof of claim 6 linked to one or more conjugate moieties

26-27. (canceled)

28. A pharmaceutical composition or kit comprising the antibody or antigen-binding fragment thereof of claim 6, and a pharmaceutically acceptable carrier.

29. (canceled)

30. An isolated polynucleotide encoding the antibody or an antigen-binding fragment thereof of claim 6.

31. A vector comprising the isolated polynucleotide of claim 30.

32. A host cell comprising the vector of claim 31.

33. A method of expressing the antibody or antigen-binding fragment thereof of claim 6, comprising culturing a host cell comprising a vector comprising an isolated polynucleotide encoding the antibody or an antigen-binding fragment thereof of claim 6 under the condition at which the vector is expressed.

34. A method of treating a GREM1-related disease or condition in a subject, or a method of inhibiting FGFR1 activation in a subject in need thereof, or a method of treating a disease or a condition associated with FGFR1 activation mediated by GREM1, comprising administering to the subject a therapeutically effective amount of the antibody or antigen-binding fragment thereof of claim 6.

35. The method of claim 34, wherein the GREM1-related disease or condition is selected from the group consisting of cancer, fibrotic disease, angiogenesis, glaucoma or retinal disease, kidney disease, pulmonary arterial hypertension, or osteoarthritis (OA), or the GREM1-related disease or condition is associated with increased level of GREM1 selected from the group consisting of scleroderma, idiopathic lung fibrosis, diabetic nephropathy, IgAN, lupus nephritis, Alport syndrome, glioma, head and neck cancer, prostate cancer, lung cancer, gastric cancer, pancreatic cancer, esophageal cancer, bladder cancer, breast cancer and colorectal cancer.

36. The method of claim 35, wherein the cancer is a GREM1-expressing cancer, optionally is PD-L1-expressing or is not a PD-L1-expressing cancer, and further optionally is resistant or refractory to the treatment with a PD-1/PD-L1 axis inhibitor.

37. The method of claim 34, wherein the subject is identified as having a GREM1-expressing cancer cell, or having GREM1 expression in cancer microenvironment.

38. (canceled)

39. The method of claim 35, wherein the cancer is prostate cancer, gastric-esophageal cancer, lung cancer (e.g., non-small cell lung cancer), liver cancer, pancreatic cancer, breast cancer, bronchial cancer, bone cancer, liver and bile duct cancer, ovarian cancer, testicle cancer, kidney cancer, bladder cancer, head and neck cancer, spine cancer, brain cancer, cervix cancer, uterine cancer, endometrial cancer, colon cancer, colorectal cancer, rectal cancer, anal cancer, gastrointestinal cancer, skin cancer, pituitary cancer, stomach cancer, vagina cancer, thyroid cancer, glioblastoma, astrocytoma, melanoma, myelodysplastic syndrome, sarcoma, teratoma, glioma, adenocarcinoma, leukemia (such as Acute lymphocytic leukemia (ALL), Acute myeloid leukemia (AML), Chronic lymphocytic leukemia (CLL), Chronic myeloid leukemia (CML)), lymphoma (such as Hodgkin's lymphoma, or Non-Hodgkin's lymphoma (e.g. Waldenstrom macroglobulinemia (WM))), or myeloma (such as multiple myeloma (MM)).

40-41. (canceled)

42. The method of claim 35, wherein the breast cancer is triple negative breast cancer.

43. The method of claim 35, wherein the cancer is esophageal cancer, optionally is resistant or refractory to the treatment with a PD-1/PD-L1 axis inhibitor, and further optionally is resistant or refractory to the treatment with an anti-PD-1 antibody (e.g. nivolumab).

44. The method of claim 35, wherein the fibrotic disease is a fibrotic disease in lungs, liver, kidney, eyes, skin, heart, gut or muscle.

45. The method of claim 34, wherein the subject is human.

46. (canceled)

47. The method of claim 34, further comprising administering a therapeutically effective amount of a second therapeutic agent, wherein the second therapeutic agent comprises an anti-cancer therapy, optionally the anti-cancer therapy is selected from a chemotherapeutic agent (e.g. cisplatin), radiation therapy, an immunotherapy agent (e.g. an immune checkpoint modulator, for example, a PD-1/PD-L1 axis inhibitor, TGF-beta inhibitor), anti-angiogenesis agent (e.g. antagonist of a VEGFR such as VEGFR-1, VEGFR-2, and VEGFR-3), a targeted therapy agent, a cellular therapy agent, a gene therapy agent, a hormonal therapy agent, cytokines, palliative care, surgery for the treatment of cancer (e.g., tumorectomy), one or more anti-emetics, treatments for complications arising from chemotherapy, a diet supplement for cancer patients (e.g. indole-3-carbinol), an agent that modulates tumor microenvironment (e.g. a bifunctional molecule comprising PD-L1 binding moiety and extracellular domain of TGF-beta receptor) or anti-fibrotic therapy (e.g., BMP7 treatment, ACE inhibitor (or ARB), anti-MASP2 antibody, endothelin receptor antagonist, NRF2 inhibitor steroid, CTLA4-IgG or TNF inhibitor).

48. (canceled)

49. The method of claim 47, wherein the anti-cancer therapy comprises an androgen axis inhibitor; and androgen synthesis inhibitor; a PARP inhibitor; or a combination thereof for treating prostate cancer, or comprises the group consisting of Abiraterone Acetate, Apalutamide, Bicalutamide, Cabazitaxel, Casodex (Bicalutamide), Darolutamide, Degarelix, Docetaxel, Eligard (Leuprolide Acetate), Enzalutamide, Erleada (Apalutamide), Firmagon (Degarelix), Flutamide, Goserelin Acetate, Jevtana (Cabazitaxel), Leuprolide Acetate, Lupron (Leuprolide Acetate), Lupron Depot (Leuprolide Acetate), Lynpanza (Olaparib), Mitoxantrone Hydrochloride, Nilandron (Nilutamide), Nilutamide, Nubeqa (Darolutamide), Olaparib, Provenge (Sipuleucel-T), Radium 223 Dichloride, Rubraca (Rucaparib Camsylate), Rucaparib Camsylate, Sipuleucel-T, Taxotere (Docetaxel), Xofigo (Radium 223 Dichloride), Xtandi (Enzalutamide), Zoladex (Goserelin Acetate) and Zytiga (Abiraterone Acetate) for treating prostate cancer.

50-51. (canceled)

52. The method of claim 49, wherein the androgen axis inhibitor is degarelix, bicalutamide, flutamide, nilutamide, apalutamide, darolutamide, enzalutamide, or abiraterone.

53-54. (canceled)

55. A method of detecting presence or amount of gremlin in a sample, comprising contacting the sample with the antibody or antigen-binding fragment thereof of claim 6, and determining the presence or the amount of gremlin in the sample.

56-58. (canceled)

59. A method of treating a disease that can benefit from increasing BMP7 activity or reducing gremlin-mediated inhibition on BMP7 activity or increasing efficacy of BMP7 treatment in a subject in need thereof, comprising administering to the subject a therapeutically effective amount of the antibody or antigen-binding fragment thereof of claim 6.

60-63. (canceled)
